(19) United States
(12) Patent Application Publication
(10) Pub. No.: US 2022/0370453 A1
(43) Pub. Date: Nov. 24, 2022

(54) BICYCLIC HETEROCYCLIC COMPOUNDS AS INHIBITORS OF BCDIN3D ACTIVITY

(71) Applicant: Storm Therapeutics Ltd, Cambridge (GB)

(72) Inventors: Wesley Peter Blackaby, Cambridge (GB); Elizabeth Jane Thomas, Cambridge (GB); David James Hardick, Cambridge (GB); Lisa Marie Frost, Oxfordshire (GB); Frederick Arthur Brookfield, Oxfordshire (GB); Boris Aillard, Oxfordshire (GB)

(21) Appl. No.: 17/620,782

(22) PCT Filed: Jun. 19, 2020
(86) PCT No.: PCT/GB20/51500

§ 371 Date: Dec. 20, 2021

(30) Foreign Application Priority Data
Jun. 20, 2019 (GB) 1908885.5

(51) Int. Cl. A61K 31/519; C07D 487/04; A61K 31/573; A61P 37/06

(52) U.S. Cl. CPC A61K 31/519; C07D 487/04; A61K 31/573; A61P 37/06

(57) ABSTRACT

The present invention relates to compounds that function as inhibitors and/or degraders of BCDIN3D activity. The present invention also relates to processes for the preparation of these compounds, to pharmaceutical compositions comprising them, and to their use in the treatment of proliferative disorders, such as cancer, as well as other diseases or conditions in which BCDIN3D activity is implicated.

### FIELD OF THE INVENTION

The present invention relates to certain compounds that function as inhibitors and/or degraders of BCDIN3D activity. The present invention also relates to processes for the preparation of these compounds, to pharmaceutical compositions comprising them, and to their use in the treatment of proliferative disorders, such as cancer, as well as other diseases or conditions in which BCDIN3D activity is implicated.

### BACKGROUND OF THE INVENTION

The Bicoid Interacting Domain containing protein, BCDIN3D, is an RNA methyl transferase which is conserved from worms to humans (Xhemalce et al 2012). BCDIN3D has been described to both mono-methylate and di-methylate the 5′-monophosphate of specific RNAs including histidinyl tRNA in HEK293T cells (Martinez et al 2017) and precursor micro RNAs (premiRNAs), such as the tumour suppressor miR145 (Sachdeva et al 2009, Shi et al 2007, Spizzo et al 2009), in MCF-7 breast cancer cells (Xhemalce et al 2012, Park et al 2011). Di-methylation of the 5′-monophosphate of the pre-miRNA negatively regulates the subsequent processing by Dicer in vitro (Xhemalce et al 2012, Park et al 2011), and results in the downregulated expression of the mature miRNA form. BCDIN3D has been identified in Genome-wide association studies (GWAS) as one of the major risk loci for obesity and is implicated in the development of type 2 diabetes (Ng et 2010; Takeuchi et al, 2011). BCDIN3D mRNA is overexpressed in human breast cancer cells, and the elevated expression of BCDIN3D is related to poor prognosis in breast cancer (Liu et al 2007, Yao et al 2016).

The shRNA-mediated depletion of BCDIN3D results in the suppression of the tumorigenic phenotype of MDA-MB-231 breast cancer cells. Thus, BCDIN3D has been proposed to promote the cellular invasion of breast cancer cells, by downregulating the expression of tumour suppressor miRNAs through the di-methylation of the 5′-monophosphate of the corresponding pre-miRNAs (Xhemalce et al 2012).

In addition, BCDIN3D mono-methylates the 5′monophosphate of histidinyl tRNA (tRNAHis) (Martinez et al 2017) and appears to be the primary target of BCDIN3D in HEK293T cells. Martinez et al, showed that cytoplasmic tRHAHis tightly binds to BCDIN3D in cells, and the 5′-monophosphate of cytoplasmic tRNAHis is monomethylated by BCDIN3D in vitro and in cells. In HEK293T, BCDIN3D monomethylates tRNAHis more efficiently than pre-miR145 by over two orders of magnitude in vitro, and never di-methylates the 5′ monophosphates of tRNAHis and pre-miR145. BCDIN3D recognizes the unique structures within cytoplasmic tRNAHis: the additional guanosine residue at position −1 (G−1) and the eight-nucleotide acceptor helix with the G−1-A73 miss pair, which are exceptional features among cytoplasmic tRNA species. The mono-methylation of the 5′-phosphate of cytoplasmic tRNAHis protects it from degradation in vitro. Therefore, BCDIN3D can also act as a cytoplasmic tRNAHis specific 5′-methylphosphate capping enzyme and may play a role in the tumorigenic phenotype of breast cancer cells.

MicroRNAs (miRNAs) are short single-stranded RNA molecules (18-24 nucleotides) that post transcriptionally interfere with gene expression in a variety of eukaryotes (Ghildiyal and Zamore, 2009). miRNAs target the RNA interference effector complex (RISC) to specific messenger RNAs (mRNAs) through partial base-paring to sequences predominantly found in the 3′ untranslated region (3′UTR). This interaction results in decreased translation of the proteins they encode and/or in the degradation of the mRNAs themselves (Fabian et al., 2010). To date, over 1,000 human miRNAs have been identified, and each of them potentially regulates many mRNAs (Friedman et al., 2009; Kozomara and Griffiths-Jones, 2011). Consequently, miRNAs have been involved in numerous cellular processes, including development, differentiation, proliferation, apoptosis, the stress response, and viral defence (Fabian et al., 2010). Importantly, altered expression of miRNAs is a common trait of cancers (Farazi et al., 2011). Indeed, deciphering regulation of miRNA expression may be important not only for diagnostic, but also for therapeutic purposes (Kasinski and Slack, 2011). Due to the way that miRNAs are generated, their expression can be regulated at different levels. The first level is transcriptional, as miRNAs are synthesized from larger transcripts by RNA polymerase II or RNA polymerase Ill complexes. The next level is post transcriptional, as these primary miRNA precursors (primiRNA) undergo at least three steps before the mature single stranded form. The pri-miRNA is first cleaved by Drosha to release a hairpin-loop-shaped RNA called pre-miRNA (Lee et al., 2003). The loop of this pre-miRNA is further cleaved by Dicer to generate a miRNA duplex (Chendrimada et al., 2005). The miRNA duplex is dissociated, and the passenger strand is discarded, whereas the guide strand is loaded onto the Argonaute (Ago) protein to form an active RISC complex (Yoda et al., 2010). Each of these steps is potentially subjected to regulation because the rate of transcription of a given pri-miRNA does not always correlate with the levels of its mature miRNA (Thomson et al., 2006). One crucial aspect of this process is related to the 5′ terminal end of these RNA molecules. Both RNase III enzymes, Drosha and Dicer, generate 5′ ends that contain a negatively charged monophosphate group. This 5′ monophosphate is bound by specific positively charged pockets in Dicer and Ago2, and these interactions are necessary for efficient and accurate processing as well as the stability of the mature RISC complex (Frank et al., 2010; Kawamata et al., 2011; Park et al., 2011).

The BCDIN3D 5-methylation activity of BCDIN3D results in a complete loss of the negative charge, resulting in reduced processing by Dicer in vitro. When BCDIN3D is depleted in cells, the association of Dicer with the product of pre-miR-145 processing increases yielding increased levels of mature miR-145. This may not be solely limited to miR-145 but may be responsible for the regulation of many other miRNAs. BCDIN3D depletion in breast cancer cells abolishes their tumorigenic phenotypes and thus has potential as a therapeutic target in cancer.

### REFERENCES

Xhemalce, B, Robson, S. C. and Kouzarides, T. (2012) Human RNA methyltransferase BCDIN3D regulates microRNA processing. Cell, 151, 278-288.
Martinez A, Yamashita S, Nagaike T, Sakaguchi Y, Suzuki T, Tomita K. (2017) Human BCDIN3D monomethylates cytoplasmic histidine transfer RNA. Nucleic Acids Res., 45, 5423-5436.
Liu, R., Wang, X., Chen, G. Y., Dalerba, P., Gurney, A., Hoey, T., Sherlock, G., Lewicki, J., Shedden, K. and Clarke, M. F. (2007) The prognostic role of a gene signature from tumorigenic breast-cancer cells. N. Engl. J. Med., 356, 217-226.
Yao, L., Chi, Y., Hu, X., Li, S., Qiao, F., Wu, J. and Shao, Z. M. (2016) Elevated expression of RNA methyltransferase BCDIN3D predicts poor prognosis in breast cancer. Oncotarget, 7, 53895-53902.
Sachdeva, M., Zhu, S., Wu, F., Wu, H., Walia, V. and Kumar, S. (2009) p53 represses c-Myc through induction of the tumor suppressor miR-145. Proc. Natl. Acad. Sci. U.S.A., 106, 3207-3212.
Shi, B., Sepp-Lorenzino, L., Prisco, M., Linsley, P., deAngelis, T. and Baserga, R. (2007) Micro RNA 145 targets the insulin receptor substrate-1 and inhibits the growth of colon cancer cells. J. Biol. Chem., 282, 32582-32590.
Spizzo, R., Nicoloso, M. S., Lupini, L., Lu, Y., Fogarty, J., Rossi, S., Zagatti, B., Fabbri, M., Veronese, A., Liu, X. et al. (2009) miR-145 participates with TP53 in a death-promoting regulatory loop and targets estrogen receptor-[alpha] in human breast cancer cells. Cell Death Differ., 17, 246-254.
Park, J. E., Heo, I., Tian, Y., Simanshu, D. K., Chang, H., Jee, D., Patel, D. J. and Kim, V. N. (2011) Dicer recognizes the 5′end of RNA for efficient and accurate processing. Nature, 475, 201-205.
Ghildiyal, M., and Zamore, P. D. (2009). Small silencing RNAs: an expanding universe. Nat. Rev. Genet. 10, 94-108.
Fabian, M. R., Sonenberg, N., and Filipowicz, W. (2010). Regulation of mRNA translation and stability by microRNAs. Annu. Rev. Biochem. 79, 351-379.
Friedman, R. C., Farh, K. K., Burge, C. B., and Bartel, D. P. (2009). Most mammalian mRNAs are conserved targets of microRNAs. Genome Res. 19, 92-105.
Kozomara, A., and Griffiths-Jones, S. (2011). miRBase: integrating microRNA annotation and deep-sequencing data. Nucleic Acids Res. 39(Database issue), D152-D157.
Farazi, T. A., Spitzer, J. I., Morozov, P., and Tuschl, T. (2011). miRNAs in human cancer. J. Pathol. 223, 102-115.
Kasinski, A. L., and Slack, F. J. (2011). Epigenetics and genetics. MicroRNAs en route to the clinic: progress in validating and targeting microRNAs for cancer therapy. Nat. Rev. Cancer 11, 849-864.
Lee, Y., Ahn, C., Han, J., Choi, H., Kim, J., Yim, J., Lee, J., Provost, P., Radmark, O., Kim, S., and Kim, V. N. (2003). The nuclear RNase III Drosha initiates microRNA processing. Nature 425, 415-419.
Chendrimada, T. P., Gregory, R. I., Kumaraswamy, E., Norman, J., Cooch, N., Nishikura, K., and Shiekhattar, R. (2005). TRBP recruits the Dicer complex to Ago2 for microRNA processing and gene silencing. Nature 436, 740-744.
Thomson, J. M., Newman, M., Parker, J. S., Morin-Kensicki, E. M., Wright, T., and Hammond, S. M. (2006). Extensive post-transcriptional regulation of microRNAs and its implications for cancer. Genes Dev. 20, 2202-2207.
Park, J. E., Heo, I., Tian, Y., Simanshu, D. K., Chang, H., Jee, D., Patel, D. J., and Kim, V. N. (2011). Dicer recognizes the 5′-end of RNA for efficient and accurate processing. Nature 475, 201-205.
Frank, F., Sonenberg, N., and Nagar, B. (2010). Structural basis for 5′-nucleotide base-specific recognition of guide RNA by human AGO2. Nature 465, 818-822.
Yoda, M., Kawamata, T., Paroo, Z., Ye, X., Iwaskai, S., Liu, Q., and Tomari, Y. (2010). ATP-dependent human RISC assembly pathways. Nature Structural & Molecular Biology, 17, 17-23.
Kawamata, T., Yoda, M., and Tomari, Y. (2011). Multilayer checkpoints for microRNA authenticity during RISC assembly. EMBO Reports 2011, 12(9), 944-949.
Ng M C, Tam C H, So W Y, Ho J S, Chan A W, Lee H M, Wang Y, Lam V K, Chan J C, Ma R C. (2010). Implication of genetic variants near NEGR1, SEC16B, TMEM18, ETV5/DGKG, GNPDA2, LIN7C/BDNF, MTCH2, BCDIN3D/FAIM2, SH2B1, FTO, MC4R, and KCTD15 with obesity and type 2 diabetes in 7705 Chinese. J Clin Endocrinol Metab. 2010 May; 95(5):2418-25.
Takeuchi F, Yamamoto K, Katsuya T, Nabika T, Sugiyama T, Fujioka A, Isono M, Ohnaka K, Fujisawa T, Nakashima E, Ikegami H, Nakamura J, Yamori Y, Yamaguchi S, Kobayashi S, Ogihara T, Takayanagi R, Kato N. (2011). Association of genetic variants for susceptibility to obesity with type 2 diabetes in Japanese individuals. Diabetologia. 2011 June; 54(6):1350-9

### SUMMARY OF THE INVENTION

In one aspect, the present invention provides a compound as defined herein, or a pharmaceutically acceptable salt thereof.

In another aspect, the present invention provides a pharmaceutical composition as defined herein which comprises a compound as defined herein, or a pharmaceutically acceptable salt thereof, and one or more pharmaceutically acceptable excipients.

In another aspect, the present invention provides a compound as defined herein, or a pharmaceutically acceptable salt thereof, or a pharmaceutical composition as defined herein, for use in therapy.

In another aspect, the present invention provides a compound as defined herein, or a pharmaceutically acceptable salt thereof, or a pharmaceutical composition as defined herein, for use in the treatment of a proliferative condition.

In another aspect, the present invention provides a compound as defined herein, or a pharmaceutically acceptable salt thereof, or a pharmaceutical composition as defined herein, for use in the treatment of cancer. In a particular embodiment, the cancer is a human cancer.

In another aspect, the present invention provides a compound as defined herein, or a pharmaceutically acceptable salt thereof, or a pharmaceutical composition as defined herein, for use in the treatment of a metabolic disease.

In another aspect, the present invention provides a compound as defined herein, or a pharmaceutically acceptable salt thereof, or a pharmaceutical composition as defined herein, for use in the inhibition and/or degradation of BCDIN3D activity.

In another aspect, the present invention provides the use of a compound as defined herein, or a pharmaceutically acceptable salt thereof, in the manufacture of a medicament for the treatment of a proliferative condition.

In another aspect, the present invention provides the use of a compound as defined herein, or a pharmaceutically acceptable salt thereof, in the manufacture of a medicament for the treatment of cancer. In a particular embodiment, the medicament is for use in the treatment of human cancers.

In another aspect, the present invention provides the use of a compound as defined herein, or a pharmaceutically acceptable salt thereof, in the manufacture of a medicament for the treatment of a metabolic disease.

In another aspect, the present invention provides the use of a compound as defined herein, or a pharmaceutically acceptable salt thereof, in the manufacture of a medicament for the inhibition and/or degradation of BCDIN3D activity.

In another aspect, the present invention provides a method of inhibiting and/or degrading BCDIN3D activity in vitro or in vivo, said method comprising contacting a cell with an effective amount of a compound as defined herein, or a pharmaceutically acceptable salt thereof, or a pharmaceutical composition as defined herein.

In another aspect, the present invention provides a method of inhibiting and/or degrading cell proliferation in vitro or in vivo, said method comprising contacting a cell with an effective amount of a compound as defined herein, or a pharmaceutically acceptable salt, or a pharmaceutical composition as defined herein.

In another aspect, the present invention provides a method of inhibiting and/or degrading metastasis in vitro or in vivo, said method comprising contacting a cell with an effective amount of a compound as defined herein, or a pharmaceutically acceptable salt, or a pharmaceutical composition as defined herein.

In another aspect, the present invention provides a method of treating a proliferative disorder, said method comprising administering to a subject in need thereof a therapeutically effective amount of a compound as defined herein, or a pharmaceutically acceptable salt, or a pharmaceutical composition as defined herein.

In another aspect, the present invention provides a method of treating cancer, said method comprising administering to a subject in need thereof a therapeutically effective amount of a compound as defined herein, or a pharmaceutically acceptable salt, or a pharmaceutical composition as defined herein.

In another aspect, the present invention provides a method of treating a metabolic disease, said method comprising administering to a subject in need thereof a therapeutically effective amount of a compound as defined herein, or a pharmaceutically acceptable salt, or a pharmaceutical composition as defined herein.

In one aspect, the present invention provides a combination comprising a compound as defined herein, or a pharmaceutically acceptable salt thereof, with one or more additional therapeutic agents.

The present invention further provides a method of synthesising a compound, or a pharmaceutically acceptable salt, as defined herein.

In another aspect, the present invention provides a compound as defined herein, or a pharmaceutically acceptable salt, obtainable by, or obtained by, or directly obtained by a method of synthesis as defined herein.

In another aspect, the present invention provides novel intermediates as defined herein which are suitable for use in any one of the synthetic methods as set out herein.

Preferred, suitable, and optional features of any one particular aspect of the present invention are also preferred, suitable, and optional features of any other aspect.

### BRIEF DESCRIPTION OF THE DRAWINGS

For a better understanding of the invention, and to show how embodiments of the same are put into effect, reference is now made, by way of example, to the following figure, in which:

FIG. 1: Western Blot results from BCDIN3D degradation assay

FIG. 2 shows qRT-PCR analysis of BCDIN3D expression in primary human CD4+ and CD8+ T-cells. The y-axis shows relative expression to TBP (TATA-box binding protein).

FIG. 3 shows the effect of BCDIN3D inhibitors (Compounds 4.53 and 4.30) on IFNγ production by activated CD4+ T-cells in vitro.

FIG. 4 shows the effect of BCDIN3D inhibitors (Compounds 4.53 and 4.30) in a delayed-type hypersensitivity in vivo model.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “C₁₋₆alkyl” includes C₁₋₄alkyl, C₁₋₃alkyl, propyl, isopropyl and t-butyl. A similar convention applies to other radicals, for example “phenyl(C₁₋₆alkyl)” includes phenyl(C₁₋₄alkyl), benzyl, 1-phenylethyl and 2-phenylethyl.

The term “(m-nC)” or “Cm-n”, or “(m-nC) group” used alone or as a prefix, refers to any group having m to n carbon atoms.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. For example, substitution of alkenyl groups by one or more alkyl, carbocyclyl, aryl, heterocyclyl, or heteroaryl groups is contemplated.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. For example, substitution of alkynyl groups by one or more alkyl, carbocyclyl, aryl, heterocyclyl, or heteroaryl groups is contemplated.

An “alkylene” group is an alkyl group that is positioned between and serves to connect two other chemical groups. Thus, “C₁₋₃alkylene” means a linear saturated divalent hydrocarbon radical of one to three carbon atoms or a branched saturated divalent hydrocarbon radical of three atoms, for example, methylene, ethylene, propylene, and the like.

The term “carbocyclic” means non-aromatic saturated or partially saturated monocyclic, fused, bridged, or spiro bicyclic hydrocarbon ring system(s). The term carbocyclic includes both monovalent species and divalent species. Monocyclic carbocyclic rings contain from about 3 to 12 (suitably from 3 to 7, most suitably from 5 to 6) ring atoms. Examples of monocyclic carbocyclic rings are cycloalkyls ring systems, for example cyclopropyl, cyclobutyl, cyclopentyl and cyclohexyl. Bicyclic carbocyclic ring systems contain from 4 to 17 member atoms, suitably 5 to 12 member atoms, in the ring. Bicyclic carbocyclic(s) rings may be fused, spiro, or bridged ring systems. Examples of bicyclic carbocyclic(s) rings include bicyclo[1.1.1]pentanyl, spiro[2.2]pentanyl, spiro[3.3]heptanyl and spiro[2.3]hexanyl.

The term “Cₘ₋ₙcarbocyclic” means a carbocylic ring containing from m to n carbon atoms, for example “C₃₋₁₀carbocyclic” means a carbocyclic ring containing from 3 to 10 carbon atoms.

The term “Cₘ₋ₙcycloalkyl” means a hydrocarbon ring containing from m to n carbon atoms, for example “C₃₋₆cycloalkyl” means a hydrocarbon ring containing from 3 to 6 carbon atoms, for example, cyclopropyl, cyclobutyl, cyclopentyl, cyclohexyl.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7, most suitably from 5 to 6) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, 2-oxaspiro[3,3]heptanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A heterocyclyl group may bear one or more oxo or thioxo groups. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include diazirinyl, furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to compounds of formula (I) shown below, or a pharmaceutically acceptable salt thereof:

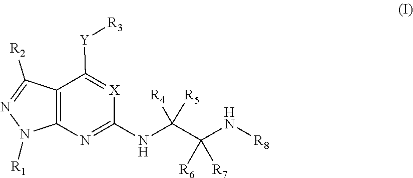

wherein:
R₁ is selected from methyl, ethyl, CH₂F, CHF₂, cyclopropyl, and CH₂CN wherein one or more hydrogen atoms in methyl, ethyl, CH₂F, CHF₂, cyclopropyl, and CH₂CN may be replaced by deuterium;
R₂ is selected from hydrogen, C₁₋₂ alkyl, C₁₋₂haloalkyl, cyano, amino, halo, trifluoromethyl, trifluoromethoxy, hydroxy, carboxy, carbamoyl, sulphamoyl, NRᵣRₛ, ORᵣ, C(O)Rᵣ, C(O)ORᵣ, OC(O)Rᵣ, C(O)N(Rᵣ)Rₛ, N(Rᵣ)C(O)Rₛ, S(O)ₐRᵣ (where a is 0, 1 or 2), SO₂N(Rᵣ)Rₛ, N(Rᵣ)SO₂Rₛ or (CH₂)_{b}NRᵣRₛ (where b is 1, 2 or 3), wherein Rᵣ and Rₛ are each independently selected from hydrogen or C₁₋₂alkyl;
R₃ is a group of the formula:

-L₁ₐ-L_{1b}-Q₁
wherein
L₁ₐ is absent or C₁₋₃alkylene, C₃₋₄cycloalkylene, optionally substituted by one or more substituents selected from C₁₋₂alkyl, halo, hydroxy, amino or oxo;
L_{1b} is absent or selected from O, S, SO, SO₂, N(Rₓ), C(O), C(O)O, OC(O), C(O)N(Rₓ), N(Rₓ)C(O), N(Rₓ)C(O)N(R_{y}), S(O)₂N(Rₓ), or N(Rₓ)SO₂, wherein Rₓ and R_{y} are each independently selected from hydrogen or C₁₋₂alkyl; and
Q₁ is hydrogen, cyano, C₁₋₆alkyl, C₃₋₁₀carbocyclic, C₂₋₃alkenyl, C₂₋₃alkynyl, aryl, heterocyclyl or heteroaryl; wherein one or more hydrogen atoms in C₁₋₆alkyl may be replaced by deuterium; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, halo, trifluoromethyl, trifluoromethoxy, amino, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, S(O)_{y}Rₜ (where y is 0, 1 or 2), SO₂N(Rₜ)Rᵤ, N(Rₜ)SO₂Rᵤ or (CH₂)_{z}NRₜRᵤ (where z is 1, 2 or 3); wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ as defined below; or
   Q₁ is optionally substituted by one or more groups of the formula:

-L_{1c}-L_{1d}-Z₁
wherein
L_{1c} is absent, C₁₋₃alkylene or O—C₁₋₃alkylene, wherein C₁₋₃alkylene or O—C₁₋₃alkylene are optionally substituted by C₁₋₂alkyl or oxo;
L_{1d} is absent or selected from O, C(O), C(O)O, OC(O), C(O)N(Rₘ), N(Rₘ)C(O), N(Rₘ)C(O)N(Rₙ), S(O)₂N(Rₘ), S(O)₂, or N(Rₘ)SO₂, wherein Rₘ and Rₙ are each independently selected from hydrogen or C₁₋₂alkyl; and
Z₁ is cyano, C₁₋₆alkyl, C₃₋₁₀carbocyclic, C₂₋₃alkenyl, C₂₋₃alkynyl, aryl, heterocyclyl or heteroaryl; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, C₂₋₄alkynyl, halo, trifluoromethyl, trifluoromethoxy, amino, cyano, hydroxy, carboxy, carbamoyl, sulphamoyl, NRᵥR_{w}, ORᵥ, C(O)Rᵥ, C(O)ORᵥ, OC(O)Rᵥ, C(O)N(Rᵥ)R_{w}, N(Rᵥ)C(O)R_{w}, S(O)ₓRᵥ (where x is 0, 1 or 2), SO₂N(Rᵥ)R_{w}, N(Rᵥ)SO₂R_{w} or (CH₂)ₛNRᵥR_{w} (where s is 1, 2 or 3), wherein Rᵥ and R_{w} are each independently selected from hydrogen or C₁₋₄alkyl;
   R₄ is hydrogen;
   R₅ is selected from hydrogen and C₁₋₄alkyl;
   R₆ is selected from hydrogen and C₁₋₄ alkyl;
   R₇ is selected from hydrogen, C₁₋₄alkyl, C₃₋₆cycloalkyl, heterocyclyl, heteroaryl and aryl, wherein C₁₋₄alkyl, C₃₋₆cycloalkyl, heterocyclyl, heteroaryl or aryl is optionally substituted with one or more substituents selected from C₁₋₄alkyl, C₂₋₃alkenyl, C₂₋₃alkynyl, halo, trifluoromethyl, trifluoromethoxy, amino, cyano, hydroxy, carboxy, carbamoyl, sulphamoyl, NRₚR_{q}, ORₚ, C(O)Rₚ, C(O)ORₚ, OC(O)Rₚ, C(O)N(Rₚ)R_{q}, N(Rₚ)C(O)R_{q}, S(O)ⱼRₚ (where j is 0, 1 or 2), SO₂N(Rₚ)R_{q}, N(Rₚ)SO₂R_{q} or (CH₂)ₖNRₚR_{q} (where k is 1, 2 or 3), wherein Rₚ and R_{q} are each independently selected from hydrogen or C₁₋₄alkyl;
   R₈ is selected from hydrogen or C₁₋₄alkyl;
   or R₆ and R₇ may be linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano;
   or R₅ and R₇ may be linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano;
   or R₇ and R₈ may be linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano;
   X is selected from N and CR₉;
   R₉ is selected from hydrogen, methyl, and fluoro;
   Y is selected from O and NR₁₀; and
   R₁₀ is selected from hydrogen and C₁₋₄alkyl;
   or R₃ and R₁₀ may be linked together to form a 4-, 5-, 6- or 7-membered heterocyclic ring system.

In an embodiment, the present invention relates to compounds of formula (I) shown below, or a pharmaceutically acceptable salt thereof:

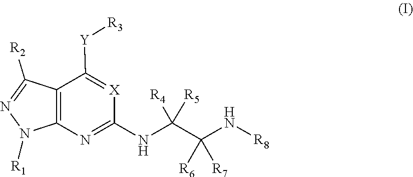

wherein:
R₁ is selected from methyl, ethyl, CH₂F, CHF₂, cyclopropyl, and CH₂CN wherein one or more hydrogen atoms in methyl, ethyl, CH₂F, CHF₂, cyclopropyl, and CH₂CN may be replaced by deuterium;
R₂ is selected from hydrogen, C₁₋₂ alkyl, C₁₋₂haloalkyl, cyano, amino, halo, trifluoromethyl, trifluoromethoxy, hydroxy, carboxy, carbamoyl, sulphamoyl, NRᵣRₛ, ORᵣ, C(O)Rᵣ, C(O)ORᵣ, OC(O)Rᵣ, C(O)N(Rᵣ)Rₛ, N(Rᵣ)C(O)Rₛ, S(O)ₐRᵣ (where a is 0, 1 or 2), SO₂N(Rᵣ)Rₛ, N(Rᵣ)SO₂Rₛ or (CH₂)_{b}NRᵣRₛ (where b is 1, 2 or 3), wherein Rᵣ and Rₛ are each independently selected from hydrogen or C₁₋₂alkyl;
R₃ is a group of the formula:

-L₁ₐ-L_{1b}-Q₁
wherein
L₁ₐ is absent or C₁₋₃alkylene, C₃₋₄cycloalkylene, optionally substituted by one or more substituents selected from C₁₋₂alkyl, halo, hydroxy, amino or oxo;
L_{1b} is absent or selected from O, S, SO, SO₂, N(Rₓ), C(O), C(O)O, OC(O), C(O)N(Rₓ), N(Rₓ)C(O), N(Rₓ)C(O)N(R_{y}), S(O)₂N(Rₓ), or N(Rₓ)SO₂, wherein Rₓ and R_{y} are each independently selected from hydrogen or C₁₋₂alkyl; and
Q₁ is hydrogen, cyano, C₁₋₆alkyl, C₃₋₁₀carbocyclic, C₂₋₃alkenyl, C₂₋₃alkynyl, aryl, heterocyclyl or heteroaryl; wherein one or more hydrogen atoms in C₁₋₆alkyl may be replaced by deuterium; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, halo, trifluoromethyl, trifluoromethoxy, amino, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, S(O)_{y}Rₜ (where y is 0, 1 or 2), SO₂N(Rₜ)Rᵤ, N(Rₜ)SO₂Rᵤ or (CH₂)_{z}NRₜRᵤ (where z is 1, 2 or 3); wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ as defined below; or
Q₁ is optionally substituted by one or more groups of the formula:

-L_{1c}-L_{1d}-Z₁
wherein
L_{1c} is absent or C₁₋₃alkylene optionally substituted by C₁₋₂alkyl or oxo;
L_{1d} is absent or selected from O, C(O), C(O)O, OC(O), C(O)N(Rₘ), N(Rₘ)C(O), N(Rₘ)C(O)N(Rₙ), S(O)₂N(Rₘ), S(O)₂, or N(Rₘ)SO₂, wherein Rₘ and Rₙ are each independently selected from hydrogen or C₁₋₂alkyl; and
Z₁ is cyano, C₁₋₆alkyl, C₃₋₁₀carbocyclic, C₂₋₃alkenyl, C₂₋₃alkynyl, aryl, heterocyclyl or heteroaryl; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, C₂₋₄alkynyl, halo, trifluoromethyl, trifluoromethoxy, amino, cyano, hydroxy, carboxy, carbamoyl, sulphamoyl, NRᵥR_{w}, ORᵥ, C(O)Rᵥ, C(O)ORᵥ, OC(O)Rᵥ, C(O)N(Rᵥ)R_{w}, N(Rᵥ)C(O)R_{w}, S(O)ₓRᵥ (where x is 0, 1 or 2), SO₂N(Rᵥ)R_{w}, N(Rᵥ)SO₂R_{w} or (CH₂)ₛNRᵥR_{w} (where s is 1, 2 or 3), wherein Rᵥ and R_{w} are each independently selected from hydrogen or C₁₋₄alkyl;
   R₄ is hydrogen;
   R₅ is selected from hydrogen and C₁₋₄alkyl;
   R₆ is selected from hydrogen and C₁₋₄ alkyl;
   R₇ is selected from hydrogen, C₁₋₄alkyl, C₃₋₆cycloalkyl, heterocyclyl, heteroaryl and aryl, wherein C₁₋₄alkyl, C₃₋₆cycloalkyl, heterocyclyl, heteroaryl or aryl is optionally substituted with one or more substituents selected from C₁₋₄alkyl, C₂₋₃alkenyl, C₂₋₃alkynyl, halo, trifluoromethyl, trifluoromethoxy, amino, cyano, hydroxy, carboxy, carbamoyl, sulphamoyl, NRₚR_{q}, ORₚ, C(O)Rₚ, C(O)ORₚ, OC(O)Rₚ, C(O)N(Rₚ)R_{q}, N(Rₚ)C(O)R_{q}, S(O)ⱼRₚ (where j is 0, 1 or 2), SO₂N(Rₚ)R_{q}, N(Rₚ)SO₂R_{q} or (CH₂)ₖNRₚR_{q} (where k is 1, 2 or 3), wherein Rₚ and R_{q} are each independently selected from hydrogen or C₁₋₄alkyl;
   R₈ is selected from hydrogen or C₁₋₄alkyl;
   or R₆ and R₇ may be linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano;
   or R₅ and R₇ may be linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano;
   or R₇ and R₈ may be linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano;
   X is selected from N and CR₉;
   R₉ is selected from hydrogen, methyl, and fluoro;
   Y is selected from O and NR₁₀; and
   R₁₀ is selected from hydrogen and C₁₋₄alkyl;
   or R₃ and R₁₀ may be linked together to form a 4-, 5-, 6- or 7-membered heterocyclic ring system.

Particular compounds of the invention include, for example, compounds of formula (I), or pharmaceutically acceptable salts thereof, wherein, unless otherwise stated, each of R₁, R₂, R₃, R₄, R₅, R₆, R₇, R₈, R₉, R₁₀, X, and Y and any associated substituent groups has any of the meanings defined hereinbefore or in any one of paragraphs (1) to (111) hereinafter:—
(1) R₁ is selected from methyl, ethyl, CH₂F, CHF₂, cyclopropyl, and CH₂CN wherein one or more hydrogen atoms in methyl, ethyl, CH₂F, CHF₂, cyclopropyl, and CH₂CN may be replaced by deuterium;
(2) R₁ is selected from methyl and ethyl wherein one or more hydrogen atoms in methyl may be replaced by deuterium;
(3) R₁ is selected from methyl, ethyl, and methyl wherein one or more hydrogen atoms in methyl is be replaced by deuterium;
(4) R₁ is selected from methyl, ethyl, and CD₃;
(5) R₁ is methyl;
(6) R₁ is ethyl;
(7) R₁ is CD₃;
(8) R₁ is cyclopropyl;
(9) R₂ is selected from hydrogen, C₁₋₂ alkyl, C₁₋₂haloalkyl, cyano, amino, halo, trifluoromethyl, trifluoromethoxy, hydroxy, carboxy, carbamoyl, sulphamoyl, NRᵣRₛ, ORᵣ, C(O)Rᵣ, C(O)ORᵣ, OC(O)Rᵣ, C(O)N(Rᵣ)Rₛ, N(Rᵣ)C(O)Rₛ, S(O)ₐRᵣ (where a is 0, 1 or 2), SO₂N(Rᵣ)Rₛ, N(Rᵣ)SO₂Rₛ or (CH₂)_{b}NRᵣRₛ (where b is 1, 2 or 3), wherein Rᵣ and Rₛ are each independently selected from hydrogen or C₁₋₂alkyl;
(10) R₂ is selected from hydrogen, methyl, C₁₋₂haloalkyl, cyano, amino, halo, trifluoromethyl, trifluoromethoxy, hydroxy, carboxy, carbamoyl, sulphamoyl, NRᵣRₛ, ORᵣ, C(O)Rᵣ, C(O)ORᵣ, OC(O)Rᵣ, C(O)N(Rᵣ)Rₛ, N(Rᵣ)C(O)Rₛ, S(O)ₐRᵣ (where a is 0, 1 or 2), SO₂N(Rᵣ)Rₛ, N(Rᵣ)SO₂Rₛ or (CH₂)_{b}NRᵣRₛ (where b is 1, 2 or 3), wherein Rᵣ and Rₛ are each independently selected from hydrogen or C₁₋₂alkyl;
(11) R₂ is selected from hydrogen and methyl;
(12) R₂ is hydrogen;
(13) R₂ is C₁₋₂ alkyl;
(14) R₂ is methyl;
(15) R₃ is a group of the formula:

-L₁ₐ-L_{1b}-Q₁
wherein
L₁ₐ is absent or C₁₋₃alkylene, C₃₋₄cycloalkylene, optionally substituted by one or more substituents selected from C₁₋₂alkyl, halo, hydroxy, amino or oxo;
L_{1b} is absent or selected from O, S, SO, SO₂, N(Rₓ), C(O), C(O)O, OC(O), C(O)N(Rₓ), N(Rₓ)C(O), N(Rₓ)C(O)N(R_{y}), S(O)₂N(Rₓ), or N(Rₓ)SO₂, wherein Rₓ and R_{y} are each independently selected from hydrogen or C₁₋₂alkyl; and
Q₁ is hydrogen, cyano, C₁₋₆alkyl, C₃₋₁₀carbocyclic, C₂₋₃alkenyl, C₂₋₃alkynyl, aryl, heterocyclyl or heteroaryl; wherein one or more hydrogen atoms in C₁₋₆alkyl may be replaced by deuterium; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, halo, trifluoromethyl, trifluoromethoxy, amino, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, S(O)_{y}Rₜ (where y is 0, 1 or 2), SO₂N(Rₜ)Rᵤ, N(Rₜ)SO₂Rᵤ or (CH₂)_{z}NRₜRᵤ (where z is 1, 2 or 3); wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ is defined below; or
Qi is optionally substituted by one or more groups of the formula:

-L_{1c}-L_{1d}-Z₁
wherein
L_{1c} is absent, C₁₋₃alkylene or O—C₁₋₃alkylene, wherein C₁₋₃alkylene or O—C₁₋₃alkylene are;
L_{1d} is absent or selected from O, C(O), C(O)O, OC(O), C(O)N(Rₘ), N(Rₘ)C(O), N(Rₘ)C(O)N(Rₙ), S(O)₂N(Rₘ), S(O)₂, or N(Rₘ)SO₂, wherein Rₘ and Rₙ are each independently selected from hydrogen or C₁₋₂alkyl; and
Z₁ is cyano, C₁₋₆alkyl, C₃₋₁₀carbocyclic, C₂₋₃alkenyl, C₂₋₃alkynyl, aryl, heterocyclyl or heteroaryl; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, C₂₋₄alkynyl, halo, trifluoromethyl, trifluoromethoxy, amino, cyano, hydroxy, carboxy, carbamoyl, sulphamoyl, NRᵥR_{w}, ORᵥ, C(O)Rᵥ, C(O)ORᵥ, OC(O)Rᵥ, C(O)N(Rᵥ)R_{w}, N(Rᵥ)C(O)R_{w}, S(O)ₓRᵥ (where x is 0, 1 or 2), SO₂N(Rᵥ)R_{w}, N(Rᵥ)SO₂R_{w} or (CH₂)ₛNRᵥR_{w} (where s is 1, 2 or 3), wherein Rᵥ and R_{w} are each independently selected from hydrogen or C₁₋₄alkyl;
(16) R₃ is a group of the formula:

-L₁ₐ-L_{1b}-Q₁
wherein
L₁ₐ is absent or C₁₋₃alkylene optionally substituted by one or more C₁₋₂alkyl substituents;
L_{1b} is absent or O; and
Q₁ is C₁₋₆alkyl, C₃₋₁₀carbocyclic, aryl, heterocyclyl or heteroaryl; wherein one or more hydrogen atoms in C₁₋₆alkyl may be replaced by deuterium; and wherein C₁₋₆alkyl, C₃₋₁₀carbocyclic, aryl, heterocyclyl or heteroaryl are optionally substituted by one or more substituents selected from C₁₋₄alkyl, halo, trifluoromethyl, trifluoromethoxy, cyano, NRₜRᵤ, or ORₜ; wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ as defined below; or
Q₁ is optionally substituted by one or more groups of the formula:

-L_{1c}-L_{1d}-Z₁
wherein
L_{1c} is absent, C₁₋₃alkylene or O—C₁₋₃alkylene;
L_{1d} is absent or selected from O, C(O), or S(O)₂; and
Z₁ is cyano, C₁₋₆alkyl, C₃₋₁₀carbocyclic, aryl, or heteroaryl; and wherein C₁₋₆alkyl, C₃₋₁₀carbocyclic, aryl, or heteroaryl are optionally substituted by one or more substituents selected from halo, C₂₋₄alkynyl, and ORᵥ wherein Rᵥ is selected from hydrogen or C₁₋₄alkyl;
(17) R₃ is a group of the formula:

-L₁ₐ-L_{1b}-Q₁
wherein
L₁ₐ is absent or C₁₋₃alkylene optionally substituted by one or more C₁₋₂alkyl substituents;
L_{1b} is absent or O; and
Q₁ is C₁₋₆alkyl, C₃₋₁₀carbocyclic, aryl, heterocyclyl or heteroaryl; wherein one or more hydrogen atoms in C₁₋₆alkyl may be replaced by deuterium; and wherein C₁₋₆alkyl, C₃₋₁₀carbocyclic, aryl, or heteroaryl are optionally substituted by one or more substituents selected from C₁₋₄alkyl, halo, trifluoromethyl, trifluoromethoxy, cyano, NRₜRᵤ, or ORₜ; wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ as defined below; or
Q₁ is optionally substituted by one or more groups of the formula:

-L_{1c}-L_{1d}-Z₁
wherein
L_{1c} is absent, C₁₋₃alkylene or O—C₁₋₃alkylene;
L_{1d} is absent or selected from O, C(O), or S(O)₂; and
Z₁ is cyano, C₁₋₆alkyl, C₃₋₁₀carbocyclic, aryl, or heteroaryl; and wherein C₁₋₆alkyl, aryl or heteroaryl are optionally substituted by one or more substituents selected from halo, C₂₋₄alkynyl and ORᵥ wherein Rᵥ is selected from hydrogen or C₁₋₄alkyl;
(17a) R₃ is a group of the formula:

-L₁ₐ-L_{1b}-Q₁
wherein
L₁ₐ is absent or C₁₋₃alkylene optionally substituted by one or more C₁₋₂alkyl substituents;
L_{1b} is absent or O; and
Q₁ is C₁₋₆alkyl, C₃₋₁₀carbocyclic or aryl; wherein one or more hydrogen atoms in C₁₋₆alkyl may be replaced by deuterium; and wherein C₁₋₆alkyl, C₃₋₁₀carbocyclic or aryl are optionally substituted by one or more substituents selected from C₁₋₄alkyl, halo, trifluoromethyl, trifluoromethoxy, cyano, NRₜRᵤ, or ORₜ; wherein Rₜ and Rᵤ are each independently selected from hydrogen or C₁₋₄alkyl wherein the C₁₋₄alkyl is optionally substituted with one or more halo substituents;
(18) R₃ is a group of the formula:

-L₁ₐ-L_{1b}-Q₁
wherein
L₁ₐ is absent, methylene or ethylene wherein methylene or ethylene are optionally substituted by one or more methyl substituents;
L_{1b} is absent or O; and
Q₁ is methyl, ethyl, propyl, i-propyl, butyl, t-butyl, C₃carbocyclic, C₄carbocyclic, C₅carbocyclic, C₆carbocyclic, C₇carbocyclic, phenyl, oxygen containing heterocyclyl, nitrogen containing heterocyclyl, pyrimidinyl, pyrazinyl, diazirinyl, pyridyl, benzo[1,3]dioxolyl, benzofuranyl, or benzimidazolyl; wherein one or more hydrogen atoms in methyl, ethyl, propyl, i-propyl, butyl or t-butyl may be replaced by deuterium; and wherein methyl, ethyl, propyl, i-propyl, butyl, t-butyl, C₃carbocyclic, C₄carbocyclic, C₅carbocyclic, C₆carbocyclic, C₇carbocyclic, phenyl, pyrimidinyl, pyrazinyl, diazirinyl, pyridyl, benzo[1,3]dioxolyl, benzofuranyl, or benzimidazolyl are optionally substituted by one or more substituents selected from methyl, ethyl, propyl, t-butyl, fluoro, chloro, bromo, trifluoromethyl, trifluoromethoxy, cyano, NRₜRᵤ, and ORₜ; wherein Rₜ and Rᵤ are each independently selected from hydrogen, methyl, ethyl, propyl or i-propyl wherein the methyl, ethyl, propyl or i-propyl is optionally substituted with one or more fluoro substituents or -L_{1c}-L_{1d}-Z₁ wherein L_{1c} is absent, L_{1d} is absent, and Z₁ is heteroaryl substituted with butynyl; or
Q₁ is optionally substituted by one or more groups of the formula:

-L_{1c}-L_{1d}-Z₁
wherein
L_{1c} is absent, methylene, or ethylene;
L_{1d} is absent or selected from O, C(O), or S(O)₂; and
Z₁ is cyano, methyl, ethyl, C₃carbocyclic, phenyl, or heteroaryl; and wherein methyl, ethyl, heteroaryl or phenyl are optionally substituted by one or more substituents selected from butynyl, fluoro, chloro and ORᵥ wherein Rᵥ is selected from hydrogen or methyl;
(19) R₃ is a group of the formula:

-L₁ₐ-L_{1b}-Q₁
wherein
L₁ₐ is absent, methylene or ethylene wherein methylene or ethylene are optionally substituted by one or more methyl substituents;
L_{1b} is absent or O; and
Q₁ is methyl, ethyl, propyl, i-propyl, butyl, t-butyl, phenyl, tetrahydrofuranyl, tetrahydropyranyl, piperidinyl, pyrimidinyl, pyrazinyl, diazirinyl, pyridyl, benzo[1,3]dioxolyl, cyclopropyl, cyclobutyl, cyclopentyl, cyclohexyl, 2-oxaspiro[3,3]heptanyl, bicyclo[1.1.1]pentanyl, spiro[2.2]pentanyl, spiro[3.3]heptanyl, spiro[2.3]hexanyl, benzofuranyl, or benzimidazolyl; wherein one or more hydrogen atoms in methyl, ethyl, propyl, i-propyl, butyl, or t-butyl may be replaced by deuterium; and wherein methyl, ethyl, propyl, i-propyl, butyl, t-butyl, phenyl, pyrimidinyl, pyrazinyl, diazirinyl, pyridyl, benzo[1,3]dioxolyl, cyclopropyl, cyclobutyl, cyclopentyl, cyclohexyl, bicyclo[1.1.1]pentanyl, spiro[3.3]heptanyl, benzofuranyl, or benzimidazolyl are optionally substituted by one or more substituents selected from methyl, ethyl, propyl, t-butyl, fluoro, chloro, bromo, trifluoromethyl, trifluoromethoxy, cyano, NRₜRᵤ, and ORₜ; wherein Rₜ and Rᵤ are each independently selected from hydrogen, methyl, ethyl or i-propyl wherein the methyl, ethyl or i-propyl is optionally substituted with one or more fluoro substituents or -L_{1c}-L_{1d}-Z₁ wherein L_{1c} is absent, L_{1d} is absent, and Z₁ is diazirinyl substituted with butynyl; or
Q₁ is optionally substituted by one or more groups of the formula:

-L_{1c}-L_{1d}-Z₁
wherein
L_{1c} is absent, methylene, or ethylene;
L_{1d} is absent or selected from O, C(O), or S(O)₂; and
Z₁ is cyano, methyl, ethyl, C₃carbocyclic, phenyl, or diazirinyl; and wherein methyl, ethyl, phenyl or diazirinyl are optionally substituted by one or more substituents selected from butynyl, fluoro, chloro and ORᵥ wherein Rᵥ is selected from hydrogen or methyl;
(20) R₃ is a group of the formula:

-L₁ₐ-L_{1b}-Q₁
wherein
L₁ₐ is absent, methylene or ethylene wherein methylene or ethylene are optionally substituted by one or more methyl substituents;
L_{1b} is absent or O; and
Q₁ is methyl, CD₃, ethyl, propyl, i-propyl, butyl, t-butyl, phenyl, tetrahydrofuranyl, tetrahydropyranyl, piperidinyl, pyrimidinyl, pyrazinyl, diazirinyl, pyridyl, benzo[1,3]dioxolyl, cyclopropyl, cyclobutyl, cyclopentyl, cyclohexyl, 2-oxaspiro[3,3]heptanyl, bicyclo[1.1.1]pentanyl, spiro[2.2]pentanyl, spiro[3.3]heptanyl, spiro[2.3]hexanyl, benzofuranyl, or benzimidazolyl; and wherein methyl, ethyl, propyl, i-propyl, t-butyl, phenyl, pyrimidinyl, pyrazinyl, pyridyl, cyclopropyl, cyclobutyl, cyclopentyl, cyclohexyl, bicyclo[1.1.1]pentanyl or spiro[3.3]heptanyl are optionally substituted by one or more substituents selected from methyl, ethyl, propyl, t-butyl, fluoro, chloro, bromo, trifluoromethyl, trifluoromethoxy, cyano, NRₜRᵤ, and ORₜ; wherein Rₜ and Rᵤ are each independently selected from hydrogen, methyl, ethyl or i-propyl wherein the methyl, ethyl or i-propyl is optionally substituted with one or more fluoro substituents or -L_{1c}-L_{1d}-Z₁ wherein L_{1c} is absent, L_{1d} is absent, and Z₁ is diazirinyl substituted with butynyl; or
Q₁ is optionally substituted by one or more groups of the formula:

-L_{1c}-L_{1d}-Z₁
wherein
L_{1c} is absent, methylene, or ethylene;
L_{1d} is absent or selected from O, C(O), or S(O)₂; and
Z₁ is cyano, methyl, ethyl, C₃carbocyclic, phenyl, or diazirinyl; and wherein methyl, ethyl, phenyl, or diazirinyl are optionally substituted by one or more substituents selected from butynyl, fluoro, chloro and ORᵥ wherein Rᵥ is selected from hydrogen or methyl;
(20a) R₃ is a group of the formula:

-L₁ₐ-L_{1b}-Q₁
wherein
L₁ₐ is absent, methylene or ethylene wherein methylene or ethylene are optionally substituted by one or more methyl substituents;
L_{1b} is absent; and
Q₁ is methyl, CD₃, ethyl, propyl, i-propyl, butyl, t-butyl, phenyl, cyclopropyl, cyclobutyl, cyclopentyl, cyclohexyl, bicyclo[1.1.1]pentanyl, spiro[2.2]pentanyl, spiro[3.3]heptanyl or spiro[2.3]hexanyl; and wherein methyl, ethyl, propyl, i-propyl, butyl, t-butyl, phenyl, cyclopropyl, cyclobutyl, cyclopentyl, cyclohexyl, bicyclo[1.1.1]pentanyl, spiro[2.2]pentanyl, spiro[3.3]heptanyl or spiro[2.3]hexanyl are optionally substituted by one or more substituents selected from methyl, ethyl, propyl, t-butyl, fluoro, chloro, bromo, trifluoromethyl, trifluoromethoxy, cyano, NRₜRᵤ, and ORₜ; wherein Rₜ and Rᵤ are each independently selected from hydrogen, methyl, ethyl or i-propyl wherein the methyl, ethyl or i-propyl is optionally substituted with one or more fluoro substituents or -L_{1c}-L_{1d}-Z₁ wherein L_{1c} is absent, L_{1d} is absent, and Z₁ is diazirinyl substituted with butynyl; or
Q₁ is optionally substituted by one or more groups of the formula:

-L_{1c}-L_{1d}-Z₁
wherein
L_{1c} is absent, methylene, or ethylene;
L_{1d} is absent or selected from O, C(O), or S(O)₂; and
Z₁ is cyano, methyl, ethyl, C₃carbocyclic, phenyl, or diazirinyl; and wherein methyl, ethyl, phenyl, or diazirinyl are optionally substituted by one or more substituents selected from butynyl, fluoro, chloro and ORᵥ wherein Rᵥ is selected from hydrogen or methyl;
(21) R₃ is selected from methyl, ethyl, isopropyl, t-butyl, cyclopropyl, cyclobutyl, phenyl,

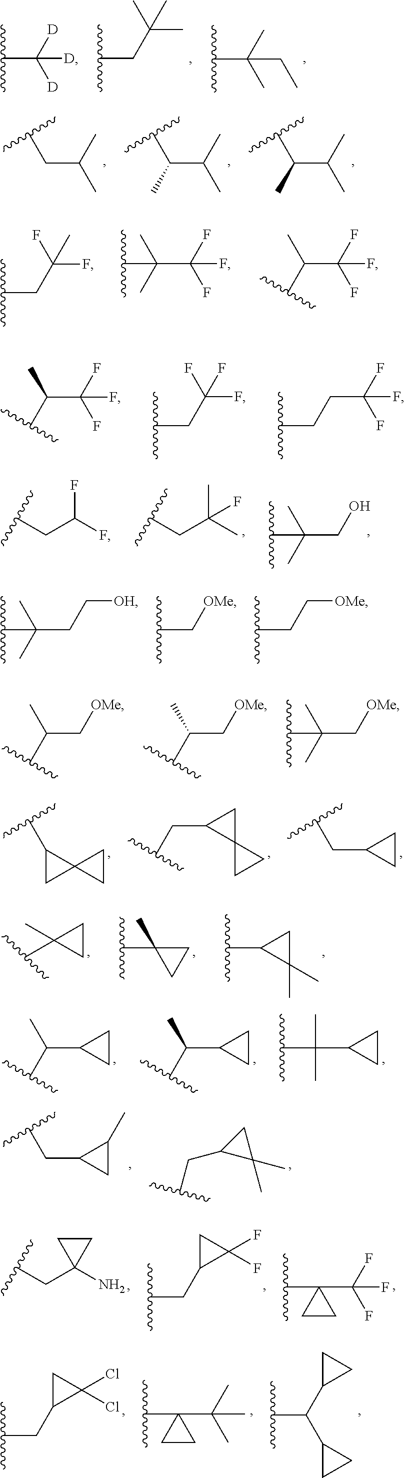

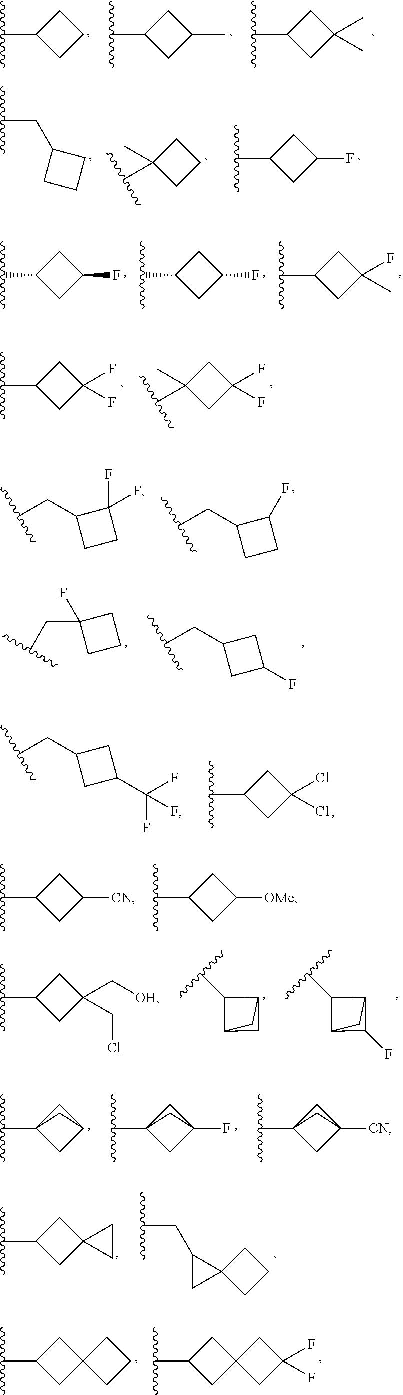

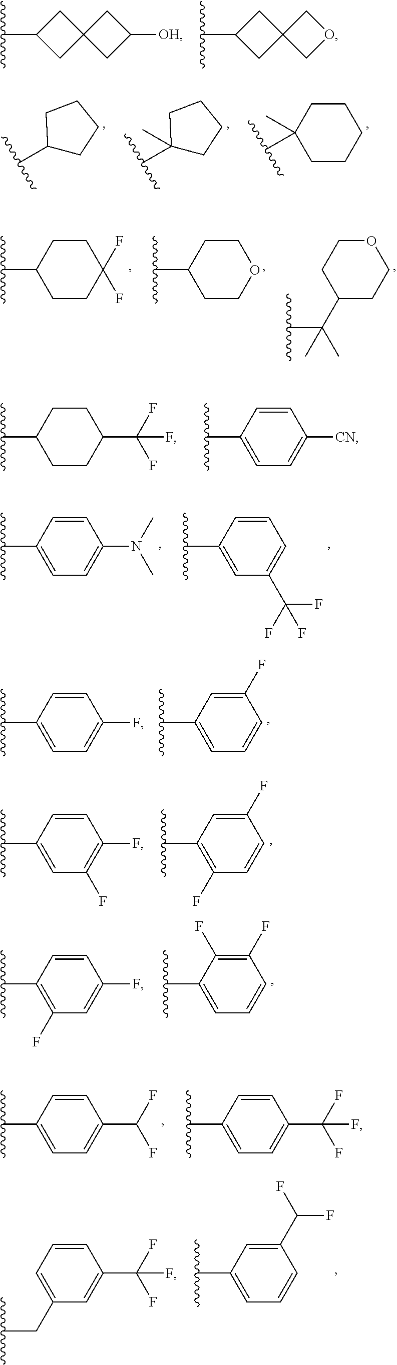

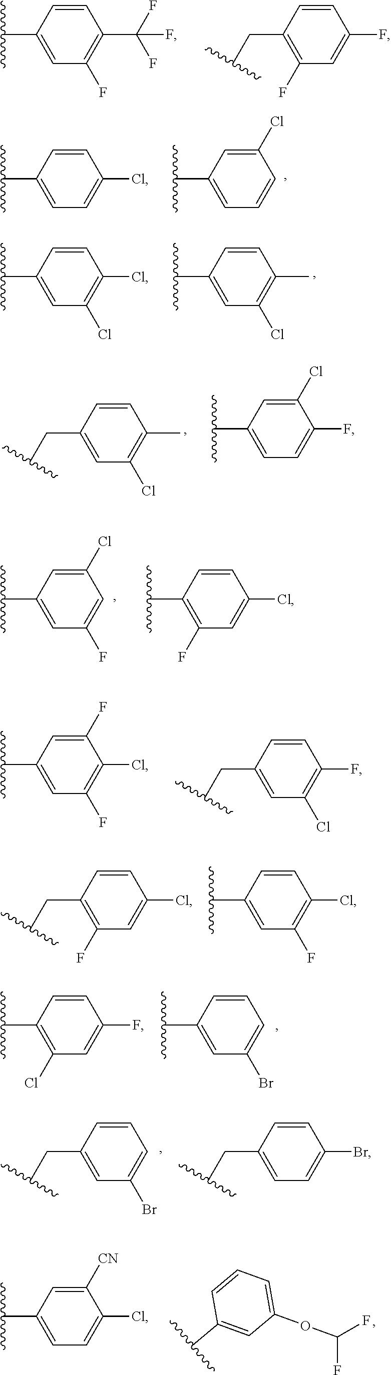

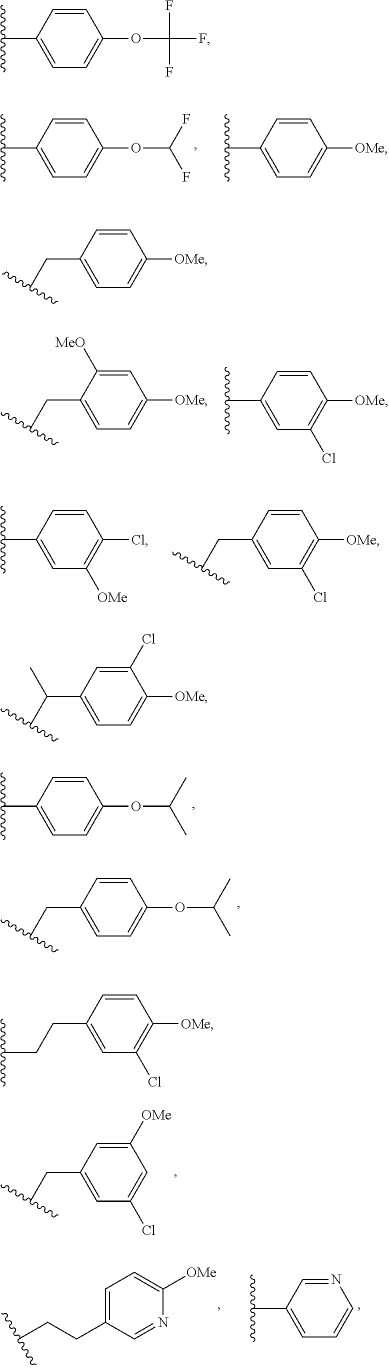

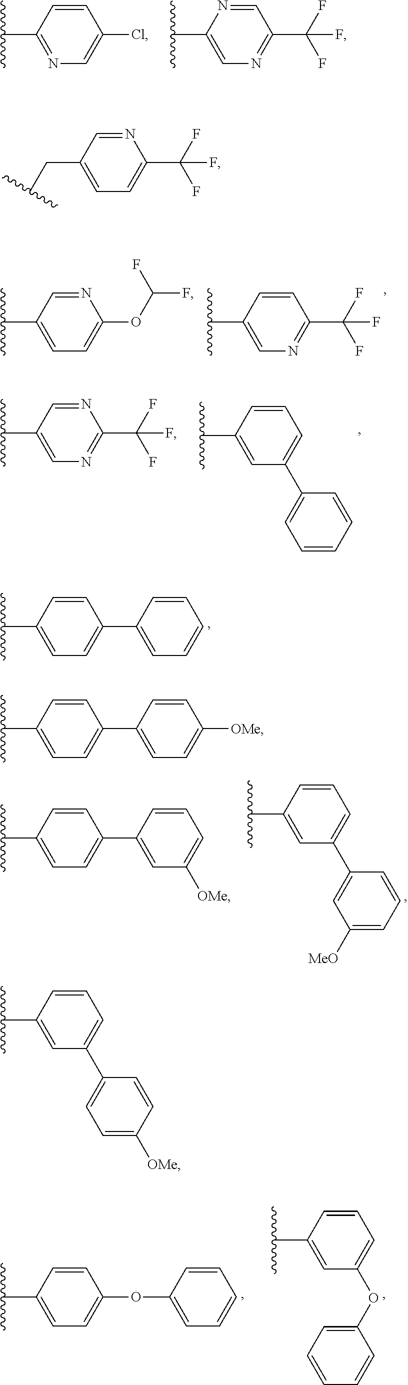

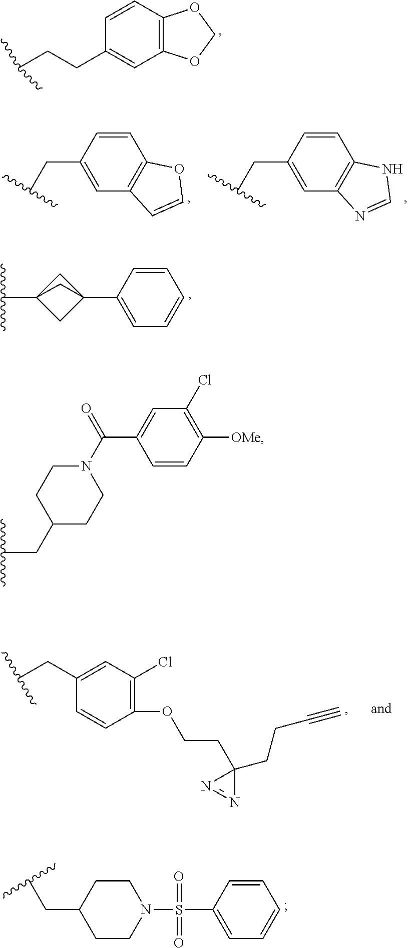

(21a) R₃ is selected from methyl, ethyl, isopropyl, t-butyl, cyclopropyl, cyclobutyl, phenyl,

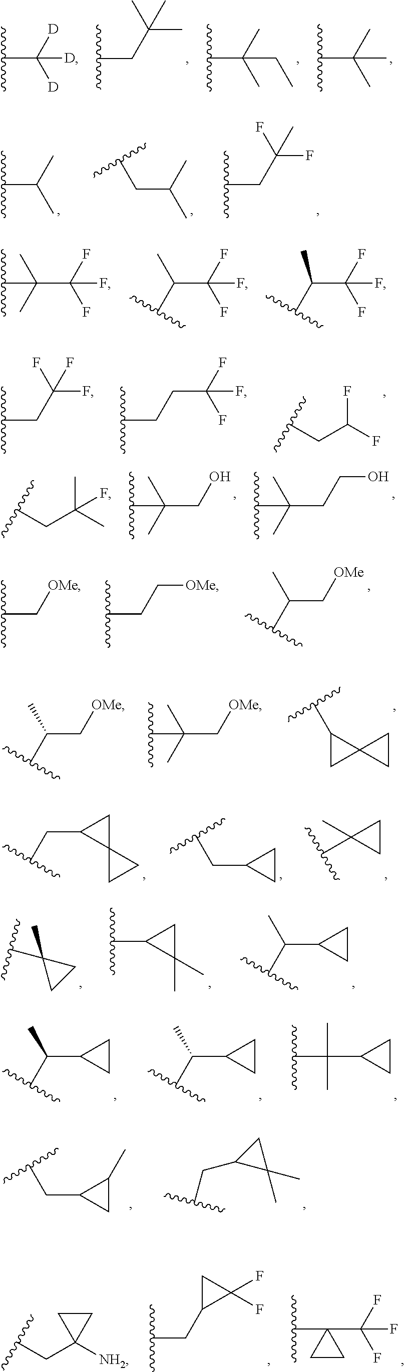

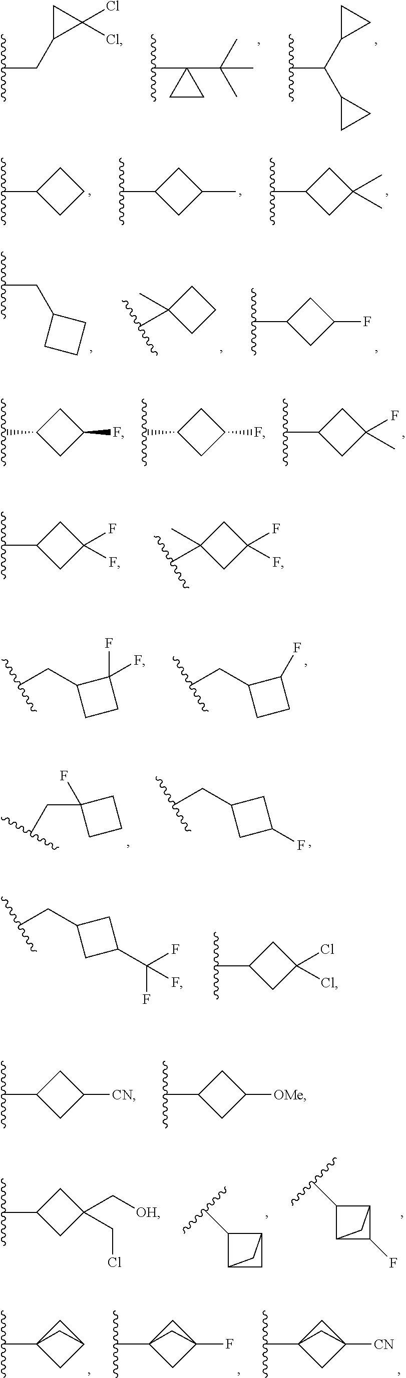

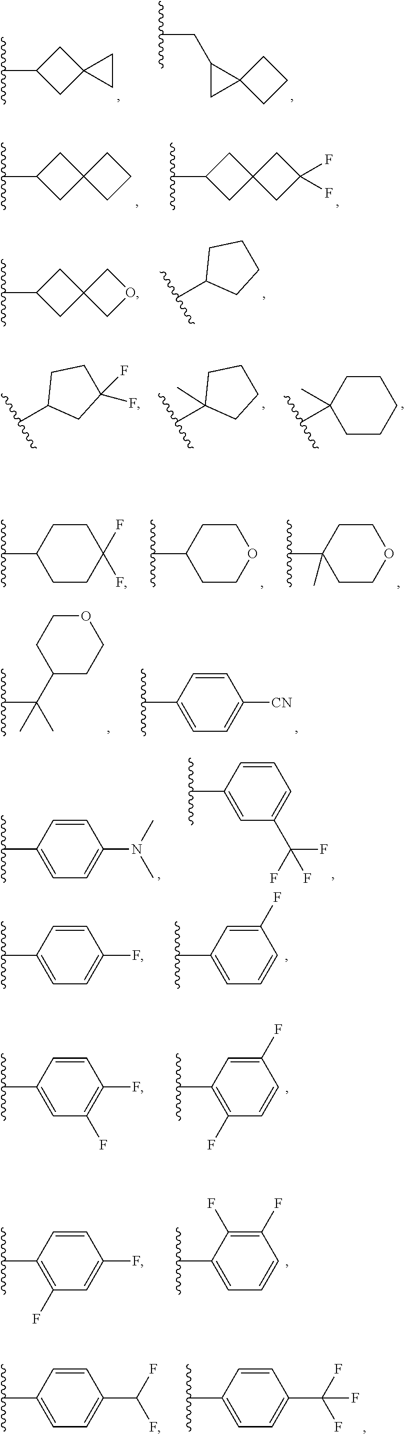

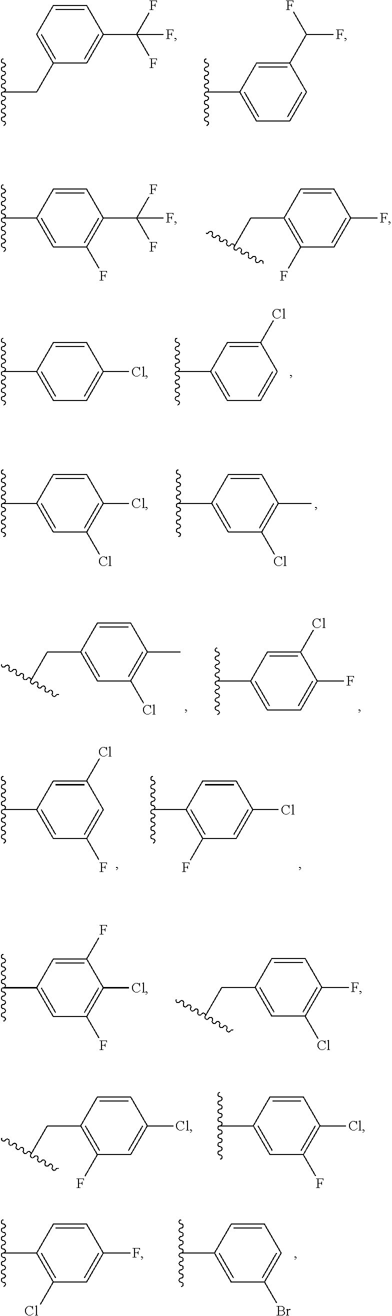

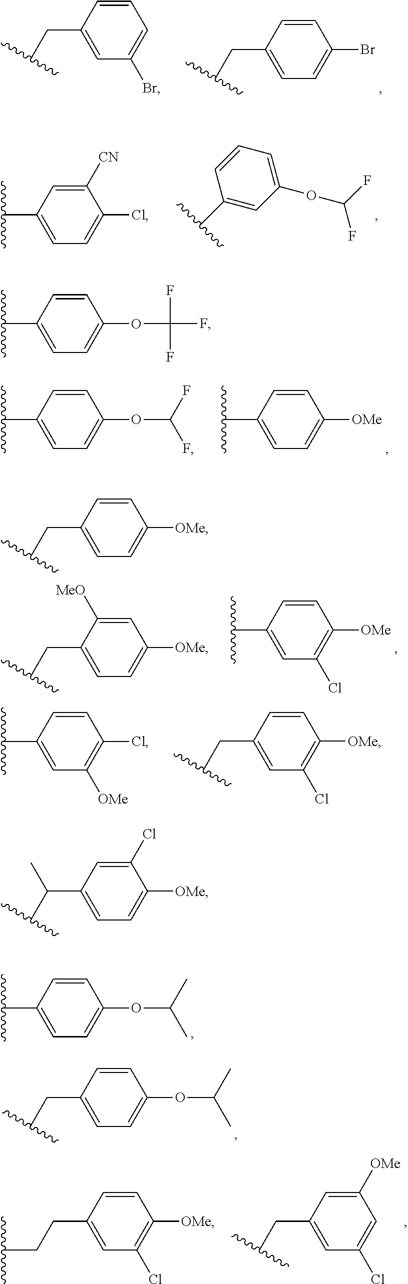

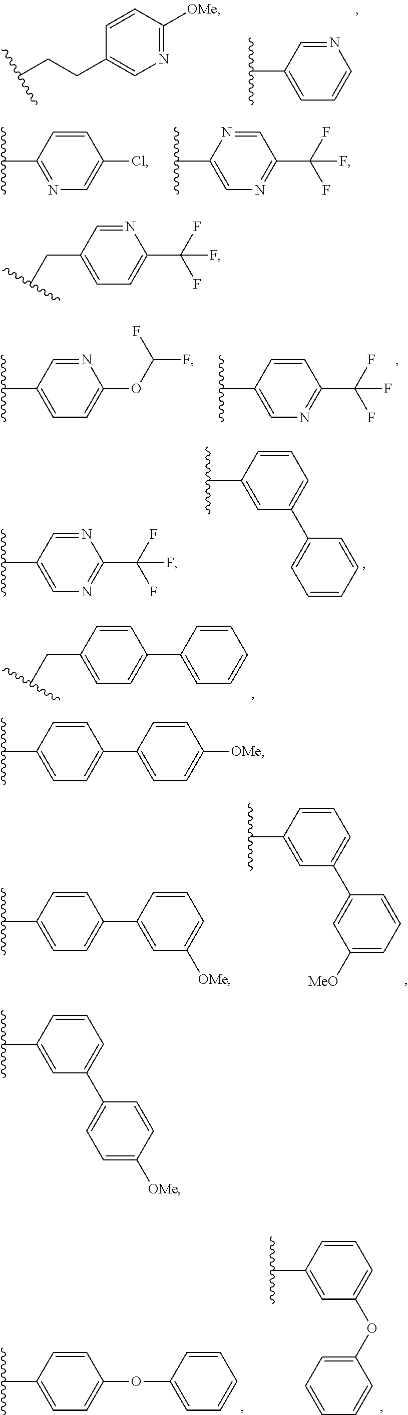

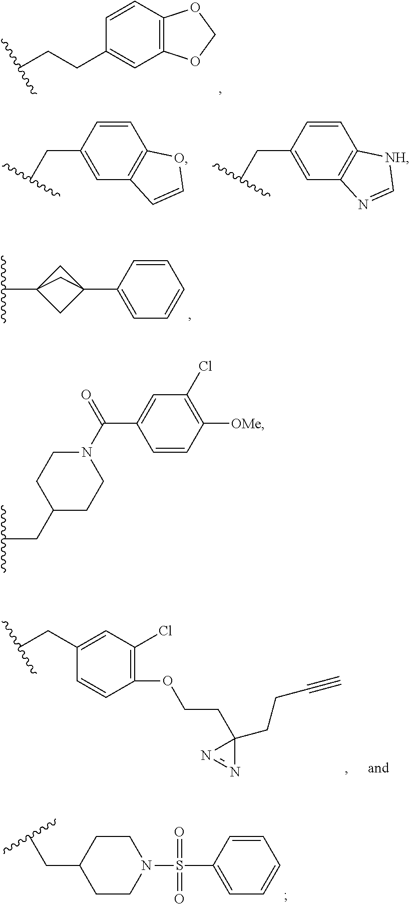

(22) R₄ is hydrogen;
(23) R₅ is selected from hydrogen and C₁₋₄alkyl;
(24) R₅ is selected from hydrogen, methyl, and ethyl;
(25) R₅ is hydrogen;
(26) R₅ is methyl;
(27) R₅ is ethyl;
(28) R₄ is hydrogen and R₅ is hydrogen;
(29) R₅ is C₁₋₄ alkyl, R₆ is hydrogen and R₇ is hydrogen;
(30) R₆ is selected from hydrogen and C₁₋₄ alkyl;
(31) R₆ is selected from hydrogen and methyl;
(32) R₆ is hydrogen;
(33) R₆ is methyl;
(34) R₄ is hydrogen, R₅ is hydrogen and R₆ is hydrogen;
(35) R₇ is selected from hydrogen, C₁₋₄alkyl, C₃₋₆cycloalkyl, heterocyclyl, heteroaryl and aryl, wherein C₁₋₄alkyl, C₃₋₆cycloalkyl, heterocyclyl, heteroaryl or aryl is optionally substituted with one or more substituents selected from C₁₋₄alkyl, C₂₋₃alkenyl, C₂₋₃alkynyl, halo, trifluoromethyl, trifluoromethoxy, amino, cyano, hydroxy, carboxy, carbamoyl, sulphamoyl, NRₚR_{q}, ORₚ, C(O)Rₚ, C(O)ORₚ, OC(O)Rₚ, C(O)N(Rₚ)R_{q}, N(Rₚ)C(O)R_{q}, S(O)ⱼRₚ (where j is 0, 1 or 2), SO₂N(Rₚ)R_{q}, N(Rₚ)SO₂R_{q} or (CH₂)ₖNRₚR_{q} (where k is 1, 2 or 3), wherein Rₚ and R_{q} are each independently selected from hydrogen or C₁₋₄alkyl;
(36) R₇ is selected from hydrogen, C₁₋₄alkyl, C₃₋₆cycloalkyl, heteroaryl and aryl, wherein C₁₋₄alkyl, C₃₋₆cycloalkyl, heteroaryl or aryl is optionally substituted with one or more substituents selected from C₁₋₄alkyl, C₂₋₃alkynyl, halo, cyano, and ORₚ, wherein Rₚ is independently selected from hydrogen or C₁₋₄alkyl;
(37) R₇ is selected from hydrogen, C₁₋₄alkyl, C₃₋₆cycloalkyl, heteroaryl and aryl, wherein C₁₋₄alkyl, C₃₋₆cycloalkyl, heteroaryl or aryl is optionally substituted with one or more substituents selected from methyl, ethynyl, fluoro, chloro, bromo, cyano and ORₚ, wherein Rₚ is independently selected from hydrogen or methyl;
(38) R₇ is selected from hydrogen, methyl, ethyl, cyclopropyl, cyclohexyl, pyridyl, and phenyl, wherein methyl, cyclopropyl, cyclohexyl, pyridyl or phenyl is optionally substituted with one or more substituents selected from methyl, ethynyl, fluoro, chloro, bromo, cyano and ORₚ, wherein Rₚ is independently selected from hydrogen or methyl;
(39) R₇ is selected from hydrogen, methyl optionally substituted with fluoro or OMe, ethyl, —CH₂F, —CH₂OMe, cyclopropyl, cyclohexyl, phenyl,

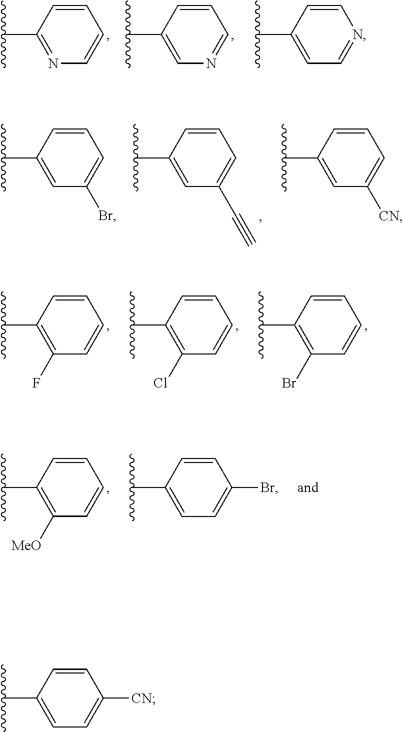

(39a) R₇ is selected from hydrogen, methyl optionally substituted with fluoro or OMe, ethyl, —CH₂F, —CH₂OMe, cyclopropyl, cyclohexyl, phenyl,

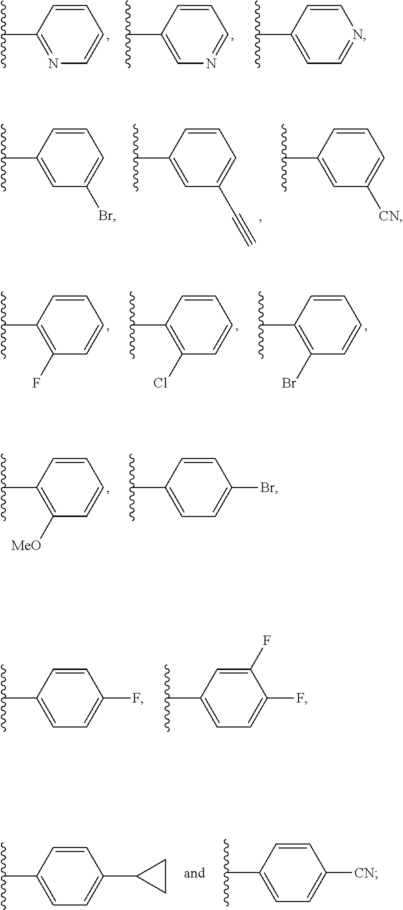

(40) R₇ is hydrogen;
(41) R₇ is methyl optionally substituted with fluoro, or OMe;
(42) R₇ is methyl;
(43) R₇ is ethyl;
(44) R₇ is —CH₂F;
(45) R₇ is —CH₂OMe;
(46) R₇ is cyclopropyl;
(47) R₇ is cyclohexyl;
(48) R₇ is pyridyl;
(49) R₇ is

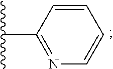 [SMILES: *c1ccccn1]

(50) R₇ is

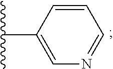 [SMILES: *c1cccnc1]

(51) R₇ is

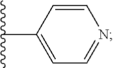 [SMILES: *c1ccncc1]

(52) R₇ is phenyl, wherein phenyl is optionally substituted with one or more substituents selected from methyl, ethynyl, fluoro, chloro, bromo, cyano and OMe;
(53) R₇ is selected from phenyl,

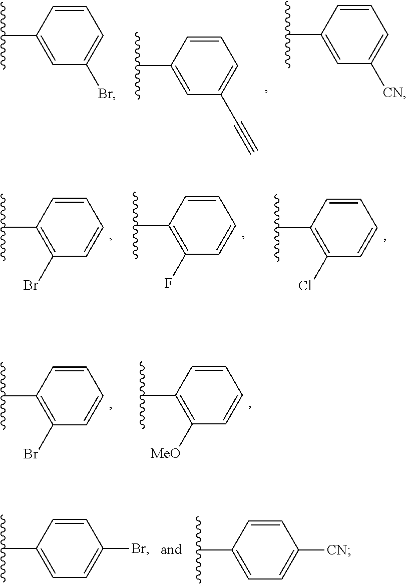

(54) R₅ is hydrogen and R₇ is selected from C₁₋₄alkyl, C₃₋₆cycloalkyl, heterocyclyl, heteroaryl and aryl, wherein C₁₋₄alkyl, C₃₋₆cycloalkyl, heterocyclyl, heteroaryl or aryl is optionally substituted with one or more substituents selected from C₁₋₄alkyl, C₂₋₃alkenyl, C₂₋₃alkynyl, halo, trifluoromethyl, trifluoromethoxy, amino, cyano, hydroxy, carboxy, carbamoyl, sulphamoyl, NRₚR_{q}, ORₚ, C(O)Rₚ, C(O)ORₚ, OC(O)Rₚ, C(O)N(Rₚ)R_{q}, N(Rₚ)C(O)R_{q}, S(O)ⱼRₚ (where j is 0, 1 or 2), SO₂N(Rₚ)R_{q}, N(Rₚ)SO₂R_{q} or (CH₂)ₖNRₚR_{q} (where k is 1, 2 or 3), wherein Rₚ and R_{q} are each independently selected from hydrogen or C₁₋₄alkyl;
(55) R₅ is hydrogen and R₇ is selected from C₁₋₄alkyl, C₃₋₆cycloalkyl, heteroaryl and aryl, wherein C₁₋₄alkyl, C₃₋₆cycloalkyl, heteroaryl or aryl is optionally substituted with one or more substituents selected from C₁₋₄alkyl, C₂₋₃alkynyl, halo, cyano, ORₚ, wherein Rₚ is C₁₋₄alkyl;
(56) R₅ is hydrogen and R₇ is aryl optionally substituted with one or more substituents selected from C₁₋₄alkyl, C₂₋₃alkynyl, halo, cyano, ORₚ, wherein Rₚ is C₁₋₄alkyl;
(57) R₃ is hydrogen;
(58) R₆ and R₇ are linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano;
(59) R₆ and R₇ are linked together to form a 3-, 4-, 5-, or 6-membered carbocyclic or oxygen containing heterocyclic ring system, wherein the 3-, 4-, 5-, or 6-membered carbocyclic or oxygen containing heterocyclic ring system is optionally substituted with one or more substituents selected from C₁₋₂alkyl, C₁₋₂alkyoxy, C₁₋₂haloalkyl, halo, and cyano;
(60) R₆ and R₇ are linked together to form a 3-, 4-, 5-, or 6-membered carbocyclic or oxygen containing heterocyclic ring system, wherein the 3-, 4-, 5-, or 6-membered carbocyclic or oxygen containing heterocyclic ring system is optionally substituted with one or more substituents selected from C₁₋₂alkyl, C₁₋₂alkyoxy, C₁₋₂haloalkyl, fluoro, and cyano;
(61) R₆ and R₇ are linked together to form a 3-, 4-, 5-, or 6-membered carbocyclic or oxygen containing heterocyclic ring system, wherein the 3-, 4-, 5-, or 6-membered carbocyclic or oxygen containing heterocyclic ring system is optionally substituted with one or more fluoro;
(61a) R₆ and R₇ are linked together to form a 3-, 4-, 5-, or 6-membered carbocyclic ring system, wherein the 3-, 4-, 5-, or 6-membered carbocyclic heterocyclic ring system is optionally substituted with one or more fluoro;
(62) R₆ and R₇ are linked together to form a 3-membered carbocyclic ring system;
(63) R₆ and R₇ are linked together to form a cyclopropyl ring system;
(64) R₆ and R₇ are linked together to form a 4-membered carbocyclic ring system optionally substituted with one or more fluoro;
(65) R₆ and R₇ are linked together to form a cyclobutyl ring system optionally substituted with one or more fluoro;
(66) R₆ and R₇ are linked together to form a 5-membered carbocyclic ring system;
(67) R₆ and R₇ are linked together to form a cyclopentyl ring system;
(68) R₆ and R₇ are linked together to form a 6-membered carbocyclic ring system;
(69) R₆ and R₇ are linked together to form a cyclohexyl ring system;
(70) R₆ and R₇ are linked together to form a 3-membered heterocyclic ring system;
(71) R₆ and R₇ are linked together to form a 3-membered oxygen containing heterocyclic ring system;
(72) R₆ and R₇ are linked together to form a 4-membered heterocyclic ring system;
(73) R₆ and R₇ are linked together to form a 4-membered oxygen containing heterocyclic ring system;
(74) R₆ and R₇ are linked together to form an oxetanyl ring system;
(75) R₆ and R₇ are linked together to form a 5-membered heterocyclic ring system;
(76) R₆ and R₇ are linked together to form a 5-membered oxygen containing heterocyclic ring system;
(77) R₆ and R₇ are linked together to form a tetrahydrofuranyl ring system;
(78) R₆ and R₇ are linked together to form a 6-membered oxygen containing heterocyclic ring system;
(79) R₆ and R₇ are linked together to form a tetrahydropyranyl ring system;
(80) R₅ is hydrogen, and R₆ and R₇ are linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano;
(81) R₅ is hydrogen, and R₆ and R₇ are linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more halo substituents;
(82) R₅ and R₇ are linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano;
(83) R₆ is hydrogen, and R₅ and R₇ are linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano;
(84) R₅ and R₇ are linked together to form a 5- or 6-membered carbocyclic or heterocyclic ring system;
(85) Rₑ is hydrogen, and R₅ and R₇ are linked together to form a 5- or 6-membered carbocyclic or heterocyclic ring system;
(86) R₅ and R₇ are linked together to form a 5-membered carbocyclic ring system;
(87) R₅ and R₇ are linked together to form a 6-membered carbocyclic ring system;
(88) R₅ and R₇ are linked together to form a cyclohexyl ring system;
(89) R₅ and R₇ are linked together to form a 5-membered heterocyclic ring system;
(90) R₅ and R₇ are linked together to form a tetrahydrofuranyl ring system;
(91) R₅ and R₇ are linked together to form a 6-membered heterocyclic ring system;
(92) R₇ and R₃ are linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂-haloalkyl, halo, and cyano;
(93) R₅ is hydrogen, and R₇ and R₃ are linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano;
(94) R₇ and R₃ are linked together to form a 5-membered heterocyclic ring system;
(95) R₇ and R₃ are linked together to form a pyrrolidinyl ring system;
(96) R₇ and R₃ are linked together to form a 6-membered heterocyclic ring system;
(97) R₇ and R₃ are linked together to form a 6-membered oxygen containing heterocyclic ring system;
(98) R₇ and R₃ are linked together to form a morpholinyl ring system;
(99) X is selected from N and CR₉ wherein R₉ is selected from hydrogen, methyl, and fluoro;
(100) X is N;
(101) X is CR₉ wherein R₉ is selected from hydrogen, methyl, and fluoro;
(102) X is CR₉ wherein R₉ is hydrogen;
(103) Y is selected from O and NR₁₀;
(104) Y is O;
(105) Y is NR₁₀;
(106) R₁₀ is selected from hydrogen and C₁₋₄ alkyl;
(107) R₁₀ is selected from hydrogen, methyl and ethyl;
(108) R₁₀ is hydrogen;
(109) R₁₀ is methyl;
(110) Y is NR₁₀ and R₁₀ is hydrogen;
(111) R₃ and R₁₀ are linked together to form a 4-, 5-, 6- or 7-membered heterocyclic ring system;
(112) R₃ and R₁₀ are linked together to form a 5-membered heterocyclic ring system;
(113) R₃ and R₁₀ are linked together to form a pyrrolidinyl ring system.

Suitably, a heteroaryl or heterocyclyl group as defined herein is a monocyclic heteroaryl or heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5- or 6-membered heterocyclyl ring comprising one, two or three heteroatoms selected from N, O or S. Most suitably, a heterocyclyl group is a 5- or 6-membered ring comprising one, two or three heteroatoms selected from N, O or S

Suitably, an aryl group is phenyl.

Suitably, R₁ is as defined in any one of paragraphs (1) to (8) above. Most suitably, R₁ is as defined in paragraph (5).

Suitably, R₂ is as defined in any one of paragraphs (9) to (14) above. Most suitably, R₂ is as defined in paragraph (12).

Suitably, R₃ is as defined in any one of paragraphs (15) to (21) and paragraphs (111) to (113) above. Most suitably, R₃ is as defined in paragraph (21).

Suitably, R₃ is as defined in any one of paragraphs (15) to (21a) and paragraphs (111) to (113) above. More suitably, R₃ is as defined in any one of paragraphs (17a) to (20a), and (21a). Most suitably, R₃ is as defined in paragraph (21a);

Suitably, R₄ is as defined in any one of paragraphs (22), (28) and (34) above. Most suitably, R₄ is as defined in paragraph (22).

Suitably, R₅ is as defined in any one of paragraphs (23) to (29), (34), (54), (55), (56), (80) to (91), and (93) above. Most suitably, R₅ is as defined in paragraph (23).

Suitably, R₆ is as defined in any one of paragraphs (29) to (34), (58) to (81), (83) and (85) above. Most suitably, R₆ is as defined in paragraph (30).

Suitably, R₇ is as defined in any one of paragraphs (29), (35) to (56), (58) to (98) above. Most suitably, R₇ is as defined in paragraph (39).

Suitably, R₈ is as defined in any one of paragraphs (57) and (92) to (98) above. Most suitably, R₈ is as defined in paragraph (57).

Suitably, R₆ is hydrogen and R₇ is as defined in any one of paragraphs (29), (35) to (53), or R₆ and R₇ are as defined in any one of paragraphs (58) to (79). More suitably, R₆ is hydrogen and R₇ is as defined in any one of paragraphs (52) to (53), or R₆ and R₇ are as defined in any one of paragraphs (58) to (61a). Most suitably, R₆ is hydrogen and R₇ is as defined in paragraph (53), or R₆ and R₇ are as defined in paragraph (61a).

Suitably, X is as defined in any one of paragraphs (99) to (102) above. Most suitably, X is as defined in paragraph (100).

Suitably, Y is as defined in any one of paragraphs (103) to (105) and (110) above. Most suitably, Y is as defined in paragraph (105).

Suitably, R₁₀ is as defined in any one of paragraphs (106) to (113) above. Most suitably R₁₀ is as defined in paragraph (108). Suitably, if R₄, R₅, R₆ and R₇ are hydrogen, then R₈ is also hydrogen;

Suitably, the compound of the invention is not:
N⁶-((1-aminocyclohexyl)methyl)-1-methyl-1H-pyrazolo[3,4-d]pyrimidine-4,6-diamine;
N⁶-((1-aminocyclobutyl)methyl)-1-methyl-1H-pyrazolo[3,4-d]pyrimidine-4,6-diamine;
1-methyl-N⁶-(pyrrolidin-2-ylmethyl)-1H-pyrazolo[3,4-d]pyrimidine-4,6-diamine.

In a particular group of compounds of the invention, R₁, R₂, X and Y are as defined in paragraphs (5), (12), (100) and (110) respectively, i.e. the compounds have the structural formula (II) (a sub-definition of formula (1)) shown below:

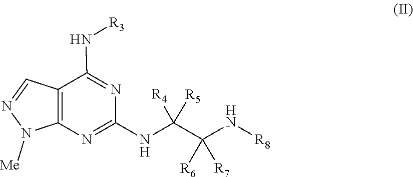

wherein R₃, R₄, R₅, R₆, R₇ and R₃ each having any one of the meanings defined herein; or a pharmaceutically acceptable salt thereof. In an embodiment of the compounds of formula (II):

R₃ is as defined in any one of paragraphs (15) to (21) and paragraphs (111) to (113) above;
R₄ is as defined in any one of paragraphs (22), (28) and (34) above;
R₅ is as defined in any one of paragraphs (23) to (29), (34), (54), (55), (56), (80) to (91), and (93) above;
R₆ is as defined in any one of paragraphs (29) to (34), (58) to (81), (83) and (85) above;
R₇ is as defined in any one of paragraphs (29), (35) to (56), (58) to (98) above; and
R₈ is as defined in any one of paragraphs (57) and (92) to (98) above.

In an embodiment of the compounds of formula (II):

R₃ is as defined in paragraph (21) above;
R₄ is as defined in paragraph (22) above;
R₅ is as defined in paragraph (23) above;
R₆ is as defined in paragraph (30) above;
R₇ is as defined in paragraph (39) above; and
R₈ is as defined in paragraph (57) above.

In another embodiment of the compounds of formula (II):

R₃ is as defined in any one of paragraphs (15) to (21a) and paragraphs (111) to (113) above;
R₄ is as defined in any one of paragraphs (22), (28) and (34) above;
R₅ is as defined in any one of paragraphs (23) to (29), (34), (54), (55), (56), (80) to (91), and (93) above;
R₆ is hydrogen and R₇ is as defined in any one of paragraphs (29), (35) to (53), or R₆ and R₇ are as defined in any one of paragraphs (58) to (79); and
R₈ is as defined in any one of paragraphs (57) and (92) to (98) above.

In an embodiment of the compounds of formula (II):

R₃ is as defined in paragraph (20a) or (21a) above;
R₄ is as defined in paragraph (22) above;
R₅ is as defined in paragraph (23) above;
R₆ is hydrogen and R₇ is as defined in paragraph (53), or R₆ and R₇ are as defined in paragraph (61a); and
R₈ is as defined in paragraph (57) above

In a particular group of compounds of the invention, R₁, R₂, R₄, R₅, X and Y are as defined in paragraphs (5), (12), (22), (25), (100) and (110) respectively, i.e. the compounds have the structural formula (III) (a sub-definition of formula (1)) shown below:

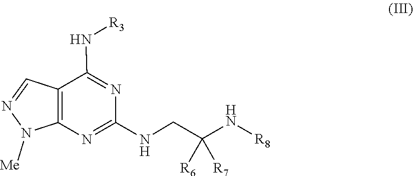

wherein R₃, R₆, R₇ and R₈ each having any one of the meanings defined herein; or a pharmaceutically acceptable salt thereof.

In an embodiment of the compounds of formula (III):

R₃ is as defined in any one of paragraphs (15) to (21) and paragraphs (111) to (113) above;
R₆ is as defined in any one of paragraphs (29) to (34), (58) to (81), (83) and (85) above;
R₇ is as defined in any one of paragraphs (29), (35) to (56), (58) to (98) above; and
R₈ is as defined in any one of paragraphs (57) and (92) to (98) above.

In an embodiment of the compounds of formula (III):

R₃ is as defined in paragraph (21) above;
R₆ is as defined in paragraph (30) above;
R₇ is as defined in paragraph (39) above; and
R₈ is as defined in paragraph (57) above.

In another embodiment of the compounds of formula (III):

R₃ is as defined in any one of paragraphs (15) to (21a) and paragraphs (111) to (113) above;
R₆ is hydrogen and R₇ is as defined in any one of paragraphs (29), (35) to (53), or R₆ and R₇ are as defined in any one of paragraphs (58) to (79); and
R₈ is as defined in any one of paragraphs (57) and (92) to (98) above.

In an embodiment of the compounds of formula (III):

R₃ is as defined in paragraph (20a) or (21a) above;
R₆ is hydrogen and R₇ is as defined in paragraph (52), or R₆ and R₇ are as defined in paragraph (61a); and
R₈ is as defined in paragraph (57) above.

In a particular group of compounds of the invention, R₁, R₂, R₄, R₅, R₆, X and Y are as defined in paragraphs (5), (12), (22), (25), (32), (100) and (110) respectively, i.e. the compounds have the structural formula (IV) (a sub-definition of formula (1)) shown below:

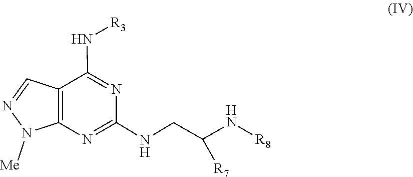

wherein R₃, R₇ and R₈ each having any one of the meanings defined herein; or a pharmaceutically acceptable salt thereof.

In an embodiment of the compounds of formula (IV):

R₃ is as defined in any one of paragraphs (15) to (21) and paragraphs (111) to (113) above;
R₇ is as defined in any one of paragraphs (29), (35) to (56), (58) to (98) above; and
R₈ is as defined in any one of paragraphs (57) and (92) to (98) above.

In an embodiment of the compounds of formula (IV):

R₃ is as defined in paragraph (21) above;
R₇ is as defined in paragraph (39) above; and
R₈ is as defined in paragraph (57) above.

In an embodiment of the compounds of formula (IV):

R₃ is as defined in any one of paragraphs (15) to (21a) and paragraphs (111) to (113) above;
R₇ is as defined in any one of paragraphs (29), (35) to (53) above; and
R₈ is as defined in any one of paragraphs (57) and (92) to (98) above.

In an embodiment of the compounds of formula (IV):

R₃ is as defined in paragraph (20a) or (21a) above;
R₇ is as defined in paragraph (52) above; and
R₈ is as defined in paragraph (57) above.

In a particular group of compounds of the invention, R₁, R₂, R₄, R₅, X and Y are as defined in paragraphs (5), (12), (22), (25), (100) and (110) respectively, i.e. the compounds have the structural formula (V) (a sub-definition of formula (1)) shown below:

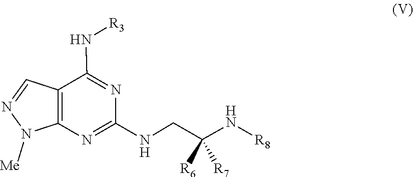

wherein R₃, R₆, R₇ and R₈ each having any one of the meanings defined herein; or a pharmaceutically acceptable salt thereof.

In an embodiment of the compounds of formula (V):

R₃ is as defined in any one of paragraphs (15) to (21) and paragraphs (111) to (113) above;
R₆ is as defined in any one of paragraphs (29) to (34), (58) to (81), (83) and (85) above;
R₇ is as defined in any one of paragraphs (29), (35) to (56), (58) to (98) above; and
R₈ is as defined in any one of paragraphs (57) and (92) to (98) above.

In an embodiment of the compounds of formula (V):

R₃ is as defined in paragraph (21) above;
R₆ is as defined in paragraph (30) above;
R₇ is as defined in paragraph (39) above; and
R₈ is as defined in paragraph (57) above.

In an embodiment of the compounds of formula (V):

R₃ is as defined in any one of paragraphs ((15) to (21a) and paragraphs (111) to (113) above;
R₆ is hydrogen and R₇ is as defined in any one of paragraphs (29), (35) to (53), or R₆ and R₇ are as defined in any one of paragraphs (58) to (79); and
R₈ is as defined in any one of paragraphs (57) and (92) to (98) above.

In an embodiment of the compounds of formula (V):

R₃ is as defined in paragraph (20a) or (21a) above;
R₆ is hydrogen and R₇ is as defined in paragraph (52), or R₆ and R₇ are as defined in paragraph (61a); and
R₈ is as defined in paragraph (57) above.

In a particular group of compounds of the invention, R₁, R₂, R₄, R₅, X and Y are as defined in paragraphs (5), (12), (22), (25), (100) and (110) respectively, i.e. the compounds have the structural formula (VI) (a sub-definition of formula (1)) shown below:

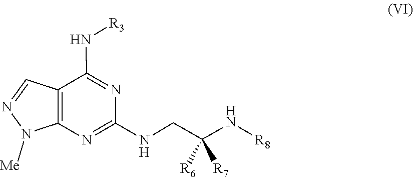

wherein R₃, R₆, R₇ and R₈ each having any one of the meanings defined herein; or a pharmaceutically acceptable salt thereof.

In an embodiment of the compounds of formula (VI):

R₃ is as defined in any one of paragraphs (15) to (21) and paragraphs (111) to (113) above;
R₆ is as defined in any one of paragraphs (29) to (34), (58) to (81), (83) and (85) above;
R₇ is as defined in any one of paragraphs (29), (35) to (56), (58) to (98) above; and
R₈ is as defined in any one of paragraphs (57) and (92) to (98) above.

In an embodiment of the compounds of formula (VI):

R₃ is as defined in paragraph (21) above;
R₆ is as defined in paragraph (30) above;
R₇ is as defined in paragraph (39) above; and
R₈ is as defined in paragraph (57) above.

In an embodiment of the compounds of formula (VI):

R₃ is as defined in any one of paragraphs ((15) to (21a) and paragraphs (111) to (113) above;
R₆ is hydrogen and R₇ is as defined in any one of paragraphs (29), (35) to (53), or R₆ and R₇ are as defined in any one of paragraphs (58) to (79); and
R₈ is as defined in any one of paragraphs (57) and (92) to (98) above.

In an embodiment of the compounds of formula (VI):

R₃ is as defined in paragraph (20a) or (21a) above;
R₆ is hydrogen and R₇ is as defined in paragraph (52), or R₆ and R₇ are as defined in paragraph (61a); and
R₈ is as defined in paragraph (57) above.

In a particular group of compounds of the invention, R₁, R₂, R₄, R₅, R₈, X and Y are as defined in paragraphs (5), (12), (22), (25), (57), (100) and (110) respectively, i.e. the compounds have the structural formula (VII) (a sub-definition of formula (1)) shown below:

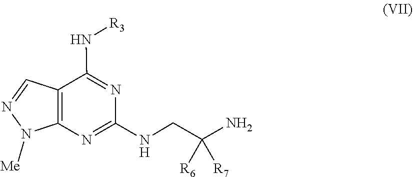

wherein R₃, R₆ and R₇ each having any one of the meanings defined herein; or a pharmaceutically acceptable salt thereof.

In an embodiment of the compounds of formula (VII):

R₃ is as defined in any one of paragraphs (15) to (21) and paragraphs (111) to (113) above;
R₆ is as defined in any one of paragraphs (29) to (34), (58) to (81), (83) and (85) above; and
R₇ is as defined in any one of paragraphs (29), (35) to (56), (58) to (98) above.

In an embodiment of the compounds of formula (VII):

R₃ is as defined in paragraph (21) above;
R₆ is as defined in paragraph (30) above; and
R₇ is as defined in paragraph (39) above.

In an embodiment of the compounds of formula (VII):

R₃ is as defined in any one of paragraphs ((15) to (21a) and paragraphs (111) to (113) above;
R₆ is hydrogen and R₇ is as defined in any one of paragraphs (29), (35) to (53), or R₆ and R₇ are as defined in any one of paragraphs (58) to (79); and
R₈ is as defined in any one of paragraphs (57) and (92) to (98) above.

In an embodiment of the compounds of formula (VII):

R₃ is as defined in paragraph (20a) or (21a) above;
R₆ is hydrogen and R₇ is as defined in paragraph (52), or R₆ and R₇ are as defined in paragraph (61a); and
R₈ is as defined in paragraph (57) above.

In a particular group of compounds of the invention, R₁, R₂, R₄, R₅, R₃, X and Y are as defined in paragraphs (5), (12), (22), (25), (55), (100) and (110) respectively and R₇ is phenyl, i.e. the compounds have the structural formula (VIII) (a sub-definition of formula (1)) shown below:

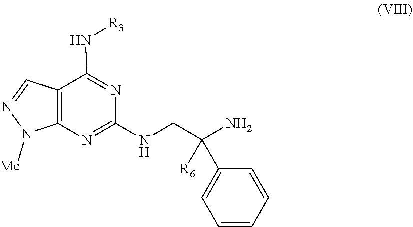

wherein R₃ and R₆ each having any one of the meanings defined herein; or a pharmaceutically acceptable salt thereof.

In an embodiment of the compounds of formula (VIII):

R₃ is as defined in any one of paragraphs (15) to (21) and paragraphs (111) to (113) above; and
R₆ is as defined in any one of paragraphs (29) to (34), (58) to (81), (83) and (85) above.

In an embodiment of the compounds of formula (VIII):

R₃ is as defined in paragraph (21) above; and
R₆ is as defined in paragraph (30) above.

In an embodiment of the compounds of formula (VIII):

R₃ is as defined in any one of paragraphs (15) to (21a) and paragraphs (111) to (113) above; and
R₆ is as defined in any one of paragraphs (29) to (34), (58) to (81), (83) and (85) above.

In an embodiment of the compounds of formula (VIII):

R₃ is as defined in paragraph (20a) or (21a) above; and
R₆ is as defined in paragraph (30) above.

In a particular group of compounds of the invention, R₁, R₂, R₄, R₅, R₈, X and Y are as defined in paragraphs (5), (12), (22), (25), (57), (100) and (110) respectively, and R₆ and R₇ as defined in paragraph (63), i.e. the compounds have the structural formula (IX) (a sub-definition of formula (1)) shown below:

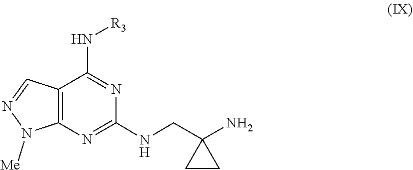

wherein R₃ has any one of the meanings defined herein; or a pharmaceutically acceptable salt thereof.

In an embodiment of the compounds of formula (IX):

R₃ is as defined in any one of paragraphs (15) to (21) and paragraphs (111) to (113) above.

In an embodiment of the compounds of formula (IX):

R₃ is as defined in paragraph (21) above.

In an embodiment of the compounds of formula (IX):

R₃ is as defined in any one of paragraphs (15) to (21a) and paragraphs (111) to (113) above.

In an embodiment of the compounds of formula (IX):

R₃ is as defined in paragraph (20a) or (21a) above.

Particular compounds of the present invention include any of the compounds exemplified in the present application, or a pharmaceutically acceptable salt thereof, and, in particular, any of the following:
N6-(2-Aminoethyl)-1-methyl-N4-[4-(trifluoromethyl)phenyl]pyrazolo[3,4-d]pyrimidine-4,6-diamine;
N6-(2-aminoethyl)-1-methyl-N4-[3-(trifluoromethyl)phenyl]-1H-pyrazolo[3,4-d]pyrimidine-4,6-diamine;
N6-(2-aminoethyl)-1-methyl-N4-(4-phenoxyphenyl)-1H-pyrazolo[3,4-d]pyrimidine-4,6-diamine;
N6-(2-aminoethyl)-1-methyl-N4-(3-phenoxyphenyl)-1H-pyrazolo[3,4-d]pyrimidine-4,6-diamine;
N6-(2-aminoethyl)-1-methyl-N4-[(4-phenylphenyl)methyl]pyrazolo[3,4-d]pyrimidine-4,6-diamine;
N6-(2-aminoethyl)-N4-[1-(3-chloro-4-methoxyphenyl)ethyl]-1-methyl-1H-pyrazolo[3,4-d]pyrimidine-4,6-diamine;
N6-(2-aminoethyl)-N4-[2-(6-methoxypyridin-3-yl)ethyl]-1-methyl-1H-pyrazolo[3,4-d]pyrimidine-4,6-diamine;
N6-(2-aminoethyl)-N4-[2-(3-chloro-4-methoxyphenyl)ethyl]-1-methyl-1H-pyrazolo[3,4-d]pyrimidine-4,6-diamine;
N6-(2-aminoethyl)-N4-[2-(2H-1,3-benzodioxol-5-yl)ethyl]-1-methyl-1H-pyrazolo[3,4-d]pyrimidine-4,6-diamine;
N6-(2-aminoethyl)-N4-[(1-benzofuran-5-yl)methyl]-1-methyl-1H-pyrazolo[3,4-d]pyrimidine-4,6-diamine;
N6-(2-aminoethyl)-N4-{3′-methoxy-[1,1′-biphenyl]-3-yl}-1-methyl-1H-pyrazolo[3,4-d]pyrimidine-4,6-diamine;
N6-(2-aminoethyl)-N4-{[1-(benzenesulfonyl)piperidin-4-yl]methyl}-1-methyl-1H-pyrazolo[3,4-d]pyrimidine-4,6-diamine;
N6-(2-aminoethyl)-N4-[(1H-1,3-benzodiazol-5-yl)methyl]-1-methyl-1H-pyrazolo[3,4-d]pyrimidine-4,6-diamine;
N6-(2-aminoethyl)-N4-{3′-methoxy-[1,1′-biphenyl]-4-yl}-1-methyl-1H-pyrazolo[3,4-d]pyrimidine-4,6-diamine;
N6-(2-aminoethyl)-N4-{4′-methoxy-[1,1′-biphenyl]-3-yl}-1-methyl-1H-pyrazolo[3,4-d]pyrimidine-4,6-diamine;
N6-(2-aminoethyl)-N4-{[1,1′-biphenyl]-3-yl}-1-methyl-1H-pyrazolo[3,4-d]pyrimidine-4,6-diamine;
N6-(2-aminoethyl)-N4-[(3-chloro-4-fluorophenyl)methyl]-1-methyl-1H-pyrazolo[3,4-d]pyrimidine-4,6-diamine;
N6-(2-aminoethyl)-N4-[(4-chloro-2-fluorophenyl)methyl]-1-methyl-1H-pyrazolo[3,4-d]pyrimidine-4,6-diamine;
N6-(2-aminoethyl)-N4-[(2,4-difluorophenyl)methyl]-1-methyl-1H-pyrazolo[3,4-d]pyrimidine-4,6-diamine;
N6-(2-aminoethyl)-N4-[(3-chloro-5-methoxyphenyl)methyl]-1-methyl-1H-pyrazolo[3,4-d]pyrimidine-4,6-diamine;
N6-(2-aminoethyl)-N4-(3-chloro-4-methoxyphenyl)-1-methyl-1H-pyrazolo[3,4-d]pyrimidine-4,6-diamine;
N6-(2-aminoethyl)-N4-[(3-chloro-4-methylphenyl)methyl]-1-methyl-1H-pyrazolo[3,4-d]pyrimidine-4,6-diamine;
N6-(2-aminoethyl)-1-methyl-N4-{[3-(trifluoromethyl)phenyl]methyl}-1H-pyrazolo[3,4-d]pyrimidine-4,6-diamine;
N6-(2-aminoethyl)-N4-[(2,4-dimethoxyphenyl)methyl]-1-methyl-1H-pyrazolo[3,4-d]pyrimidine-4,6-diamine;
N6-(2-aminoethyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(4-methoxyphenyl)methyl]-1-methyl-1H-pyrazolo[3,4-d]pyrimidine-4,6-diamine;
N6-(2-amino-2-methylpropyl)-N4-({[1,1′-biphenyl]-4-yl}methyl)-1-methyl-1H-pyrazolo[3,4-d]pyrimidine-4,6-diamine;
N6-(2-aminoethyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-({[1,1′-biphenyl]-4-yl}methyl)-1,3-dimethyl-1H-pyrazolo[3,4-d]pyrimidine-4,6-diamine;
N6-[(1-aminocyclohexyl)methyl]-1-methyl-N4-[4-(trifluoromethyl)phenyl]pyrazolo[3,4-d]pyrimidine-4,6-diamine;
N6-(2-amino-2-methylpropyl)-1,3-dimethyl-N4-[3-(trifluoromethyl)phenyl]-1H-pyrazolo[3,4-d]pyrimidine-4,6-diamine;
6-N-(2-aminocyclohexyl)-4-N-[(3-chloro-4-methylphenyl)methyl]-1-methylpyrazolo[3,4-d]pyrimidine-4,6-diamine;
6-N-[(4-aminooxan-4-yl)methyl]-1-methyl-4-N-[6-(trifluoromethyl)pyridin-3-yl]pyrazolo[3,4-d]pyrimidine-4,6-diamine;
N6-[(3R,4S)-4-aminotetrahydrofuran-3-yl]-1-methyl-N4-[4-(trifluoromethyl)phenyl]pyrazolo[3,4-d]pyrimidine-4,6-diamine;
N6-(2-aminoethyl)-N4-{[1-(3-chloro-4-methoxybenzoyl)piperidin-4-yl]methyl}-1-methyl-1H-pyrazolo[3,4-d]pyrimidine-4,6-diamine;
6-N-(2-Amino-2-methylpropyl)-1,3-dimethyl-4-N-[[3-(trifluoromethyl)phenyl]methyl]pyrazolo[3,4-b]pyridine-4,6-diamine;
6-N-[(1-Aminocyclopropyl)methyl]-4-N-[(3-chloro-4-methylphenyl)methyl]-1-methylpyrazolo[3,4-d]pyrimidine-4,6-diamine;
6-N-[(1-Aminocyclopropyl)methyl]-4-N-[(3-chloro-4-fluorophenyl)methyl]-1-methylpyrazolo[3,4-d]pyrimidine-4,6-diamine;
N6-[(1-aminocyclopropyl)methyl]-N4-[3-fluoro-4-(trifluoromethyl)phenyl]-1-methyl-pyrazolo[3,4-d]pyrimidine-4,6-diamine;
6-N-[(1-aminocyclopropyl)methyl]-4-N-(3-chloro-4-methoxyphenyl)-1-methylpyrazolo[3,4-d]pyrimidine-4,6-diamine;
6-N-[(1-aminocyclopropyl)methyl]-4-N-(2,4-difluorophenyl)-1-methylpyrazolo[3,4-d]pyrimidine-4,6-diamine;
6-N-[(1-aminocyclopropyl)methyl]-1-methyl-4-N-[3-(trifluoromethyl)phenyl]pyrazolo[3,4-d]pyrimidine-4,6-diamine;
6-N-[(1-aminocyclopropyl)methyl]-4-N-[3-(difluoromethyl)phenyl]-1-methylpyrazolo[3,4-d]pyrimidine-4,6-diamine;
6-N-[(1-aminocyclopropyl)methyl]-4-N-(4-fluorophenyl)-1-methylpyrazolo[3,4-d]pyrimidine-4,6-diamine;
6-N-[(1-aminocyclopropyl)methyl]-4-N-(3-chloro-4-methylphenyl)-1-methylpyrazolo[3,4-d]pyrimidine-4,6-diamine;
6-N-[(1-aminocyclopropyl)methyl]-1-methyl-4-N-pyridin-3-ylpyrazolo[3,4-d]pyrimidine-4,6-diamine;
6-N-[(1-aminocyclopropyl)methyl]-1-methyl-4-N-phenylpyrazolo[3,4-d]pyrimidine-4,6-diamine;
6-N-[(1-aminocyclopropyl)methyl]-4-N-[(3-bromophenyl)methyl]-1-methylpyrazolo[3,4-d]pyrimidine-4,6-diamine;
6-N-[(1-aminocyclopropyl)methyl]-4-N-[(4-bromophenyl)methyl]-1-methylpyrazolo[3,4-d]pyrimidine-4,6-diamine;
6-N-[(1-aminocyclopropyl)methyl]-4-N-(2-chloro-4-fluorophenyl)-1-methylpyrazolo[3,4-d]pyrimidine-4,6-diamine;
6-N-[(1-aminocyclopropyl)methyl]-4-N-(4-chloro-2-fluorophenyl)-1-methylpyrazolo[3,4-d]pyrimidine-4,6-diamine;
6-N-[(1-aminocyclopropyl)methyl]-4-N-(3-chloro-5-fluorophenyl)-1-methylpyrazolo[3,4-d]pyrimidine-4,6-diamine;
5-[[6-[(1-aminocyclopropyl)methylamino]-1-methyl-pyrazolo[3,4-d]pyrimidin-4-yl]amino]-2-chloro-benzonitrile;
N6-[(1-aminocyclopropyl)methyl]-1-methyl-N4-(1-phenyl-3-bicyclo[1.1.1]pentanyl)pyrazolo[3,4-d]pyrimidine-4,6-diamine;
6-N-[(1-aminocyclopropyl)methyl]-1-methyl-4-N-[[6-(trifluoromethyl)pyridin-3-yl]methyl]pyrazolo[3,4-d]pyrimidine-4,6-diamine;
6-N-[(1-aminocyclopropyl)methyl]-4-N-(4-chloro-3,5-difluorophenyl)-1-methylpyrazolo[3,4-d]pyrimidine-4,6-diamine;
6-N-[(1-aminocyclopropyl)methyl]-4-N-[3-(difluoromethoxy)phenyl]-1-methylpyrazolo[3,4-d]pyrimidine-4,6-diamine;
6-N-[(1-aminocyclopropyl)methyl]-1-methyl-4-N-(4-propan-2-yloxyphenyl)pyrazolo[3,4-d]pyrimidine-4,6-diamine;
6-N-[(1-aminocyclopropyl)methyl]-4-N-[(4-chloro-3-fluorophenyl)methyl]-1-methylpyrazolo[3,4-d]pyrimidine-4,6-diamine;
6-N-[(1-aminocyclopropyl)methyl]-4-N-(2,3-difluorophenyl)-1-methylpyrazolo[3,4-d]pyrimidine-4,6-diamine;
6-N-[(1-aminocyclopropyl)methyl]-4-N-(2,5-difluorophenyl)-1-methylpyrazolo[3,4-d]pyrimidine-4,6-diamine;
6-N-[(1-aminocyclopropyl)methyl]-4-N-[(2,4-difluorophenyl)methyl]-1-methylpyrazolo[3,4-d]pyrimidine-4,6-diamine;
6-N-[(1-aminocyclopropyl)methyl]-4-N-(3-chloro-4-fluorophenyl)-1-methylpyrazolo[3,4-d]pyrimidine-4,6-diamine;
6-N-[(1-aminocyclopropyl)methyl]-4-N-(4-chlorophenyl)-1-methylpyrazolo[3,4-d]pyrimidine-4,6-diamine;
6-N-[(1-aminocyclopropyl)methyl]-4-N-(3-fluorophenyl)-1-methylpyrazolo[3,4-d]pyrimidine-4,6-diamine;
6-N-[(1-aminocyclopropyl)methyl]-1,3-dimethyl-4-N-[4-(trifluoromethyl)phenyl]pyrazolo[3,4-d]pyrimidine-4,6-diamine;
4-[[6-[(1-aminocyclopropyl)methylamino]-1-methylpyrazolo[3,4-d]pyrimidin-4-yl]amino]benzonitrile;
6-N-[(1-aminocyclopropyl)methyl]-1-methyl-4-N-[4-(trifluoromethoxy)phenyl]pyrazolo[3,4-d]pyrimidine-4,6-diamine;
6-N-[(1-aminocyclopropyl)methyl]-4-N-(4-methoxyphenyl)-1-methylpyrazolo[3,4-d]pyrimidine-4,6-diamine;
6-N-[(1-aminocyclopropyl)methyl]-4-N-[4-(difluoromethoxy)phenyl]-1-methylpyrazolo[3,4-d]pyrimidine-4,6-diamine;
6-N-[(1-aminocyclopropyl)methyl]-4-N-(3-chlorophenyl)-1-methylpyrazolo[3,4-d]pyrimidine-4,6-diamine;
6-N-[(1-aminocyclopropyl)methyl]-4-N-[4-(difluoromethyl)phenyl]-1-methylpyrazolo[3,4-d]pyrimidine-4,6-diamine;
3-[[6-[(1-aminocyclopropyl)methylamino]-1-methylpyrazolo[3,4-d]pyrimidin-4-yl]amino]bicyclo[1.1.1]pentane-1-carbonitrile;
6-N-[(1-aminocyclopropyl)methyl]-4-N-(3-bicyclo[1.1.1]pentanyl)-1-methylpyrazolo[3,4-d]pyrimidine-4,6-diamine;
6-N-[(1-aminocyclopropyl)methyl]-4-N-(3,4-difluorophenyl)-1-methylpyrazolo[3,4-d]pyrimidine-4,6-diamine;
6-N-[(1-aminocyclopropyl)methyl]-4-N-(3,4-dichlorophenyl)-1-methylpyrazolo[3,4-d]pyrimidine-4,6-diamine;
6-N-[(1-aminocyclopropyl)methyl]-4-N-(4-chloro-3-methoxyphenyl)-1-methylpyrazolo[3,4-d]pyrimidine-4,6-diamine;
N6-[(1-aminocyclopropyl)methyl]-N4-[(2,4-dimethoxyphenyl)methyl]-1-methyl-pyrazolo[3,4-d]pyrimidine-4,6-diamine;
N6-[(1-aminocyclopropyl)methyl]-N4-{3-fluorobicyclo[1.1.1]pentan-1-yl}-1-methyl-1H-pyrazolo[3,4-d]pyrimidine-4,6-diamine;
6-N-[(1-aminocyclopropyl)methyl]-1-methyl-4-N-[4-(trifluoromethyl)phenyl]pyrazolo[3,4-d]pyrimidine-4,6-diamine;
6-N-[(1-aminocyclopropyl)methyl]-1-methyl-4-N-[6-(trifluoromethyl)pyridin-3-yl]pyrazolo[3,4-d]pyrimidine-4,6-diamine;
6-N-[(1-aminocyclopropyl)methyl]-4-N-(5-chloropyridin-2-yl)-1-methylpyrazolo[3,4-d]pyrimidine-4,6-diamine;
N6-[(1-aminocyclopropyl)methyl]-1-methyl-N4-[5-(trifluoromethyl)pyrazin-2-yl]pyrazolo[3,4-d]pyrimidine-4,6-diamine;
6-N-[(1-aminocyclopropyl)methyl]-1-ethyl-4-N-[4-(trifluoromethyl)phenyl]pyrazolo[3,4-d]pyrimidine-4,6-diamine;
N6-[(1-aminocyclopropyl)methyl]-1-(trideuteriomethyl)-N4-[4-(trifluoromethyl)phenyl]pyrazolo[3,4-d]pyrimidine-4,6-diamine;
N6-[(1-aminocyclopropyl)methyl]-1-(trideuteriomethyl)-N4-[6-(trifluoromethyl)-3-pyridyl]pyrazolo[3,4-d]pyrimidine-4,6-diamine;
6-N-[(1-aminocyclopropyl)methyl]-4-N-[[4-[2-(3-but-3-ynyldiazirin-3-yl)ethoxy]-3-chlorophenyl]methyl]-1-methylpyrazolo[3,4-d]pyrimidine-4,6-diamine;
N6-[(1-Aminocyclopropyl)methyl]-1-methyl-N4-[4-(trifluoromethyl)phenyl]pyrazolo[3,4-b]pyridine-4,6-diamine;
6-N-(2-aminopropyl)-4-N-[(3-chloro-4-methylphenyl)methyl]-1-methylpyrazolo[3,4-d]pyrimidine-4,6-diamine;
6-N-(1-aminopropan-2-yl)-4-N-[(3-chloro-4-methylphenyl)methyl]-1-methylpyrazolo[3,4-d]pyrimidine-4,6-diamine;
6-N-[(2S)-2-aminopropyl]-4-N-[(3-chloro-4-methylphenyl)methyl]-1-methylpyrazolo[3,4-d]pyrimidine-4,6-diamine;
6-N-[(2R)-2-aminopropyl]-4-N-[(3-chloro-4-methylphenyl)methyl]-1-methylpyrazolo[3,4-d]pyrimidine-4,6-diamine;
6-N-[(2S)-1-aminobutyl]-4-N-[(3-chloro-4-methylphenyl)methyl]-1-methylpyrazolo[3,4-d]pyrimidine-4,6-diamine;
6-N-[(2S)-2-aminobutyl]-4-N-[(3-chloro-4-methylphenyl)methyl]-1-methylpyrazolo[3,4-d]pyrimidine-4,6-diamine;
6-N-[(2R)-2-aminobutyl]-4-N-[(3-chloro-4-methylphenyl)methyl]-1-methylpyrazolo[3,4-d]pyrimidine-4,6-diamine;
N6-[(1-aminocyclobutyl)methyl]-1-methyl-N4-[4-(trifluoromethyl)phenyl]pyrazolo[3,4-d]pyrimidine-4,6-diamine;
N6-[(1-aminocyclobutyl)methyl]-N4-[4-(difluoromethoxy)phenyl]-1-methyl-pyrazolo[3,4-d]pyrimidine-4,6-diamine;
N6-(2-amino-2-cyclohexyl-ethyl)-N4-tert-butyl-1-methyl-pyrazolo[3,4-d]pyrimidine-4,6-diamine;
N6-(2-amino-2-cyclohexyl-ethyl)-1-methyl-N4-[4-(trifluoromethyl)phenyl]pyrazolo[3,4-d]pyrimidine-4,6-diamine;
6-N-[(1-amino-3,3-difluorocyclobutyl)methyl]-1-methyl-4-N-[4-(trifluoromethyl)phenyl]pyrazolo[3,4-d]pyrimidine-4,6-diamine;
6-N-(2-amino-2-cyclopropylethyl)-1-methyl-4-N-[4-(trifluoromethyl)phenyl]pyrazolo[3,4-d]pyrimidine-4,6-diamine;
6-N-[(3-aminooxolan-3-yl)methyl]-1-methyl-4-N-[4-(trifluoromethyl)phenyl]pyrazolo[3,4-d]pyrimidine-4,6-diamine;
N6-[(3-aminotetrahydrofuran-3-yl)methyl]-1-methyl-N4-[6-(trifluoromethyl)-3-pyridyl]pyrazolo[3,4-d]pyrimidine-4,6-diamine;
1-methyl-N6-[(morpholin-3-yl)methyl]-N4-[4-(trifluoromethyl)phenyl]-1H-pyrazolo[3,4-d]pyrimidine-4,6-diamine;
4-N-[(3-chloro-4-methylphenyl)methyl]-1-methyl-6-N-[[(2R)-pyrrolidin-2-yl]methyl]pyrazolo[3,4-d]pyrimidine-4,6-diamine;
6-N-[(1-aminocyclopentyl)methyl]-1-methyl-4-N-[4-(trifluoromethyl)phenyl]pyrazolo[3,4-d]pyrimidine-4,6-diamine;
6-N-[(1-aminocyclopentyl)methyl]-1-methyl-4-N-[6-(trifluoromethyl)pyridin-3-yl]pyrazolo[3,4-d]pyrimidine-4,6-diamine;
6-N-[(1-aminocyclopentyl)methyl]-4-N-[4-(difluoromethoxy)phenyl]-1-methylpyrazolo[3,4-d]pyrimidine-4,6-diamine;
6-N-[(1-aminocyclopentyl)methyl]-1-methyl-4-N-phenylpyrazolo[3,4-d]pyrimidine-4,6-diamine;
4-[[6-[(1-aminocyclopentyl)methylamino]-1-methylpyrazolo[3,4-d]pyrimidin-4-yl]amino]benzonitrile;
6-N-[(1-aminocyclopentyl)methyl]-1-methyl-4-N-[4-(trifluoromethoxy)phenyl]pyrazolo[3,4-d]pyrimidine-4,6-diamine;
N6-[(1-aminocyclobutyl)methyl]-1-(trideuteriomethyl)-N4-[4-(trifluoromethyl)phenyl]pyrazolo[3,4-d]pyrimidine-4,6-diamine;
N6-(2-Amino-3-fluoropropyl)-N4-[(3-chloro-4-methylphenyl)methyl]-1-methyl-1H-pyrazolo[3,4-d]pyrimidine-4,6-diamine;
6-N-(2-amino-3-fluoropropyl)-1-methyl-4-N-[4-(trifluoromethyl)phenyl]pyrazolo[3,4-d]pyrimidine-4,6-diamine;
6-N-(2-amino-3-methoxypropyl)-4-N-[(3-chloro-4-methylphenyl)methyl]-1-methylpyrazolo[3,4-d]pyrimidine-4,6-diamine;
6-N-[(3-Aminooxetan-3-yl)methyl]-1-methyl-4-N-[4-(trifluoromethyl)phenyl]pyrazolo[3,4-d]pyrimidine-4,6-diamine;
6-N-[(3-Aminooxetan-3-yl)methyl]-1-methyl-4-N-phenylpyrazolo[3,4-d]pyrimidine-4,6-diamine;
N6-(2-Amino-2-phenyl-propyl)-N4-tert-butyl-1-(trideuteriomethyl)pyrazolo[3,4-d]pyrimidine-4,6-diamine;
6-N-(2-Amino-2-phenylethyl)-1-ethyl-4-N-propan-2-ylpyrazolo[3,4-d]pyrimidine-4,6-diamine;
6-N-(2-amino-2-phenylethyl)-4-N-[(3-chloro-4-methylphenyl)methyl]-1-methylpyrazolo[3,4-d]pyrimidine-4,6-diamine;
6-N-(2-amino-2-phenylethyl)-4-N-[(2,4-difluorophenyl)methyl]-1-methylpyrazolo[3,4-d]pyrimidine-4,6-diamine;
6-N-(2-amino-2-phenylethyl)-4-N-[(3-chloro-4-fluorophenyl)methyl]-1-methylpyrazolo[3,4-d]pyrimidine-4,6-diamine;
6-N-(2-amino-2-phenylethyl)-4-N-[(3-bromophenyl)methyl]-1-methylpyrazolo[3,4-d]pyrimidine-4,6-diamine;
6-N-(2-amino-2-phenylethyl)-4-N-[(4-bromophenyl)methyl]-1-methylpyrazolo[3,4-d]pyrimidine-4,6-diamine;
6-N-(2-amino-2-phenylethyl)-1-methyl-4-N-[[6-(trifluoromethyl)pyridin-3-yl]methyl]pyrazolo[3,4-d]pyrimidine-4,6-diamine;
N6-(2-amino-2-phenyl-ethyl)-N4-[(2,4-dimethoxyphenyl)methyl]-1-methyl-pyrazolo[3,4-d]pyrimidine-4,6-diamine;
6-N-(2-amino-2-phenylethyl)-4-N-(4-fluorophenyl)-1-methylpyrazolo[3,4-d]pyrimidine-4,6-diamine;
N6-(2-amino-2-phenyl-propyl)-1-methyl-N4-[2-(trifluoromethyl)pyrimidin-5-yl]pyrazolo[3,4-d]pyrimidine-4,6-diamine;
N6-(2-amino-2-phenyl-propyl)-1-methyl-N4-[4-(trifluoromethoxy)phenyl]pyrazolo[3,4-d]pyrimidine-4,6-diamine;
N6-(2-amino-2-phenyl-propyl)-N4-(3,4-dichlorophenyl)-1-methyl-pyrazolo[3,4-d]pyrimidine-4,6-diamine;
6-N-(2-amino-2-phenylethyl)-4-N-[4-(difluoromethoxy)phenyl]-1-methylpyrazolo[3,4-d]pyrimidine-4,6-diamine;
6-N-(2-amino-2-phenylpropyl)-4-N-[4-(difluoromethoxy)phenyl]-1-methylpyrazolo[3,4-d]pyrimidine-4,6-diamine;
N6-[(2R)-2-amino-2-phenyl-ethyl]-N4-[4-(difluoromethoxy)phenyl]-1-methyl-pyrazolo[3,4-d]pyrimidine-4,6-diamine;
6-N-(2-amino-2-phenylethyl)-1-methyl-4-N-[4-(trifluoromethyl)phenyl]pyrazolo[3,4-d]pyrimidine-4,6-diamine;
6-N-(2-amino-2-phenylethyl)-1,3-dimethyl-4-N-[4-(trifluoromethyl)phenyl]pyrazolo[3,4-d]pyrimidine-4,6-diamine;
N6-(2-amino-2-phenyl-propyl)-1-methyl-N4-[4-(trifluoromethyl)phenyl]pyrazolo[3,4-d]pyrimidine-4,6-diamine;
N6-(2-amino-2-phenyl-ethyl)-1-(trideuteriomethyl)-N4-[4-(trifluoromethyl)phenyl]pyrazolo[3,4-d]pyrimidine-4,6-diamine;
N6-(2-amino-2-phenyl-propyl)-1-(trideuteriomethyl)-N4-[4-(trifluoromethyl)phenyl]pyrazolo[3,4-d]pyrimidine-4,6-diamine;
6-N-[(2R)-2-amino-2-phenylethyl]-1-methyl-4-N-[4-(trifluoromethyl)phenyl]pyrazolo[3,4-d]pyrimidine-4,6-diamine;
6-N-[(2S)-2-amino-2-phenylethyl]-1-methyl-4-N-[4-(trifluoromethyl)phenyl]pyrazolo[3,4-d]pyrimidine-4,6-diamine;
N6-[(2R)-2-amino-2-phenyl-ethyl]-1-(trideuteriomethyl)-N4-[4-(trifluoromethyl)phenyl]pyrazolo[3,4-d]pyrimidine-4,6-diamine;
6-N-(2-amino-2-phenylethyl)-1-methyl-4-N-[6-(trifluoromethyl)pyridin-3-yl]pyrazolo[3,4-d]pyrimidine-4,6-diamine;
6-N-(2-amino-2-phenylpropyl)-1-methyl-4-N-[6-(trifluoromethyl)pyridin-3-yl]pyrazolo[3,4-d]pyrimidine-4,6-diamine;
N6-(2-amino-2-phenyl-propyl)-1-(trideuteriomethyl)-N4-[6-(trifluoromethyl)-3-pyridyl]pyrazolo[3,4-d]pyrimidine-4,6-diamine;
N6-(2-amino-2-phenyl-ethyl)-N4-[6-(difluoromethoxy)-3-pyridyl]-1-methyl-pyrazolo[3,4-d]pyrimidine-4,6-diamine;
N6-(2-amino-2-phenyl-propyl)-N4-[6-(difluoromethoxy)-3-pyridyl]-1-methyl-pyrazolo[3,4-d]pyrimidine-4,6-diamine;
N6-[(2R)-2-amino-2-phenyl-ethyl]-N4-(4-chlorophenyl)-1-methyl-pyrazolo[3,4-d]pyrimidine-4,6-diamine;
N6-[(2S)-2-amino-2-phenyl-ethyl]-N4-(4-chlorophenyl)-1-methyl-pyrazolo[3,4-d]pyrimidine-4,6-diamine;
N6-[(2R)-2-amino-2-phenyl-ethyl]-N4-(4-methoxyphenyl)-1-methyl-pyrazolo[3,4-d]pyrimidine-4,6-diamine;
N6-[(2R)-2-amino-2-phenyl-ethyl]-N4-(4-isopropoxyphenyl)-1-methyl-pyrazolo[3,4-d]pyrimidine-4,6-diamine;
N6-[(2R)-2-amino-2-phenyl-ethyl]-N4-(3,4-difluorophenyl)-1-methyl-pyrazolo[3,4-d]pyrimidine-4,6-diamine;
N6-[(2R)-2-amino-2-phenyl-ethyl]-N4-[4-(dimethylamino)phenyl]-1-methyl-pyrazolo[3,4-d]pyrimidine-4,6-diamine;
6-N-(2-amino-2-phenylethyl)-4-N,1-dimethylpyrazolo[3,4-d]pyrimidine-4,6-diamine;
6-N-(2-amino-2-phenylethyl)-1-methyl-4-N-(trideuteriomethyl)pyrazolo[3,4-d]pyrimidine-4,6-diamine;
6-N-(2-amino-2-phenylethyl)-4-N-methyl-1-(trideuteriomethyl)pyrazolo[3,4-d]pyrimidine-4,6-diamine;
6-N-(2-amino-2-phenylpropyl)-4-N,1-dimethylpyrazolo[3,4-d]pyrimidine-4,6-diamine;
6-N-[(2R)-2-amino-2-phenylethyl]-4-N,1-dimethylpyrazolo[3,4-d]pyrimidine-4,6-diamine;
6-N-(2-amino-2-phenylethyl)-4-N-ethyl-1-methylpyrazolo[3,4-d]pyrimidine-4,6-diamine;
6-N-(2-amino-2-phenylethyl)-4-N-ethyl-4-N,1-dimethylpyrazolo[3,4-d]pyrimidine-4,6-diamine;
6-N-(2-amino-2-phenylethyl)-4-N-cyclopropyl-1-methylpyrazolo[3,4-d]pyrimidine-4,6-diamine;
6-N-(2-amino-2-phenylethyl)-1-methyl-4-N-(2,2,2-trifluoroethyl)pyrazolo[3,4-d]pyrimidine-4,6-diamine;
N6-[(2R)-2-amino-2-phenyl-ethyl]-1-methyl-N4-(2,2,2-trifluoroethyl)pyrazolo[3,4-d]pyrimidine-4,6-diamine;
6-N-(2-amino-2-phenylethyl)-1-methyl-4-N-propan-2-ylpyrazolo[3,4-d]pyrimidine-4,6-diamine;
6-N-[(2R)-2-amino-2-phenylethyl]-1-methyl-4-N-propan-2-ylpyrazolo[3,4-d]pyrimidine-4,6-diamine;
N′-(1-methyl-4-pyrrolidin-1-ylpyrazolo[3,4-d]pyrimidin-6-yl)-1-phenylethane-1,2-diamine;
6-N-(2-amino-2-phenylethyl)-4-N-(2-methoxyethyl)-1-methylpyrazolo[3,4-d]pyrimidine-4,6-diamine;
6-N-(2-amino-2-phenylethyl)-4-N-tert-butyl-1-methylpyrazolo[3,4-d]pyrimidine-4,6-diamine;
N6-(2-amino-2-phenyl-propyl)-N4-tert-butyl-1-methyl-pyrazolo[3,4-d]pyrimidine-4,6-diam
N6-(2-amino-2-phenyl-ethyl)-N4-tert-butyl-1-(trideuteriomethyl)pyrazolo[3,4-d]pyrimidine-4,6-diamine;
6-N-[(2R)-2-amino-2-phenylethyl]-4-N-tert-butyl-1-methylpyrazolo[3,4-d]pyrimidine-4,6-diamine;
6-N-[(2S)-2-amino-2-phenylethyl]-4-N-tert-butyl-1-methylpyrazolo[3,4-d]pyrimidine-4,6-diamine;
N6-[(2R)-2-amino-2-phenyl-ethyl]-N4-tert-butyl-1-(trideuteriomethyl)pyrazolo[3,4-d]pyrimidine-4,6-diamine;
6-N-(2-amino-2-phenylethyl)-4-N-(cyclopropylmethyl)-1-methylpyrazolo[3,4-d]pyrimidine-4,6-diamine;
N6-(2-amino-2-phenyl-ethyl)-N4-(dicyclopropylmethyl)-1-methyl-pyrazolo[3,4-d]pyrimidine-4,6-diamine;
N6-[(2R)-2-amino-2-phenyl-ethyl]-1-methyl-N4-[(2-methylcyclopropyl)methyl]pyrazolo[3,4-d]pyrimidine-4,6-diamine;
N6-[(2R)-2-amino-2-phenyl-ethyl]-N4-(1-cyclopropyl-1-methyl-ethyl)-1-methyl-pyrazolo[3,4-d]pyrimidine-4,6-diamine;
N6-[(2R)-2-amino-2-phenyl-ethyl]-1-methyl-N4-(spiro[2.2]pentan-2-ylmethyl)pyrazolo[3,4-d]pyrimidine-4,6-diamine;
N6-[(2R)-2-amino-2-phenyl-ethyl]-N4-[(2,2-difluorocyclopropyl)methyl]-1-methyl-pyrazolo[3,4-d]pyrimidine-4,6-diamine;
N6-[(2R)-2-amino-2-phenyl-ethyl]-N4-[(2,2-dimethylcyclopropyl)methyl]-1-methyl-pyrazolo[3,4-d]pyrimidine-4,6-diamine;
6-N-(2-amino-2-phenylethyl)-4-N-(2-bicyclo[1.1.1]pentanyl)-1-methylpyrazolo[3,4-d]pyrimidine-4,6-diamine;
3-[[6-[(2-amino-2-phenylethyl)amino]-1-methylpyrazolo[3,4-d]pyrimidin-4-yl]amino]bicyclo[1.1.1]pentane-1-carbonitrile;
N6-(2-amino-2-phenylethyl)-N4-{3-fluorobicyclo[1.1.1]pentan-1-yl}-1-methyl-1H-pyrazolo[3,4-d]pyrimidine-4,6-diamine;
N6-[(2R)-2-amino-2-phenyl-ethyl]-N4-(3-bicyclo[1.1.1]pentanyl)-1-methyl-pyrazolo[3,4-d]pyrimidine-4,6-diamine;
N6-[(2R)-2-amino-2-phenyl-ethyl]-N4-(3-fluoro-1-bicyclo[1.1.1]pentanyl)-1-methyl-pyrazolo[3,4-d]pyrimidine-4,6-diamine;
6-N-(2-amino-2-phenylethyl)-4-N-(4,4-difluorocyclohexyl)-1-methylpyrazolo[3,4-d]pyrimidine-4,6-diamine;
N6-[(2R)-2-amino-2-phenyl-ethyl]-1-methyl-N4-tetrahydropyran-4-yl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]-3-methyl-butan-1-ol;
2-[[6-[[(2R)-2-amino-2-phenyl-ethyl]amino]-1-methyl-pyrazolo[3,4-d]pyrimidin-4-yl]amino]-2-methyl-propan-1-ol;
N6-[(2R)-2-amino-2-phenyl-ethyl]-N4-(2,2-dimethylpropyl)-1-methyl-pyrazolo[3,4-d]pyrimidine-4,6-diamine;
N6-[(2R)-2-amino-2-phenyl-ethyl]-N4-(2,2-difluoroethyl)-1-methyl-pyrazolo[3,4-d]pyrimidine-4,6-diamine;
N6-[(2R)-2-amino-2-phenyl-ethyl]-N4-(2,2-difluoropropyl)-1-methyl-pyrazolo[3,4-d]pyrimidine-4,6-diamine;
N6-[(2R)-2-amino-2-phenyl-ethyl]-1-methyl-N4-(2,2,2-trifluoro-1,1-dimethyl-ethyl)pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-(1-methylcyclopropyl)pyrazolo[3,4-d]pyrimidine-4,6-diamine;
N6-[(2R)-2-amino-2-phenyl-ethyl]-1-methyl-N4-[1-(trifluoromethyl)cyclopropyl]pyrazolo[3,4-d]pyrimidine-4,6-diamine;
N6-[(2R)-2-amino-2-phenyl-ethyl]-N4-(1-tert-butylcyclopropyl)-1-methyl-pyrazolo[3,4-d]pyrimidine-4,6-diamine;
N6-[(2R)-2-amino-2-phenyl-ethyl]-1-methyl-N4-spiro[2.2]pentan-2-yl-pyrazolo[3,4-d]pyrimidine-4,6-diamine;
[3-[[6-[(2-amino-2-phenyl-ethyl)amino]-1-methyl-pyrazolo[3,4-d]pyrimidin-4-yl]amino]-1-(chloromethyl)cyclobutyl]methanol;
N6-(2-amino-2-phenyl-ethyl)-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-N4-cyclobutyl-1-methyl-pyrazolo[3,4-d]pyrimidine-4,6-diamine;
N6-[(2R)-2-amino-2-phenyl-ethyl]-1-methyl-N4-(1-methylcyclobutyl)pyrazolo[3,4-d]pyrimidine-4,6-diamine;
N6-[(2R)-2-amino-2-phenyl-ethyl]-N4-(3,3-difluorocyclobutyl)-1-methyl-pyrazolo[3,4-d]pyrimidine-4,6-diamine;
N6-[(2R)-2-amino-2-phenyl-ethyl]-N4-(3,3-difluoro-1-methyl-cyclobutyl)-1-methyl-pyrazolo[3,4-d]pyrimidine-4,6-diamine;
N6-[(2R)-2-amino-2-phenyl-ethyl]-1-methyl-N4-(3-methylcyclobutyl)pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]cyclobutanecarbonitrile;
N6-[(2R)-2-amino-2-phenyl-ethyl]-N4-(3-methoxycyclobutyl)-1-methyl-pyrazolo[3,4-d]pyrimidine-4,6-diamine;
N6-[(2R)-2-amino-2-phenyl-ethyl]-N4-(3,3-dichlorocyclobutyl)-1-methyl-pyrazolo[3,4-d]pyrimidine-4,6-diamine;
N6-[(2R)-2-amino-2-phenyl-ethyl]-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-1-methyl-N4-(2-oxaspiro[3.3]heptan-6-yl)pyrazolo[3,4-d]pyrimidine-4,6-diamine;
N6-[(2R)-2-amino-2-phenyl-ethyl]-N4-(3,3-dimethylcyclobutyl)-1-methyl-pyrazolo[3,4-d]pyrimidine-4,6-diamine;
N6-[(2R)-2-amino-2-phenyl-ethyl]-N4-(3-fluoro-3-methyl-cyclobutyl)-1-methyl-pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-spiro[3.3]heptan-2-yl-pyrazolo[3,4-d]pyrimidine-4,6-diamine;
N6-[(2R)-2-amino-2-phenyl-ethyl]-N4-(2-methoxy-1,1-dimethyl-ethyl)-1-methyl-pyrazolo[3,4-d]pyrimidine-4,6-diamine;
N6-[(2R)-2-amino-2-phenyl-ethyl]-N4-(1,1-dimethylpropyl)-1-methyl-pyrazolo[3,4-d]pyrimidine-4,6-diamine;
N6-[(2R)-2-amino-2-phenyl-ethyl]-N4-[(1S)-1-cyclopropylethyl]-1-methyl-pyrazolo[3,4-d]pyrimidine-4,6-diamine:
N6-[(2R)-2-amino-2-phenyl-ethyl]-N4-isobutyl-1-methyl-pyrazolo[3,4-d]pyrimidine-4,6-diamine;
N6-[(2R)-2-amino-2-phenyl-ethyl]-1-methyl-N4-(spiro[2.3]hexan-2-ylmethyl)pyrazolo[3,4-d]pyrimidine-4,6-diamine;
N6-[(2R)-2-amino-2-phenyl-ethyl]-N4-(cyclobutylmethyl)-1-methyl-pyrazolo[3,4-d]pyrimidine-4,6-diamine;
N6-[(2R)-2-amino-2-phenyl-ethyl]-N4-[(2,2-difluorocyclobutyl)methyl]-1-methyl-pyrazolo[3,4-d]pyrimidine-4,6-diamine;
N6-[(2R)-2-amino-2-phenyl-ethyl]-N4-[(1-fluorocyclobutyl)methyl]-1-methyl-pyrazolo[3,4-d]pyrimidine-4,6-diamine;
N6-[(2R)-2-amino-2-phenyl-ethyl]-N4-[(3-fluorocyclobutyl)methyl]-1-methyl-pyrazolo[3,4-d]pyrimidine-4,6-diamine;
N6-[(2R)-2-amino-2-phenyl-ethyl]-1-methyl-N4-[3-(trifluoromethyl)cyclobutyl]pyrazolo[3,4-d]pyrimidine-4,6-diamine;
N6-[(2R)-2-amino-2-phenyl-ethyl]-N4-(2-fluoro-2-methyl-propyl)-1-methyl-pyrazolo[3,4-d]pyrimidine-4,6-diamine;
N6-[(2R)-2-amino-2-phenyl-ethyl]-1-methyl-N4-(1-methylcyclohexyl)pyrazolo[3,4-d]pyrimidine-4,6-diamine;
N6-[(2R)-2-amino-2-phenyl-ethyl]-N4-[(2-fluorocyclobutyl)methyl]-1-methyl-pyrazolo[3,4-d]pyrimidine-4,6-diamine;
N6-[(2R)-2-amino-2-phenyl-ethyl]-N4-cyclopentyl-1-methyl-pyrazolo[3,4-d]pyrimidine-4,6-diamine;
N6-[(2R)-2-amino-2-phenyl-ethyl]-1-methyl-N4-(3,3,3-trifluoropropyl)pyrazolo[3,4-d]pyrimidine-4,6-diamine;
N6-[(2R)-2-amino-2-phenyl-ethyl]-N4-[(2,2-dichlorocyclopropyl)methyl]-1-methyl-pyrazolo[3,4-d]pyrimidine-4,6-diamine;
N6-[(2R)-2-amino-2-phenyl-ethyl]-1-methyl-N4-(1-methylcyclopentyl)pyrazolo[3,4-d]pyrimidine-4,6-diamine;
N6-[(2R)-2-amino-2-phenyl-ethyl]-N4-[(1R)-2-methoxy-1-methyl-ethyl]-1-methyl-pyrazolo[3,4-d]pyrimidine-4,6-diamine;
N6-[(2R)-2-amino-2-phenyl-ethyl]-1-methyl-N4-(1-methyl-1-tetrahydropyran-4-yl-ethyl)pyrazolo[3,4-d]pyrimidine-4,6-diamine;
1-methyl-N6-[(2R)-2-amino-2-phenyl-ethyl]-N4-spiro[2.3]hexan-5-yl-pyrazolo[3,4-d]pyrimidine-4,6-diamine;
N6-[(2R)-2-amino-2-phenyl-ethyl]-N4-(2,2-dimethylcyclopropyl)-1-methyl-pyrazolo[3,4-d]pyrimidine-4,6-diamine;
N6-[(2R)-2-amino-2-phenyl-ethyl]-N4-tert-butyl-1-cyclopropyl-pyrazolo[3,4-d]pyrimidine-4,6-diamine;
6-N-[2-amino-2-(2-methoxyphenyl)ethyl]-4-N-[(3-chloro-4-methylphenyl)methyl]-1-methylpyrazolo[3,4-d]pyrimidine-4,6-diamine;
6-N-(2-Amino-2-phenylethyl)-4-N-[(3-chloro-4-methylphenyl)methyl]-1-methylpyrazolo[3,4-d]pyrimidine-4,6-diamine;
6-N-(2-amino-2-pyridin-2-ylethyl)-1-methyl-4-N-[4-(trifluoromethyl)phenyl]pyrazolo[3,4-d]pyrimidine-4,6-diamine;
3-[1-Amino-2-[[1-methyl-4-(methylamino)pyrazolo[3,4-d]pyrimidin-6-yl]amino]ethyl]benzonitrile;
N6-[2-Amino-2-(3-ethynylphenyl)ethyl]-N4-tert-butyl-1-methyl-pyrazolo[3,4-d]pyrimidine-4,6-diamine;
N6-[2-amino-2-(2-pyridyl)ethyl]-N4-tert-butyl-1-methyl-pyrazolo[3,4-d]pyrimidine-4,6-diamine;
6-N-(2-amino-2-pyridin-3-ylethyl)-4-N-[(3-chloro-4-methylphenyl)methyl]-1-methylpyrazolo[3,4-d]pyrimidine-4,6-diamine;
N6-[2-amino-2-(3-pyridyl)ethyl]-N4-tert-butyl-1-methyl-pyrazolo[3,4-d]pyrimidine-4,6-diamine;
6-N-[2-amino-2-(2-chlorophenyl)ethyl]-1-methyl-4-N-[4-(trifluoromethyl)phenyl]pyrazolo[3,4-d]pyrimidine-4,6-diamine;
N6-[2-amino-2-(2-chlorophenyl)ethyl]-N4-tert-butyl-1-methyl-pyrazolo[3,4-d]pyrimidine-4,6-diamine;
N6-[(2R)-2-amino-2-(2-chlorophenyl)ethyl]-N4-tert-butyl-1-methyl-pyrazolo[3,4-d]pyrimidine-4,6-diamine;
6-N-[2-amino-2-(2-fluorophenyl)ethyl]-4-N-[(3-chloro-4-methylphenyl)methyl]-1-methylpyrazolo[3,4-d]pyrimidine-4,6-diamine;
N6-[2-amino-2-(2-fluorophenyl)ethyl]-N4-tert-butyl-1-methyl-pyrazolo[3,4-d]pyrimidine-4,6-diamine;
N6-[2-amino-2-(2-fluorophenyl)ethyl]-N4-tert-butyl-1-(trideuteriomethyl)pyrazolo[3,4-d]pyrimidine-4,6-diamine;
N6-[(2R)-2-amino-2-(3-bromophenyl)ethyl]-N4-isopropyl-1-methyl-pyrazolo[3,4-d]pyrimidine-4,6-diamine;
N6-[(2R)-2-amino-2-(3-bromophenyl)ethyl]-1-methyl-N4-(2,2,2-trifluoro-1,1-dimethyl-ethyl)pyrazolo[3,4-d]pyrimidine-4,6-diamine;
6-N-[2-amino-2-(3-bromophenyl)ethyl]-4-N,1-dimethylpyrazolo[3,4-d]pyrimidine-4,6-diamine;
N6-[2-amino-2-(3-bromophenyl)ethyl]-N4-tert-butyl-1-methyl-pyrazolo[3,4-d]pyrimidine-4,6-diamine;
6-N-[2-amino-2-(4-bromophenyl)ethyl]-1-methyl-4-N-propan-2-ylpyrazolo[3,4-d]pyrimidine-4,6-diamine;
N6-[2-amino-2-(2-bromophenyl)ethyl]-N4-tert-butyl-1-methyl-pyrazolo[3,4-d]pyrimidine-4,6-diamine;
4-[1-amino-2-[[1-methyl-4-(methylamino)pyrazolo[3,4-d]pyrimidin-6-yl]amino]ethyl]benzonitrile;
3-[1-amino-2-[[4-(tert-butylamino)-1-methyl-pyrazolo[3,4-d]pyrimidin-6-yl]amino]ethyl]benzonitrile;
3-[(1R)-1-amino-2-[[1-methyl-4-[(2,2,2-trifluoro-1,1-dimethyl-ethyl)amino]pyrazolo[3,4-d]pyrimidin-6-yl]amino]ethyl]benzonitrile;
N6-[2-amino-2-(3-pyridyl)ethyl]-N4-tert-butyl-1-methyl-pyrazolo[3,4-d]pyrimidine-4,6-diamine;
N6-[2-amino-2-(3-pyridyl)ethyl]-N4-(3,3-difluorocyclobutyl)-1-methyl-pyrazolo[3,4-d]pyrimidine-4,6-diamine;
N6-[2-amino-2-(4-pyridyl)ethyl]-N4-tert-butyl-1-methyl-pyrazolo[3,4-d]pyrimidine-4,6-diamine;
N6-[2-amino-2-(4-pyridyl)ethyl]-N4-(3,3-difluorocyclobutyl)-1-methyl-pyrazolo[3,4-d]pyrimidine-4,6-diamine;
N′-(4-Ethoxy-1-methylpyrazolo[3,4-d]pyrimidin-6-yl)-1-phenylethane-1,2-diamine;
N′-(1-methyl-4-propan-2-yloxypyrazolo[3,4-d]pyrimidin-6-yl)-1-phenylethane-1,2-diamine;
N′-(4-tert-butoxy-1-methyl-pyrazolo[3,4-d]pyrimidin-6-yl)-1-phenyl-ethane-1,2-diamine;
1-methyl-N6-{[(3R)-morpholin-3-yl]methyl}-N4-[4-(trifluoromethyl)phenyl]-1H-pyrazolo[3,4-d]pyrimidine-4,6-diamine;
6-N-[(1S,2R)-2-aminocyclohexyl]-4-N-[(3-chloro-4-methylphenyl)methyl]-1-methylpyrazolo[3,4-d]pyrimidine-4,6-diamine;
6-N-[(1R,2R)-2-aminocyclohexyl]-4-N-[(3-chloro-4-methylphenyl)methyl]-1-methylpyrazolo[3,4-d]pyrimidine-4,6-diamine;
N6-[(2R)-2-amino-2-phenyl-ethyl]-N4-cyclopropyl-1-methyl-pyrazolo[3,4-d]pyrimidine-4,6-diamine;
N6-[(2R)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-1-methyl-N4-(2,2,2-trifluoroethyl)pyrazolo[3,4-d]pyrimidine-4,6-diamine;
N6-[(2R)-2-amino-2-phenyl-ethyl]-1-methyl-N4-[6-(trifluoromethyl)-3-pyridyl]pyrazolo[3,4-d]pyrimidine-4,6-diamine;
N6-[(2S)-2-amino-2-phenyl-ethyl]-1-methyl-N4-[6-(trifluoromethyl)-3-pyridyl]pyrazolo[3,4-d]pyrimidine-4,6-diamine;
rel-6-N-[(2S)-2-amino-2-phenylpropyl]-4-N,1-dimethylpyrazolo[3,4-d]pyrimidine-4,6-diamine;
N6-[(2S)-2-amino-2-(2-fluorophenyl)ethyl]-N4-tert-butyl-1-methyl-pyrazolo[3,4-d]pyrimidine-4,6-diamine;
N6-[(2R)-2-amino-2-(2-fluorophenyl)ethyl]-N4-tert-butyl-1-methyl-pyrazolo[3,4-d]pyrimidine-4,6-diamine;
rel-N6-[(2R)-2-amino-2-phenyl-propyl]-1-methyl-N4-[6-(trifluoromethyl)-3-pyridyl]pyrazolo[3,4-d]pyrimidine-4,6-diamine;
6-N-[(2R)-2-amino-3-fluoropropyl]-4-N-[(3-chloro-4-methylphenyl)methyl]-1-methylpyrazolo[3,4-d]pyrimidine-4,6-diamine;
rel-N6-[(2S)-2-amino-2-phenyl-propyl]-1-methyl-N4-[6-(trifluoromethyl)-3-pyridyl]pyrazolo[3,4-d]pyrimidine-4,6-diamine;
N6-[(2R)-2-amino-2-(2-fluorophenyl)ethyl]-N4-tert-butyl-1-(trideuteriomethyl)pyrazolo[3,4-d]pyrimidine-4,6-diamine; and
N6-[(2S)-2-amino-2-(2-fluorophenyl)ethyl]-N4-tert-butyl-1-(trideuteriomethyl)pyrazolo[3,4-d]pyrimidine-4,6-diamine.

Particular compounds of the present invention include any of the compounds exemplified in the present application, or a pharmaceutically acceptable salt thereof, and, in particular, any of the following:
N6-(2-Aminoethyl)-1-methyl-N4-[4-(trifluoromethyl)phenyl]pyrazolo[3,4-d]pyrimidine-4,6-diamine;
N6-(2-aminoethyl)-1-methyl-N4-[3-(trifluoromethyl)phenyl]-1H-pyrazolo[3,4-d]pyrimidine-4,6-diamine;
N6-(2-aminoethyl)-1-methyl-N4-(4-phenoxyphenyl)-1H-pyrazolo[3,4-d]pyrimidine-4,6-diamine;
N6-(2-aminoethyl)-1-methyl-N4-(3-phenoxyphenyl)-1H-pyrazolo[3,4-d]pyrimidine-4,6-diamine;
N6-(2-aminoethyl)-1-methyl-N4-[(4-phenylphenyl)methyl]pyrazolo[3,4-d]pyrimidine-4,6-diamine;
N6-(2-aminoethyl)-N4-[1-(3-chloro-4-methoxyphenyl)ethyl]-1-methyl-1H-pyrazolo[3,4-d]pyrimidine-4,6-diamine;
N6-(2-aminoethyl)-N4-[2-(6-methoxypyridin-3-yl)ethyl]-1-methyl-1H-pyrazolo[3,4-d]pyrimidine-4,6-diamine;
N6-(2-aminoethyl)-N4-[2-(3-chloro-4-methoxyphenyl)ethyl]-1-methyl-1H-pyrazolo[3,4-d]pyrimidine-4,6-diamine;
N6-(2-aminoethyl)-N4-[2-(2H-1,3-benzodioxol-5-yl)ethyl]-1-methyl-1H-pyrazolo[3,4-d]pyrimidine-4,6-diamine:
N6-(2-aminoethyl)-N4-[(1-benzofuran-5-yl)methyl]-1-methyl-1H-pyrazolo[3,4-d]pyrimidine-4,6-diamine;
N6-(2-aminoethyl)-N4-{3′-methoxy-[1,1′-biphenyl]-3-yl}-1-methyl-1H-pyrazolo[3,4-d]pyrimidine-4,6-diamine;
N6-(2-aminoethyl)-N4-{[1-(benzenesulfonyl)piperidin-4-yl]methyl}-1-methyl-1H-pyrazolo[3,4-d]pyrimidine-4,6-diamine;
N6-(2-aminoethyl)-N4-[(1H-1,3-benzodiazol-5-yl)methyl]-1-methyl-1H-pyrazolo[3,4-d]pyrimidine-4,6-diamine;
N6-(2-aminoethyl)-N4-{3′-methoxy-[1,1′-biphenyl]-4-yl}-1-methyl-1H-pyrazolo[3,4-d]pyrimidine-4,6-diamine;
N6-(2-aminoethyl)-N4-{4′-methoxy-[1,1′-biphenyl]-3-yl}-1-methyl-1H-pyrazolo[3,4-d]pyrimidine-4,6-diamine;
N6-(2-aminoethyl)-N4-{[1,1′-biphenyl]-3-yl}-1-methyl-1H-pyrazolo[3,4-d]pyrimidine-4,6-diamine;
N6-(2-aminoethyl)-N4-[(3-chloro-4-fluorophenyl)methyl]-1-methyl-1H-pyrazolo[3,4-d]pyrimidine-4,6-diamine;
N6-(2-aminoethyl)-N4-[(4-chloro-2-fluorophenyl)methyl]-1-methyl-1H-pyrazolo[3,4-d]pyrimidine-4,6-diamine;
N6-(2-aminoethyl)-N4-[(2,4-difluorophenyl)methyl]-1-methyl-1H-pyrazolo[3,4-d]pyrimidine-4,6-diamine;
N6-(2-aminoethyl)-N4-[(3-chloro-5-methoxyphenyl)methyl]-1-methyl-1H-pyrazolo[3,4-d]pyrimidine-4,6-diamine;
N6-(2-aminoethyl)-N4-(3-chloro-4-methoxyphenyl)-1-methyl-1H-pyrazolo[3,4-d]pyrimidine-4,6-diamine;
N6-(2-aminoethyl)-N4-[(3-chloro-4-methylphenyl)methyl]-1-methyl-1H-pyrazolo[3,4-d]pyrimidine-4,6-diamine;
N6-(2-aminoethyl)-1-methyl-N4-{[3-(trifluoromethyl)phenyl]methyl}-1H-pyrazolo[3,4-d]pyrimidine-4,6-diamine;
N6-(2-aminoethyl)-N4-[(2,4-dimethoxyphenyl)methyl]-1-methyl-1H-pyrazolo[3,4-d]pyrimidine-4,6-diamine;
N6-(2-aminoethyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(4-methoxyphenyl)methyl]-1-methyl-1H-pyrazolo[3,4-d]pyrimidine-4,6-diamine:
N6-(2-amino-2-methylpropyl)-N4-({[1,1′-biphenyl]-4-yl}methyl)-1-methyl-1H-pyrazolo[3,4-d]pyrimidine-4,6-diamine;
N6-(2-aminoethyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-({[1,1′-biphenyl]-4-yl}methyl)-1,3-dimethyl-1H-pyrazolo[3,4-d]pyrimidine-4,6-diamine;
N6-[(1-aminocyclohexyl)methyl]-1-methyl-N4-[4-(trifluoromethyl)phenyl]pyrazolo[3,4-d]pyrimidine-4,6-diamine;
N6-(2-amino-2-methylpropyl)-1,3-dimethyl-N4-[3-(trifluoromethyl)phenyl]-1H-pyrazolo[3,4-d]pyrimidine-4,6-diamine;
6-N-(2-aminocyclohexyl)-4-N-[(3-chloro-4-methylphenyl)methyl]-1-methylpyrazolo[3,4-d]pyrimidine-4,6-diamine;
6-N-[(4-aminooxan-4-yl)methyl]-1-methyl-4-N-[6-(trifluoromethyl)pyridin-3-yl]pyrazolo[3,4-d]pyrimidine-4,6-diamine;
N6-[(3R,4S)-4-aminotetrahydrofuran-3-yl]-1-methyl-N4-[4-(trifluoromethyl)phenyl]pyrazolo[3,4-d]pyrimidine-4,6-diamine;
N6-(2-aminoethyl)-N4-[(4-methoxyphenyl)methyl]-1-methyl-1H-pyrazolo[3,4-d]pyrimidine-4,6-diamine;
tert-butyl 4-[({6-[(2-aminoethyl)amino]-1-methyl-1H-pyrazolo[3,4-d]pyrimidin-4-yl}amino)methyl]piperidine-1-carboxylate;
N6-(2-aminoethyl)-N4-[(1-benzylpiperidin-4-yl)methyl]-1-methyl-1H-pyrazolo[3,4-d]pyrimidine-4,6-diamine;
N6-(2-aminoethyl)-N4-[(1-benzoylpiperidin-4-yl)methyl]-1-methyl-1H-pyrazolo[3,4-d]pyrimidine-4,6-diamine;
N6-(2-aminoethyl)-N4-{[1-(3-chloro-4-methoxybenzoyl)piperidin-4-yl]methyl}-1-methyl-1H-pyrazolo[3,4-d]pyrimidine-4,6-diamine;
6-N-(2-Amino-2-methylpropyl)-1,3-dimethyl-4-N-[[3-(trifluoromethyl)phenyl]methyl]pyrazolo[3,4-b]pyridine-4,6-diamine;
6-N-[(1-Aminocyclopropyl)methyl]-4-N-[(3-chloro-4-methylphenyl)methyl]-1-methylpyrazolo[3,4-d]pyrimidine-4,6-diamine;
6-N-[(1-Aminocyclopropyl)methyl]-4-N-[(3-chloro-4-fluorophenyl)methyl]-1-methylpyrazolo[3,4-d]pyrimidine-4,6-diamine;
N6-[(1-aminocyclopropyl)methyl]-N4-[3-fluoro-4-(trifluoromethyl)phenyl]-1-methyl-pyrazolo[3,4-d]pyrimidine-4,6-diamine;
6-N-[(1-aminocyclopropyl)methyl]-4-N-(3-chloro-4-methoxyphenyl)-1-methylpyrazolo[3,4-d]pyrimidine-4,6-diamine;
6-N-[(1-aminocyclopropyl)methyl]-4-N-(2,4-difluorophenyl)-1-methylpyrazolo[3,4-d]pyrimidine-4,6-diamine;
6-N-[(1-aminocyclopropyl)methyl]-1-methyl-4-N-[3-(trifluoromethyl)phenyl]pyrazolo[3,4-d]pyrimidine-4,6-diamine;
6-N-[(1-aminocyclopropyl)methyl]-4-N-[3-(difluoromethyl)phenyl]-1-methylpyrazolo[3,4-d]pyrimidine-4,6-diamine;
6-N-[(1-aminocyclopropyl)methyl]-4-N-(4-fluorophenyl)-1-methylpyrazolo[3,4-d]pyrimidine-4,6-diamine;
6-N-[(1-aminocyclopropyl)methyl]-4-N-(3-chloro-4-methylphenyl)-1-methylpyrazolo[3,4-d]pyrimidine-4,6-diamine;
6-N-[(1-aminocyclopropyl)methyl]-1-methyl-4-N-pyridin-3-ylpyrazolo[3,4-d]pyrimidine-4,6-diamine;
6-N-[(1-aminocyclopropyl)methyl]-1-methyl-4-N-phenylpyrazolo[3,4-d]pyrimidine-4,6-diamine;
6-N-[(1-aminocyclopropyl)methyl]-4-N-[(3-bromophenyl)methyl]-1-methylpyrazolo[3,4-d]pyrimidine-4,6-diamine;
6-N-[(1-aminocyclopropyl)methyl]-4-N-[(4-bromophenyl)methyl]-1-methylpyrazolo[3,4-d]pyrimidine-4,6-diamine;
6-N-[(1-aminocyclopropyl)methyl]-4-N-(2-chloro-4-fluorophenyl)-1-methylpyrazolo[3,4-d]pyrimidine-4,6-diamine;
6-N-[(1-aminocyclopropyl)methyl]-4-N-(4-chloro-2-fluorophenyl)-1-methylpyrazolo[3,4-d]pyrimidine-4,6-diamine;
6-N-[(1-aminocyclopropyl)methyl]-4-N-(3-chloro-5-fluorophenyl)-1-methylpyrazolo[3,4-d]pyrimidine-4,6-diamine;
5-[[6-[(1-aminocyclopropyl)methylamino]-1-methyl-pyrazolo[3,4-d]pyrimidin-4-yl]amino]-2-chloro-benzonitrile;
N6-[(1-aminocyclopropyl)methyl]-1-methyl-N4-(1-phenyl-3-bicyclo[1.1.1]pentanyl)pyrazolo[3,4-d]pyrimidine-4,6-diamine;
6-N-[(1-aminocyclopropyl)methyl]-1-methyl-4-N-[[6-(trifluoromethyl)pyridin-3-yl]methyl]pyrazolo[3,4-d]pyrimidine-4,6-diamine;
6-N-[(1-aminocyclopropyl)methyl]-4-N-(4-chloro-3,5-difluorophenyl)-1-methylpyrazolo[3,4-d]pyrimidine-4,6-diamine;
6-N-[(1-aminocyclopropyl)methyl]-4-N-[3-(difluoromethoxy)phenyl]-1-methylpyrazolo[3,4-d]pyrimidine-4,6-diamine:
6-N-[(1-aminocyclopropyl)methyl]-1-methyl-4-N-(4-propan-2-yloxyphenyl)pyrazolo[3,4-d]pyrimidine-4,6-diamine;
6-N-[(1-aminocyclopropyl)methyl]-4-N-[(4-chloro-3-fluorophenyl)methyl]-1-methylpyrazolo[3,4-d]pyrimidine-4,6-diamine;
6-N-[(1-aminocyclopropyl)methyl]-4-N-(2,3-difluorophenyl)-1-methylpyrazolo[3,4-d]pyrimidine-4,6-diamine;
6-N-[(1-aminocyclopropyl)methyl]-4-N-(2,5-difluorophenyl)-1-methylpyrazolo[3,4-d]pyrimidine-4,6-diamine;
6-N-[(1-aminocyclopropyl)methyl]-4-N-[(2,4-difluorophenyl)methyl]-1-methylpyrazolo[3,4-d]pyrimidine-4,6-diamine;
6-N-[(1-aminocyclopropyl)methyl]-4-N-(3-chloro-4-fluorophenyl)-1-methylpyrazolo[3,4-d]pyrimidine-4,6-diamine;
6-N-[(1-aminocyclopropyl)methyl]-4-N-(4-chlorophenyl)-1-methylpyrazolo[3,4-d]pyrimidine-4,6-diamine;
6-N-[(1-aminocyclopropyl)methyl]-4-N-(3-fluorophenyl)-1-methylpyrazolo[3,4-d]pyrimidine-4,6-diamine;
6-N-[(1-aminocyclopropyl)methyl]-1,3-dimethyl-4-N-[4-(trifluoromethyl)phenyl]pyrazolo[3,4-d]pyrimidine-4,6-diamine;
4-[[6-[(1-aminocyclopropyl)methylamino]-1-methylpyrazolo[3,4-d]pyrimidin-4-yl]amino]benzonitrile;
6-N-[(1-aminocyclopropyl)methyl]-1-methyl-4-N-[4-(trifluoromethoxy)phenyl]pyrazolo[3,4-d]pyrimidine-4,6-diamine;
6-N-[(1-aminocyclopropyl)methyl]-4-N-(4-methoxyphenyl)-1-methylpyrazolo[3,4-d]pyrimidine-4,6-diamine;
6-N-[(1-aminocyclopropyl)methyl]-4-N-[4-(difluoromethoxy)phenyl]-1-methylpyrazolo[3,4-d]pyrimidine-4,6-diamine;
6-N-[(1-aminocyclopropyl)methyl]-4-N-(3-chlorophenyl)-1-methylpyrazolo[3,4-d]pyrimidine-4,6-diamine;
6-N-[(1-aminocyclopropyl)methyl]-4-N-[4-(difluoromethyl)phenyl]-1-methylpyrazolo[3,4-d]pyrimidine-4,6-diamine;
3-[[6-[(1-aminocyclopropyl)methylamino]-1-methylpyrazolo[3,4-d]pyrimidin-4-yl]amino]bicyclo[1.1.1]pentane-1-carbonitrile;
6-N-[(1-aminocyclopropyl)methyl]-4-N-(3-bicyclo[1.1.1]pentanyl)-1-methylpyrazolo[3,4-d]pyrimidine-4,6-diamine;
6-N-[(1-aminocyclopropyl)methyl]-4-N-(3,4-difluorophenyl)-1-methylpyrazolo[3,4-d]pyrimidine-4,6-diamine;
6-N-[(1-aminocyclopropyl)methyl]-4-N-(3,4-dichlorophenyl)-1-methylpyrazolo[3,4-d]pyrimidine-4,6-diamine;
6-N-[(1-aminocyclopropyl)methyl]-4-N-(4-chloro-3-methoxyphenyl)-1-methylpyrazolo[3,4-d]pyrimidine-4,6-diamine;
N6-[(1-aminocyclopropyl)methyl]-N4-[(2,4-dimethoxyphenyl)methyl]-1-methyl-pyrazolo[3,4-d]pyrimidine-4,6-diamine;
N6-[(1-aminocyclopropyl)methyl]-N4-{3-fluorobicyclo[1.1.1]pentan-1-yl}-1-methyl-1H-pyrazolo[3,4-d]pyrimidine-4,6-diamine;
6-N-[(1-aminocyclopropyl)methyl]-1-methyl-4-N-[4-(trifluoromethyl)phenyl]pyrazolo[3,4-d]pyrimidine-4,6-diamine;
6-N-[(1-aminocyclopropyl)methyl]-1-methyl-4-N-[6-(trifluoromethyl)pyridin-3-yl]pyrazolo[3,4-d]pyrimidine-4,6-diamine;
6-N-[(1-aminocyclopropyl)methyl]-4-N-(5-chloropyridin-2-yl)-1-methylpyrazolo[3,4-d]pyrimidine-4,6-diamine;
N6-[(1-aminocyclopropyl)methyl]-1-methyl-N4-[5-(trifluoromethyl)pyrazin-2-yl]pyrazolo[3,4-d]pyrimidine-4,6-diamine;
6-N-[(1-aminocyclopropyl)methyl]-1-ethyl-4-N-[4-(trifluoromethyl)phenyl]pyrazolo[3,4-d]pyrimidine-4,6-diamine;
N6-[(1-aminocyclopropyl)methyl]-1-(trideuteriomethyl)-N4-[4-(trifluoromethyl)phenyl]pyrazolo[3,4-d]pyrimidine-4,6-diamine;
N6-[(1-aminocyclopropyl)methyl]-1-(trideuteriomethyl)-N4-[6-(trifluoromethyl)-3-pyridyl]pyrazolo[3,4-d]pyrimidine-4,6-diamine;
6-N-[(1-aminocyclopropyl)methyl]-4-N-[[4-[2-(3-but-3-ynyldiazirin-3-yl)ethoxy]-3-chlorophenyl]methyl]-1-methylpyrazolo[3,4-d]pyrimidine-4,6-diamine;
Compound 2.52: N6-[(1-Aminocyclopropyl)methyl]-1-methyl-N4-[4-(trifluoromethyl)phenyl]pyrazolo[3,4-b]pyridine-4,6-diamine;
6-N-(2-aminopropyl)-4-N-[(3-chloro-4-methylphenyl)methyl]-1-methylpyrazolo[3,4-d]pyrimidine-4,6-diamine;
6-N-(1-aminopropan-2-yl)-4-N-[(3-chloro-4-methylphenyl)methyl]-1-methylpyrazolo[3,4-d]pyrimidine-4,6-diamine;
6-N-[(2S)-2-aminopropyl]-4-N-[(3-chloro-4-methylphenyl)methyl]-1-methylpyrazolo[3,4-d]pyrimidine-4,6-diamine;
6-N-[(2R)-2-aminopropyl]-4-N-[(3-chloro-4-methylphenyl)methyl]-1-methylpyrazolo[3,4-d]pyrimidine-4,6-diamine;
6-N-[(2S)-1-aminobutyl]-4-N-[(3-chloro-4-methylphenyl)methyl]-1-methylpyrazolo[3,4-d]pyrimidine-4,6-diamine;
6-N-[(2S)-2-aminobutyl]-4-N-[(3-chloro-4-methylphenyl)methyl]-1-methylpyrazolo[3,4-d]pyrimidine-4,6-diamine;
6-N-[(2R)-2-aminobutyl]-4-N-[(3-chloro-4-methylphenyl)methyl]-1-methylpyrazolo[3,4-d]pyrimidine-4,6-diamine;
N6-[(1-aminocyclobutyl)methyl]-1-methyl-N4-[4-(trifluoromethyl)phenyl]pyrazolo[3,4-d]pyrimidine-4,6-diamine;
N6-[(1-aminocyclobutyl)methyl]-N4-[4-(difluoromethoxy)phenyl]-1-methyl-pyrazolo[3,4-d]pyrimidine-4,6-diamine;
N6-(2-amino-2-cyclohexyl-ethyl)-N4-tert-butyl-1-methyl-pyrazolo[3,4-d]pyrimidine-4,6-diamine;
N6-(2-amino-2-cyclohexyl-ethyl)-1-methyl-N4-[4-(trifluoromethyl)phenyl]pyrazolo[3,4-d]pyrimidine-4,6-diamine;
6-N-[(1-amino-3,3-difluorocyclobutyl)methyl]-1-methyl-4-N-[4-(trifluoromethyl)phenyl]pyrazolo[3,4-d]pyrimidine-4,6-diamine;
6-N-(2-amino-2-cyclopropylethyl)-1-methyl-4-N-[4-(trifluoromethyl)phenyl]pyrazolo[3,4-d]pyrimidine-4,6-diamine;
6-N-[(3-aminooxolan-3-yl)methyl]-1-methyl-4-N-[4-(trifluoromethyl)phenyl]pyrazolo[3,4-d]pyrimidine-4,6-diamine;
N6-[(3-aminotetrahydrofuran-3-yl)methyl]-1-methyl-N4-[6-(trifluoromethyl)-3-pyridyl]pyrazolo[3,4-d]pyrimidine-4,6-diamine;
1-methyl-N6-[(morpholin-3-yl)methyl]-N4-[4-(trifluoromethyl)phenyl]-1H-pyrazolo[3,4-d]pyrimidine-4,6-diamine;
4-N-[(3-chloro-4-methylphenyl)methyl]-1-methyl-6-N-[[(2R)-pyrrolidin-2-yl]methyl]pyrazolo[3,4-d]pyrimidine-4,6-diamine;
6-N-[(1-aminocyclopentyl)methyl]-1-methyl-4-N-[4-(trifluoromethyl)phenyl]pyrazolo[3,4-d]pyrimidine-4,6-diamine;
6-N-[(1-aminocyclopentyl)methyl]-1-methyl-4-N-[6-(trifluoromethyl)pyridin-3-yl]pyrazolo[3,4-d]pyrimidine-4,6-diamine;
6-N-[(1-aminocyclopentyl)methyl]-4-N-[4-(difluoromethoxy)phenyl]-1-methylpyrazolo[3,4-d]pyrimidine-4,6-diamine;
6-N-[(1-aminocyclopentyl)methyl]-1-methyl-4-N-phenylpyrazolo[3,4-d]pyrimidine-4,6-diamine
4-[[6-[(1-aminocyclopentyl)methylamino]-1-methylpyrazolo[3,4-d]pyrimidin-4-yl]amino]benzonitrile;
6-N-[(1-aminocyclopentyl)methyl]-1-methyl-4-N-[4-(trifluoromethoxy)phenyl]pyrazolo[3,4-d]pyrimidine-4,6-diamine;
N6-[(1-aminocyclobutyl)methyl]-1-(trideuteriomethyl)-N4-[4-(trifluoromethyl)phenyl]pyrazolo[3,4-d]pyrimidine-4,6-diamine;
N6-(2-aminocyclohexyl)-N4-[(3-chloro-4-methylphenyl)methyl]-1-methyl-1H-pyrazolo[3,4-d]pyrimidine-4,6-diamine;
N6-(2-Amino-3-fluoropropyl)-N4-[(3-chloro-4-methylphenyl)methyl]-1-methyl-1H-pyrazolo[3,4-d]pyrimidine-4,6-diamine;
6-N-(2-amino-3-fluoropropyl)-1-methyl-4-N-[4-(trifluoromethyl)phenyl]pyrazolo[3,4-d]pyrimidine-4,6-diamine;
6-N-(2-amino-3-methoxypropyl)-4-N-[(3-chloro-4-methylphenyl)methyl]-1-methylpyrazolo[3,4-d]pyrimidine-4,6-diamine;
6-N-[(3-Aminooxetan-3-yl)methyl]-1-methyl-4-N-[4-(trifluoromethyl)phenyl]pyrazolo[3,4-d]pyrimidine-4,6-diamine;
6-N-[(3-Aminooxetan-3-yl)methyl]-1-methyl-4-N-phenylpyrazolo[3,4-d]pyrimidine-4,6-diamine;
N6-(2-Amino-2-phenyl-propyl)-N4-tert-butyl-1-(trideuteriomethyl)pyrazolo[3,4-d]pyrimidine-4,6-diamine;
6-N-(2-Amino-2-phenylethyl)-1-ethyl-4-N-propan-2-ylpyrazolo[3,4-d]pyrimidine-4,6-diamine;
6-N-(2-amino-2-phenylethyl)-4-N-[(3-chloro-4-methylphenyl)methyl]-1-methylpyrazolo[3,4-d]pyrimidine-4,6-diamine;
6-N-(2-amino-2-phenylethyl)-4-N-[(2,4-difluorophenyl)methyl]-1-methylpyrazolo[3,4-d]pyrimidine-4,6-diamine;
6-N-(2-amino-2-phenylethyl)-4-N-[(3-chloro-4-fluorophenyl)methyl]-1-methylpyrazolo[3,4-d]pyrimidine-4,6-diamine;
6-N-(2-amino-2-phenylethyl)-4-N-[(3-bromophenyl)methyl]-1-methylpyrazolo[3,4-d]pyrimidine-4,6-diamine;
6-N-(2-amino-2-phenylethyl)-4-N-[(4-bromophenyl)methyl]-1-methylpyrazolo[3,4-d]pyrimidine-4,6-diamine;
6-N-(2-amino-2-phenylethyl)-1-methyl-4-N-[[6-(trifluoromethyl)pyridin-3-yl]methyl]pyrazolo[3,4-d]pyrimidine-4,6-diamine;
N6-(2-amino-2-phenyl-ethyl)-N4-[(2,4-dimethoxyphenyl)methyl]-1-methyl-pyrazolo[3,4-d]pyrimidine-4,6-diamine;
6-N-(2-amino-2-phenylethyl)-4-N-(4-fluorophenyl)-1-methylpyrazolo[3,4-d]pyrimidine-4,6-diamine;
N6-(2-amino-2-phenyl-propyl)-1-methyl-N4-[2-(trifluoromethyl)pyrimidin-5-yl]pyrazolo[3,4-d]pyrimidine-4,6-diamine;
N6-(2-amino-2-phenyl-propyl)-1-methyl-N4-[4-(trifluoromethoxy)phenyl]pyrazolo[3,4-d]pyrimidine-4,6-diamine;
N6-(2-amino-2-phenyl-propyl)-N4-(3,4-dichlorophenyl)-1-methyl-pyrazolo[3,4-d]pyrimidine-4,6-diamine;
6-N-(2-amino-2-phenylethyl)-4-N-[4-(difluoromethoxy)phenyl]-1-methylpyrazolo[3,4-d]pyrimidine-4,6-diamine;
6-N-(2-amino-2-phenylpropyl)-4-N-[4-(difluoromethoxy)phenyl]-1-methylpyrazolo[3,4-d]pyrimidine-4,6-diamine;
N6-[(2R)-2-amino-2-phenyl-ethyl]-N4-[4-(difluoromethoxy)phenyl]-1-methyl-pyrazolo[3,4-d]pyrimidine-4,6-diamine;
6-N-(2-amino-2-phenylethyl)-1-methyl-4-N-[4-(trifluoromethyl)phenyl]pyrazolo[3,4-d]pyrimidine-4,6-diamine;
6-N-(2-amino-2-phenylethyl)-1,3-dimethyl-4-N-[4-(trifluoromethyl)phenyl]pyrazolo[3,4-d]pyrimidine-4,6-diamine;
N6-(2-amino-2-phenyl-propyl)-1-methyl-N4-[4-(trifluoromethyl)phenyl]pyrazolo[3,4-d]pyrimidine-4,6-diamine;
N6-(2-amino-2-phenyl-ethyl)-1-(trideuteriomethyl)-N4-[4-(trifluoromethyl)phenyl]pyrazolo[3,4-d]pyrimidine-4,6-diamine;
N6-(2-amino-2-phenyl-propyl)-1-(trideuteriomethyl)-N4-[4-(trifluoromethyl)phenyl]pyrazolo[3,4-d]pyrimidine-4,6-diamine;
6-N-[(2R)-2-amino-2-phenylethyl]-1-methyl-4-N-[4-(trifluoromethyl)phenyl]pyrazolo[3,4-d]pyrimidine-4,6-diamine;
6-N-[(2S)-2-amino-2-phenylethyl]-1-methyl-4-N-[4-(trifluoromethyl)phenyl]pyrazolo[3,4-d]pyrimidine-4,6-diamine;
N6-[(2R)-2-amino-2-phenyl-ethyl]-1-(trideuteriomethyl)-N4-[4-(trifluoromethyl)phenyl]pyrazolo[3,4-d]pyrimidine-4,6-diamine;
6-N-(2-amino-2-phenylethyl)-1-methyl-4-N-[6-(trifluoromethyl)pyridin-3-yl]pyrazolo[3,4-d]pyrimidine-4,6-diamine;
6-N-(2-amino-2-phenylpropyl)-1-methyl-4-N-[6-(trifluoromethyl)pyridin-3-yl]pyrazolo[3,4-d]pyrimidine-4,6-diamine;
N6-(2-amino-2-phenyl-propyl)-1-(trideuteriomethyl)-N4-[6-(trifluoromethyl)-3-pyridyl]pyrazolo[3,4-d]pyrimidine-4,6-diamine;
N6-(2-amino-2-phenyl-ethyl)-N4-[6-(difluoromethoxy)-3-pyridyl]-1-methyl-pyrazolo[3,4-d]pyrimidine-4,6-diamine;
N6-(2-amino-2-phenyl-propyl)-N4-[6-(difluoromethoxy)-3-pyridyl]-1-methyl-pyrazolo[3,4-d]pyrimidine-4,6-diamine;
N6-[(2R)-2-amino-2-phenyl-ethyl]-N4-(4-chlorophenyl)-1-methyl-pyrazolo[3,4-d]pyrimidine-4,6-diamine;
N6-[(2S)-2-amino-2-phenyl-ethyl]-N4-(4-chlorophenyl)-1-methyl-pyrazolo[3,4-d]pyrimidine-4,6-diamine;
N6-[(2R)-2-amino-2-phenyl-ethyl]-N4-(4-methoxyphenyl)-1-methyl-pyrazolo[3,4-d]pyrimidine-4,6-diamine;
N6-[(2R)-2-amino-2-phenyl-ethyl]-N4-(4-isopropoxyphenyl)-1-methyl-pyrazolo[3,4-d]pyrimidine-4,6-diamine;
N6-[(2R)-2-amino-2-phenyl-ethyl]-N4-(3,4-difluorophenyl)-1-methyl-pyrazolo[3,4-d]pyrimidine-4,6-diamine;
N6-[(2R)-2-amino-2-phenyl-ethyl]-N4-[4-(dimethylamino)phenyl]-1-methyl-pyrazolo[3,4-d]pyrimidine-4,6-diamine;
6-N-(2-amino-2-phenylethyl)-4-N,1-dimethylpyrazolo[3,4-d]pyrimidine-4,6-diamine;
6-N-(2-amino-2-phenylethyl)-1-methyl-4-N-(trideuteriomethyl)pyrazolo[3,4-d]pyrimidine-4,6-diamine;
6-N-(2-amino-2-phenylethyl)-4-N-methyl-1-(trideuteriomethyl)pyrazolo[3,4-d]pyrimidine-4,6-diamine;
6-N-(2-amino-2-phenylpropyl)-4-N,1-dimethylpyrazolo[3,4-d]pyrimidine-4,6-diamine;
6-N-[(2R)-2-amino-2-phenylethyl]-4-N,1-dimethylpyrazolo[3,4-d]pyrimidine-4,6-diamine;
6-N-(2-amino-2-phenylethyl)-4-N-ethyl-1-methylpyrazolo[3,4-d]pyrimidine-4,6-diamine;
6-N-(2-amino-2-phenylethyl)-4-N-ethyl-4-N,1-dimethylpyrazolo[3,4-d]pyrimidine-4,6-diamine;
6-N-(2-amino-2-phenylethyl)-4-N-cyclopropyl-1-methylpyrazolo[3,4-d]pyrimidine-4,6-diamine;
6-N-(2-amino-2-phenylethyl)-1-methyl-4-N-(2,2,2-trifluoroethyl)pyrazolo[3,4-d]pyrimidine-4,6-diamine;
N6-[(2R)-2-amino-2-phenyl-ethyl]-1-methyl-N4-(2,2,2-trifluoroethyl)pyrazolo[3,4-d]pyrimidine-4,6-diamine;
6-N-(2-amino-2-phenylethyl)-1-methyl-4-N-propan-2-ylpyrazolo[3,4-d]pyrimidine-4,6-diamine;
6-N-[(2R)-2-amino-2-phenylethyl]-1-methyl-4-N-propan-2-ylpyrazolo[3,4-d]pyrimidine-4,6-diamine;
N′-(1-methyl-4-pyrrolidin-1-ylpyrazolo[3,4-d]pyrimidin-6-yl)-1-phenylethane-1,2-diamine;
6-N-(2-amino-2-phenylethyl)-4-N-(2-methoxyethyl)-1-methylpyrazolo[3,4-d]pyrimidine-4,6-diamine;
6-N-(2-amino-2-phenylethyl)-4-N-tert-butyl-1-methylpyrazolo[3,4-d]pyrimidine-4,6-diamine;
N6-(2-amino-2-phenyl-propyl)-N4-tert-butyl-1-methyl-pyrazolo[3,4-d]pyrimidine-4,6-diamine;
N6-(2-amino-2-phenyl-ethyl)-N4-tert-butyl-1-(trideuteriomethyl)pyrazolo[3,4-d]pyrimidine-4,6-diamine;
6-N-[(2R)-2-amino-2-phenylethyl]-4-N-tert-butyl-1-methylpyrazolo[3,4-d]pyrimidine-4,6-diamine;
6-N-[(2S)-2-amino-2-phenylethyl]-4-N-tert-butyl-1-methylpyrazolo[3,4-d]pyrimidine-4,6-diamine;
N6-[(2R)-2-amino-2-phenyl-ethyl]-N4-tert-butyl-1-(trideuteriomethyl)pyrazolo[3,4-d]pyrimidine-4,6-diamine;
6-N-(2-amino-2-phenylethyl)-4-N-(cyclopropylmethyl)-1-methylpyrazolo[3,4-d]pyrimidine-4,6-diamine;
N6-(2-amino-2-phenyl-ethyl)-N4-(dicyclopropylmethyl)-1-methyl-pyrazolo[3,4-d]pyrimidine-4,6-diamine;
N6-[(2R)-2-amino-2-phenyl-ethyl]-1-methyl-N4-[(2-methylcyclopropyl)methyl]pyrazolo[3,4-d]pyrimidine-4,6-diamine;
N6-[(2R)-2-amino-2-phenyl-ethyl]-N4-(1-cyclopropyl-1-methyl-ethyl)-1-methyl-pyrazolo[3,4-d]pyrimidine-4,6-diamine;
N6-[(2R)-2-amino-2-phenyl-ethyl]-1-methyl-N4-(spiro[2.2]pentan-2-ylmethyl)pyrazolo[3,4-d]pyrimidine-4,6-diamine
N6-[(2R)-2-amino-2-phenyl-ethyl]-N4-[(2,2-difluorocyclopropyl)methyl]-1-methyl-pyrazolo[3,4-d]pyrimidine-4,6-diamine;
N6-[(2R)-2-amino-2-phenyl-ethyl]-N4-[(2,2-dimethylcyclopropyl)methyl]-1-methyl-pyrazolo[3,4-d]pyrimidine-4,6-diamine;
6-N-(2-amino-2-phenylethyl)-4-N-(2-bicyclo[1.1.1]pentanyl)-1-methylpyrazolo[3,4-d]pyrimidine-4,6-diamine;
3-[[6-[(2-amino-2-phenylethyl)amino]-1-methylpyrazolo[3,4-d]pyrimidin-4-yl]amino]bicyclo[1.1.1]pentane-1-carbonitrile;
N6-(2-amino-2-phenylethyl)-N4-{3-fluorobicyclo[1.1.1]pentan-1-yl}-1-methyl-1H-pyrazolo[3,4-d]pyrimidine-4,6-diamine;
N6-[(2R)-2-amino-2-phenyl-ethyl]-N4-(3-bicyclo[1.1.1]pentanyl)-1-methyl-pyrazolo[3,4-d]pyrimidine-4,6-diamine;
N6-[(2R)-2-amino-2-phenyl-ethyl]-N4-(3-fluoro-1-bicyclo[1.1.1]pentanyl)-1-methyl-pyrazolo[3,4-d]pyrimidine-4,6-diamine;
6-N-(2-amino-2-phenylethyl)-4-N-(4,4-difluorocyclohexyl)-1-methylpyrazolo[3,4-d]pyrimidine-4,6-diamine;
N6-[(2R)-2-amino-2-phenyl-ethyl]-1-methyl-N4-tetrahydropyran-4-yl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]-3-methyl-butan-1-ol;
2-[[6-[[(2R)-2-amino-2-phenyl-ethyl]amino]-1-methyl-pyrazolo[3,4-d]pyrimidin-4-yl]amino]-2-methyl-propan-1-ol;
N6-[(2R)-2-amino-2-phenyl-ethyl]-N4-(2,2-dimethylpropyl)-1-methyl-pyrazolo[3,4-d]pyrimidine-4,6-diamine;
N6-[(2R)-2-amino-2-phenyl-ethyl]-N4-(2,2-difluoroethyl)-1-methyl-pyrazolo[3,4-d]pyrimidine-4,6-diamine;
N6-[(2R)-2-amino-2-phenyl-ethyl]-N4-(2,2-difluoropropyl)-1-methyl-pyrazolo[3,4-d]pyrimidine-4,6-diamine;
N6-[(2R)-2-amino-2-phenyl-ethyl]-1-methyl-N4-(2,2,2-trifluoro-1,1-dimethyl-ethyl)pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-(1-methylcyclopropyl)pyrazolo[3,4-d]pyrimidine-4,6-diamine;
N6-[(2R)-2-amino-2-phenyl-ethyl]-1-methyl-N4-[1-(trifluoromethyl)cyclopropyl]pyrazolo[3,4-d]pyrimidine-4,6-diamine;
N6-[(2R)-2-amino-2-phenyl-ethyl]-N4-(1-tert-butylcyclopropyl)-1-methyl-pyrazolo[3,4-d]pyrimidine-4,6-diamine;
N6-[(2R)-2-amino-2-phenyl-ethyl]-1-methyl-N4-spiro[2.2]pentan-2-yl-pyrazolo[3,4-d]pyrimidine-4,6-diamine;
[3-[[6-[(2-amino-2-phenyl-ethyl)amino]-1-methyl-pyrazolo[3,4-d]pyrimidin-4-yl]amino]-1-(chloromethyl)cyclobutyl]methanol;
N6-(2-amino-2-phenyl-ethyl)-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-N4-cyclobutyl-1-methyl-pyrazolo[3,4-d]pyrimidine-4,6-diamine;
N6-[(2R)-2-amino-2-phenyl-ethyl]-1-methyl-N4-(1-methylcyclobutyl)pyrazolo[3,4-d]pyrimidine-4,6-diamine;
N6-[(2R)-2-amino-2-phenyl-ethyl]-N4-(3,3-difluorocyclobutyl)-1-methyl-pyrazolo[3,4-d]pyrimidine-4,6-diamine;
N6-[(2R)-2-amino-2-phenyl-ethyl]-N4-(3,3-difluoro-1-methyl-cyclobutyl)-1-methyl-pyrazolo[3,4-d]pyrimidine-4,6-diamine;
N6-[(2R)-2-amino-2-phenyl-ethyl]-1-methyl-N4-(3-methylcyclobutyl)pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]cyclobutanecarbonitrile;
N6-[(2R)-2-amino-2-phenyl-ethyl]-N4-(3-methoxycyclobutyl)-1-methyl-pyrazolo[3,4-d]pyrimidine-4,6-diamine;
N6-[(2R)-2-amino-2-phenyl-ethyl]-N4-(3,3-dichlorocyclobutyl)-1-methyl-pyrazolo[3,4-d]pyrimidine-4,6-diamine;
N6-[(2R)-2-amino-2-phenyl-ethyl]-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-1-methyl-N4-(2-oxaspiro[3.3]heptan-6-yl)pyrazolo[3,4-d]pyrimidine-4,6-diamine;
N6-[(2R)-2-amino-2-phenyl-ethyl]-N4-(3,3-dimethylcyclobutyl)-1-methyl-pyrazolo[3,4-d]pyrimidine-4,6-diamine;
N6-[(2R)-2-amino-2-phenyl-ethyl]-N4-(3-fluoro-3-methyl-cyclobutyl)-1-methyl-pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-spiro[3.3]heptan-2-yl-pyrazolo[3,4-d]pyrimidine-4,6-diamine;
N6-[(2R)-2-amino-2-phenyl-ethyl]-N4-(2-methoxy-1,1-dimethyl-ethyl)-1-methyl-pyrazolo[3,4-d]pyrimidine-4,6-diamine;
N6-[(2R)-2-amino-2-phenyl-ethyl]-N4-(1,1-dimethylpropyl)-1-methyl-pyrazolo[3,4-d]pyrimidine-4,6-diamine;
N6-[(2R)-2-amino-2-phenyl-ethyl]-N4-[(1S)-1-cyclopropylethyl]-1-methyl-pyrazolo[3,4-d]pyrimidine-4,6-diamine;
N6-[(2R)-2-amino-2-phenyl-ethyl]-N4-isobutyl-1-methyl-pyrazolo[3,4-d]pyrimidine-4,6-diamine;
N6-[(2R)-2-amino-2-phenyl-ethyl]-1-methyl-N4-(spiro[2.3]hexan-2-ylmethyl)pyrazolo[3,4-d]pyrimidine-4,6-diamine;
N6-[(2R)-2-amino-2-phenyl-ethyl]-N4-(cyclobutylmethyl)-1-methyl-pyrazolo[3,4-d]pyrimidine-4,6-diamine;
N6-[(2R)-2-amino-2-phenyl-ethyl]-N4-[(2,2-difluorocyclobutyl)methyl]-1-methyl-pyrazolo[3,4-d]pyrimidine-4,6-diamine;
N6-[(2R)-2-amino-2-phenyl-ethyl]-N4-[(1-fluorocyclobutyl)methyl]-1-methyl-pyrazolo[3,4-d]pyrimidine-4,6-diamine;
N6-[(2R)-2-amino-2-phenyl-ethyl]-N4-[(3-fluorocyclobutyl)methyl]-1-methyl-pyrazolo[3,4-d]pyrimidine-4,6-diamine;
N6-[(2R)-2-amino-2-phenyl-ethyl]-1-methyl-N4-[3-(trifluoromethyl)cyclobutyl]pyrazolo[3,4-d]pyrimidine-4,6-diamine;
N6-[(2R)-2-amino-2-phenyl-ethyl]-N4-(2-fluoro-2-methyl-propyl)-1-methyl-pyrazolo[3,4-d]pyrimidine-4,6-diamine;
N6-[(2R)-2-amino-2-phenyl-ethyl]-1-methyl-N4-(1-methylcyclohexyl)pyrazolo[3,4-d]pyrimidine-4,6-diamine;
N6-[(2R)-2-amino-2-phenyl-ethyl]-N4-[(2-fluorocyclobutyl)methyl]-1-methyl-pyrazolo[3,4-d]pyrimidine-4,6-diamine;
N6-[(2R)-2-amino-2-phenyl-ethyl]-N4-cyclopentyl-1-methyl-pyrazolo[3,4-d]pyrimidine-4,6-diamine;
N6-[(2R)-2-amino-2-phenyl-ethyl]-1-methyl-N4-(3,3,3-trifluoropropyl)pyrazolo[3,4-d]pyrimidine-4,6-diamine;
N6-[(2R)-2-amino-2-phenyl-ethyl]-N4-[(2,2-dichlorocyclopropyl)methyl]-1-methyl-pyrazolo[3,4-d]pyrimidine-4,6-diamine;
N6-[(2R)-2-amino-2-phenyl-ethyl]-1-methyl-N4-(1-methylcyclopentyl)pyrazolo[3,4-d]pyrimidine-4,6-diamine;
N6-[(2R)-2-amino-2-phenyl-ethyl]-N4-[(1R)-2-methoxy-1-methyl-ethyl]-1-methyl-pyrazolo[3,4-d]pyrimidine-4,6-diamine;
N6-[(2R)-2-amino-2-phenyl-ethyl]-1-methyl-N4-(1-methyl-1-tetrahydropyran-4-yl-ethyl)pyrazolo[3,4-d]pyrimidine-4,6-diamine;
1-methyl-N6-[(2R)-2-amino-2-phenyl-ethyl]-N4-spiro[2.3]hexan-5-yl-pyrazolo[3,4-d]pyrimidine-4,6-diamine;
N6-[(2R)-2-amino-2-phenyl-ethyl]-N4-(2,2-dimethylcyclopropyl)-1-methyl-pyrazolo[3,4-d]pyrimidine-4,6-diamine;
N6-[(2R)-2-amino-2-phenylethyl]-1-methyl-N4-[(2R)-3-methylbutan-2-yl]-1H-pyrazolo[3,4-d]pyrimidine-4,6-diamine;
N6-[(2R)-2-amino-2-phenylethyl]-1-methyl-N4-[(2S)-3-methylbutan-2-yl]-1H-pyrazolo[3,4-d]pyrimidine-4,6-diamine;
6-[(6-{[(2R)-2-amino-2-phenylethyl]amino}-1-methyl-1H-pyrazolo[3,4-d]pyrimidin-4-yl)amino]spiro[3.3]heptan-2-ol;
N6-[(2R)-2-amino-2-phenylethyl]-1-methyl-N4-[4-(trifluoromethyl)cyclohexyl]-1H-pyrazolo[3,4-d]pyrimidine-4,6-diamine;
N6-[(2R)-2-amino-2-phenylethyl]-N4-[(1S)-3,3-difluorocyclopentyl]-1-methyl-1H-pyrazolo[3,4-d]pyrimidine-4,6-diamine;
N6-[(2R)-2-amino-2-phenylethyl]-1-methyl-N4-(4-methyloxan-4-yl)-1H-pyrazolo[3,4-d]pyrimidine-4,6-diamine;
N6-[(2R)-2-amino-2-phenylethyl]-N4-tert-butyl-1,3-dimethyl-1H-pyrazolo[3,4-d]pyrimidine-4,6-diamine;
N6-[(2R)-2-amino-2-phenylethyl]-N4-[(1R)-1-cyclopropylethyl]-1-methyl-1H-pyrazolo[3,4-d]pyrimidine-4,6-diamine;
N6-[(2R)-2-amino-2-phenyl-ethyl]-N4-tert-butyl-1-cyclopropyl-pyrazolo[3,4-d]pyrimidine-4,6-diamine;
N2-[1-methyl-4-(2-methylpropyl)-1H-pyrazolo[3,4-d]pyrimidin-6-yl]-1-phenylethane-1,2-diamine;
6-N-[2-amino-2-(2-methoxyphenyl)ethyl]-4-N-[(3-chloro-4-methylphenyl)methyl]-1-methylpyrazolo[3,4-d]pyrimidine-4,6-diamine;
6-N-(2-Amino-2-phenylethyl)-4-N-[(3-chloro-4-methylphenyl)methyl]-1-methylpyrazolo[3,4-d]pyrimidine-4,6-diamine;
6-N-(2-amino-2-pyridin-2-ylethyl)-1-methyl-4-N-[4-(trifluoromethyl)phenyl]pyrazolo[3,4-d]pyrimidine-4,6-diamine;
3-[1-Amino-2-[[1-methyl-4-(methylamino)pyrazolo[3,4-d]pyrimidin-6-yl]amino]ethyl]benzonitrile;
N6-[2-Amino-2-(3-ethynylphenyl)ethyl]-N4-tert-butyl-1-methyl-pyrazolo[3,4-d]pyrimidine-4,6-diamine;
N6-[2-amino-2-(2-pyridyl)ethyl]-N4-tert-butyl-1-methyl-pyrazolo[3,4-d]pyrimidine-4,6-diamine;
6-N-(2-amino-2-pyridin-3-ylethyl)-4-N-[(3-chloro-4-methylphenyl)methyl]-1-methylpyrazolo[3,4-d]pyrimidine-4,6-diamine;
N6-[2-amino-2-(3-pyridyl)ethyl]-N4-tert-butyl-1-methyl-pyrazolo[3,4-d]pyrimidine-4,6-diamine;
6-N-[2-amino-2-(2-chlorophenyl)ethyl]-1-methyl-4-N-[4-(trifluoromethyl)phenyl]pyrazolo[3,4-d]pyrimidine-4,6-diamine;
N6-[2-amino-2-(2-chlorophenyl)ethyl]-N4-tert-butyl-1-methyl-pyrazolo[3,4-d]pyrimidine-4,6-diamine;
N6-[(2R)-2-amino-2-(2-chlorophenyl)ethyl]-N4-tert-butyl-1-methyl-pyrazolo[3,4-d]pyrimidine-4,6-diamine;
6-N-[2-amino-2-(2-fluorophenyl)ethyl]-4-N-[(3-chloro-4-methylphenyl)methyl]-1-methylpyrazolo[3,4-d]pyrimidine-4,6-diamine;
N6-[2-amino-2-(2-fluorophenyl)ethyl]-N4-tert-butyl-1-methyl-pyrazolo[3,4-d]pyrimidine-4,6-diamine;
N6-[2-amino-2-(2-fluorophenyl)ethyl]-N4-tert-butyl-1-(trideuteriomethyl) pyrazolo[3,4-d]pyrimidine-4,6-diamine;
N6-[(2R)-2-amino-2-(3-bromophenyl)ethyl]-N4-isopropyl-1-methyl-pyrazolo[3,4-d]pyrimidine-4,6-diamine;
N6-[(2R)-2-amino-2-(3-bromophenyl)ethyl]-1-methyl-N4-(2,2,2-trifluoro-1,1-dimethyl-ethyl)pyrazolo[3,4-d]pyrimidine-4,6-diamine;
6-N-[2-amino-2-(3-bromophenyl)ethyl]-4-N,1-dimethylpyrazolo[3,4-d]pyrimidine-4,6-diamine;
N6-[2-amino-2-(3-bromophenyl)ethyl]-N4-tert-butyl-1-methyl-pyrazolo[3,4-d]pyrimidine-4,6-diamine;
6-N-[2-amino-2-(4-bromophenyl)ethyl]-1-methyl-4-N-propan-2-ylpyrazolo[3,4-d]pyrimidine-4,6-diamine;
N6-[2-amino-2-(2-bromophenyl)ethyl]-N4-tert-butyl-1-methyl-pyrazolo[3,4-d]pyrimidine-4,6-diamine;
4-[1-amino-2-[[1-methyl-4-(methylamino)pyrazolo[3,4-d]pyrimidin-6-yl]amino]ethyl]benzonitrile;
3-[1-amino-2-[[4-(tert-butylamino)-1-methyl-pyrazolo[3,4-d]pyrimidin-6-yl]amino]ethyl]benzonitrile;
3-[(1R)-1-amino-2-[[1-methyl-4-[(2,2,2-trifluoro-1,1-dimethyl-ethyl)amino]pyrazolo[3,4-d]pyrimidin-6-yl]amino]ethyl]benzonitrile;
N6-[2-amino-2-(3-pyridyl)ethyl]-N4-tert-butyl-1-methyl-pyrazolo[3,4-d]pyrimidine-4,6-diamine;
N6-[2-amino-2-(3-pyridyl)ethyl]-N4-(3,3-difluorocyclobutyl)-1-methyl-pyrazolo[3,4-d]pyrimidine-4,6-diamine;
N6-[2-amino-2-(4-pyridyl)ethyl]-N4-tert-butyl-1-methyl-pyrazolo[3,4-d]pyrimidine-4,6-diamine;
N6-[2-amino-2-(4-pyridyl)ethyl]-N4-(3,3-difluorocyclobutyl)-1-methyl-pyrazolo[3,4-d]pyrimidine-4,6-diamine;
4-[(1R)-1-amino-2-{[4-(tert-butylamino)-1-methyl-1H-pyrazolo[3,4-d]pyrimidin-6-yl]amino}ethyl]benzonitrile;
N4-tert-butyl-1-methyl-N6-[(2R)-2-(methylamino)-2-phenylethyl]-1H-pyrazolo[3,4-d]pyrimidine-4,6-diamine;
N6-[2-amino-2-(4-fluorophenyl)ethyl]-N4-tert-butyl-1-methyl-1H-pyrazolo[3,4-d]pyrimidine-4,6-diamine;
N6-[2-amino-2-(3,4-difluorophenyl)ethyl]-N4-tert-butyl-1-methyl-pyrazlo[3,4-d]pyrimidine-4,6-diamine;
N6-[2-amino-2-(3-cyclopropylphenyl)ethyl]-N4-tert-butyl-1-methyl-1H-pyrazolo[3,4-d]pyrimidine-4,6-diamine;
N6-[2-amino-2-(4-cyclopropylphenyl)ethyl]-N4-tert-butyl-1-methyl-1H-pyrazolo[3,4-d]pyrimidine-4,6-diamine;
N′-(4-Ethoxy-1-methylpyrazolo[3,4-d]pyrimidin-6-yl)-1-phenylethane-1,2-diamine;
N′-(1-methyl-4-propan-2-yloxypyrazolo[3,4-d]pyrimidin-6-yl)-1-phenylethane-1,2-diamine;
N′-(4-tert-butoxy-1-methyl-pyrazolo[3,4-d]pyrimidin-6-yl)-1-phenyl-ethane-1,2-diamine;
1-methyl-N6-{[(3R)-morpholin-3-yl]methyl}-N4-[4-(trifluoromethyl)phenyl]-1H-pyrazolo[3,4-d]pyrimidine-4,6-diamine;
6-N-[(1S,2R)-2-aminocyclohexyl]-4-N-[(3-chloro-4-methylphenyl)methyl]-1-methylpyrazolo[3,4-d]pyrimidine-4,6-diamine;
6-N-[(1R,2R)-2-aminocyclohexyl]-4-N-[(3-chloro-4-methylphenyl)methyl]-1-methylpyrazolo[3,4-d]pyrimidine-4,6-diamine;
N6-[(2R)-2-amino-2-phenyl-ethyl]-N4-cyclopropyl-1-methyl-pyrazolo[3,4-d]pyrimidine-4,6-diamine;
N6-[(2R)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-1-methyl-N4-(2,2,2-trifluoroethyl)pyrazolo[3,4-d]pyrimidine-4,6-diamine;
N6-[(2R)-2-amino-2-phenyl-ethyl]-1-methyl-N4-[6-(trifluoromethyl)-3-pyridyl]pyrazolo[3,4-d]pyrimidine-4,6-diamine;
N6-[(2S)-2-amino-2-phenyl-ethyl]-1-methyl-N4-[6-(trifluoromethyl)-3-pyridyl]pyrazolo[3,4-d]pyrimidine-4,6-diamine;
rel-6-N-[(2S)-2-amino-2-phenylpropyl]-4-N,1-dimethylpyrazolo[3,4-d]pyrimidine-4,6-diamine;
N6-[(2S)-2-amino-2-(2-fluorophenyl)ethyl]-N4-tert-butyl-1-methyl-pyrazolo[3,4-d]pyrimidine-4,6-diamine;
N6-[(2R)-2-amino-2-(2-fluorophenyl)ethyl]-N4-tert-butyl-1-methyl-pyrazolo[3,4-d]pyrimidine-4,6-diamine;
rel-N6-[(2R)-2-amino-2-phenyl-propyl]-1-methyl-N4-[6-(trifluoromethyl)-3-pyridyl]pyrazolo[3,4-d]pyrimidine-4,6-diamine;
6-N-[(2R)-2-amino-3-fluoropropyl]-4-N-[(3-chloro-4-methylphenyl)methyl]-1-methylpyrazolo[3,4-d]pyrimidine-4,6-diamine;
rel-N6-[(2S)-2-amino-2-phenyl-propyl]-1-methyl-N4-[6-(trifluoromethyl)-3-pyridyl]pyrazolo[3,4-d]pyrimidine-4,6-diamine;
N6-[(2R)-2-amino-2-(2-fluorophenyl)ethyl]-N4-tert-butyl-1-(trideuteriomethyl)pyrazolo[3,4-d]pyrimidine-4,6-diamine;
N6-[(2S)-2-amino-2-(2-fluorophenyl)ethyl]-N4-tert-butyl-1-(trideuteriomethyl)pyrazolo[3,4-d]pyrimidine-4,6-diamine;
N6-[(2R)-2-amino-2-(4-fluorophenyl)ethyl]-N4-tert-butyl-1-methyl-1H-pyrazolo[3,4-d]pyrimidine-4,6-diamine;
N6-[(2R)-2-amino-2-(2-fluorophenyl)ethyl]-N4-tert-butyl-1-methyl-pyrazolo[3,4-d]pyrimidine-4,6-diamine;
N6-[(2R)-2-amino-2-(3,4-difluorophenyl)ethyl]-N4-tert-butyl-1-methyl-1H-pyrazolo[3,4-d]pyrimidine-4,6-diamine;
N6-[(2S)-2-amino-2-(3,4-difluorophenyl)ethyl]-N4-tert-butyl-1-methyl-1H-pyrazolo[3,4-d]pyrimidine-4,6-diamine;
N6-[(2R)-2-amino-2-cyclohexylethyl]-N4-tert-butyl-1-methyl-1H-pyrazolo[3,4-d]pyrimidine-4,6-diamine; and
N6-[(2S)-2-amino-2-cyclohexylethyl]-N4-tert-butyl-1-methyl-1H-pyrazolo[3,4-d]pyrimidine-4,6-diamine.

The various functional groups and substituents making up the compounds of the formula (I) are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ¹H, ²H(D), and ³H (T); C may be in any isotopic form, including ¹²C, ¹³C, and ¹⁴C; and O may be in any isotopic form, including ¹⁶O and ¹⁸O; and the like.

It is also to be understood that certain compounds of the formula (I) (and compounds of formula (II), (Ill), (IV), (V), (VI), (VII), (VIII) and (IX)) may exist in solvated as well as unsolvated forms such as, for example, hydrated forms. It is to be understood that the invention encompasses all such solvated forms that possess antiproliferative activity.

It is also to be understood that certain compounds of the formula (I) (and compounds of formula (II), (Ill), (IV), (V), (VI), (VII), (VIII) and (IX)) may exhibit polymorphism, and that the invention encompasses all such forms that possess antiproliferative activity.

Compounds of the formula (I) (and compounds of formula (II), (Ill), (IV), (V), (VI), (VII), (VIII) and (IX)) may exist in a number of different tautomeric forms and references to compounds of the formula (I) include all such forms. For the avoidance of doubt, where a compound can exist in one of several tautomeric forms, and only one is specifically described or shown, all others are nevertheless embraced by formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

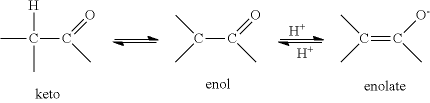

Compounds of the formula (I) containing an amine function may also form N-oxides. A reference herein to a compound of the formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula (I) may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (I) and in-vivo cleavable amide derivatives that may be formed at a carboxy group or an amino group in a compound of the formula (I).

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically acceptable pro-drug of a compound of the formula (I) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula (I) that possesses a carboxy group is, for example, an in vivo cleavable ester thereof. An in vivo cleavable ester of a compound of the formula (I) containing a carboxy group is, for example, a pharmaceutically acceptable ester which is cleaved in the human or animal body to produce the parent acid. Suitable pharmaceutically acceptable esters for carboxy include C₁₋₆alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆alkyl esters such as methoxycarbonyloxymethyl and 1-methoxycarbonyloxyethyl esters.

A suitable pharmaceutically acceptable pro-drug of a compound of the formula (I) that possesses a hydroxy group is, for example, an in vivo cleavable ester or ether thereof. An in vivo cleavable ester or ether of a compound of the formula (I) containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula (I) that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄alkylamine such as 2-methoxyethylamine, a phenyl-C₁₋₄ alkylamine such as benzylamine and amino acids such as glycine or an ester thereof.

A suitable pharmaceutically acceptable pro-drug of a compound of the formula (I) that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula (I) may be exerted in part by one or more metabolites that are formed within the human or animal body after administration of a compound of the formula (I). As stated hereinbefore, the in vivo effects of a compound of the formula (I) may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I will vary depending on the nature of the variable groups. Suitable processes for their preparation are described further in the accompanying Examples.

Once a compound of formula (I) has been synthesised by any one of the processes defined herein, the processes may then further comprise the additional steps of:

(i) removing any protecting groups present;
(ii) converting the compound formula (I) into another compound of formula I;
(iii) forming a pharmaceutically acceptable salt, hydrate or solvate thereof; and/or
(iv) forming a prodrug thereof.

The resultant compounds of formula (I) can be isolated and purified using techniques well known in the art.

### Biological Activity

The BCDIN3D enzyme assays described in accompanying Example section may be used to measure the pharmacological effects of the compounds of the present invention.

Although the pharmacological properties of the compounds of formula I vary with structural change, as expected, the compounds of the invention were found to be active in these BCDIN3D assays.

In general, the compounds of the invention demonstrate an IC₅₀ of 10 μM or less in the BCDIN3D enzyme assay described herein, with preferred compounds of the invention demonstrating an IC₅₀ of 5 μM or less and the most preferred compounds of the invention demonstrating an IC₅₀ of 2 μM or less.

In general, the compounds of the invention demonstrate an IC₅₀ of 10 μM or less in the BCDIN3D thermal stability assay described herein, with preferred compounds of the invention demonstrating an IC₅₀ of 5 μM or less and the most preferred compounds of the invention demonstrating an IC₅₀ of 2 μM or less.

### Pharmaceutical Compositions

According to a further aspect of the invention there is provided a pharmaceutical composition which comprises a compound of the invention as defined hereinbefore, or a pharmaceutically acceptable salt thereof, and one or more pharmaceutically acceptable excipients.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition and/or disease.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula (I) will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors and/or degraders of BCDIN3D activity.

The present invention therefore provides a method of inhibiting and/or degrading BCDIN3D activity in vitro or in vivo, said method comprising contacting a cell with an effective amount of a compound as defined herein, or a pharmaceutically acceptable salt thereof, or a pharmaceutical composition as defined herein.

The present invention also provides a method of treating a disease or disorder in which BCDIN3D activity is implicated in a patient in need of such treatment, said method comprising administering to said patient a therapeutically effective amount of a compound as defined herein, or a pharmaceutically acceptable salt thereof, or a pharmaceutical composition as defined herein. Suitably, the disease or disorder in which BCDIN3D activity is implicated is cancer and/or a metabolic disease. Suitably, the cancer is breast cancer (such as triple negative breast cancer) or lung cancer (such as non-small cell lung cancer). Suitably the metabolic disease is obesity or diabetes.

The present invention provides a method of inhibiting and/or degrading cell proliferation in vitro or in vivo, said method comprising contacting a cell with an effective amount of a compound as defined herein, or a pharmaceutically acceptable salt thereof, or a pharmaceutical composition as defined herein.

In another aspect, the present invention provides a method of inhibiting and/or degrading metastasis in vitro or in vivo, said method comprising contacting a cell with an effective amount of a compound as defined herein, or a pharmaceutically acceptable salt, or a pharmaceutical composition as defined herein.

The present invention provides a method of treating a proliferative disorder, said method comprising administering to a subject in need thereof a therapeutically effective amount of a compound as defined herein, or a pharmaceutically acceptable salt thereof, or a pharmaceutical composition as defined herein.

The present invention provides a method of treating cancer, said method comprising administering to a subject in need thereof a therapeutically effective amount of a compound as defined herein, or a pharmaceutically acceptable salt thereof, or a pharmaceutical composition as defined herein. Suitably the cancer is breast cancer, such as triple negative breast cancer. Suitably the cancer is lung cancer, such as non-small cell lung cancer.

The present invention provides a method of treating breast cancer, said method comprising administering to a subject in need thereof a therapeutically effective amount of a compound, or a pharmaceutically acceptable salt thereof, or a pharmaceutical composition as defined herein.

The present invention provides a method of treating triple negative breast cancer, said method comprising administering to a subject in need thereof a therapeutically effective amount of a compound, or a pharmaceutically acceptable salt thereof, or a pharmaceutical composition as defined herein.

The present invention provides a method of treating lung cancer, said method comprising administering to a subject in need thereof a therapeutically effective amount of a compound, or a pharmaceutically acceptable salt thereof, or a pharmaceutical composition as defined herein.

The present invention provides a method of treating non-small cell lung cancer, said method comprising administering to a subject in need thereof a therapeutically effective amount of a compound, or a pharmaceutically acceptable salt thereof, or a pharmaceutical composition as defined herein.

The present invention provides a method of treating a metabolic disease, said method comprising administering to a subject in need thereof a therapeutically effective amount of a compound as defined herein, or a pharmaceutically acceptable salt thereof, or a pharmaceutical composition as defined herein. Suitably the metabolic disease is obesity or diabetes.

The present invention provides a compound, or a pharmaceutically acceptable salt thereof, or a pharmaceutical composition as defined herein for use in therapy.

The present invention provides a compound as defined herein, or a pharmaceutically acceptable salt thereof, or a pharmaceutical composition as defined herein, for use in the treatment of a proliferative condition.

The present invention provides a compound as defined herein, or a pharmaceutically acceptable salt thereof, or a pharmaceutical composition as defined herein, for use in the treatment of cancer. In a particular embodiment, the cancer is human cancer. Suitably the cancer is breast cancer, such as triple negative breast cancer. Suitably the cancer is lung cancer, such as triple negative lung cancer.

The present invention provides a compound, or a pharmaceutically acceptable salt thereof, or a pharmaceutical composition as defined herein for use in the treatment of breast cancer, such as triple negative breast cancer.

The present invention provides a compound, or a pharmaceutically acceptable salt thereof, or a pharmaceutical composition as defined herein for use in the treatment of lung cancer, such as non-small cell lung cancer.

The present invention provides a compound as defined herein, or a pharmaceutically acceptable salt thereof, or a pharmaceutical composition as defined herein, for use in the treatment of a metabolic disease. In a particular embodiment, the metabolic disease is obesity or diabetes.

The present invention provides a compound as defined herein, or a pharmaceutically acceptable salt thereof, or a pharmaceutical composition as defined herein, for use in the inhibition and/or degradation of BCDIN3D activity.

The present invention provides a compound as defined herein, or a pharmaceutically acceptable salt thereof, for use in the treatment of a disease or disorder in which BCDIN3D activity is implicated. Suitably, the disease or disorder in which BCDIN3D activity is implicated is cancer and/or a metabolic disease. Suitably the cancer is breast cancer or lung cancer. Suitably the breast cancer is triple negative breast cancer. Suitably the lung cancer is non-small cell lung cancer. Suitably the metabolic disease is obesity or diabetes.

The present invention provides a use of a compound as defined herein, or a pharmaceutically acceptable salt thereof, in the manufacture of a medicament for the treatment of a proliferative condition.

The present invention provides a use of a compound as defined herein, or a pharmaceutically acceptable salt thereof, in the manufacture of a medicament for the treatment of cancer. Suitably, the medicament is for use in the treatment of human cancers. Suitably the cancer is breast cancer, such as triple negative breast cancer. Suitably the cancer is lung cancer, such as non-small cell lung cancer.

The present invention provides a use of a compound as defined herein, or a pharmaceutically acceptable salt thereof, in the manufacture of a medicament for the treatment of breast cancer, such as triple negative breast cancer.

The present invention provides a use of a compound as defined herein, or a pharmaceutically acceptable salt thereof, in the manufacture of a medicament for the treatment of lung cancer, such as non-small cell lung cancer.

The present invention provides a use of a compound as defined herein, or a pharmaceutically acceptable salt thereof, in the manufacture of a medicament for the treatment of a metabolic disease. Suitably the metabolic disease is obesity or diabetes.

The present invention provides a use of a compound as defined herein, or a pharmaceutically acceptable salt thereof, in the manufacture of a medicament for the inhibition and/or degradation of BCDIN3D activity.

The present invention provides a use of a compound as defined herein, or a pharmaceutically acceptable salt thereof, in the manufacture of a medicament for the treatment of a disease or disorder in which BCDIN3D activity is implicated. Suitably, the disease or disorder in which BCDIN3D activity is implicated is cancer, such as breast cancer or lung cancer. Suitably the breast cancer is triple negative breast cancer. Suitably the lung cancer is non-small cell lung cancer. Suitably, the disease or disorder in which BCDIN3D activity is implicated is a metabolic disease, such as obesity or diabetes.

The term “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers (including breast cancer such as triple negative breast cancer, bladder cancer, kidney cancer, lung cancer such as non-small cell lung cancer (NSCLC), squamous cell carcinomas (SCC) (including SCC of the head and neck, oesophagus, lung and ovary), leukemias (including acute lymphoblastic leukaemia (ALL) and chronic myeloid leukaemia (CML)), lymphomas (including acute lymphoblastic leukaemia (ALL), chronic myeloid leukaemia (CML), diffuse Large B Cell lymphoma, Mantle-Cell lymphoma, Follicular lymphoma) and sarcomas), psoriasis, bone diseases, fibroproliferative disorders (e.g., of connective tissues), and atherosclerosis. Any type of cell may be treated, including but not limited to, lymphatic, blood, lung, colon, breast, ovarian, prostate, liver, pancreas, brain, kidney, bladder and skin.

The anti-proliferative effects of the compounds of the present invention have particular application in the treatment of human cancers (by virtue of their inhibition and/or degradation of BCDIN3D activity).

The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), the promotion of apoptosis (programmed cell death), and/or the induction or enhancement of an anti-tumour immune response.

In a particular embodiment of the invention, the proliferative condition to be treated is cancer.

### Anti-Inflammatory Properties

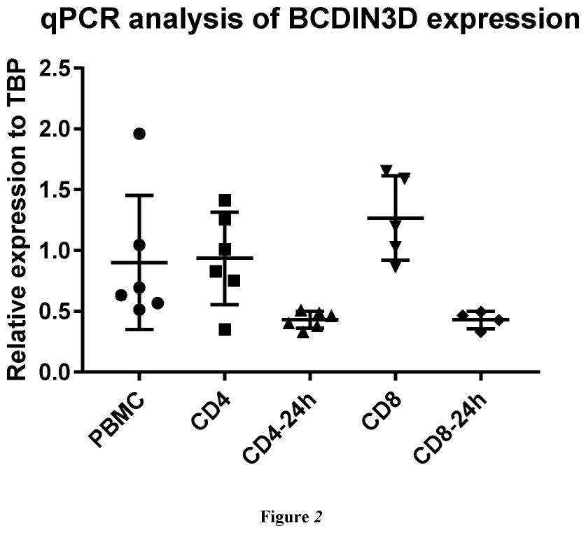

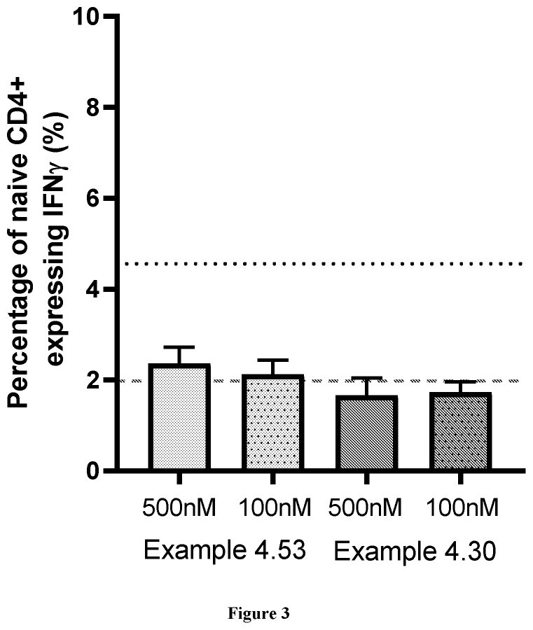

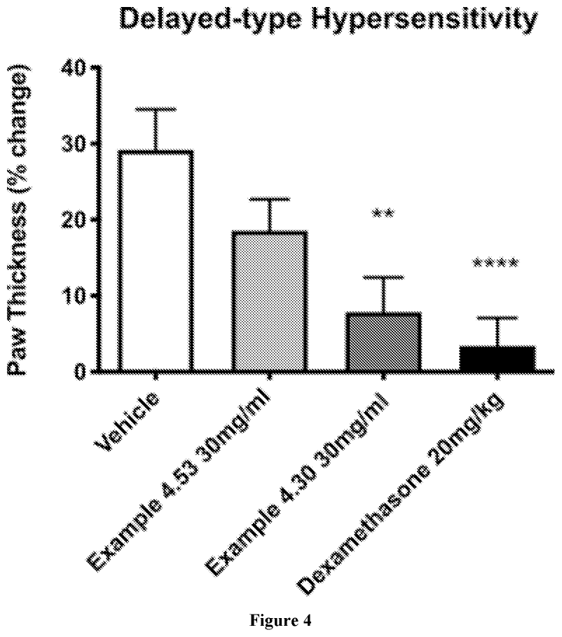

Data presented in the biological example section herein shows that BCDIN3D expression in primary human CD4+ and CD8+ T-cells is downregulated in response to activation and differentiation of naïve T-cells (see FIG. 2). This suggests BCDIN3D levels and activity are linked to T-cell maintenance and differentiation. It has also been shown that the pharmacological inhibition of BCDIN3D blocks IFN gamma production by activated T-cells (see FIG. 3). IFN gamma is a key cytokine released by activated T-cells upon antigen recognition, driving activation of downstream immune responses by immune cells such as monocytes and macrophages. Inhibition of IFN gamma demonstrates the immunosuppressive and anti-inflammatory activity of BCDIN3D inhibitors in vitro. In addition, BCDIN3D inhibitors were tested in an in vivo model of delayed-type hypersensitivity, which is a well-established in vivo inflammation model dependent on T-cell and monocyte/macrophage activation. Hyperactivation of these cell types is a key hall mark of immuno-inflammatory diseases. This test confirmed that BCDIN3D inhibitors demonstrated significant anti-inflammatory activity in vivo (see FIG. 4).

Together, these data support the development of BCDIN3D inhibitors for acute and chronic inflammatory diseases and disorders, including T-cell and monocyte-driven auto-immune diseases such as rheumatoid arthritis, inflammatory bowel disorders, multiple sclerosis, systemic lupus erythematosus, psoriasis, atopic dermatitis.

Thus, in one particular aspect, the present invention provides a BCDIN3D inhibitor, or a pharmaceutically acceptable salt thereof, for use in the treatment of inflammation.

In another aspect, the present invention provides the use of a BCDIN3D inhibitor, or a pharmaceutically acceptable salt thereof, in the manufacture of a medicament for use in the treatment of inflammation.

In yet another aspect, the present invention provides a method of treating inflammation, the method comprising administering a therapeutically effective amount of a BCDIN3D inhibitor, or a pharmaceutically acceptable salt thereof.

In another aspect, the present invention provides a compound of formula I as defined herein, or a pharmaceutically acceptable salt thereof, for use in the treatment of inflammation.

In another aspect, the present invention provides the use of a compound of formula I as defined herein, or a pharmaceutically acceptable salt thereof, in the manufacture of a medicament for use in the treatment of inflammation.

In yet another aspect, the present invention provides a method of treating inflammation, the method comprising administering a therapeutically effective amount of a compound of formula I as defined herein, or a pharmaceutically acceptable salt thereof.

Inflammation involves the activation of the immune system in response to harmful stimuli, such as, e.g., a pathogen, infection, irritant, or damage to cells. As a stereotyped response, inflammation is a mechanism of innate immunity, as compared to adaptive immunity, which is specific for each pathogen. Inflammation can be classified as either acute or chronic. Generally speaking, acute inflammation is mediated by granulocytes, while chronic inflammation is mediated by mononuclear cells such as monocytes and lymphocytes.

Acute inflammation is an initial protective response of the body to remove an injurious stimulus by maintaining tissue integrity and contributing to tissue repair. It a part of the body's natural defense system against injury and disease, and in the absence of acute inflammation, wounds and infections would never heal and progressive destruction of the tissue would compromise the survival of the organism.

The process of acute inflammation is initiated by cells already present in all tissues, mainly resident macrophages, dendritic cells, histiocytes, Kupffer cells, mastocytes, vascular endothelial cells, and vascular smooth muscle cells. At the onset of a harmful stimulus, these cells undergo activation and release inflammatory mediating and sensitizing molecules, such as, e.g., pro-inflammatory cytokines, pro-inflammatory prostaglandins, leukotrienes, histamine, serotonin, neutral proteases, bradykinin and nitric oxide. These inflammatory molecules modulate a complex series of biological events involving cellular and acellular components of the local vascular system, the immune system, and the injured tissue site to propagate and mature the inflammatory response. These events are responsible for eliciting an acute inflammatory response, typically characterized by 1) vasodilatation which increases blood flow into the tissue thereby causing erythema (redness and warmth), which may extend beyond this site (the flare response); 2) blood vessel permeability which increases plasma leakage into the tissue thereby causing edema (swelling); 3) alter the excitability of certain sensory neurons causing hypersensitivity and pain; 4) stimulate the release of inflammation inducing molecules such as, e.g., neuropeptides like substance P (SP) and calcitonin gene-related peptide (CGRP), prostaglandins, and amino acids like glutamate, from the peripheral nerve endings; and 5) increase migration of leukocytes, mainly granulocytes, from the blood vessels into the tissue. An acute inflammatory response requires constant stimulation to be sustained and must be actively terminated when no longer needed. Hence, acute inflammation ceases once the injurious stimulus has been removed.

However, severe or prolonged noxious stimulation results in a chronic inflammatory response that leads to a progressive shift in the type of cells present at the site of tissue injury.

Chronic inflammation may be characterized as the simultaneous destruction and healing of tissue from the inflammatory process, with the net result of provoking injury rather than mediating repair. As such, chronic inflammation is a disease. As an inflammatory response can occur anywhere in the body, chronic inflammation has been implicated in the pathophysiology of a wide range of seemingly unrelated disorders which underlay a large and varied group of human diseases. For example, chronic inflammation is involved in diseases as diverse as cardiovascular diseases, cancers, allergies, obesity, diabetes, digestive system diseases, degenerative diseases, auto-immune disorders, and Alzheimer's disease.

BCDIN3D inhibitors are potential therapeutic treatments for inflammation, particularly chronic inflammation. The administration of a BCDIN3D inhibitor is anticipated to reduce a symptom associated with chronic inflammation, thereby treating the individual.

Aspects of the present specification disclose, in part, treating an individual suffering from a chronic inflammation. As used herein, the term “treating,” refers to reducing or eliminating in an individual a clinical symptom of a chronic inflammation; or delaying or preventing in an individual the onset of a clinical symptom of a chronic inflammation. For example, the term “treating” can mean reducing a symptom of a condition characterized by a chronic inflammation by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with chronic inflammation are well known and can be determined by a person of ordinary skill in the art by taking into account factors, including, without limitation, the location of the chronic inflammation, the cause of the chronic inflammation, the severity of the chronic inflammation, and/or the tissue or organ affected by the chronic inflammation. Those of skill in the art will know the appropriate symptoms or indicators associated with a specific type of chronic inflammation and will know how to determine if an individual is a candidate for treatment as disclosed herein.

Chronic inflammation symptoms include, without limitation, edema, hyperemia, erythema, bruising, tenderness, stiffness, swollenness, fever, chills, stuffy nose, stuffy head, breathing problems, fluid retention, blood clots, loss of appetite, increased heart rate, formation of granulomas, fibrinous, pus, non-viscous serous fluid, or ulcer and pain. The actual symptoms associated with a chronic inflammation are well known and can be determined by a person of ordinary skill in the art by taking into account factors, including, without limitation, the location of the inflammation, the cause of the inflammation, the severity of the inflammation, the tissue or organ affected, and the associated disorder.

Specific patterns of chronic inflammation are seen during particular situations that arise in the body, such as when inflammation occurs on an epithelial surface, or pyogenic bacteria are involved. For example, granulomatous inflammation is an inflammation resulting from the formation of granulomas arising from a limited but diverse number of diseases, include, without limitation, tuberculosis, leprosy, sarcoidosis, and syphilis. Purulent inflammation is an inflammation resulting in large amount of pus, which consists of neutrophils, dead cells, and fluid. Infection by pyogenic bacteria such as staphylococci is characteristic of this kind of inflammation. Serous inflammation is an inflammation resulting from copious effusion of non-viscous serous fluid, commonly produced by mesothelial cells of serous membranes, but may be derived from blood plasma. Skin blisters exemplify this pattern of inflammation. Ulcerative inflammation is an inflammation resulting from the necrotic loss of tissue from the epithelial surface, exposing lower layers and forming an ulcer.

A chronic inflammation symptom can be associated with a large, unrelated group of disorders which underlay a variety of diseases and disorders. The immune system is often involved with chronic inflammatory disorders, demonstrated in both allergic reactions and some myopathies, with many immune system disorders resulting in abnormal inflammation. Non-immune diseases with etiological origins in chronic inflammatory processes include cancer, atherosclerosis, and ischaemic heart disease. Non-limiting examples of disorders exhibiting chronic inflammation as a symptom include, without limitation, acne, acid reflux/heartburn, age related macular degeneration (AMD), allergy, allergic rhinitis, Alzheimer's disease, amyotrophic lateral sclerosis, anemia, appendicitis, arteritis, arthritis, asthma, atherosclerosis, autoimmune disorders, balanitis, blepharitis, bronchiolitis, bronchitis, a bullous pemphigoid, burn, bursitis, cancer, cardiac arrest, carditis, celiac disease, cellulitis, cervicitis, cholangitis, cholecystitis, chorioamnionitis, chronic obstructive pulmonary disease (COPD), cirrhosis, colitis, congestive heart failure, conjunctivitis, cyclophosphamide-induced cystitis, cystic fibrosis, cystitis, common cold, dacryoadenitis, dementia, dermatitis, dermatomyositis, diabetes, diabetic neuropathy, diabetic retinopathy, diabetic nephropathy, diabetic ulcer, digestive system disease, eczema, emphysema, encephalitis, endocarditis, endometritis, enteritis, enterocolitis, epicondylitis, epididymitis, fasciitis, fibromyalgia, fibrosis, fibrositis, gastritis, gastroenteritis, gingivitis, glomerulonephritis, glossitis, heart disease, heart valve dysfunction, hepatitis, hidradenitis suppurativa, Huntington's disease, hyperlipidemic pancreatitis, hypertension, ileitis, infection, inflammatory bowel disease, inflammatory cardiomegaly, inflammatory neuropathy, insulin resistance, interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), a migraine, multiple sclerosis, myelitis, myocarditis, myositis, nephritis, non-alcoholic steatohepatitis, obesity, omphalitis, oophoritis, orchitis, osteochondritis, osteopenia, osteomyelitis, osteoporosis, osteitis, otitis, pancreatitis, Parkinson's disease, parotitis, pelvic inflammatory disease, pemphigus vularis, pericarditis, peritonitis, pharyngitis, phlebitis, pleuritis, pneumonitis, polycystic nephritis, proctitis, prostatitis, psoriasis, pulpitis, pyelonephritis, pylephlebitis, renal failure, reperfusion injury, retinitis, rheumatic fever, rhinitis, salpingitis, sarcoidosis, sialadenitis, sinusitis, spastic colon, stenosis, stomatitis, stroke, surgical complication, synovitis, tendonitis, tendinosis, tenosynovitis, thrombophlebitis, tonsillitis, trauma, traumatic brain injury, transplant rejection, trigonitis, tuberculosis, tumor, urethritis, ursitis, uveitis, vaginitis, vasculitis, and vulvitis. See also, Eric R. First, Application of Botulinum Toxin to the Management of Neurogenic Inflammatory Disorders, U.S. Pat. No. 6,063,768, which is hereby incorporated by reference in its entirety.

In one embodiment, a chronic inflammation comprises a tissue inflammation. Tissue inflammation is a chronic inflammation that is confined to a particular tissue or organ. In aspect of this embodiment, a tissue inflammation comprises, e.g., a skin inflammation, a muscle inflammation, a tendon inflammation, a ligament inflammation, a bone inflammation, a cartilage inflammation, a lung inflammation, a heart inflammation, a liver inflammation, a pancreatic inflammation, a kidney inflammation, a bladder inflammation, a stomach inflammation, an intestinal inflammation, a neuron inflammation, and a brain inflammation.

In another embodiment, a chronic inflammation comprises a systemic inflammation. Although the processes involved are identical to tissue inflammation, systemic inflammation is not confined to a particular tissue but in fact overwhelms the body, involving the endothelium and other organ systems. When it is due to infection, the term sepsis is applied, with the terms bacteremia being applied specifically for bacterial sepsis and viremia specifically to viral sepsis. Vasodilation and organ dysfunction are serious problems associated with widespread infection that may lead to septic shock and death.

In another embodiment, a chronic inflammation comprises an arthritis. Arthritis includes a group of conditions involving damage to the joints of the body due to the inflammation of the synovium including, without limitation osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease), and Still's disease. Arthritis can affect a single joint (monoarthritis), two to four joints (oligoarthritis) or five or more joints (polyarthritis) and can be either an auto-immune disease or a non-autoimmune disease.

In another embodiment, a chronic inflammation comprises an autoimmune disorder. Autoimmune diseases can be broadly divided into systemic and organ-specific autoimmune disorders, depending on the principal clinico-pathologic features of each disease. Systemic autoimmune diseases include, without limitation, systemic lupus erythematosus (SLE), Sjögren's syndrome, Scleroderma, rheumatoid arthritis and polymyositis. Local autoimmune diseases may be endocrinologic (Diabetes Mellitus Type 1, Hashimoto's thyroiditis, Addison's disease etc.), dermatologic (pemphigus vulgaris), hematologic (autoimmune haemolytic anemia), neural (multiple sclerosis) or can involve virtually any circumscribed mass of body tissue. Types of autoimmune disorders include, without limitation, acute disseminated encephalomyelitis (ADEM), Addison's disease, an allergy or sensitivity, amyotrophic lateral sclerosis, anti-phospholipid antibody syndrome (APS), arthritis, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD), diabetes mellitus type 1 (IDDM), endometriosis, fibromyalgia, Goodpasture's syndrome, Graves' disease, Guillain-Barré syndrome (GBS), Hashimoto's thyroiditis, hidradenitis suppurativa, idiopathic thrombocytopenic purpura, inflammatory bowel disease, interstitial cystitis, lupus (including discoid lupus erythematosus, drug-induced lupus erythematosus, lupus nephritis, neonatal lupus, subacute cutaneous lupus erythematosus and systemic lupus erythematosus), morphea, multiple sclerosis (MS), myasthenia gravis, myopathies, narcolepsy, neuromyotonia, pemphigus vulgaris, pernicious anaemia, primary biliary cirrhosis, recurrent disseminated encephalomyelitis (multiphasic disseminated encephalomyelitis), rheumatic fever, schizophrenia, scleroderma, Sjögren's syndrome, tenosynovitis, vasculitis, and vitiligo. See Pamela D. Van Schaack & Kenneth L. Tong, Treatment of Autoimmune Disorder with a Neurotoxin, U.S. Patent Publication 2006/138059, which is hereby incorporated by reference in its entirety.

In another embodiment, a chronic inflammation comprises a myopathy. Myopathies are caused when the immune system inappropriately attacks components of the muscle, leading to inflammation in the muscle. A myopathy includes an inflammatory myopathy and an auto-immune myopathy. Myopathies include, without limitation, dermatomyositis, inclusion body myositis, and polymyositis.

In another embodiment, a chronic inflammation comprises a vasculitis. Vasculitis is a varied group of disorders featuring inflammation of a vessel wall including lymphatic vessels and blood vessels like veins (phlebitis), arteries (arteritis) and capillaries due to leukocyte migration and resultant damage. The inflammation may affect any size blood vessel, anywhere in the body. It may affect either arteries and/or veins. The inflammation may be focal, meaning that it affects a single location within a vessel; or it may be widespread, with areas of inflammation scattered throughout a particular organ or tissue, or even affecting more than one organ system in the body. Vasculitis include, without limitation, Buerger's disease (thromboangiitis obliterans), cerebral vasculitis (central nervous system vasculitis), Churg-Strauss arteritis, cryoglobulinemia, essential cryoglobulinemic vasculitis, giant cell (temporal) arteritis, Golfer's vasculitis, Henoch-Schonlein purpura, hypersensitivity vasculitis (allergic vasculitis), Kawasaki disease, microscopic polyarteritis/polyangiitis, polyarteritis nodosa, polymyalgia rheumatica (PMR), rheumatoid vasculitis, Takayasu arteritis, Wegener's granulomatosis, and vasculitis secondary to connective tissue disorders like systemic lupus erythematosus (SLE), rheumatoid arthritis (RA), relapsing polychondritis, Behget's disease, or other connective tissue disorders, vasculitis secondary to viral infection.

In another embodiment, a chronic inflammation comprises a skin disorder. Skin disorders include, without limitation, an acne, including acne vulgaris, a bullous phemigoid, a dermatitis, including atopic dermatitis and chronic actinic dermatitis, an eczema like atopic eczema, contact eczema, xerotic eczema, seborrhoeic dermatitis, dyshidrosis, discoid eczema, venous eczema, dermatitis herpetiformis, neurodermatitis, and autoeczematization, and statis dermatitis, hidradenitis suppurativa, lichen planus, psoriasis including plaqure psoriasis, nail psoriasis, guttate psoriasis, scalp psoriasis, inverse psoriasis, pustular psoriasis, erythrodermis psoriasis, and psoriatic arthritis, rosacea and scleroderma including morphea.

In another embodiment, a chronic inflammation comprises a gastrointestinal disorder. A gastrointestinal disorder includes, without limitation, irritable bowel disease, an inflammatory bowel disease including Crohn's disease and an ulcerative colitis like ulcerative proctitis, left-sided colitis, pancolitis and fulminant colitis.

In another embodiment, a chronic inflammation comprises a cardiovascular disease. When LDL cholesterol becomes embedded in arterial walls, it can invoke an immune response. Chronic inflammation eventually can damage the arteries, which can cause them to burst. Cardiovascular disease is any of a number of specific diseases that affect the heart itself and/or the blood vessel system, especially the veins and arteries leading to and from the heart. There are more than 60 types of cardiovascular disorders including, without limitation, a hypertension, endocarditis, myocarditis, heart valve dysfunction, congestive heart failure, myocardial infarction, a diabetic cardiac conditions, blood vessel inflammation like arteritis, phlebitis, vasculitis; arterial occlusive disease like arteriosclerosis and stenosis, inflammatory cardiomegaly, a peripheral arterial disease; an aneurysm; an embolism; a dissection; a pseudoaneurysm; a vascular malformation; a vascular nevus; a thrombosis; a thrombphlebitis; a varicose veins; a stroke. Symptoms of a cardiovascular disorder affecting the heart include, without limitation, chest pain or chest discomfort (angina), pain in one or both arms, the left shoulder, neck, jaw, or back, shortness of breath, dizziness, faster heartbeats, nausea, abnormal heartbeats, feeling fatigued. Symptoms of a cardiovascular disorder affecting the brain include, without limitation, sudden numbness or weakness of the face, arm, or leg, especially on one side of the body, sudden confusion or trouble speaking or understanding speech, sudden trouble seeing in one or both eyes, sudden dizziness, difficulty walking, or loss of balance or coordination, sudden severe headache with no known cause. Symptoms of a cardiovascular disorder affecting the legs, pelvis and/or arm include, without limitation, claudication, which is a pain, ache, or cramp in the muscles, and cold or numb feeling in the feet or toes, especially at night.

In another embodiment, a chronic inflammation comprises a cancer. Inflammation orchestrates the microenvironment around tumors, contributing to proliferation, survival and migration. For example, fibrinous inflammation results from a large increase in vascular permeability which allows fibrin to pass through the blood vessels. If an appropriate procoagulative stimulus is present, such as cancer cells, a fibrinous exudate is deposited. This is commonly seen in serous cavities, where the conversion of fibrinous exudate into a scar can occur between serous membranes, limiting their function. In another example, a cancer is an inflammatory cancer like a NF-κB-driven inflammatory cancer.

In another embodiment, a chronic inflammation comprises a pharmacologically-induced inflammation. Certain drugs or exogenic chemical compounds are known to affect inflammation. For example, Vitamin A deficiency causes an increase in an inflammatory response. Certain illicit drugs such as cocaine and ecstasy may exert some of their detrimental effects by activating transcription factors intimately involved with inflammation (e.g. NF-κB).

In another embodiment, a chronic inflammation comprises an infection. An infectious organism can escape the confines of the immediate tissue via the circulatory system or lymphatic system, where it may spread to other parts of the body. If an organism is not contained by the actions of acute inflammation it may gain access to the lymphatic system via nearby lymph vessels. An infection of the lymph vessels is known as lymphangitis, and infection of a lymph node is known as lymphadenitis. A pathogen can gain access to the bloodstream through lymphatic drainage into the circulatory system. Infections include, without limitation, bacterial cystitis, bacterial encephalitis, pandemic influenza, viral encephalitis, and viral hepatitis (A, B and C).

In another embodiment, a chronic inflammation comprises a tissue or organ injury. Tissue or organ injuries include, without limitation, a burn, a laceration, a wound, a puncture, or a trauma.

In another embodiment, a chronic inflammation comprises a transplant rejection. Transplant rejection occurs when a transplanted organ or tissue is not accepted by the body of the transplant recipient because the immune system of the recipient attacks the transplanted organ or tissue. An adaptive immune response, transplant rejection is mediated through both T cell mediated and humoral immune (antibodies) mechanisms. A transplant rejection can be classified as a hyperacute rejection, an acute rejection, or a chronic rejection. Chronic rejection of a transplanted organ or tissue is where the rejection is due to a poorly understood chronic inflammatory and immune response against the transplanted tissue. Also included in the term “transplant rejection” is a graft-versus-host disease (GVHD). GVHD is a common complication of allogeneic bone marrow transplantation in which functional immune cells in the transplanted marrow recognize the recipient as “foreign” and mount an immunologic attack. It can also take place in a blood transfusion under certain circumstances. GVHD is divided into acute and chronic forms. Acute and chronic GVHD appear to involve different immune cell subsets, different cytokine profiles, somewhat different host targets, and respond differently to treatment.

In another embodiment, a chronic inflammation comprises a Th1-mediated inflammatory disease. In a well-functioning immune system, an immune response should result in a well balanced pro-inflammatory Th1 response and anti-inflammatory Th2 response that is suited to address the immune challenge. Generally speaking, once a pro-inflammatory Th1 response is initiated, the body relies on the anti-inflammatory response invoked by a Th2 response to counteract this Th1 response. This counteractive response includes the release of Th2 type cytokines such as, e.g., IL-4, IL-5, and IL-13 which are associated with the promotion of IgE and eosinophilic responses in atopy, and also IL-10, which has an anti-inflammatory response. A Th1-mediated inflammatory disease involves an excessive pro-inflammatory response produced by Th1 cells that leads to chronic inflammation. The Th1-mediated disease may be virally, bacterially or chemically (e.g. environmentally) induced. For example, a virus causing the Th1-mediated disease may cause a chronic or acute infection, which may cause a respiratory disorder or influenza.

In another embodiment, a chronic inflammation comprises a chronic neurogenic inflammation. Chronic neurogenic Inflammation refers to an inflammatory response initiated and/or maintained through the release of inflammatory molecules like SP or CGRP which released from peripheral sensory nerve terminals (i.e., an efferent function, in contrast to the normal afferent signaling to the spinal cord in these nerves). Chronic neurogenic inflammation includes both primary inflammation and secondary neurogenic inflammation. As used herein, the term “primary” neurogenic inflammation refers to tissue inflammation (inflammatory symptoms) that is initiated by, or results from, the release of substances from primary sensory nerve terminals (such as C and A-delta fibers). As used herein, the term “secondary” neurogenic inflammation” refers to tissue inflammation initiated by non-neuronal sources (e.g., extravasation from vascular bed or tissue interstitium-derived, such as from mast cells or immune cells) of inflammatory mediators, such as peptides or cytokines, stimulating sensory nerve terminals and causing a release of inflammatory mediators from the nerves. The net effect of both forms (primary and secondary) of chronic neurogenic inflammation is to have an inflammatory state that is maintained by the sensitization of the peripheral sensory nerve fibers. The physiological consequence of the resulting chronic neurogenic inflammation depends on the tissue in question, producing, such as, e.g., cutaneous pain (allodynia, hyperalgesia), joint pain and/or arthritis, visceral pain and dysfunction, pulmonary dysfunction (asthma, COPD), and bladder dysfunction (pain, overactive bladder).

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

In one aspect, the present invention provides a combination comprising a compound as defined herein, or a pharmaceutically acceptable salt thereof, with one or more additional therapeutic agents.

The antiproliferative treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride:
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;
(v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, for example those which are directed to the targets listed above, such as ISIS 2503, an anti-ras antisense;
(ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies, and other immunotherarpy approaches including tumour vaccinations, CAR-T therapy, stimulation of innate immunity such as the use of STING agonists, TLR9 agonists and bispecifics; and
(xi) immune checkpoint inhibitors for example PD1 inhibitors (such as pembrolizumab, nivolumab and cemiplimab), PDL1 inhibitors (such as atezolizumab, avelumab and durvalumab), CTLA4 inhibitors (such as ipilimumab), LAG3 inhibitors, GITR inhibitors, and TIGIT inhibitors.

In a particular embodiment, the antiproliferative treatment defined hereinbefore may involve, in addition to the compound of the invention, conventional surgery or radiotherapy or chemotherapy.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example breast cancer) comprising a compound of the invention as defined hereinbefore, or a pharmaceutically acceptable salt thereof, and another anti-tumour agent.

According to this aspect of the invention there is provided a combination for use in the treatment of a proliferative condition, such as cancer (for example breast cancer), comprising a compound of the invention as defined herein, or a pharmaceutically acceptable salt thereof, and any one of the anti-tumour agents listed herein above.

In a further aspect of the invention there is provided a compound of the invention or a pharmaceutically acceptable salt thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt thereof, in combination with an anti-tumour agent (optionally selected from one listed herein above), in association with a pharmaceutically acceptable diluent or carrier.

### EXAMPLES

Where reference is made to the use of a “similar” procedure, as will be appreciated by those skilled in the art, such a procedure may involve minor variation, for example reaction temperature, reagent/solvent amount, reaction time, work-up conditions or chromatographic purification conditions.

The following abbreviations have been used in the Examples:
BOC tert-butyloxycarbonyl
BrettPhos 2-(Dicyclohexylphosphino)3,6-dimethoxy-2′,4′,6′-triisopropyl-1,1′-biphenyl
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate methanesulfonate
BSA bovine serum albumin
CO₂ carbon dioxide
° C. degrees Celsius
d doublet
D deuterium
DCM dichloromethane
dd doublet of doublets
DEA diethylamine
DMF dimethylformamide
DMSO dimethylsulfoxide
dt doublet of triplets
DTT Dithiolthreitol
EDTA Ethylenediaminetetraacetic acid
equiv. equivalents
g gram
HCl Hydrochloric acid
HPLC High performance liquid chromatography
Hz Hertz
IPA Isopropyl alcohol
LCMS liquid chromatography mass spectroscopy
M Molar
m multiplet
MeOH Methanol
mg Milligram
MHz megahertz
min minutes
mL millilitre
μL microlitre
mm millimeter
μm micrometre
mM millimolar
nm nanometres
mmol millimolar
NH₄OH ammonium hydroxide
NMP N-Methyl-2-pyrrolidone
NMR nuclear magnetic resonance
Pd/C Palladium on charcoal
q quartet
s singlet
SAH S-adenosylhomocysteine
SAM S-adenosylmethionine
SCX Strong cation exchange
sept septet
SFC Super critical fluid chromatography
t triplet
tert tertiary
TFA Trifluoroacetic acid
t_{R} Retention time
Tris tris(hydroxymethyl)aminomethane
tt triplet of triplets
UV Ultra violet
vol volumes
v:v volume to volume

### Example 1: Spectroscopic Methods

The following HPLC methodology was used.

### Analytical HPLC

LCMS Method A refers to low pH analysis using a mobile phase consisting of 0.1% formic acid in a gradient of 5-100% acetonitrile in water over 1.2 minutes at a flow rate of 1.2 mL/min. The stationary phase consisted of a Kinetex Core-Shell C18, 2.1 mm×50 mm, 5 μm. The experiment was run at 40° C.

LCMS Method B refers to high pH analysis using a mobile phase consisting of 2 mM ammonium bicarbonate, buffered to pH 10 in a gradient of 5-100% acetonitrile in water over 2.1 minutes at a flow rate of 1.0 mL/min. The stationary phase consisted of a Phenomenex Gemini-NX C18, 2.0×50 mm, 3 μm. The experiment was run at 40° C.

LCMS Method C refers to high pH analysis using a mobile phase consisting of 2 mM ammonium bicarbonate, buffered to pH 10 in a gradient of 5-100% acetonitrile in water over 5.8 minutes at a flow rate of 0.6 mL/min. The stationary phase consisted of a Waters UPLC® BEH™ 018, 2.1×100 mm, 1.7 μm. The experiment was run at 40° C.

LCMS Method D refers to high pH analysis using a mobile phase consisting of 2 mM ammonium bicarbonate, buffered to pH 10 in a gradient of 5-100% acetonitrile in water over 5.9 minutes at a flow rate of 0.6 mL/min. The stationary phase consisted of a Phenomenex Gemini-NX C18, 2.0×100 mm, 3 μm. The experiment was run at 40° C.

LCMS Method E refers to low pH analysis using a mobile phase consisting of 0.1% formic acid in a gradient of 5-100% acetonitrile in water over 5.3 minutes at a flow rate of 0.6 mL/min. The stationary phase consisted of a Phenomenex Kinetix-XB C18, 2.1 mm×100 mm, 1.7 μm. The experiment was run at 40° C.

LCMS Method F refers to SFC chiral analysis using a mobile phase consisting of 40% IPA+0.2% DEA: 60% CO₂ at a flow rate of 4 mL/min. The stationary phase consisted of a Chiralpak AD-H 25 cm column.

LCMS Method G refers to SFC chiral analysis using a mobile phase consisting of 70/30% CO₂/(IPA+0.5% iPrNH₂) at a flow rate of 2.4 mL/min. The stationary phase consisted of a CHIRALPAK IC (250 mm×4.6), 5 μm. The experiment was run at 40° C.

LCMS Method H refers to SFC chiral analysis using a mobile phase consisting of 35% ethanol: 65% CO₂ at a flow rate of 4 mL/min. The stationary phase consisted of a Chiralpak AD-H 25 cm column with a run time of 10 minutes and UV at 230 nm

LCMS Method J refers to SFC chiral analysis using a mobile phase consisting of 35% methanol+0.2% DEA: 65% CO₂ at a flow rate of 4 mL/min. The stationary phase consisted of a Chiralcel OJ-H 25 cm column with a run time of 6 minutes and UV at 230 nm.

LCMS Method K refers to SFC chiral analysis using a mobile phase consisting of 25% ethanol+0.2% DEA: 75% CO₂ at a flow rate of 4 mL/min. The stationary phase consisted of a Chiralpak IC 25 cm column with a run time of 15 minutes and UV at 230 nm.

LCMS Method L refers to low pH analysis using a mobile phase consisting of 0.1% formic acid in a gradient of 5-100% acetonitrile in water over 2.26 minutes at a flow rate of 1.2 mL/min. The stationary phase consisted of a Phenomenex Kinetex Core-Shell C8, 2.1 mm×50 mm, 5 μm. The experiment was run at 40° C.

### Preparative HPLC

Method A refers to low pH purification using a mobile phase consisting of 0.1% formic acid in a gradient of 10-95% acetonitrile in water over 14.4 minutes at a flow rate of 40 mL/min. The stationary phase consisted of a Waters Sunfire C18, 30×100 mm, 10 μm.

Method B refers to high pH purification using a mobile phase consisting of 0.2% ammonium hydroxide in a gradient of 30-95% acetonitrile in water over 10 minutes at a flow rate of 40 mL/min. The stationary phase consisted of a Waters XBridge™ C18 OBD™, 30×100 mm, 10 μm.

Method C (SFC) refers to chiral separation using a mobile phase consisting of CO₂/isopropanol and 0.5% isopropylamine 70/30 over 30 minutes at a flow rate of 50 mL/min. The stationary phase consisted of a Chiralpak IC 5 μm, 250×20 mm

Method D (SFC) refers to chiral separation using a mobile phase consisting of CO₂/methanol and 0.2% diethylamine over 30 minutes at a flow rate of 15 mL/min. The stationary phase consisted of a Chiralpak AD-H, 250 mm.

Method E (SFC) refers to chiral separation using a mobile phase consisting of 65% CO₂/35% ethanol over 30 minutes at a flow rate of 15 mL/min. The stationary phase consisted of a Chiralpak AD-H, 250 mm

Method F (SFC) refers to chiral separation using a mobile phase consisting of 25% methanol+0.2% DEA: 75% CO₂ over 30 minutes with a flow rate of 15 mL/min. The stationary phase consisted of a Chiralcel OJ-H, 25 cm column.

Method G (SFC) refers to chiral separation using a mobile phase consisting of 25% ethanol+0.2% DEA: 75% CO₂ over 30 minutes with a flow rate of 15 mL/min. The stationary phase consisted of a Chiralpak IC 25 cm column.

Where compounds were purified by liquid chromatography, a Biotage Isolera™ chromatography system using pre-packed silica and pre-packed modified silica cartridges was used.

### Example 2: Chemical Synthesis and Characterisation

### General Method 1

To a solution of a 4,6-dichloro-1-substituted-pyrazolo[3,4-d]pyrimidine (1 equiv.) in acetonitrile (20 vol) was added an amine or a HCl salt of an amine (1.1-1.2 equiv.) and N, N-diisopropylethylamine (1.5-3.0 equiv.) and the resulting reaction mixture was stirred at room temperature for 18 hours. Subsequently the reaction mixture was heated to 50° C. or 100° C. if the reaction profile by LCMS showed incomplete conversion to the desired compound. The reaction mixture was quenched by the addition of saturated ammonium chloride solution or 10% citric acid solution and the organics were extracted into DCM and filtered through a TELOS™ phase separator to yield the organics which were concentrated in vacuo and purified by either silica gel chromatography eluting with 0-100% heptane:ethyl acetate or via reverse phase chromatography or preparative HPLC as necessary

### General Method 2

To a solution of 4,6-dichloro-1-methyl-1H-pyrazolo[3,4-d]pyrimidine (1 equiv.) and an amine/aniline (1 equiv.) in 1,4 dioxane (10 vol) was added a solution of sodium;2-methylpropan-2-olate (2 equiv., 2M) and the resulting reaction mixture was heated to 100° C. for 30 minutes where upon the reaction was deemed complete by LCMS. The reaction mixture was loaded onto a silica gel column and purified by eluting with 20-60% heptane:ethyl acetate to yield the desired compound which was used directly according to one of the following General Methods.

### General Method 3

A compound synthesised via either General Method 1 or General Method 2 was suspended in a diamine (5-10 equiv.) or the diamine (3 equiv.) in 1-butanol (10 vol) and the resulting reaction mixture was heated to 120-125° C. for 18 hours. The resulting product was purified by preparative HPLC (General Method A or General Method B) and lyophilised to yield the desired compound.

### General Method 4

To a suspension of a compound synthesised via either General Method 1 or General Method 2 (1 equiv.) in 1-butanol (20 vol) or NMP (10 vol) was added a BOC diamine (1.5-3 equiv.) and the resulting reaction mixture was heated to 110-125° C. for 18 hours. The resulting intermediate product was purified by either preparative HPLC (General Method A or General Method B), silica gel chromatography or reverse phase C18 chromatography to yield a BOC protected compound which was stirred in 4N HCl in 1,4 dioxane (10 vol) until complete consumption of the starting material was observed by LCMS (1-4 hours). The resulting HCl salt was loaded onto an SCX column and eluted with 0-100% (MeOH: 7N ammonia in MeOH) to yield the desired compound which was purified by preparative HPLC (Method A or Method B) or lyophilised directly depending on purity.

### General Method 5

To a suspension of a compound synthesised via either General Method 1 or General Method 2 (1 equiv.) in n-butanol (20 vol) was added a mono BOC protected diamine (1.5-3 equiv.) and the resulting reaction mixture was heated to 110-125° C. for 18 hours. The resulting intermediate product was purified by preparative HPLC (General Method A or General Method B), silica gel chromatography or reverse phase C18 chromatography to yield a BOC protected compound which was stirred in DCM:TFA (10:1 v:v; 10 vol) until complete consumption of the starting material was observed by LCMS (1-18 hours). The resulting salt was loaded onto an SCX column and eluted with 0-100% (Methanol: 7N ammonia in methanol) to yield the desired compound which was purified by preparative HPLC (Method A or Method B) or lyophilised directly depending on purity.

### Compound 1.01: N6-(2-Aminoethyl)-1-methyl-N4-[4-(trifluoromethyl)phenyl]pyrazolo[3,4-d]pyrimidine-4,6-diamine

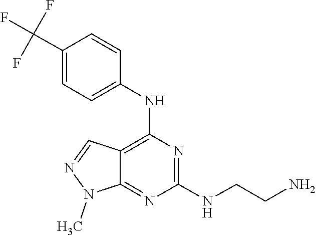 [SMILES: Cn1ncc2c(Nc3ccc(C(F)(F)F)cc3)nc(NCCN)nc21]

Step 1: To a solution of 4,6-dichloro-1-methyl-pyrazolo[3,4-d]pyrimidine (50.0 mg, 0.25 mmol) in acetonitrile (1 mL) was added 4-(trifluoromethyl)aniline (0.05 mL, 0.37 mmol) and N, N-diisopropylethylamine (0.07 mL, 0.37 mmol) and the resulting reaction mixture was stirred at room temperature for 18 hours. Due to incomplete conversion, the reaction mixture was heated to 50° C. and stirring continued for a further 18 hours, followed by heating to 100° C. for a further 8 hours. The crude reaction mixture was purified by low pH preparative HPLC (Method C) to yield the desired intermediate compound as a yellow solid (20 mg, 24%). LCMS (Analytical Method C) t_{R}=1.23 min, [M+H]⁺=327.9/330.05; 1H NMR (500 MHz, DMSO-d6) δ 8.33 (s, 1H), 8.03 (d, J=8.6 Hz, 2H), 7.81 (d, J=8.6 Hz, 2H), 3.94 (s, 3H).

Step 2: 6-Chloro-1-methyl-N-[4-(trifluoromethyl)phenyl]pyrazolo[3,4-d]pyrimidin-4-amine (98%, 20.0 mg, 0.06 mmol) and ethane-1,2-diamine (0.1 mL, 1.5 mmol) were heated to 125° C. for 18 hours before purification via high pH preparative HPLC (Method B) to yield the title compound as an off white solid (5 mg, 22%). LCMS (Analytical Method E) t_{R}=4.69 min, [M+H]⁺=352.2; 1H NMR (500 MHz, DMSO-d6) δ 9.99 (s, 1H), 8.39 (s, 1H), 8.17 (s, 2H), 8.08 (s, 1H), 7.69 (s, 2H), 7.20 (s, 1H), 3.78 (s, 3H), 3.54-3.47 (m, 2H), 2.93 (s, 2H).

The compounds in Table 1 were prepared in an analogous manner to Compound 1.01 according to General Method 3 using the appropriate diamine and 4-substituted pyrazolopyrimidine intermediate which was in turn synthesised according to General Method 1 or General Method 2

TABLE 1

| Compound | | | Analytical | t_{R} | |
|---|---|---|---|---|---|
| No. | Name | Structure | Method | (min) | [M + H]⁺ |
| 1.02 | N6-(2- aminoethyl)-1- methyl-N4-[3- (trifluoromethyl) phenyl]-1H- pyrazolo[3,4- d]pyrimidine-4,6- diamine | 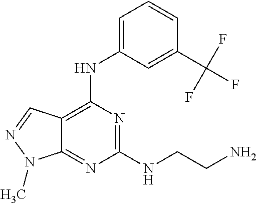 [SMILES: Cn1ncc2c(Nc3cccc(C(F)(F)F)c3)nc(NCCN)nc21] | E | 1.69 | 352.1 |
| 1.03 | N6-(2- aminoethyl)-1- methyl-N4-(4- phenoxyphenyl)- 1H-pyrazolo[3,4- d]pyrimidine-4,6- diamine | 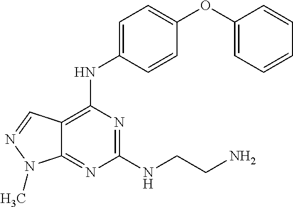 [SMILES: Cn1ncc2c(Nc3ccc(Oc4ccccc4)cc3)nc(NCCN)nc21] | D | 5.25 | 376.2 |
| 1.04 | N6-(2- aminoethyl)-1- methyl-N4-(3- phenoxyphenyl)- 1H-pyrazolo[3,4- d]pyrimidine-4,6- diamine | 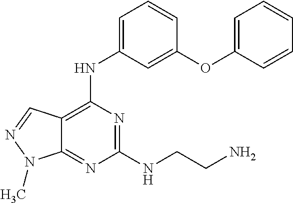 [SMILES: Cn1ncc2c(Nc3cccc(Oc4ccccc4)c3)nc(NCCN)nc21] | D | 5.02 | 376.2 |
| 1.05 | N6-(2- aminoethyl)-1- methyl-N4-[(4- phenylphenyl) methyl]pyrazolo[3,4- d]pyrimidine-4,6- diamine | 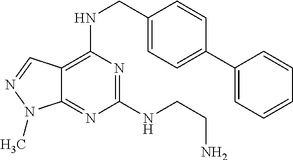 [SMILES: Cn1ncc2c(NCc3ccc(-c4ccccc4)cc3)nc(NCCN)nc21] | D | 5.33 | 374.2 |
| 1.06 | N6-(2- aminoethyl)-N4- [1-(3-chloro-4- methoxyphenyl) ethyl]-1-methyl-1H- pyrazolo[3,4- d]pyrimidine-4,6- diamine | 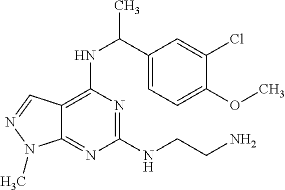 [SMILES: COc1ccc(C(C)Nc2nc(NCCN)nc3c2cnn3C)cc1Cl] | E | 1.40 | 376.2 |
| 1.07 | N6-(2- aminoethyl)-N4- [2-(6- methoxypyridin-3- yl)ethyl]-1-methyl- 1H-pyrazolo[3,4- d]pyrimidine-4,6- diamine | 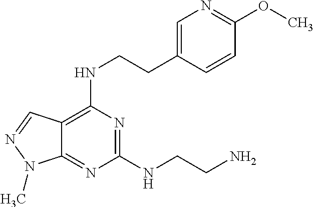 [SMILES: COc1ccc(CCNc2nc(NCCN)nc3c2cnn3C)cn1] | C | 0.96 | 343.2 |
| 1.08 | N6-(2- aminoethyl)-N4- [2-(3-chloro-4- methoxyphenyl) ethyl]-1-methyl-1H- pyrazolo[3,4- d]pyrimidine-4,6- diamine | 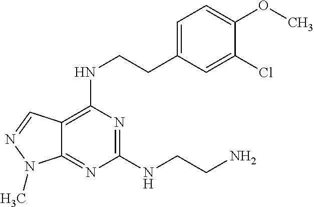 [SMILES: COc1ccc(CCNc2nc(NCCN)nc3c2cnn3C)cc1Cl] | E | 1.35 | 376.2 |
| 1.09 | N6-(2- aminoethyl)-N4- [2-(2H-1,3- benzodioxol-5- yl)ethyl]-1-methyl- 1H-pyrazolo[3,4- d]pyrimidine-4,6- diamine | 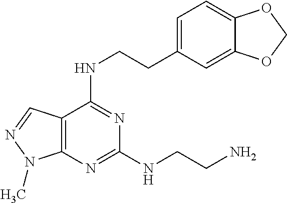 [SMILES: Cn1ncc2c(NCCc3ccc4c(c3)OCO4)nc(NCCN)nc21] | D | 4.23 | 356.2 |
| 1.10 | N6-(2- aminoethyl)-N4- [(1-benzofuran-5- yl)methyl]-1- methyl-1H- pyrazolo[3,4- d]pyrimidine-4,6- diamine | 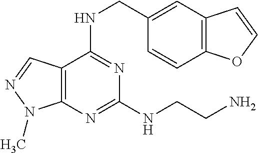 [SMILES: Cn1ncc2c(NCc3ccc4occc4c3)nc(NCCN)nc21] | D | 4.24 | 338.2 |
| 1.11 | N6-(2- aminoethyl)-N4- {3′-methoxy-[1,1′- biphenyl]-3-yl}-1- methyl-1H- pyrazolo[3,4- d]pyrimidine-4,6- diamine | 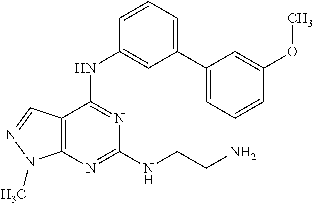 [SMILES: COc1cccc(-c2cccc(Nc3nc(NCCN)nc4c3cnn4C)c2)c1] | D | 5.01 | 390.2 |
| 1.12 | N6-(2- aminoethyl)-N4- {[1- (benzenesulfonyl) piperidin-4- yl]methyl}-1- methyl-1H- pyrazolo[3,4- d]pyrimidine-4,6- diamine | 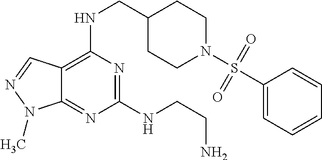 [SMILES: Cn1ncc2c(NCC3CCN(S(=O)(=O)c4ccccc4)CC3)nc(NCCN)nc21] | D | 4.36 | 445.2 |
| 1.13 | N6-(2- aminoethyl)-N4- [(1H-1,3- benzodiazol-5- yl)methyl]-1- methyl-1H- pyrazolo[3,4- d]pyrimidine-4,6- diamine | 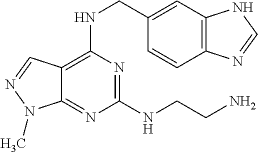 [SMILES: Cn1ncc2c(NCc3ccc4nc[nH]c4c3)nc(NCCN)nc21] | D | 2.88 | 338.2 |
| 1.14 | N6-(2- aminoethyl)-N4- {3′-methoxy-[1,1′- biphenyl]-4-yl}-1- methyl-1H- pyrazolo[3,4- d]pyrimidine-4,6- diamine | 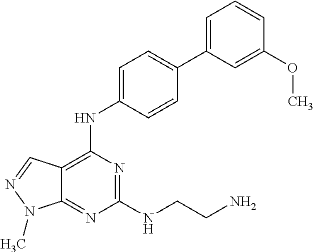 [SMILES: COc1cccc(-c2ccc(Nc3nc(NCCN)nc4c3cnn4C)cc2)c1] | D | 4.99 | 390.1 |
| 1.15 | N6-(2- aminoethyl)-N4- {4′-methoxy-[1,1′- biphenyl]-3-yl}-1- methyl-1H- pyrazolo[3,4- d]pyrimidine-4,6- diamine | 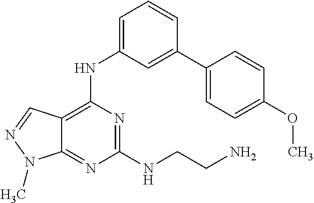 [SMILES: COc1ccc(-c2cccc(Nc3nc(NCCN)nc4c3cnn4C)c2)cc1] | D | 4.93 | 390.1 |
| 1.16 | N6-(2- aminoethyl)-N4- {[1,1′-biphenyl]-3- yl}-1-methyl-1H- pyrazolo[3,4- d]pyrimidine-4,6- diamine | 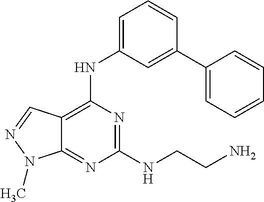 [SMILES: Cn1ncc2c(Nc3cccc(-c4ccccc4)c3)nc(NCCN)nc21] | D | 4.95 | 360.1 |
| 1.17 | N6-(2- aminoethyl)-N4- [(3-chloro-4- fluorophenyl) methyl]-1-methyl-1H- pyrazolo[3,4- d]pyrimidine-4,6- diamine | 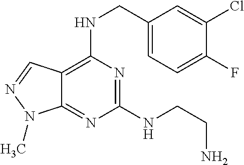 [SMILES: Cn1ncc2c(NCc3ccc(F)c(Cl)c3)nc(NCCN)nc21] | D | 4.35 | 350.1 |
| 1.18 | N6-(2- aminoethyl)-N4- [(4-chloro-2- fluorophenyl) methyl]-1-methyl-1H- pyrazolo[3,4- d]pyrimidine-4,6- diamine | 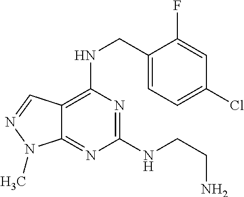 [SMILES: Cn1ncc2c(NCc3ccc(Cl)cc3F)nc(NCCN)nc21] | D | 4.48 | 350.1 |
| 1.19 | N6-(2- aminoethyl)-N4- [(2,4- difluorophenyl) methyl]-1-methyl-1H- pyrazolo[3,4- d]pyrimidine-4,6- diamine | 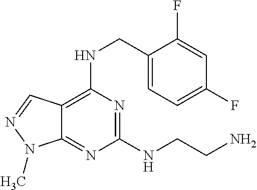 [SMILES: Cn1ncc2c(NCc3ccc(F)cc3F)nc(NCCN)nc21] | D | 4.01 | 334.1 |
| 1.20 | N6-(2- aminoethyl)-N4- [(3-chloro-5- methoxyphenyl) methyl]-1-methyl- 1H-pyrazolo[3,4- d]pyrimidine-4,6- diamine | 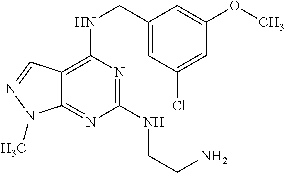 [SMILES: COc1cc(Cl)cc(CNc2nc(NCCN)nc3c2cnn3C)c1] | C | 2.74 | 362.2 |
| 1.21 | N6-(2- aminoethyl)-N4- (3-chloro-4- methoxyphenyl)- 1-methyl-1H- pyrazolo[3,4- d]pyrimidine-4,6- diamine | 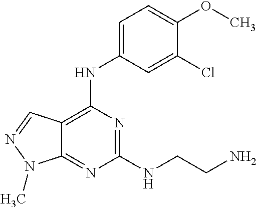 [SMILES: COc1ccc(Nc2nc(NCCN)nc3c2cnn3C)cc1Cl] | D | 4.03 | 348.1 |
| 1.22 | N6-(2- aminoethyl)-N4- [(3-chloro-4- methylphenyl) methyl]-1-methyl-1H- pyrazolo[3,4- d]pyrimidine-4,6- diamine | 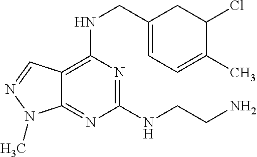 [SMILES: CC1=CC=C(CNc2nc(NCCN)nc3c2cnn3C)CC1Cl] | D | 4.58 | 388.1 |
| 1.23 | N6-(2- aminoethyl)-1- methyl-N4-{[3- (trifluoromethyl) phenyl]methyl}-1H- pyrazolo[3,4- d]pyrimidine-4,6- diamine | 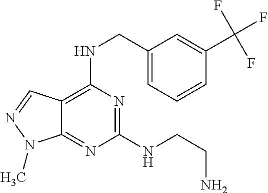 [SMILES: Cn1ncc2c(NCc3cccc(C(F)(F)F)c3)nc(NCCN)nc21] | D | 4.52 | 366.1 |
| 1.24 | N6-(2- aminoethyl)-N4- [(2,4- dimethoxyphenyl) methyl]-1-methyl- 1H-pyrazolo[3,4- d]pyrimidine-4,6- diamine | 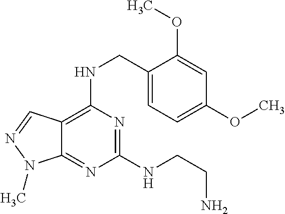 [SMILES: COc1ccc(CNc2nc(NCCN)nc3c2cnn3C)c(OC)c1] | D | 4.16 | 358.2 |
| 1.25 | N6-(2- aminoethyl)-N4- [(3-chloro-4- methoxyphenyl) methyl]-1methyl- 1H-pyrazolo[3,4- d]pyrimidine-4,6- diamine | 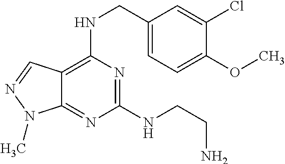 [SMILES: COc1ccc(CNc2nc(NCCN)nc3c2cnn3C)cc1Cl] | C | 2.86 | 362.1 |
| 1.26 | N6-(2-amino-2- methylpropyl)-N4- [(3-chloro-4- methoxyphenyl) methyl]-1-methyl- 1H-pyrazolo[3,4- d]pyrimidine-4,6- diamine | 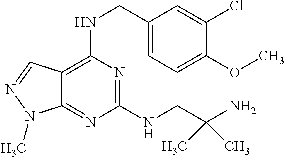 [SMILES: COc1ccc(CNc2nc(NCC(C)(C)N)nc3c2cnn3C)cc1Cl] | C | 3.14 | 390.2/ 392.1 |
| 1.27 | N6-(2-amino-2- methylpropyl)-N4- [(4- methoxyphenyl) methyl]-1-methyl- 1H-pyrazolo[3,4- d]pyrimidine-4,6- diamine | 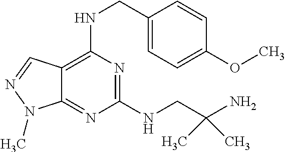 [SMILES: COc1ccc(CNc2nc(NCC(C)(C)N)nc3c2cnn3C)cc1] | C | 1.30 | 356.2 |
| 1.28 | N6-(2-amino-2- methylpropyl)-N4- ({[1,1′-biphenyl]- 4-yl}methyl)-1- methyl-1H- pyrazolo[3,4- d]pyrimidine-4,6- diamine | 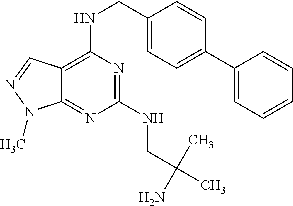 [SMILES: Cn1ncc2c(NCc3ccc(-c4ccccc4)cc3)nc(NCC(C)(C)N)nc21] | D | 5.76 | 402.2 |
| 1.29 | N6-(2- aminoethyl)-N4- [(3-chloro-4- methoxyphenyl) methyl]-1,3- dimethyl-1H- pyrazolo[3,4- d]pyrimidine-4,6- diamine | 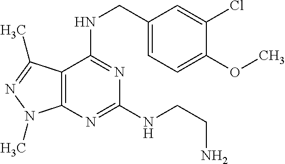 [SMILES: COc1ccc(CNc2nc(NCCN)nc3c2c(C)nn3C)cc1Cl] | D | 4.39 | 376.1/ 378.1 |
| 1.30 | N6-(2-amino-2- methylpropyl)-N4- [(3-chloro-4- methoxyphenyl) methyl]-1,3- dimethyl-1H- pyrazolo[3,4- d]pyrimidine-4,6- diamine | 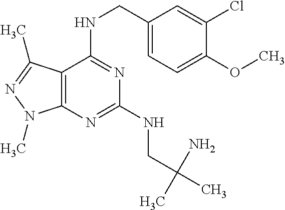 [SMILES: COc1ccc(CNc2nc(NCC(C)(C)N)nc3c2c(C)nn3C)cc1Cl] | D | 4.85 | 404.1/ 406.2 |
| 1.31 | N6-(2-amino-2- methylpropyl)-N4- ({[1,1′-biphenyl]- 4-yl}methyl)-1,3- dimethyl-1H- pyrazolo[3,4- d]pyrimidine-4,6- diamine | 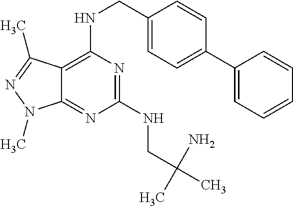 [SMILES: Cc1nn(C)c2nc(NCC(C)(C)N)nc(NCc3ccc(-c4ccccc4)cc3)c12] | E | 2.01 | 416.3 |
| 1.32 | N6-[(1- aminocyclohexyl) methyl]-1-methyl- N4-[4- (trifluoromethyl) phenyl]pyrazolo[3, 4-d]pyrimidine- 4,6-diamine | 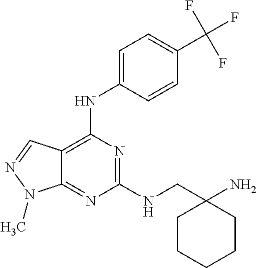 [SMILES: Cn1ncc2c(Nc3ccc(C(F)(F)F)cc3)nc(NCC3(N)CCCCC3)nc21] | D | 4.85 | 420.2 |
| 1.33 | N6-(2-amino-2- methylpropyl)- 1,3-dimethyl-N4- [3- (trifluoromethyl) phenyl]-1H- pyrazolo[3,4- d]pyrimidine-4,6- diamine | 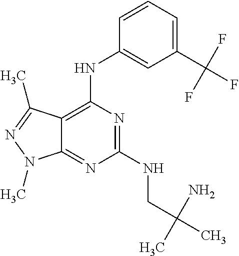 [SMILES: Cc1nn(C)c2nc(NCC(C)(C)N)nc(Nc3cccc(C(F)(F)F)c3)c12] | E | 1.93 | 394.2 |
| 1.34 | 6-N-(2- aminocyclohexyl)- 4-N-[(3-chloro-4- methylphenyl) methyl]-1- methylpyrazolo[3, 4-d]pyrimidine- 4,6-diamine | 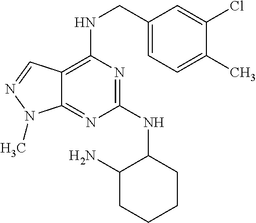 [SMILES: Cc1ccc(CNc2nc(NC3CCCCC3N)nc3c2cnn3C)cc1Cl] | E | 1.87 | 400.3/ 402.3 |
| 1.35 | 6-N-[(4- aminooxan-4- yl)methyl]-1- methyl-4-N-[6- (trifluoromethyl) pyridin-3- yl]pyrazolo[3,4- d]pyrimidine-4,6- diamine | 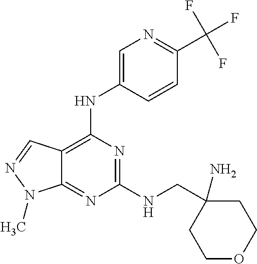 [SMILES: Cn1ncc2c(Nc3ccc(C(F)(F)F)nc3)nc(NCC3(N)CCOCC3)nc21] | D | 3.32 | 4.23.2 |
| 1.36 | N6-[(3R,4S)-4- aminotetrahydrofuran- 3-yl]-1- methyl-N4-[4- (trifluoromethyl) phenyl]pyrazolo[3, 4-d]pyrimidine- 4,6-diamine | 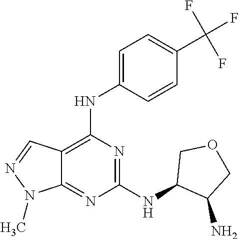 [SMILES: Cn1ncc2c(Nc3ccc(C(F)(F)F)cc3)nc(N[C@H]3COC[C@H]3N)nc21] | D | 3.61 | 394.2 |
| 1.39 | N6-(2- aminoethyl)-N4- [(4- methoxyphenyl) methyl]-1-methyl- 1H-pyrazolo[3,4- d]pyrimidine-4,6- diamine | 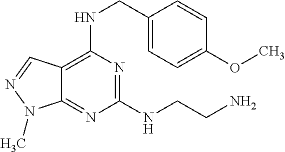 [SMILES: COc1ccc(CNc2nc(NCCN)nc3c2cnn3C)cc1] | D | 3.77 | 328.2 |
| 1.40 | tert-butyl 4-[({6- [(2- aminoethyl)amino]- 1-methyl-1H- pyrazolo[3,4- d]pyrimidin-4- yl}amino)methyl] piperidine-1- carboxylate | 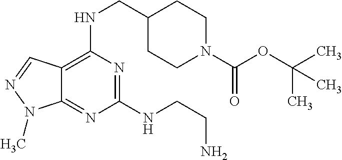 [SMILES: Cn1ncc2c(NCC3CCN(C(=O)OC(C)(C)C)CC3)nc(NCCN)nc21] | D | 4.56 | 405.2 |
| 1.41 | N6-(2- aminoethyl)-N4- [(1- benzylpiperidin-4- yl)methyl]-1- methyl-1H- pyrazolo[3,4- d]pyrimidine-4,6- diamine | 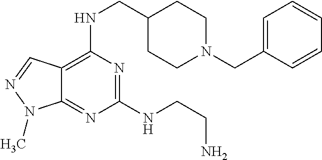 [SMILES: Cn1ncc2c(NCC3CCN(Cc4ccccc4)CC3)nc(NCCN)nc21] | D | 5.18 | 395.3 |
| 1.42 | N6-(2- aminoethyl)-N4- [(1- benzoylpiperidin- 4-yl)methyl]-1- methyl-1H- pyrazolo[3,4- d]pyrimidine-4,6- diamine | 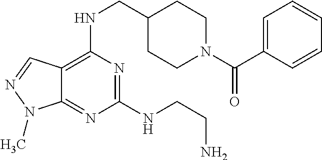 [SMILES: Cn1ncc2c(NCC3CCN(C(=O)c4ccccc4)CC3)nc(NCCN)nc21] | D | 3.67 | 409.2 |

### Compound 1.37: N6-(2-aminoethyl)-N4-{[1-(3-chloro-4-methoxybenzoyl)piperidin-4-yl]methyl}-1-methyl-1H-pyrazolo[3,4-d]pyrimidine-4,6-diamine

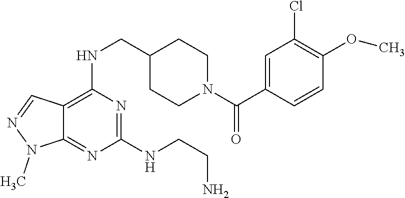 [SMILES: COc1ccc(C(=O)N2CCC(CNc3nc(NCCN)nc4c3cnn4C)CC2)cc1Cl]

Step 1: A solution of 3-chloro-4-methoxybenzoic acid (100 mg, 0.54 mmol), tert-butyl (piperidin-4-ylmethyl)carbamate (114.9 mg, 0.54 mmol), HATU (244.5 mg, 0.64 mmol) and N, N-diisopropylethylamine (115 μl, 0.64 mmol) in DCM (5 mL) was stirred at room temperature for 6 hours. After this time, the reaction mixture was quenched by the addition of water (5 mL) and the organics were extracted into DCM (5 mL). The resulting organics were passed through a TELOS™ phase separator and were concentrated in vacuo to yield the crude product as an orange oil. The oil was suspended in 4 M HCl in 1,4 dioxane (1.4 mL) and the reaction mixture was stirred at room temperature for 1 hour. The reaction mixture was concentrated under reduced pressure to afford [1-(3-chloro-4-methoxybenzoyl)piperidin-4-yl]methanamine.HCl as an orange oil (210 mg, 92%). LCMS (Analytical Method A) t_{R}=0.74 min, [M+H]⁺=283.1/285.0.

Step 2: 6-Chloro-N-{[1-(3-chloro-4-methoxybenzoyl)piperidin-4-yl]methyl}-1-methyl-1H-pyrazolo[3,4-d]pyrimidin-4-amine was synthesised according to General Method 1 at room temperature and was used without purification for Step 3, (120 mg, 60%). LCMS (Analytical Method A) t_{R}=1.12 min, [M+H]⁺=449.1.

Step 3: N6-(2-aminoethyl)-N4-{[1-(3-chloro-4-methoxybenzoyl)piperidin-4-yl]methyl}-1-methyl-1H-pyrazolo[3,4-d]pyrimidine-4,6-diamine was synthesised according to General Method 3 and was purified using high pH preparative HPLC (Method B) to yield the title compound as a white solid (10.0 mg, 9.5%). LCMS (Analytical Method D) t_{R}=4.08 min, [M+H]⁺=473.1/475.1; 1H NMR (500 MHz, DMSO-d6) δ 7.78 (s, 1H), 7.73 (s, 1H), 7.44 (d, J=2.0 Hz, 1H), 7.35 (dd, J=8.4, 2.0 Hz, 1H), 7.19 (d, J=8.5 Hz, 1H), 6.51 (s, 1H), 3.89 (s, 3H), 3.67 (s, 3H), 3.37-3.26 (m, 8H), 2.69 (t, J=6.3 Hz, 2H), 1.95-1.67 (m, 4H), 1.32-1.06 (m, 4H).

### Compound 1.38: 6-N-(2-Amino-2-methylpropyl)-1,3-dimethyl-4-N-[[3-(trifluoromethyl)phenyl]methyl]pyrazolo[3,4-b]pyridine-4,6-diamine

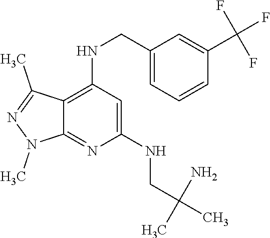 [SMILES: Cc1nn(C)c2nc(NCC(C)(C)N)cc(NCc3cccc(C(F)(F)F)c3)c12]

Step 1: A solution of 4,6-dichloro-1,3-dimethyl-1H-pyrazolo[3,4-b]pyridine (400.0 mg, 1.85 mmol), 3-(trifluoromethyl)benzylamine (390.0 mg, 2.23 mmol) and N-ethyl-N-isopropyl-propan-2-amine (325 uL, 1.86 mmol) in acetonitrile (5 mL) was heated to 100° C. for 18 hours. The reaction mixture was quenched by the addition of 10% citric acid solution (2 mL) and the organics were extracted into DCM (7 mL) and passed through a TELOS™ phase separator. The organic phase was evaporated to dryness under reduced pressure and the crude material was purified by silica gel chromatography eluting with heptane/ethyl acetate (0-100%) to yield 6-chloro-1,3-dimethyl-N-[[3-(trifluoromethyl)phenyl]methyl]pyrazolo[3,4-b]pyridin-4-amine (85 mg, 12.3%) as an off-white solid. LCMS (Analytical Method A) t_{R}=1.25 min, [M+H]⁺=355.0/357.0; 1H NMR (500 MHz, DMSO-d6) δ 7.77 (s, 1H), 7.70 (d, J=7.4 Hz, 1H), 7.61 (dt, J=15.2, 7.7 Hz, 2H), 7.37 (t, J=6.3 Hz, 1H), 6.04 (s, 1H), 4.65 (d, J=6.2 Hz, 2H), 3.78 (s, 3H), 2.63 (s, 3H).

Step 2: To a solution of 2-methylpropane-1,2-diamine (117 uL, 1.13 mmol) and 2-(2-dicyclohexylphosphanylphenyl)-N,N-dimethyl-aniline (9.0 mg, 0.02 mmol) in 1,4-dioxane (2 mL) was added potassium;2-methylpropan-2-olate (76.0 mg, 0.68 mmol) and 6-chloro-1,3-dimethyl-N-[[3-(trifluoromethyl)phenyl]methyl]pyrazolo[3,4-b]pyridin-4-amine (80.0 mg, 0.23 mmol) and the resulting reaction mixture was degassed for 5 minutes. (1{E},4{E})-1,5-Diphenylpenta-1,4-dien-3-one;palladium (21.0 mg, 0.02 mmol) was added and the reaction mixture was heated to 100° C. under thermal conditions for 1 hour. The reaction mixture was allowed to cool to room temperature and purified by low pH preparative HPLC (Method B) to yield N6-(2-amino-2-methyl-propyl)-1,3-dimethyl-N4-[[3-(trifluoromethyl)phenyl]methyl]pyrazolo[3,4-b]pyridine-4,6-diamine (32 mg, 34.2%) as an off white solid. LCMS (Analytical Method E) t_{R}=1.72 min, [M+H]⁺=407.3; 1H NMR (500 MHz, DMSO-d6) δ 8.39 (s, 1H), 7.73 (s, 1H), 7.68 (d, J=7.1 Hz, 1H), 7.63-7.54 (m, 2H), 6.65 (t, J=5.9 Hz, 1H), 6.59 (t, J=6.2 Hz, 1H), 5.27 (s, 1H), 4.50 (d, J=6.1 Hz, 2H), 3.67 (s, 3H), 3.33 (s, 3H), 3.32 (d, J=6.1 Hz, 2H), 1.14 (s, 6H).

### Compound 2.01: 6-N-[(1-Aminocyclopropyl)methyl]-4-N-[(3-chloro-4-methylphenyl)methyl]-1-methylpyrazolo[3,4-d]pyrimidine-4,6-diamine

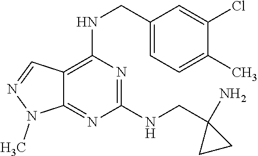 [SMILES: Cc1ccc(CNc2nc(NCC3(N)CC3)nc3c2cnn3C)cc1Cl]

Step 1: To a solution of 4,6-dichloro-1-methyl-pyrazolo[3,4-d]pyrimidine (1.3 g, 6.4 mmol) and N, N-diisopropylethylamine (3.43 mL, 19.21 mmol) in acetonitrile (22 mL) cooled to 0° C. was added (3-chloro-4-methyl-phenyl)methanamine (996 mg, 6.4 mmol) and the reaction mixture was allowed to warm to ambient temperature overnight. The reaction mixture was quenched by the addition of water and the organics were extracted into ethyl acetate (20 mL). The organic layer was washed with brine (10 mL), dried over magnesium sulfate, filtered and concentrated in vacuo to afford the desired compound as a yellow oil which was purified by silica gel chromatography eluting with 0-100% ethyl acetate:heptane to afford the desired compound as a yellow solid (1.02 g, 47.6%). LCMS (Analytical Method A) t_{R}=1.26 min, [M+H]⁺=321.95/323.95; 1H NMR (250 MHz, DMSO-d6) δ 9.16 (t, J=5.7 Hz, 1H), 8.12 (s, 1H), 7.41 (d, J=1.3 Hz, 1H), 7.32 (d, J=7.8 Hz, 1H), 7.22 (dd, J=7.8, 1.5 Hz, 1H), 4.66 (d, J=5.8 Hz, 2H), 3.85 (s, 3H), 2.30 (s, 3H).

Step 2: To a solution of 6-chloro-N-[(3-chloro-4-methyl-phenyl)methyl]-1-methyl-pyrazolo[3,4-d]pyrimidin-4-amine (50.0 mg, 0.16 mmol) in 1-butanol (0.25 mL) was added tert-butyl N-[1-(aminomethyl)cyclopropyl]carbamate (122 uL, 0.54 mmol) and the resulting reaction mixture was heated to 110° C. for 18 hours. The resulting intermediate product was purified by high pH preparative HPLC (Method B) to afford the BOC intermediate as a white solid; LCMS (Analytical Method B) t_{R}=1.55 min, [M+H]⁺=472.2/474.2. The BOC intermediate was stirred in DCM (2 mL) and TFA (0.23 mL, 3.10 mmol) until complete consumption of the starting material was observed by LCMS (12 hours). The reaction mixture was concentrated in vacuo and loaded on to an SCX cartridge eluting with methanol followed by 7N ammonia in methanol. The organics were concentrated in vacuo and lyophilised to afford the title compound as an off-white solid, (39.8 mg, 57%). LCMS (Analytical Method D) t_{R}=3.93 min, [M+H]⁺=372.2/374.2. 1H NMR (250 MHz, DMSO-d6) δ 7.93 (t, 1H), 7.78 (s, 1H), 7.38 (s, 1H), 7.33-7.15 (m, 2H), 6.31 (t, J=5.6 Hz, 1H), 4.63 (d, J=6.0 Hz, 2H), 3.69 (s, 3H), 3.35 (d, J=5.8 Hz, 2H), 2.31 (s, 3H), 0.55-0.44 (m, 2H), 0.44-0.34 (m, 2H).

### Compound 2.02: 6-N-[(1-Aminocyclopropyl)methyl]-4-N-[(3-chloro-4-fluorophenyl)methyl]-1-methylpyrazolo[3,4-d]pyrimidine-4,6-diamine

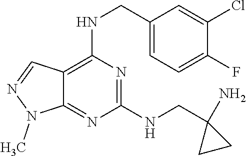 [SMILES: Cn1ncc2c(NCc3ccc(F)c(Cl)c3)nc(NCC3(N)CC3)nc21]

Step 1: To a solution of 4,6-dichloro-1-methyl-pyrazolo[3,4-d]pyrimidine (500.0 mg, 2.46 mmol) and N, N-diisopropylethylamine (660 uL, 3.7 mmol) in acetonitrile (20 mL) at 0° C. was added 1-(3-chloro-4-fluorophenyl)methanamine (345 uL, 2.75 mmol) and the reaction mixture was stirred at room temperature for 72 hours. The reaction mixture was quenched by the addition of 10% citric acid solution (10 mL) and the organics extracted into ethyl acetate (2×30 mL). The desired product was isolated as a yellow solid after purification by silica gel chromatography eluting with ethyl acetate:heptane (0-100%) to yield the desired compound of sufficient purity for further manipulation (880 mg, 100%). LCMS (Analytical Method A) t_{R}=1.17 min, [M+H]⁺=325.9/329.9; 1H NMR (500 MHz, DMSO-d6) δ 9.19 (t, J=5.7 Hz, 1H), 8.12 (s, 1H), 7.60 (dd, J=7.2, 1.6 Hz, 1H), 7.46-7.34 (m, 2H), 4.69 (d, J=5.8 Hz, 2H), 3.86 (s, 3H).

Step 2: A mixture of 6-chloro-N-[(3-chloro-4-fluoro-phenyl)methyl]-1-methyl-pyrazolo[3,4-d]pyrimidin-4-amine (100 mg, 0.307 mmol) and tert-butyl N-[1-(aminomethyl)cyclopropyl]carbamate (114 mg, 0.612 mmol) in 1-butanol (1 mL) was heated 30 to 125° C. for 24 hours. The solvent was removed in vacuo and the crude reaction mixture was purified by silica gel chromatography eluting with 0-100% ethyl acetate:heptane to yield the intermediate BOC product as a yellow solid (78 mg, 49%). LCMS (Analytical Method A) t_{R}=1.13 min, [M+H]⁺=476.15/478.15; 1H NMR (250 MHz, Chloroform-d) δ 7.60 (s, 1H), 7.45-7.35 (m, 1H), 7.24-7.17 (m, 1H), 7.10 (t, J=8.6 Hz, 1H), 5.54 (s, 1H), 5.30 (s, 1H), 5.01 (s, 1H), 4.71 (d, J=5.9 Hz, 2H), 3.84 (s, 3H), 3.57 (d, J=5.6 Hz, 2H), 1.41 (s, 9H), 0.83 (s, 4H). tert-Butyl N-[1-[[[4-[(3-chloro-4-fluoro-phenyl)methylamino]-1-methyl-pyrazolo[3,4-d]pyrimidin-6-yl]amino]methyl]cyclopropyl]carbamate (78 mg, 0.164 mmol) was suspended in 4M HCl in dioxane (1.6 mL) and the reaction mixture was stirred at room temperature for 1 hour and concentrated in vacuo. The material was dissolved in the minimum amount of methanol, loaded onto an SCX cartridge, washed with methanol and eluted with 2 N NH₃ in methanol. The resulting material was lyophilised to yield the title compound as an off-white solid (42 mg, 68%). LCMS (Analytical Method D) t_{R}=3.60 min, [M+H]⁺=376.2; 1 H NMR (500 MHz, Chloroform-d) δ 7.61 (s, 1H), 7.42 (dd, J=6.9, 2.2 Hz, 1H), 7.25-7.20 (m, 1H), 7.11 (t, J=8.6 Hz, 1H), 5.32 (d, J=23.0 Hz, 1H), 5.17 (s, 1H), 4.73 (s, 2H), 3.85 (s, 3H), 3.48 (d, J=6.1 Hz, 2H), 0.69-0.52 (m, 4H). 19F NMR (235 MHz, Chloroform-d) δ −117.08

### Compound 2.03: N6-[(1-aminocyclopropyl)methyl]-N4-[3-fluoro-4-(trifluoromethyl)phenyl]-1-methyl-pyrazolo[3,4-d]pyrimidine-4,6-diamine

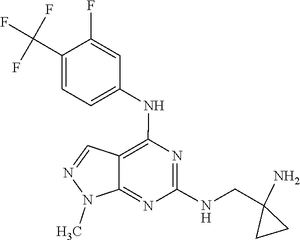 [SMILES: Cn1ncc2c(Nc3ccc(C(F)(F)F)c(F)c3)nc(NCC3(N)CC3)nc21]

Step 1: To a solution of 4,6-dichloro-1-methyl-1H-pyrazolo[3,4-d]pyrimidine (200 mg, 0.985 mmol) and 3-fluoro-4-(trifluoromethyl)aniline (176 mg, 0.985 mmol) in 1,4-dioxane (2 mL) was added 2 M sodium;2-methylpropan-2-olate (0.99 mL, 1.97 mmol) and the resulting reaction mixture was stirred at 100° C. for 30 minutes whereupon the reaction was deemed complete by LCMS. The reaction mixture was loaded onto a silica gel column and purified by eluting with 20-60% heptane:ethyl acetate to yield the desired compound as a pale brown solid (218 mg, 62.7%). LCMS (Analytical Method B) t_{R}=1.34 min, [M+H]⁺=345.9; 1H NMR (400 MHz, DMSO-d6) δ 10.88 (s, 1H), 8.34 (s, 1H), 8.17-8.10 (m, 1H), 7.83 (t, J=8.6 Hz, 1H), 7.71 (d, J=8.4 Hz, 1H), 3.94 (s, 3H); 19F NMR (376 MHz, DMSO-d6) δ −59.14, −113.98.

Step 2: A solution of 6-chloro-N-[3-fluoro-4-(trifluoromethyl)phenyl]-1-methyl-pyrazolo[3,4-d]pyrimidin-4-amine (50.0 mg, 0.14 mmol) and tert-butyl N-[1-(aminomethyl)cyclopropyl]carbamate (80.0 mg, 0.43 mmol) in 1-butanol (0.25 mL) was heated to 110° C. for 18 hours. The resulting product was purified high pH preparative HPLC (Method B) to yield the BOO intermediate. The BOO intermediate was dissolved in 4M HCl in dioxane (10 mL) and was stirred for 1 hour at room temperature. The solution was concentrated in vacuo and dissolved in a minimum amount of methanol (5 mL). The compound was loaded onto an SCX column and eluted with 7N ammonia in methanol to yield N6-[(1-aminocyclopropyl)methyl]-N4-[3-fluoro-4-(trifluoromethyl)phenyl]-1-methyl-pyrazolo[3,4-d]pyrimidine-4,6-diamine (31 mg, 54%) as an off white solid. LOMS (Analytical Method C) t_{R}=3.15 min [M+H]⁺=396.2; 1H NMR (400 MHz, DMSO-d6) b 9.88 (s, 1H), 8.31 (d, J=14.4 Hz, 1H), 8.03 (s, 1H), 7.78-7.72 (m, 1H), 7.67 (t, J=8.5 Hz, 1H), 6.95 (t, J=5.6 Hz, 1H), 3.77 (s, 3H), 3.45 (d, J=5.8 Hz, 2H), 1.91 (s, 2H), 0.61-0.56 (m, 2H), 0.50-0.45 (in, 2H); 19F NMR (376 MHz, DMSO-d6) δ −58.8, −113.79.

The compounds in Table 2 were prepared in an analogous manner to Compounds 2.01-2.03 according to either General Method 4 or General Method 5 using ter-butyl N-[1-(aminomethyl)cyclopropyl]carbamate and the appropriate 4-substituted pyrazolopyrimidine intermediate which was in turn synthesised according to General Method 1 or General Method 2.

TABLE 2

| Compound | | | Analytical | t_{R} | |
|---|---|---|---|---|---|
| No. | Name | Structure | Method | (min) | [M + H]⁺ |
| 2.04 | 6-N-[(1- aminocyclopropyl) methyl]-4-N-(3- chloro-4- methoxyphenyl)- 1- methylpyrazolo[3, 4-d]pyrimidine- 4,6-diamine | 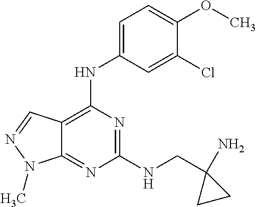 [SMILES: COc1ccc(Nc2nc(NCC3(N)CC3)nc3c2cnn3C)cc1Cl] | D | 3.55 | 374.2/ 376.2 |
| 2.05 | 6-N-[(1- aminocyclopropyl) methyl]-4-N-(2,4- difluorophenyl)-1- methylpyrazolo[3, 4-d]pyrimidine- 4,6-diamine | 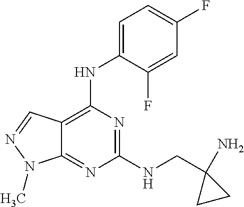 [SMILES: Cn1ncc2c(Nc3ccc(F)cc3F)nc(NCC3(N)CC3)nc21] | D | 3.37 | 346.2 |
| 2.06 | 6-N-[(1- aminocyclopropyl) methyl]-1- methyl-4-N-[3- (trifluoromethyl) phenyl]pyrazolo[3, 4-d]pyrimidine- 4,6-diamine | 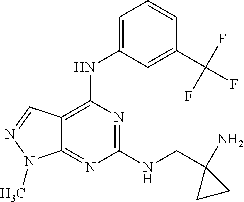 [SMILES: Cn1ncc2c(Nc3cccc(C(F)(F)F)c3)nc(NCC3(N)CC3)nc21] | D | 3.90 | 378.3 |
| 2.07 | 6-N-[(1- aminocyclopropyl) methyl]-4-N-[3- (difluoromethyl) phenyl]-1- methylpyrazolo[3, 4-d]pyrimidine- 4,6-diamine | 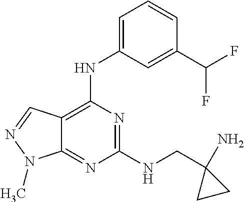 [SMILES: Cn1ncc2c(Nc3cccc(C(F)F)c3)nc(NCC3(N)CC3)nc21] | D | 3.59 | 360.3 |
| 2.08 | 6-N-[(1- aminocyclopropyl) methyl]-4-N-(4- fluorophenyl)-1- methylpyrazolo[3, 4-d]pyrimidine- 4,6-diamine | 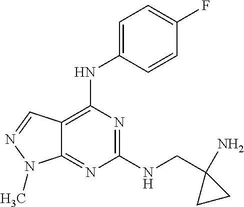 [SMILES: Cn1ncc2c(Nc3ccc(F)cc3)nc(NCC3(N)CC3)nc21] | D | 3.43 | 328.3 |
| 2.09 | 6-N-[(1- aminocyclopropyl) methyl]-4-N-(3- chloro-4- methylphenyl)-1- methylpyrazolo[3, 4-d]pyrimidine- 4,6-diamine | 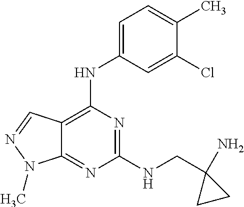 [SMILES: Cc1ccc(Nc2nc(NCC3(N)CC3)nc3c2cnn3C)cc1Cl] | E | 3.07 | 358.2/ 360.2 |
| 2.10 | 6-N-[(1- aminocyclopropyl) methyl]-1- methyl-4-N- pyridin-3- ylpyrazolo[3,4- d]pyrimidine-4,6- diamine | 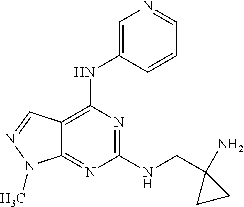 [SMILES: Cn1ncc2c(Nc3cccnc3)nc(NCC3(N)CC3)nc21] | D | 2.85 | 311.3 |
| 2.11 | 6-N-[(1- aminocyclopropyl) methyl]-1- methyl-4-N- phenylpyrazolo[3, 4-d]pyrimidine- 4,6-diamine | 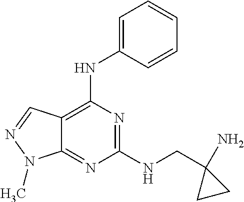 [SMILES: Cn1ncc2c(Nc3ccccc3)nc(NCC3(N)CC3)nc21] | C | 2.46 | 310.2 |
| 2.12 | 6-N-[(1- aminocyclopropyl) methyl]-4-N-[(3- bromophenyl) methyl]-1- methylpyrazolo[3, 4-d]pyrimidine- 4,6-diamine | 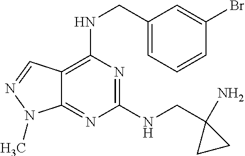 [SMILES: Cn1ncc2c(NCc3cccc(Br)c3)nc(NCC3(N)CC3)nc21] | C | 2.82 | 402.2/ 404.2 |
| 2.13 | 6-N-[(1- aminocyclopropyl) methyl]-4-N-[(4- bromophenyl) methyl]-1- methylpyrazolo[3, 4-d]pyrimidine- 4,6-diamine | 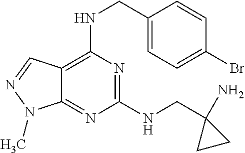 [SMILES: Cn1ncc2c(NCc3ccc(Br)cc3)nc(NCC3(N)CC3)nc21] | C | 2.87 | 402.2/ 404.2 |
| 2.14 | 6-N-[(1- aminocyclopropyl) methyl]-4-N-(2- chloro-4- fluorophenyl)-1- methylpyrazolo[3, 4-d]pyrimidine- 4,6-diamine | 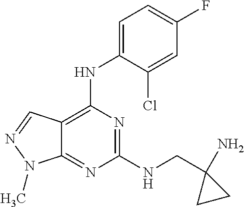 [SMILES: Cn1ncc2c(Nc3ccc(F)cc3Cl)nc(NCC3(N)CC3)nc21] | C | 2.62 | 362.2/ 364.1 |
| 2.15 | 6-N-[(1- aminocyclopropyl) methyl]-4-N-(4- chloro-2- fluorophenyl)-1- methylpyrazolo[3, 4-d]pyrimidine- 4,6-diamine | 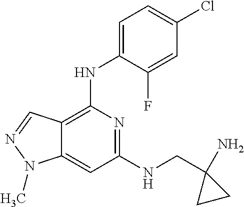 [SMILES: Cn1ncc2c(Nc3ccc(Cl)cc3F)nc(NCC3(N)CC3)cc21] | D | 3.60 | 362.2/ 364.2 |
| 2.16 | 6-N-[(1- aminocyclopropyl) methyl]-4-N-(3- chloro-5- fluorophenyl)-1- methylpyrazolo[3, 4-d]pyrimidine- 4,6-diamine | 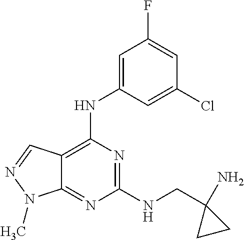 [SMILES: Cn1ncc2c(Nc3cc(F)cc(Cl)c3)nc(NCC3(N)CC3)nc21] | D | 3.84 | 362.2/ 364.2 |
| 2.17 | 5-[[6-[(1- aminocyclopropyl) methylamino]-1- methyl- pyrazolo[3,4- d]pyrimidin-4- yl]amino]-2- chloro- benzonitrile | 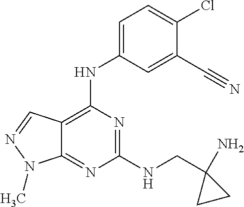 [SMILES: Cn1ncc2c(Nc3ccc(Cl)c(C#N)c3)nc(NCC3(N)CC3)nc21] | E | 3.66 | 369.2/ 371.2 |
| 2.18 | N6-[(1- aminocyclopropyl) methyl]-1- methyl-N4-(1- phenyl-3- bicyclo[1.1.1] pentanyl)pyrazolo [3,4-d]pyrimidine- 4,6-diamine | 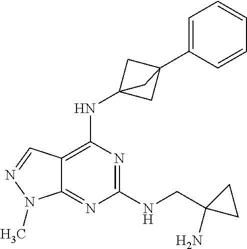 [SMILES: Cn1ncc2c(NC34CC(c5ccccc5)(C3)C4)nc(NCC3(N)CC3)nc21] | D | 3.93 | 376.3 |
| 2.19 | 6-N-[(1- aminocyclopropyl) methyl]-1- methyl-4-N-[[6- (trifluoromethyl) pyridin-3- yl]methyl]pyrazolo [3,4-d]pyrimidine- 4,6-diamine | 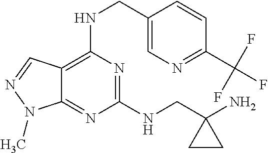 [SMILES: Cn1ncc2c(NCc3ccc(C(F)(F)F)nc3)nc(NCC3(N)CC3)nc21] | D | 3.27 | 393.2 |
| 2.20 | 6-N-[(1- aminocyclopropyl) methyl]-4-N-(4- chloro-3,5- difluorophenyl)-1- methylpyrazolo[3, 4-d]pyrimidine- 4,6-diamine | 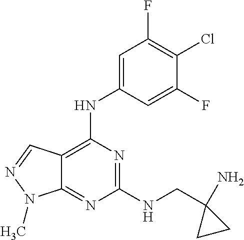 [SMILES: Cn1ncc2c(Nc3cc(F)c(Cl)c(F)c3)nc(NCC3(N)CC3)nc21] | D | 3.97 | 380.2/ 382.2 |
| 2.21 | 6-N-[(1- aminocyclopropyl) methyl]-4-N-[3- (difluoromethoxy) phenyl]-1- methylpyrazolo[3, 4-d]pyrimidine- 4,6-diamine | 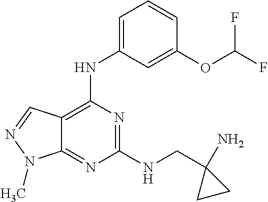 [SMILES: Cn1ncc2c(Nc3cccc(OC(F)F)c3)nc(NCC3(N)CC3)nc21] | D | 3.59 | 376.3 |
| 2.22 | 6-N-[(1- aminocyclopropyl) methyl]-1- methyl-4-N-(4- propan-2- yloxyphenyl) pyrazolo[3,4- d]pyrimidine-4,6- diamine | 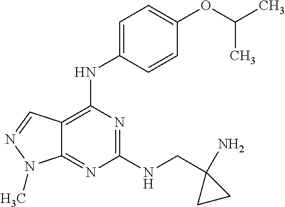 [SMILES: CC(C)Oc1ccc(Nc2nc(NCC3(N)CC3)nc3c2cnn3C)cc1] | D | 3.64 | 368.3 |
| 2.23 | 6-N-[(1- aminocyclopropyl) methyl]-4-N-[(4- chloro-3- fluorophenyl) methyl]-1- methylpyrazolo[3, 4-d]pyrimidine- 4,6-diamine | 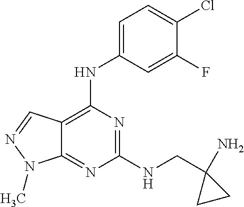 [SMILES: Cn1ncc2c(Nc3ccc(Cl)c(F)c3)nc(NCC3(N)CC3)nc21] | D | 3.11 | 362.2/ 364.2 |
| 2.24 | 6-N-[(1- aminocyclopropyl) methyl]-4-N-(2,3- difluorophenyl)-1- methylpyrazolo[3, 4-d]pyrimidine- 4,6-diamine | 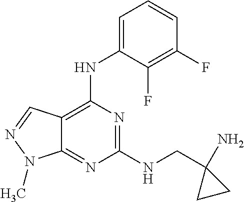 [SMILES: Cn1ncc2c(Nc3cccc(F)c3F)nc(NCC3(N)CC3)nc21] | D | 3.36 | 346.3 |
| 2.25 | 6-N-[(1- aminocyclopropyl) methyl]-4-N-(2,5- difluorophenyl)-1- methylpyrazolo[3, 4-d]pyrimidine- 4,6-diamine | 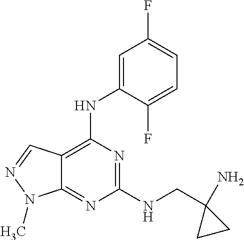 [SMILES: Cn1ncc2c(Nc3cc(F)ccc3F)nc(NCC3(N)CC3)nc21] | D | 3.45 | 346.2 |
| 2.26 | 6-N-[(1- aminocyclopropyl) methyl]-4-N- 1(2,4- difluorophenyl) methyl]-1- methylpyrazolo[3, 4-d]pyrimidine- 4,6-diamine | 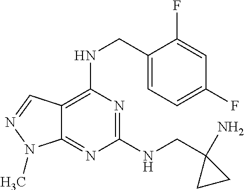 [SMILES: Cn1ncc2c(NCc3ccc(F)cc3F)nc(NCC3(N)CC3)nc21] | D | 3.42 | 360.3 |
| 2.27 | 6-N-[(1- aminocyclopropyl) methyl]-4-N-(3- chloro-4- fluorophenyl)-1- methylpyrazolo[3, 4-d]pyrimidine- 4,6-diamine | 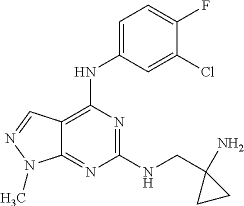 [SMILES: Cn1ncc2c(Nc3ccc(F)c(Cl)c3)nc(NCC3(N)CC3)nc21] | D | 3.79 | 362.3/ 364.2 |
| 2.28 | 6-N-[(1- aminocyclopropyl) methyl]-4-N-(4- chlorophenyl)-1- methylpyrazolo[3, 4-d]pyrimidine- 4,6-diamine | 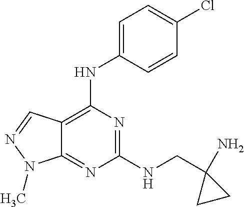 [SMILES: Cn1ncc2c(Nc3ccc(Cl)cc3)nc(NCC3(N)CC3)nc21] | D | 3.71 | 344.3/ 346.3 |
| 2.29 | 6-N-[(1- aminocyclopropyl) methyl]-4-N-(3- fluorophenyl)-1- methylpyrazolo[3, 4-d]pyrimidine- 4,6-diamine | 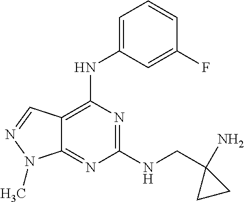 [SMILES: Cn1ncc2c(Nc3cccc(F)c3)nc(NCC3(N)CC3)nc21] | D | 3.58 | 328.2 |
| 2.30 | 6-N-[(1- aminocyclopropyl) methyl]-1,3- dimethyl-4-N-[4- (trifluoromethyl) phenyl]pyrazolo[3, 4-d]pyrimidine- 4,6-diamine | 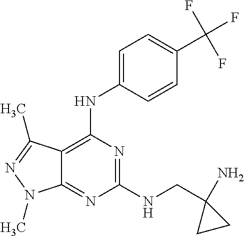 [SMILES: Cc1nn(C)c2nc(NCC3(N)CC3)nc(Nc3ccc(C(F)(F)F)cc3)c12] | D | 3.98 | 392.3 |
| 2.31 | 4-[[6-[(1- aminocyclopropyl) methylamino]-1- methylpyrazolo[3, 4-d]pyrimidin-4- yl]amino] benzonitrile | 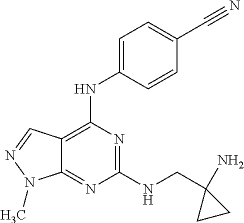 [SMILES: Cn1ncc2c(Nc3ccc(C#N)cc3)nc(NCC3(N)CC3)nc21] | C | 2.39 | 335.2 |
| 2.32 | 6-N-[(1- aminocyclopropyl) methyl]-1- methyl-4-N-[4- (trifluoromethoxy) phenyl]pyrazolo [3,4-d]pyrimidine- 4,6-diamine | 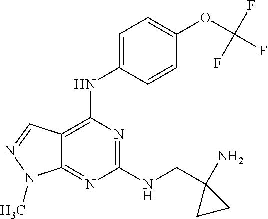 [SMILES: Cn1ncc2c(Nc3ccc(OC(F)(F)F)cc3)nc(NCC3(N)CC3)nc21] | D | 4.04 | 394.2 |
| 2.33 | 6-N-[(1- aminocyclopropyl) methyl]-4-N-(4- methoxyphenyl)- 1- methylpyrazolo[3, 4-d]pyrimidine- 4,6-diamine | 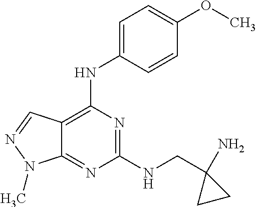 [SMILES: COc1ccc(Nc2nc(NCC3(N)CC3)nc3c2cnn3C)cc1] | D | 3.30 | 340.3 |
| 2.34 | 6-N-[(1- aminocyclopropyl) methyl]-4-N-[4- (difluoromethoxy) phenyl]-1- methylpyrazolo[3, 4-d]pyrimidine- 4,6-diamine | 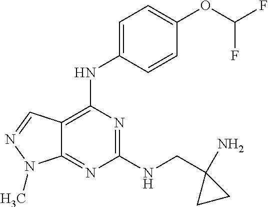 [SMILES: Cn1ncc2c(Nc3ccc(OC(F)F)cc3)nc(NCC3(N)CC3)nc21] | D | 3.72 | 376.2 |
| 2.35 | 6-N-[(1- aminocyclopropyl) methyl]-4-N-(3- chlorophenyl)-1- methylpyrazolo[3, 4-d]pyrimidine- 4,6-diamine | 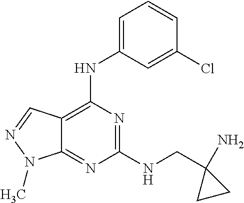 [SMILES: Cn1ncc2c(Nc3cccc(Cl)c3)nc(NCC3(N)CC3)nc21] | D | 3.87 | 344.2/ 346.2 |
| 2.36 | 6-N-[(1- aminocyclopropyl) methyl]-4-N-[4- (difluoromethyl) phenyl]-1- methylpyrazolo[3, 4-d]pyrimidine- 4,6-diamine | 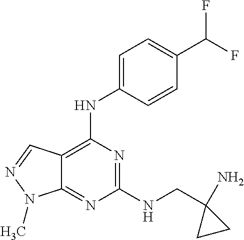 [SMILES: Cn1ncc2c(Nc3ccc(C(F)F)cc3)nc(NCC3(N)CC3)nc21] | C | 2.71 | 360.2 |
| 2.37 | 3-[[6-[(1- aminocyclopropyl) methylamino]-1- methylpyrazolo[3, 4-d]pyrimidin-4- yl]amino]bicyclo [1.1.1]pentane-1- carbonitrile | 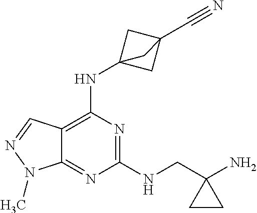 [SMILES: Cn1ncc2c(NC34CC(C#N)(C3)C4)nc(NCC3(N)CC3)nc21] | C | 2.20 | 325.3 |
| 2.38 | 6-N-[(1- aminocyclopropyl) methyl]-4-N-(3- bicyclo[1.1.1] pentanyl)-1- methylpyrazolo[3, 4-d]pyrimidine- 4,6-diamine | 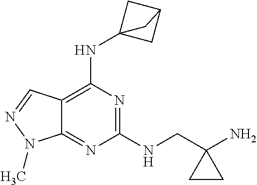 [SMILES: Cn1ncc2c(NC34CC(C3)C4)nc(NCC3(N)CC3)nc21] | D | 3.32 | 300.3 |
| 2.39 | 6-N-[(1- aminocyclopropyl) methyl]-4-N-(3,4- difluorophenyl)-1- methylpyrazolo[3, 4-d]pyrimidine- 4,6-diamine | 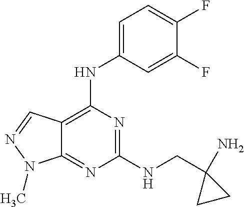 [SMILES: Cn1ncc2c(Nc3ccc(F)c(F)c3)nc(NCC3(N)CC3)nc21] | D | 3.63 | 346.3 |
| 2.40 | 6-N-[(1- aminocyclopropyl) methyl]-4-N-(3,4- dichlorophenyl)-1- methylpyrazolo[3, 4-d]pyrimidine- 4,6-diamine | 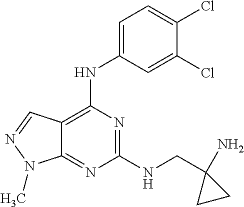 [SMILES: Cn1ncc2c(Nc3ccc(Cl)c(Cl)c3)nc(NCC3(N)CC3)nc21] | C | 3.10 | 378.2/ 382.1 |
| 2.41 | 6-N-[(1- aminocyclopropyl) methyl]-4-N-(4- chloro-3- methoxyphenyl)- 1- methylpyrazolo[3, 4-d]pyrimidine- 4,6-diamine | 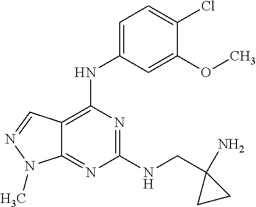 [SMILES: COc1cc(Nc2nc(NCC3(N)CC3)nc3c2cnn3C)ccc1Cl] | C | 2.78 | 374.2/ 376.1 |
| 2.42 | N6-[(1- aminocyclopropyl) methyl]-N4-[(2,4- dimethoxyphenyl) methyl]-1-methyl- pyrazolo[3,4- d]pyrimidine-4,6- diamine | 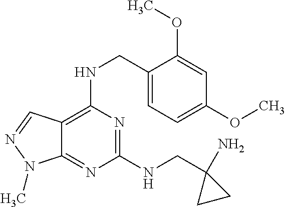 [SMILES: COc1ccc(CNc2nc(NCC3(N)CC3)nc3c2cnn3C)c(OC)c1] | D | 3.36 | 384.3 |
| 2.43 | N6-[(1- aminocyclopropyl) methyl]-N4-{3- fluorobicyclo [1.1.1]pentan-1-yl}- 1-methyl-1H- pyrazolo[3,4- d]pyrimidine-4,6- diamine | 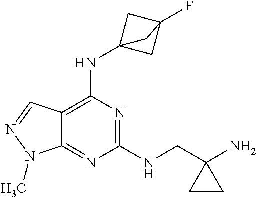 [SMILES: Cn1ncc2c(NC34CC(F)(C3)C4)nc(NCC3(N)CC3)nc21] | E | 1.24 | 318.2 |
| 2.44 | 6-N-[(1- aminocyclopropyl) methyl]-1- methyl-4-N-[4- (trifluoromethyl) phenyl]pyrazolo[3, 4-d]pyrimidine- 4,6-diamine | 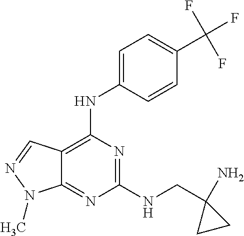 [SMILES: Cn1ncc2c(Nc3ccc(C(F)(F)F)cc3)nc(NCC3(N)CC3)nc21] | D | 3.98 | 378.2 |
| 2.45 | 6-N-[(1- aminocyclopropyl) methyl]-1- methyl-4-N-[6- (trifluoromethyl) pyridin-3- yl]pyrazolo[3,4- d]pyrimidine-4,6- diamine | 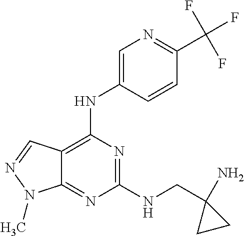 [SMILES: Cn1ncc2c(Nc3ccc(C(F)(F)F)nc3)nc(NCC3(N)CC3)nc21] | D | 3.53 | 379.3 |
| 2.46 | 6-N-[(1- aminocyclopropyl) methyl]-4-N-(5- chloropyridin-2- yl)-1- methylpyrazolo[3, 4-d]pyrimidine- 4,6-diamine | 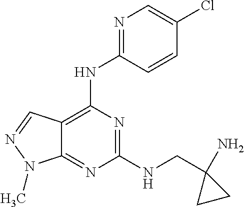 [SMILES: Cn1ncc2c(Nc3ccc(Cl)cn3)nc(NCC3(N)CC3)nc21] | C | 2.56 | 345.1/ 347.1 |
| 2.47 | N6-[(1- aminocyclopropyl) methyl]-1- methyl-N4-[5- (trifluoromethyl) pyrazin-2- yl]pyrazolo[3,4- d]pyrimidine-4,6- diamine | 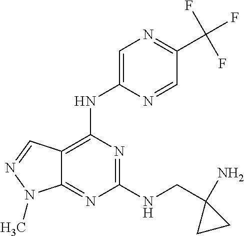 [SMILES: Cn1ncc2c(Nc3cnc(C(F)(F)F)cn3)nc(NCC3(N)CC3)nc21] | D | 3.55 | 380.2 |
| 2.48 | 6-N-[(1- aminocyclopropyl) methyl]-1-ethyl- 4-N-[4- (trifluoromethyl) phenyl]pyrazolo[3, 4-d]pyrimidine- 4,6-diamine | 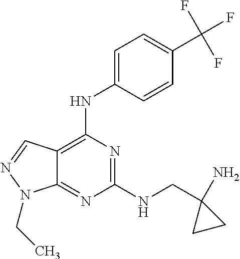 [SMILES: CCn1ncc2c(Nc3ccc(C(F)(F)F)cc3)nc(NCC3(N)CC3)nc21] | C | 3.24 | 392.3 |
| 2.49 | N6-[(1- aminocyclopropyl) methyl]-1- (trideuteriomethyl)- N4-[4- (trifluoromethyl) phenyl]pyrazolo [3,4-d]pyrimidine- 4,6-diamine | 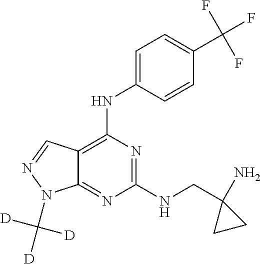 [SMILES: [2H]C([2H])([2H])n1ncc2c(Nc3ccc(C(F)(F)F)cc3)nc(NCC3(N)CC3)nc21] | D | 3.91 | 381.3 |
| 2.50 | N6-[(1- aminocyclopropyl) methyl]-1- (trideuteriomethyl)- N4-[6- (trifluoromethyl)- 3- pyridyl]pyrazolo [3,4-d]pyrimidine- 4,6-diamine | 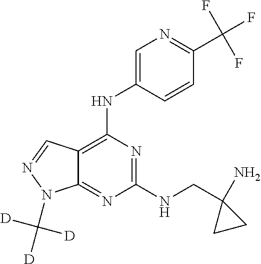 [SMILES: [2H]C([2H])([2H])n1ncc2c(Nc3ccc(C(F)(F)F)nc3)nc(NCC3(N)CC3)nc21] | D | 3.49 | 382.2 |
| 2.51 | 6-N-[(1- aminocyclopropyl) methyl]-4-N-[[4- [2-(3-but-3- ynyldiazirin-3- yl)ethoxy]-3- chlorophenyl] methyl]-1- methylpyrazolo[3, 4-d]pyrimidine- | 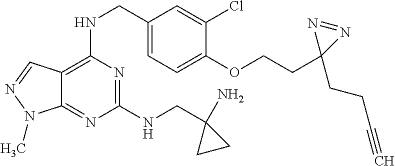 [SMILES: C#CCCC1(CCOc2ccc(CNc3nc(NCC4(N)CC4)nc4c3cnn4C)cc2Cl)N=N1] | C | 3.32 | 494.3/ 496.3 |
| | 4,6-diamine | | | | |

The following compounds were synthesised from tert-butyl N-[1-(aminomethyl)cyclopropyl]carbamate but do not conform with the General methods detailed in Table 2.

### Compound 2.52: N6-[(1-Aminocyclopropyl)methyl]-1-methyl-N4-[4-(trifluoromethyl)phenyl]pyrazolo[3,4-b]pyridine-4,6-diamine

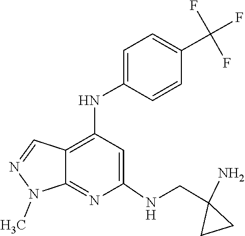 [SMILES: Cn1ncc2c(Nc3ccc(C(F)(F)F)cc3)cc(NCC3(N)CC3)nc21]

Step 1: A mixture of 4-bromo-2-fluoro-pyridine-3-carbaldehyde (500 mg, 2.45 mmol), methyl hydrazine (226 mg, 4.90 mmol) and N-ethyl-N-isopropyl-propan-2-amine (0.86 mL, 4.90 mmol) was heated to 50° C. for 1 hour. The reaction mixture was cooled to room temperature and partitioned between ethyl acetate (10 mL) and water (10 mL). The layers were separated and the aqueous layer extracted twice more with ethyl acetate (2×10 mL). The combined organic layers were washed with brine (20 mL), dried over sodium sulfate, filtered and concentrated in vacuo to give the crude material as a beige solid which was purified by silica gel chromatography eluting with ethyl acetate:heptane (0:100 to 60:40) to give 4-bromo-1-methyl-pyrazolo[3,4-b]pyridine (425 mg, 82%) as a white powder. LCMS (Analytical Method B) t_{R}=1.40 min, [M+H]⁺=212.1/214.1; 1H NMR (400 MHz, Chloroform-d) δ 8.26 (d, J=4.9 Hz, 1H), 7.94 (s, 1H), 7.24 (d, J=4.9 Hz, 1H), 4.09 (s, 3H).

Step 2: To a solution of 4-bromo-1-methyl-pyrazolo[3,4-b]pyridine (420 mg, 1.98 mmol) in DCM (10 mL) was added portionwise 3-chlorobenzenecarboperoxoic acid (70%, 1.46 g, 5.94 mmol) at 0° C. The reaction mixture was allowed to warm to room temperature and stirring continued for 18 hours. Additional 3-chlorobenzenecarboperoxoic acid (70%, 1.46 g, 5.94 mmol) was added and stirring continued for a further 18 hours. The yellow mixture was quenched with 10% Na₂S₂O₃ solution (5 mL) and partitioned between 10% potassium carbonate solution (10 mL) and DCM (10 mL). The layers were separated and the aqueous layer extracted twice more with DCM (2×10 mL). The organic layers were combined and washed with brine (20 mL), dried over sodium sulfate, filtered and concentrated in vacuo to give the crude material as a pale yellow powder which was purified by KP-NH silica gel chromatography eluting with ethyl acetate:heptane (10-100%) to yield 4-bromo-1-methyl-7-oxido-pyrazolo[3,4-b]pyridin-7-ium (200 mg, 44%) as a white powder. LCMS (Analytical Method B) t_{R}=1.11 min, [M+H]⁺=228.1/230.1; 1H NMR (500 MHz, Chloroform-d) δ 8.09 (d, J=6.5 Hz, 1H), 7.99 (s, 1H), 7.15 (d, J=6.5 Hz, 1H), 4.53 (d, J=2.4 Hz, 3H).

Step 3: To a solution of 4-bromo-1-methyl-7-oxido-pyrazolo[3,4-b]pyridin-7-ium (50 mg, 0.219 mmol) and tert-butyl N-[1-(aminomethyl)cyclopropyl]carbamate (82 mg, 0.439 mmol) in DMF (2 mL) was added N,N-dimethylcarbamoyl chloride (47 mg, 0.439 mmol) and the reaction mixture was warmed to 40° C. After 3 hours, additional N,N-dimethylcarbamoyl chloride (47 mg, 0.439 mmol) and 4-bromo-1-methyl-7-oxido-pyrazolo[3,4-b]pyridin-7-ium (50 mg, 0.219 mmol) were added and heating continued at 40° C. for 6 hours. The solvent was removed in vacuo and the crude material was purified via high pH C18 chromatography to give tert-butyl N-[1-[[(4-bromo-1-methyl-pyrazolo[3,4-b]pyridin-6-yl)amino]methyl]cyclopropyl]carbamate (20 mg, 20%) as a white powder. LCMS (Analytical Method B) t_{R}=1.69 min, [M+H]⁺=396.2/398.2; 1H NMR (500 MHz, Chloroform-d) δ 7.70 (s, 1H), 6.25 (s, 1H), 6.06 (s, 1H), 4.98 (s, 1H), 3.88 (s, 3H), 3.45 (d, J=4.1 Hz, 2H), 1.36 (s, 9H), 0.83 (s, 4H).

Step 4: A solution of tert-butyl N-[1-[[(4-bromo-1-methyl-pyrazolo[3,4-b]pyridin-6-yl)amino]methyl]cyclopropyl]carbamate (30 mg, 0.0757 mmol), 4-(trifluoromethyl)aniline (26 uL, 0.114 mmol) and caesium carbonate (0.071 mL, 0.114 mmol) in 1,4 dioxane (0.5 mL) was degassed for 2 minutes. BrettPhos Pd G3 was added and the reaction mixture was heated to 100° C. for 1 hour. The reaction mixture was degassed and additional BrettPhos Pd G3 (6.9 mg, 7.57 μmol) was added and the heating continued at 100° C. for a further 1 hour. The reaction mixture was degassed and BrettPhos Pd G3 (6.9 mg, 7.57 μmol) was added and the reaction mixture heated to 100° C. for 18 hours. The reaction mixture was cooled to room temperature and the solvent removed in vacuo. The crude product was purified by silica gel chromatography eluting with 5-100% ethyl acetate:heptane to yield the desired BOC protected compound as a beige solid.

To the BOO intermediate was added 4 M hydrogen chloride in 1,4 dioxane (0.38 mL, 1.51 mmol) and the reaction mixture was stirred at room temperature for 2 hours. The reaction mixture was concentrated in vacuo to give an orange solid which was purified by eluting through an SX cartridge with methanol: 2N ammonia in methanol followed by high pH 018 chromatography eluting with 10-100% acetonitrile:water (+0.1% NH₄OH) to give N6-[(1-aminocyclopropyl)methyl]-1-methyl-N4-[4-(trifluoromethyl)phenyl]pyrazolo[3,4-b]pyridine-4,6-diamine (7.5 mg, 26%) as a white powder. LMS (Analytical Method C) t_{R}=3.02 min, [M+H]⁺=377.2; 1H NMR (400 MHz, DMSO-d6) b 9.14 (s, 1H), 7.85 (s, 1H), 7.71 (d, J=8.5 Hz, 2H), 7.45 (d, J=8.5 Hz, 2H), 6.94 (t, J=5.6 Hz, 1H), 6.22 (s, 1H), 3.82 (s, 3H), 3.48 (d, J=5.6 Hz, 2H), 1.27 (s, 1H), 0.84-0.65 (in, 4H).

The compounds in Table 3 were prepared in an analogous manner to the above compounds according to either General Method 3, General Method 4 or General Method 5 using the appropriate diamine or BOO protected diamine and 4-substituted pyrazolopyrimidine intermediate which were in turn synthesised according to General Method 1 or General Method 2.

TABLE 3

| Compound | | | Analytical | t_{R} | |
|---|---|---|---|---|---|
| No. | Name | Structure | Method | (min) | [M + H]⁺ |
| 3.01 | 6-N-(2- aminopropyl)-4- N-[(3-chloro-4- methylphenyl) methyl]-1- methylpyrazolo[3, 4-d]pyrimidine- 4,6-diamine | 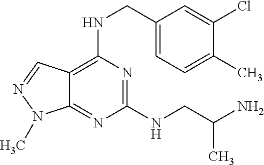 [SMILES: Cc1ccc(CNc2nc(NCC(C)N)nc3c2cnn3C)cc1Cl] | E | 1.60 | 360.2/ 362.2 |
| 3.02 | 6-N-(1- aminopropan-2- yl)-4-N-[(3-chloro- 4- methylphenyl) methyl]-1- methylpyrazolo[3, 4-d]pyrimidine- 4,6-diamine | 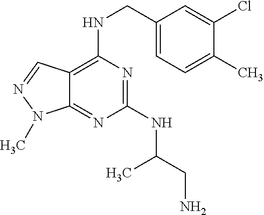 [SMILES: Cc1ccc(CNc2nc(NC(C)CN)nc3c2cnn3C)cc1Cl] | E | 1.66 | 360.2/ 362.2 |
| 3.03 | 6-N-[(2S)-2- aminopropyl]-4-N- [(3-chloro-4- methylphenyl) methyl]-1- methylpyrazolo[3, 4-d]pyrimidine- 4,6-diamine | 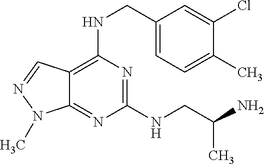 [SMILES: Cc1ccc(CNc2nc(NC[C@H](C)N)nc3c2cnn3C)cc1Cl] | E | 1.61 | 360.2/ 362.2 |
| 3.04 | 6-N-[(2R)-2- aminopropyl]-4-N- [(3-chloro-4- methylphenyl) methyl]-1- methylpyrazolo[3, 4-d]pyrimidine- 4,6-diamine | 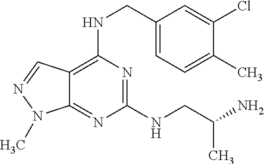 [SMILES: Cc1ccc(CNc2nc(NC[C@@H](C)N)nc3c2cnn3C)cc1Cl] | E | 1.60 | 360.2/ 362.2 |
| 3.05 | 6-N-[(2S)-1- aminobutyl]-4-N- [(3-chloro-4- methylphenyl) methyl]-1- methylpyrazolo[3, 4-d]pyrimidine- 4,6-diamine | 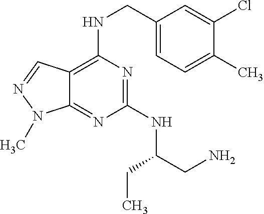 [SMILES: CC[C@@H](CN)Nc1nc(NCc2ccc(C)c(Cl)c2)c2cnn(C)c2n1] | E | 1.78 | 374.2/ 376.2 |
| 3.06 | 6-N-[(2S)-2- aminobutyl]-4-N- [(3-chloro-4- methylphenyl) methyl]-1- methylpyrazolo[3, 4-d]pyrimidine- 4,6-diamine | 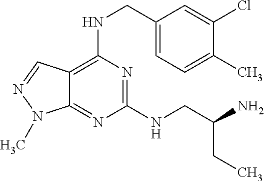 [SMILES: CC[C@H](N)CNc1nc(NCc2ccc(C)c(Cl)c2)c2cnn(C)c2n1] | E | 1.74 | 374.2/ 376.2 |
| 3.07 | 6-N-[(2R)-2- aminobutyl]-4-N- [(3-chloro-4- methylphenyl) methyl]-1- methylpyrazolo[3, 4-d]pyrimidine- 4,6-diamine | 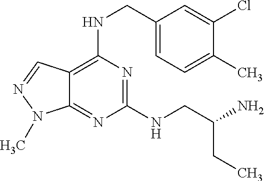 [SMILES: CC[C@@H](N)CNc1nc(NCc2ccc(C)c(Cl)c2)c2cnn(C)c2n1] | E | 1.76 | 374.3/ 376.3 |
| 3.08 | N6-[(1- aminocyclobutyl) methyl]-1-methyl- N4-[4- (trifluoromethyl) phenyl]pyrazolo[3, 4-d]pyrimidine- 4,6-diamine | 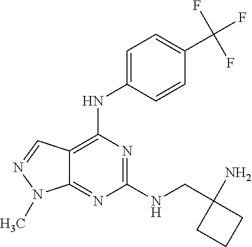 [SMILES: Cn1ncc2c(Nc3ccc(C(F)(F)F)cc3)nc(NCC3(N)CCC3)nc21] | D | 4.45 | 392.2 |
| 3.09 | N6-[(1- aminocyclobutyl) methyl]-N4-[4- (difluoromethoxy) phenyl]-1-methyl- pyrazolo[3,4- d]pyrimidine-4,6- diamine | 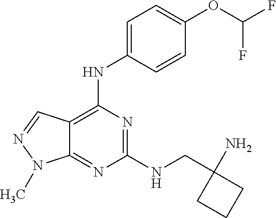 [SMILES: Cn1ncc2c(Nc3ccc(OC(F)F)cc3)nc(NCC3(N)CCC3)nc21] | D | 4.00 | 390.2 |
| 3.10 | N6-(2-amino-2- cyclohexyl-ethyl)- N4-tert-butyl-1- methyl- pyrazolo[3,4- d]pyrimidine-4,6- diamine | 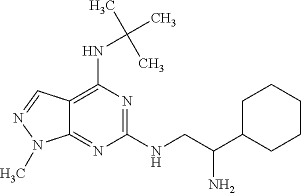 [SMILES: Cn1ncc2c(NC(C)(C)C)nc(NCC(N)C3CCCCC3)nc21] | E | 1.77 | 346.3 |
| 3.11 | N6-(2-amino-2- cyclohexyl-ethyl)- 1-methyl-N4-[4- (trifluoromethyl) phenyl]pyrazolo[3, 4-d]pyrimidine- 4,6-diamine | 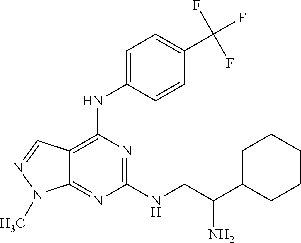 [SMILES: Cn1ncc2c(Nc3ccc(C(F)(F)F)cc3)nc(NCC(N)C3CCCCC3)nc21] | E | 2.33 | 434.3 |
| 3.12 | 6-N-[(1-amino- 3,3- difluorocyclobutyl) methyl]-1-methyl- 4-N-[4- (trifluoromethyl) phenyl]pyrazolo[3, 4-d]pyrimidine- 4,6-diamine | 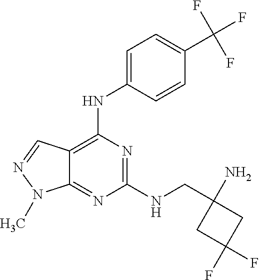 [SMILES: Cn1ncc2c(Nc3ccc(C(F)(F)F)cc3)nc(NCC3(N)CC(F)(F)C3)nc21] | E | 2.01 | 428.3 |
| 3.13 | 6-N-(2-amino-2- cyclopropylethyl)- 1-methyl-4-N-[4- (trifluoromethyl) phenyl]pyrazolo[3, 4-d]pyrimidine- 4,6-diamine | 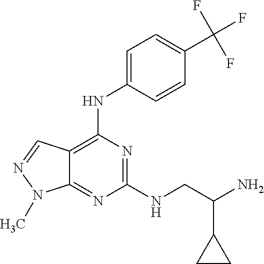 [SMILES: Cn1ncc2c(Nc3ccc(C(F)(F)F)cc3)nc(NCC(N)C3CC3)nc21] | C | 3.34 | 392.3 |
| 3.14 | 6-N-[(3- aminooxolan-3- yl)methyl]-1- methyl-4-N-[4- (trifluoromethyl) phenyl]pyrazolo[3, 4-d]pyrimidine- 4,6-diamine | 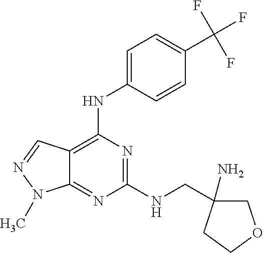 [SMILES: Cn1ncc2c(Nc3ccc(C(F)(F)F)cc3)nc(NCC3(N)CCOC3)nc21] | C | 2.78 | 408.3 |
| 3.15 | N6-[(3- aminotetrahydrofuran- 3-yl)methyl]- 1-methyl-N4-[6- (trifluoromethyl)- 3- pyridyl]pyrazolo[3, 4-d]pyrimidine- 4,6-diamine | 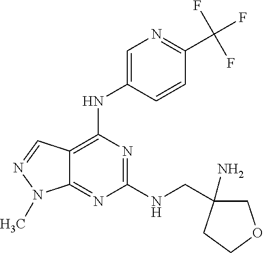 [SMILES: Cn1ncc2c(Nc3ccc(C(F)(F)F)nc3)nc(NCC3(N)CCOC3)nc21] | E | 1.52 | 409.1 |
| 3.16 | 1-methyl-N6- [(morpholin-3- yl)methyl]-N4-[4- (trifluoromethyl) phenyl]-1H- pyrazolo[3,4- d]pyrimidine-4,6- diamine | 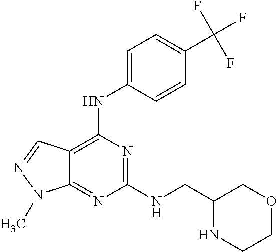 [SMILES: Cn1ncc2c(Nc3ccc(C(F)(F)F)cc3)nc(NCC3COCCN3)nc21] | D | 3.84 | 408.1 |
| 3.17 | 4-N-[(3-chloro-4- methylphenyl)methyl]- 1-methyl-6-N- [[(2R)-pyrrolidin- 2- yl]methyl]pyrazolo [3,4-d]pyrimidine- 4,6-diamine | 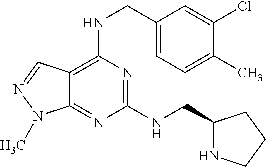 [SMILES: Cc1ccc(CNc2nc(NC[C@H]3CCCN3)nc3c2cnn3C)cc1Cl] | E | 1.71 | 386.2/ 388.2 |
| 3.18 | 6-N-[(1- aminocyclopentyl) methyl]-1-methyl- 4-N-[4- (trifluoromethyl) phenyl]pyrazolo[3, 4-d]pyrimidine- 4,6-diamine | 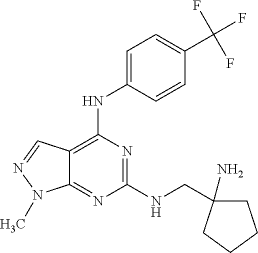 [SMILES: Cn1ncc2c(Nc3ccc(C(F)(F)F)cc3)nc(NCC3(N)CCCC3)nc21] | E | 2.07 | 406.3 |
| 3.19 | 6-N-[(1- aminocyclopentyl) methyl]-1-methyl- 4-N-[6- (trifluoromethyl) pyridin-3- yl]pyrazolo[3,4- d]pyrimidine-4,6- diamine | 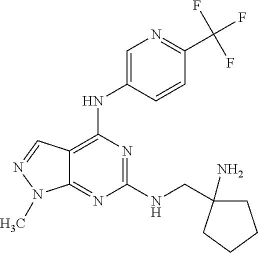 [SMILES: Cn1ncc2c(Nc3ccc(C(F)(F)F)nc3)nc(NCC3(N)CCCC3)nc21] | D | 4.18 | 407.3 |
| 3.20 | 6-N-[(1- aminocyclopentyl) methyl]-4-N-[4- (difluoromethoxy) phenyl]-1- methylpyrazolo[3, 4-d]pyrimidine- 4,6-diamine | 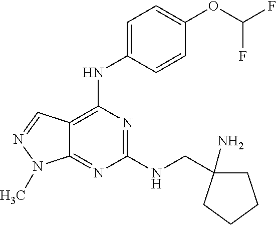 [SMILES: Cn1ncc2c(Nc3ccc(OC(F)F)cc3)nc(NCC3(N)CCCC3)nc21] | D | 4.46 | 404.3 |
| 3.21 | 6-N-[(1- aminocyclopentyl) methyl]-1-methyl- 4-N- phenylpyrazolo[3, 4-d]pyrimidine- 4,6-diamine | 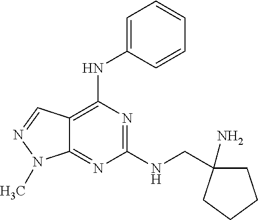 [SMILES: Cn1ncc2c(Nc3ccccc3)nc(NCC3(N)CCCC3)nc21] | C | 2.94 | 338.2 |
| 3.22 | 4-[[6-[(1- aminocyclopentyl) methylamino]-1- methylpyrazolo[3, 4-d]pyrimidin-4- yl]amino]benzonitrile | 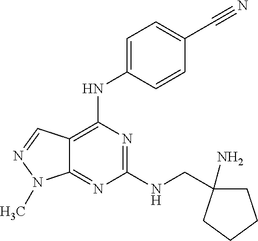 [SMILES: Cn1ncc2c(Nc3ccc(C#N)cc3)nc(NCC3(N)CCCC3)nc21] | E | 1.63 | 363.2 |
| 3.23 | 6-N-[(1- aminocyclopentyl) methyl]-1-methyl- 4-N-[4- (trifluoromethoxy) phenyl]pyrazolo[3, 4-d]pyrimidine- 4,6-diamine | 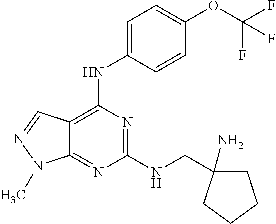 [SMILES: Cn1ncc2c(Nc3ccc(OC(F)(F)F)cc3)nc(NCC3(N)CCCC3)nc21] | D | 4.92 | 422.2 |
| 3.24 | N6-[(1- aminocyclobutyl) methyl]-1- (trideuteriomethyl)- N4-[4- (trifluoromethyl) phenyl]pyrazolo[3, 4-d]pyrimidine- 4,6-diamine | 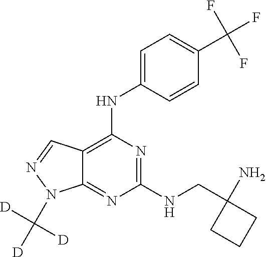 [SMILES: [2H]C([2H])([2H])n1ncc2c(Nc3ccc(C(F)(F)F)cc3)nc(NCC3(N)CCC3)nc21] | C | 3.26 | 395.4 |
| 3.30 | N6-(2- aminocyclohexyl)- N4-[(3-chloro-4- methylphenyl) methyl]-1-methyl-1H- pyrazolo[3,4- d]pyrimidine-4,6- diamine | 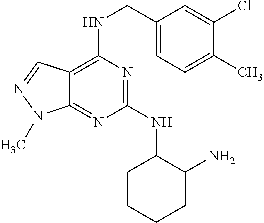 [SMILES: Cc1ccc(CNc2nc(NC3CCCCC3N)nc3c2cnn3C)cc1Cl] | E | 1.87 | 400.3 |

### Compound 3.25: N6-(2-Amino-3-fluoropropyl)-N4-[(3-chloro-4-methylphenyl)methyl]-1-methyl-1H-pyrazolo[3,4-d]pyrimidine-4,6-diamine

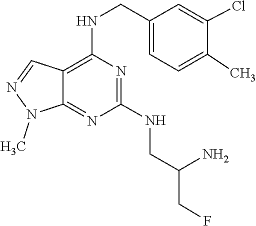 [SMILES: Cc1ccc(CNc2nc(NCC(N)CF)nc3c2cnn3C)cc1Cl]

Step 1: To a solution of potassium phthalimide (1.65 g, 8.89 mmol) in DMF (10 mL) was added 1-chloro-3-fluoropropan-2-ol (1.0 g, 8.89 mmol), and the mixture was stirred at 130° C. for 18 hours. After cooling, the mixture was diluted with ethyl acetate (100 mL) and washed with water (20 mL). The organic layer was separated and concentrated in vacuo. The residue was triturated with diethyl ether and the white solid was filtered and the filtrate was evaporated. The residue was purified by silica gel chromatography eluting with 5-100% heptane:ethyl acetate to give 2-(3-fluoro-2-hydroxy-propyl)isoindoline-1,3-dione (700 mg, 35.3%) as a white solid. LCMS (Analytical Method B) t_{R}=1.28 min, [M+H]⁺=224.2; 1H NMR (250 MHz, Chloroform-d) δ 7.79 (td, J=5.2, 2.0 Hz, 2H), 7.67 (dd, J=5.5, 3.1 Hz, 2H), 4.60-4.41 (m, 1H), 4.42-4.23 (m, 1H), 4.09 (dp, J=19.9, 4.7, 4.3 Hz, 1H), 3.84 (d, J=5.7 Hz, 2H), 2.75 (s, 1H).

Step 2: To a solution of 2-(3-fluoro-2-hydroxy-propyl)isoindoline-1,3-dione (700.0 mg, 3.14 mmol) in DCM (5 mL) was added triethylamine (0.96 mL, 6.9 mmol). The reaction mixture was cooled to 0° C. and 4-methylbenzenesulfonyl chloride (717.49 mg, 3.76 mmol) was added portion-wise. The resulting mixture was allowed to stir at room temperature for 18 hours. The reaction mixture was quenched by the addition of water and extracted into ethyl acetate (×3). The organic extracts were dried over magnesium sulfate, filtered and concentrated under reduced pressure to give [1-[(1,3-dioxoisoindolin-2-yl)methyl]-2-fluoro-ethyl] 4-methylbenzenesulfonate (1000 mg, 84.5%) as a white solid. LCMS (Analytical Method A) t_{R}=1.18 min, [M+H]⁺=378.0; 1H NMR (250 MHz, Chloroform-d) δ 7.78-7.58 (m, 4H), 7.61-7.49 (m, 2H), 6.97 (d, J=8.0 Hz, 2H), 4.91 (ddq, J=20.3, 9.3, 3.7 Hz, 1H), 4.76-4.56 (m, 1H), 4.57-4.37 (m, 1H), 4.07 (dd, J=14.6, 9.3 Hz, 1H), 3.66 (dd, J=14.6, 3.3 Hz, 1H), 2.14 (s, 3H).

Step 3: To a solution of [1-[(1,3-dioxoisoindolin-2-yl)methyl]-2-fluoro-ethyl] 4-methylbenzenesulfonate (1000.0 mg, 2.65 mmol) in DMF (4.3 mL) was added sodium azide (250.0 mg, 3.85 mmol) and the reaction mixture was heated at 60° C. for 18 hours, followed by heating to 120° C. for 6 hours. The reaction mixture was quenched by the slow addition of water (exothermic) and the organics were extracted into DCM (×3). The organics were dried over magnesium sulfate, filtered and concentrated under reduced pressure and purified by silica gel chromatography eluting with 5-100% heptane:ethyl acetate to give 2-(2-azido-3-fluoro-propyl)isoindoline-1,3-dione (500 mg, 76%) as a colourless oil. LCMS (Analytical Method A) t_{R}=1.10 min, [M+H]⁺=248.85; 1H NMR (500 MHz, Chloroform-d) δ 7.82 (tt, J=5.1, 2.5 Hz, 2H), 7.69 (dd, J=5.5, 3.0 Hz, 2H), 4.61 (dd, J=10.0, 3.7 Hz, 1H), 4.56-4.48 (m, 1H), 4.43 (dd, J=10.0, 6.1 Hz, 1H), 3.99-3.95 (m, 1H), 3.86 (dd, J=14.1, 7.8 Hz, 1H), 3.72 (dd, J=14.1, 5.6 Hz, 1H).

Step 4: To a solution of 2-(2-azido-3-fluoro-propyl)isoindoline-1,3-dione (400.0 mg, 1.61 mmol) and tert-butoxycarbonyl tert-butyl carbonate (528.0 mg, 2.42 mmol) in anhydrous THF (40 mL) was added Pd/C (10%, 85.75 mg, 0.08 mmol) and the resulting reaction mixture was stirred at room temperature and atmospheric pressure under a hydrogen atmosphere for 18 hours. The reaction mixture was filtered through Celite™ and the filtrate was concentrated under reduced pressure. The residue was taken up in ethyl acetate and washed with water. The organic extracts were combined, dried over magnesium sulfate, filtered and concentrated under reduced pressure to give tert-butyl N-[1-[(1,3-dioxoisoindolin-2-yl)methyl]-2-fluoro-ethyl]carbamate (519 mg, 99%) as a white solid. LCMS (Analytical Method A) t_{R}=1.09 min, [M+Na]⁺=345.0; 1H NMR (250 MHz, Chloroform-d) δ 7.85 (dt, J=7.0, 3.5 Hz, 2H), 7.71 (dd, J=5.4, 3.1 Hz, 2H), 5.03 (d, J=7.5 Hz, 1H), 4.73-4.54 (m, 1H), 4.54-4.35 (m, 1H), 4.21 (d, J=25.1 Hz, 1H), 4.07-3.89 (m, 1H), 3.82 (dd, J=13.9, 3.9 Hz, 1H), 1.26 (s, 9H).

Step 5: To a solution of tert-butyl N-[1-[(1,3-dioxoisoindolin-2-yl)methyl]-2-fluoro-ethyl]carbamate (464.0 mg, 1.44 mmol) in THF (10 mL) was added hydrazine (2.0 mL, 63.09 mmol) and the resulting reaction mixture was stirred at room temperature for 18 hours. The reaction mixture was concentrated in vacuo to yield a white solid. The white solid was triturated with DCM and the white solid was filtered. The filtrate was concentrated under reduced pressure to give tert-butyl N-[1-(aminomethyl)-2-fluoro-ethyl]carbamate (229 mg, 82.8%) as a colourless oil. LCMS (Analytical Method B) t_{R}=1.34 min, [M+H]⁺=193.3; 1H NMR (250 MHz, Chloroform-d) δ 4.99 (s, 1H), 4.52-4.45 (m, 2H), 3.73 (d, J=28.0 Hz, 1H), 3.00-2.76 (m, 2H), 2.00-1.66 (m, 2H), 1.45 (d, J=1.9 Hz, 9H).

Step 6: 6-chloro-N-[(3-chloro-4-methyl-phenyl)methyl]-1-methyl-pyrazolo[3,4-d]pyrimidin-4-amine (100.0 mg, 0.31 mmol), tert-butyl N-[1-(aminomethyl)-2-fluoro-ethyl]carbamate (119.3 mg, 0.62 mmol), cesium carbonate (303 mg, 0.93 mmol) and BrettPhos (33 mg, 0.06 mmol) were suspended in 1,4-Dioxane (2 mL). The mixture was degassed for 10 minutes before the addition of Pd(OAc)₂ (7.0 mg, 0.03 mmol) and the reaction mixture was degassed for a further 10 minutes and the resulting mixture was heated to 110° C. for 2 hours. The reaction mixture was quenched by the addition of water and the organics were extracted into DCM. The organic layer was separated from the aqueous by the aid of a TELOS™ phase separator and the solvents were removed in vacuo. The crude material was purified by silica gel chromatography eluting with heptane:ethyl acetate (0-100%) to yield tert-butyl N-[1-[[[4-[(3-chloro-4-methyl-phenyl)methylamino]-1-methyl-pyrazolo[3,4-d]pyrimidin-6-yl]amino]methyl]-2-fluoro-ethyl]carbamate (90 mg, 61%) as an off-white solid which was used without further purification for Step 7. LCMS (Analytical Method B) t_{R}=1.83 min, [M+H]⁺=478.3.

Step 7: N6-(2-amino-3-fluoropropyl)-N4-[(3-chloro-4-methylphenyl)methyl]-1-methyl-1H-pyrazolo[3,4-d]pyrimidine-4,6-diamine was synthesised from tert-butyl N-[1-[[[4-[(3-chloro-4-methyl-phenyl)methylamino]-1-methyl-pyrazolo[3,4-d]pyrimidin-6-yl]amino]methyl]-2-fluoro-ethyl]carbamate according to General Method 4 and was purified by reverse phase C18 chromatography eluting with water/acetonitrile+0.1% ammonia to yield the title compound as a white solid (25 mg, 33%). LCMS (Analytical Method C) t_{R}=2.83 min, [M+H]⁺=378.2/380.3; 1H NMR (250 MHz, DMSO-d6) δ 8.27 (s, 1H), 7.81 (s, 1H), 7.38 (d, J=1.3 Hz, 1H), 7.36-7.16 (m, 2H), 6.67 (s, 1H), 4.63 (d, J=5.9 Hz, 2H), 4.37 (s, 1H), 4.18 (s, 1H), 3.70 (s, 3H), 3.48-3.35 (m, 1H), 3.18 (dd, J=13.3, 6.4 Hz, 2H), 2.30 (s, 3H), 1.76 (s, 2H).

### Compound 3.26: 6-N-(2-amino-3-fluoropropyl)-1-methyl-4-N-[4-(trifluoromethyl)phenyl]pyrazolo[3,4-d]pyrimidine-4,6-diamine

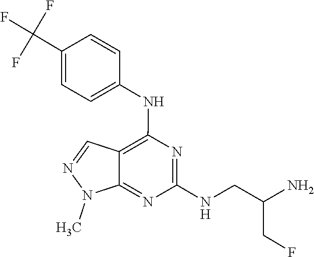 [SMILES: Cn1ncc2c(Nc3ccc(C(F)(F)F)cc3)nc(NCC(N)CF)nc21]

6-N-(2-amino-3-fluoropropyl)-1-methyl-4-N-[4-(trifluoromethyl)phenyl]pyrazolo[3,4-d]pyrimidine-4,6-diamine was synthesised in a similar fashion to Compound 3.25 from 1-methyl-6-methylsulfonyl-N-[4-(trifluoromethyl)phenyl]pyrazolo[3,4-d]pyrimidin-4-amine and tert-butyl N-[1-(aminomethyl)-2-fluoro-ethyl]carbamate using General Method 4 and was purified by reverse phase C18 chromatography eluting with water/acetonitrile+0.1% ammonia to yield the title compound as a light yellow solid (8.3 mg, 27%). LCMS (Analytical Method C) t_{R}=1.80 min, [M+H]⁺=384.2; 1H NMR (250 MHz, DMSO-d6) δ 9.93 (s, 1H), 8.19 (d, J=8.3 Hz, 2H), 8.06 (s, 1H), 7.68 (d, J=8.6 Hz, 2H), 7.11 (s, 1H), 4.45 (d, J=3.2 Hz, 1H), 4.25 (d, J=5.1 Hz, 1H), 3.77 (s, 3H), 3.49 (s, 1H), 3.19 (dd, J=11.7, 6.6 Hz, 2H), 1.73 (s, 2H); 19F NMR (235 MHz, DMSO-d6) δ −231.1, −60.1

### Compound 3.27: 6-N-(2-amino-3-methoxypropyl)-4-N-[(3-chloro-4-methylphenyl)methyl]-1-methylpyrazolo[3,4-d]pyrimidine-4,6-diamine

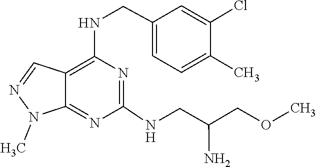 [SMILES: COCC(N)CNc1nc(NCc2ccc(C)c(Cl)c2)c2cnn(C)c2n1]

6-N-(2-Amino-3-methoxypropyl)-4-N-[(3-chloro-4-methylphenyl)methyl]-1-methylpyrazolo[3,4-d]pyrimidine-4,6-diamine was synthesised from tert-butyl N-(1-hydroxy-3-methoxypropan-2-yl)carbamate and 6-chloro-N-[(3-chloro-4-methylphenyl)methyl]-1-methyl-1H-pyrazolo[3,4-d]pyrimidin-4-amine via the above methods and General Method 4 and was purified by reverse phase C18 chromatography eluting with water/acetonitrile+0.1% ammonia to yield the title compound as a white solid (2.4 mg, 3%). LCMS (Analytical Method C) t_{R}=2.85 min, [M+H]⁺=390.3/392.3; 1H NMR (500 MHz, Chloroform-d) δ 7.60 (s, 1H), 7.35 (d, J=1.5 Hz, 1H), 7.20 (d, J=7.8 Hz, 1H), 7.14 (dd, J=7.8, 1.7 Hz, 1H), 5.27 (s, 1H), 4.70 (s, 2H), 3.84 (s, 3H), 3.62-3.54 (m, 1H), 3.47-3.38 (m, 2H), 3.37 (s, 3H), 3.36-3.31 (m, 2H), 3.23 (s, 1H), 2.36 (s, 3H).

### Compound 3.28: 6-N-[(3-Aminooxetan-3-yl)methyl]-1-methyl-4-N-[4-(trifluoromethyl)phenyl]pyrazolo[3,4-d]pyrimidine-4,6-diamine

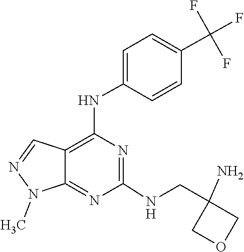 [SMILES: Cn1ncc2c(Nc3ccc(C(F)(F)F)cc3)nc(NCC3(N)COC3)nc21]

6-Chloro-1-methyl-N-[4-(trifluoromethyl)phenyl]pyrazolo[3,4-d]pyrimidin-4-amine was synthesised according to the method detailed in Compound 1.01

Step 2: To a solution of 6-chloro-1-methyl-N-[4-(trifluoromethyl)phenyl]pyrazolo[3,4-d]pyrimidin-4-amine (200.0 mg, 0.61 mmol) and 3-(aminomethyl)-N,N-dibenzyloxetan-3-amine (355.0 mg, 1.26 mmol) in 1,4-dioxane (6 mL) was added palladium(II) acetate (16.0 mg, 0.07 mmol), BrettPhos (68.0 mg, 0.13 mmol) and cesium carbonate (600.0 mg, 1.84 mmol) and the resulting reaction mixture was heated to 110° C. for 4 hours. The reaction was quenched by the addition of water and the organics extracted into ethyl acetate (×3). The combined organic extracts were dried over magnesium sulfate, filtered and concentrated under reduced pressure and the crude product was purified by silica gel chromatography eluting with heptane/ethyl acetate (5-100%) yielding N6-[[3-(dibenzylamino)oxetan-3-yl]methyl]-1-methyl-N4-[4-(trifluoromethyl)phenyl]pyrazolo[3,4-d]pyrimidine-4,6-diamine as a yellow glue (111 mg, 32%). LCMS (Analytical Method B) t_{R}=2.08 min, [M+H]⁺=574.5; 1H NMR (250 MHz, Chloroform-d) δ 7.74 (d, J=8.7 Hz, 2H), 7.63-7.51 (m, 3H), 7.26-7.15 (d, J=8.3 Hz, 10H), 5.50 (s, 1H), 4.53 (d, J=6.6 Hz, 2H), 4.14 (d, J=5.9 Hz, 4H), 3.85 (s, 3H), 3.72 (s, 4H); 19F NMR (235 MHz, Chloroform-d) 5-62.04.

Step 3: N6-[[3-(Dibenzylamino)oxetan-3-yl]methyl]-1-methyl-N4-[4-(trifluoromethyl)phenyl]pyrazolo[3,4-d]pyrimidine-4,6-diamine (106.0 mg, 0.18 mmol) and dihydroxypalladium (13.2 mg, 0.09 mmol) were suspended in ethanol (5 mL) and ethyl acetate (2.5 mL) and stirred at room temperature under a hydrogen atmosphere for 36 hours. The reaction mixture was filtered through Celite™, the pad was washed with methanol (2×10 mL) and the solvents were evaporated to give the crude product which was purified by reverse phase C18 chromatography eluting with water/acetonitrile 5-100%+0.1% ammonia to yield the title compound as an off white solid (40.4 mg, 55%). LCMS (Analytical Method E) t_{R}=1.77 min, [M+H]⁺=394.2; 1H NMR (500 MHz, DMSO-d6) δ 9.93 (s, 1H), 8.19 (d, J=8.4 Hz, 2H), 8.06 (s, 1H), 7.68 (d, J=8.6 Hz, 2H), 7.09 (s, 1H), 4.47 (d, J=5.9 Hz, 2H), 4.31 (d, J=5.8 Hz, 2H), 3.77 (s, 3H), 3.70 (d, J=6.0 Hz, 2H), 2.23 (s, 2H); 19 F NMR (235 MHz, DMSO-d6) δ −60.09 ppm

### Compound 3.29: 6-N-[(3-Aminooxetan-3-yl)methyl]-1-methyl-4-N-phenylpyrazolo[3,4-d]pyrimidine-4,6-diamine

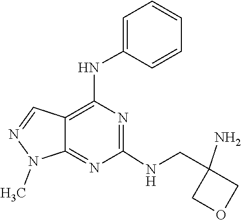 [SMILES: Cn1ncc2c(Nc3ccccc3)nc(NCC3(N)COC3)nc21]

Synthesis of Compound 3.29 was carried in a similar fashion to the above compound using 6-chloro-N-(4-chlorophenyl)-1-methyl-1H-pyrazolo[3,4-d]pyrimidin-4-amine and 3-aminomethyl-3-[bis(phenylmethyl)amino]oxetane. LCMS (Analytical Method D) t_{R}=2.93 min, [M+H]⁺=326.2; 1H NMR (500 MHz, DMSO-d6) δ 9.57 (s, 1H), 7.99 (m, 2H), 7.91 (d, J=7.8 Hz, 2H), 7.40-7.30 (m, 2H), 7.05 (t, J=7.3 Hz, 1H), 6.94 (s, 1H), 4.45 (d, J=5.2 Hz, 2H), 4.30 (d, J=5.8 Hz, 2H), 3.75 (s, 3H), 3.69 (d, J=6.0 Hz, 2H).

### Compound 4.01: N6-(2-Amino-2-phenyl-propyl)-N4-tert-butyl-1-(trideuteriomethyl)pyrazolo[3,4-d]pyrimidine-4,6-diamine

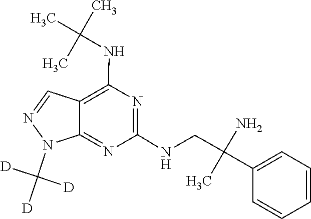 [SMILES: [2H]C([2H])([2H])n1ncc2c(NC(C)(C)C)nc(NCC(C)(N)c3ccccc3)nc21]

Step 1: 5-Amino-1H-pyrazole-4-carbonitrile (1.00 g, 9.25 mmol) was dissolved in DMF (10 mL) and caesium carbonate (3.32 g, 10.2 mmol) was added. The reaction mixture was cooled to 0° C. and trideuterio(iodo)methane (0.60 mL, 9.71 mmol) was added slowly at 0-10° C. and stirring was continued for 6 hours at 0-10° C. The reaction mixture was concentrated in vacuo and DCM (10 mL) added, the solution was filtered and the cake washed with DCM (2×5 mL). The cake was dried to give the desired compound containing excess caesium carbonate as a white powder (4.5 g). The white powder was stirred in ethyl acetate (20 mL) for 10 minutes and filtered. This process was repeated twice and the combined filtrates were concentrated in vacuo to give 5-amino-1-(trideuteriomethyl)pyrazole-4-carbonitrile (485 mg, 42%) as a crystalline white powder. LCMS (Analytical Method A) t_{R}=0.31 min, [M+H]⁺=125.90; 1H NMR (400 MHz, DMSO-d6) δ 7.50 (s, 1H), 6.52 (s, 2H)

Step 2: To a solution of 5-amino-1-(trideuteriomethyl)pyrazole-4-carbonitrile (485 mg, 3.88 mmol) in acetonitrile (10 mL) was added trichloromethyl carbonochloridate (0.70 mL, 5.81 mmol) and the reaction mixture was allowed to heat to 130° C. for 18 hours. The reaction mixture was cooled to 0° C. and ice was carefully added to the reaction mixture with stirring for 30 minutes. The resulting mixture was filtered to separate out the precipitate and filtrate. The filtrate was diluted with ethyl acetate (20 mL) and washed with water (20 mL). The layers were separated and the aqueous layer was extracted with ethyl acetate (2×20 mL). The combined organic layers were dried over magnesium sulfate, filtered and concentrated in vacuo to give an orange residue which was purified by silica gel chromatography eluting with 0-50% ethyl acetate:heptane to give the desired compound as a white fluffy powder (300 mg, 36%). LCMS (Analytical Method A) t_{R}=1.05 min, [M+H]⁺=205.85/207.85; 1 H NMR (500 MHz, DMSO-d6) δ 8.07 (s, 1H).

Step 3: N-tert-Butyl-6-chloro-1-(trideuteriomethyl)pyrazolo[3,4-d]pyrimidin-4-amine was synthesised from 4,6-dichloro-1-(trideuteriomethyl)pyrazolo[3,4-d]pyrimidine and tert-butylamine according to General Method 1 (70 mg, 70%). LCMS (Analytical Method B) t_{R}=1.61 min, [M+H]⁺=243.2/245.2; 1H NMR (500 MHz, DMSO-d6) δ 8.21 (s, 1H), 8.10 (s, 1H), 1.49 (s, 9H).

Step 4: N6-(2-Amino-2-phenyl-propyl)-N4-tert-butyl-1-(trideuteriomethyl)pyrazolo[3,4-d]pyrimidine-4,6-diamine was synthesised from N-tert-butyl-6-chloro-1-(2H3)-methyl-1H-pyrazolo[3,4-d]pyrimidin-4-amine and 2-phenyl-propane-1,2-diamine according to General Method 3 (18 mg, 25%) as a white powder. LCMS (Analytical Method D) t_{R}=3.98 min, [M+H]⁺=357.3; 1H NMR (500 MHz, DMSO-d6) δ 7.89 (s, 1H), 7.56 (d, J=7.5 Hz, 2H), 7.31 (t, J=7.5 Hz, 2H), 7.19 (t, J=7.5 Hz, 1H), 7.09 (s, 1H), 6.17 (s, 1H), 3.69 (s, 1H), 3.49 (dd, J=13.0, 5.4 Hz, 1H), 2.00 (s, 2H), 1.49 (s, 9H), 1.35 (s, 3H).

### Compound 4.02: 6-N-(2-Amino-2-phenylethyl)-1-ethyl-4-N-propan-2-ylpyrazolo[3,4-d]pyrimidine-4,6-diamine

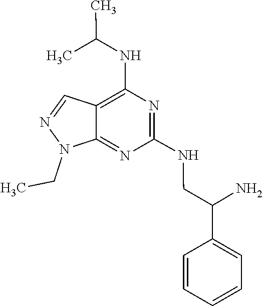 [SMILES: CCn1ncc2c(NC(C)C)nc(NCC(N)c3ccccc3)nc21]

Step 1: To a stirred solution of 2,4,6-trichloropyrimidine-5-carbaldehyde (1.00 g, 4.73 mmol) and ethyl hydrazine ethanedioate (1:1) (710 mg, 4.73 mmol) in ethanol (10 mL) cooled to 0° C. was added triethylamine (1.0 mL, 7.17 mmol) and the reaction mixture was allowed to warm to room temperature for 18 hours. The reaction mixture was concentrated under reduced pressure, diluted with water (10 mL) and extracted into ethyl acetate (20 mL×3). The combined organic layers were washed with brine (20 mL), dried over magnesium sulfate, filtered and concentrated in vacuo. The crude material was purified by silica gel chromatography eluting with 0-100% ethyl acetate in heptane to afford 4,6-dichloro-1-ethyl-pyrazolo[3,4-d]pyrimidine as a pink solid (600 mg, 58%). LCMS (Analytical Method A) t_{R}=1.17 min, [M+H]⁺=216.85/218.85; 1H NMR (500 MHz, Chloroform-d) δ 8.07 (s, 1H), 4.44 (q, J=7.3 Hz, 2H), 1.47 (t, J=7.3 Hz, 3H).

Step 2: 6-Chloro-1-ethyl-N-isopropyl-pyrazolo[3,4-d]pyrimidin-4-amine was synthesised from 4,6-dichloro-1-ethyl-pyrazolo[3,4-d]pyrimidine and isopropylamine according to General Method 1 and was used directly in Step 3 (160 mg, 97%). LCMS (Analytical Method B) t_{R}=1.50 min, [M+H]⁺=240.2/242.2; 1H NMR (500 MHz, DMSO-d6) δ 8.51 (d, J=7.5 Hz, 1H), 8.12 (s, 1H), 4.33 (sept, 6.6 Hz, 1H), 4.24 (q, J=7.2 Hz, 2H), 1.35 (t, J=7.2 Hz, 3H), 1.23 (d, J=6.6 Hz, 6H).

Step 3: 6-N-(2-Amino-2-phenylethyl)-1-ethyl-4-N-propan-2-ylpyrazolo[3,4-d]pyrimidine-4,6-diamine was synthesised from 4,6-dichloro-1-ethyl-pyrazolo[3,4-d]pyrimidine and 2-(BOC-amino)-2-phenylethylamine according to General Method 4 (51 mg, 51%). LCMS (Analytical Method 0) t_{R}=3.76 min, [M+H]⁺=340.3; 1 H NMR (250 MHz, DMSO-d6) b 7.81 (s, 1H), 7.47-7.40 (m, 2H), 7.32 (t, J=7.4 Hz, 2H), 7.28-7.14 (m, 2H), 6.18 (t, J=5.4 Hz, 1H), 4.43-4.27 (m, 1H), 4.20-4.07 (m, 3H), 3.70-3.57 (m, 1H), 3.36-3.23 (m, 1H), 1.34 (t, J=7.2 Hz, 3H), 1.24 (d, J=6.5 Hz, 6H).

The compounds in Table 4 were prepared in an analogous manner to the above compounds according to either General Method 3, General Method 4 or General Method 5 using either (1R)-1-phenylethane-1,2-diamine, (1S)-1-phenylethane-1,2-diamine tert-butyl N-[(1R)-2-amino-1-phenylethyl]carbamate, tert-butyl N-(2-amino-1-phenylethyl)carbamate or 2-phenyl-propane-1,2-diamine and the corresponding 4-substituted pyrazolopyrimidine intermediate which were in turn synthesised according to General Method 1 or General Method 2.

TABLE 4

| Compound | | | Analytical | t_{R} | |
|---|---|---|---|---|---|
| No. | Name | Structure | Method | (min) | [M + H]⁺ |
| 4.03 | 6-N-(2-amino-2- phenylethyl)-4-N- [(3-chloro-4- methylphenyl) methyl]-1- methylpyrazolo[3, 4-d]pyrimidine- 4,6-diamine | 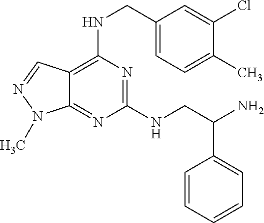 [SMILES: Cc1ccc(CNc2nc(NCC(N)c3ccccc3)nc3c2cnn3C)cc1Cl] | D | 4.39 | 422.2/ 424.3 |
| 4.04 | 6-N-(2-amino-2- phenylethyl)-4-N- [(2,4- difluorophenyl) methyl]-1- methylpyrazolo[3, 4-d]pyrimidine- 4,6-diamine | 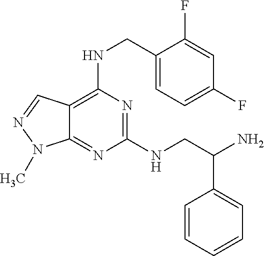 [SMILES: Cn1ncc2c(NCc3ccc(F)cc3F)nc(NCC(N)c3ccccc3)nc21] | D | 4.02 | 410.3 |
| 4.05 | 6-N-(2-amino-2- phenylethyl)-4-N- [(3-chloro-4- fluorophenyl) methyl]-1- methylpyrazolo[3, 4-d]pyrimidine- 4,6-diamine | 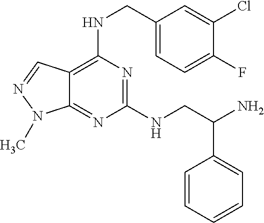 [SMILES: Cn1ncc2c(NCc3ccc(F)c(Cl)c3)nc(NCC(N)c3ccccc3)nc21] | E | 1.89 | 423.3/ 428.3 |
| 4.06 | 6-N-(2-amino-2- phenylethyl)-4-N- [(3- bromophenyl) methyl]-1- methylpyrazolo[3, 4-d]pyrimidine- 4,6-diamine | 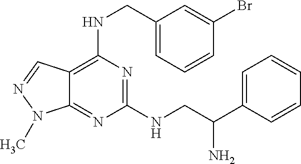 [SMILES: Cn1ncc2c(NCc3cccc(Br)c3)nc(NCC(N)c3ccccc3)nc21] | D | 3.71 | 452.2/ 454.2 |
| 4.07 | 6-N-(2-amino-2- phenylethyl)-4-N- [(4- bromophenyl) methyl]-1- methylpyrazolo[3, 4-d]pyrimidine- 4,6-diamine | 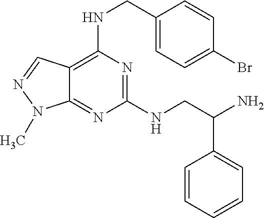 [SMILES: Cn1ncc2c(NCc3ccc(Br)cc3)nc(NCC(N)c3ccccc3)nc21] | D | 3.78 | 452.2/ 454.2 |
| 4.08 | 6-N-(2-amino-2- phenylethyl)-1- methyl-4-N-[[6- (trifluoromethyl) pyridin-3- yl]methyl]pyrazolo [3,4-d]pyrimidine- 4,6-diamine | 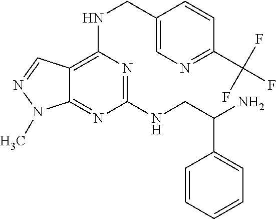 [SMILES: Cn1ncc2c(NCc3ccc(C(F)(F)F)nc3)nc(NCC(N)c3ccccc3)nc21] | D | 3.72 | 443.2 |
| 4.09 | N6-(2-amino-2- phenyl-ethyl)-N4- [(2,4- dimethoxyphenyl) methyl]-1-methyl- pyrazolo[3,4- d]pyrimidine-4,6- diamine | 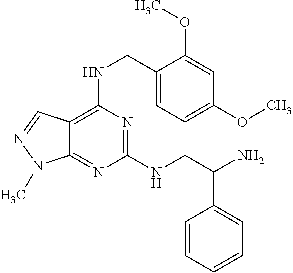 [SMILES: COc1ccc(CNc2nc(NCC(N)c3ccccc3)nc3c2cnn3C)c(OC)c1] | D | 3.79 | 434.3 |
| 4.10 | 6-N-(2-amino-2- phenylethyl)-4-N- (4-fluorophenyl)- 1- methylpyrazolo[3, 4-d]pyrimidine- 4,6-diamine | 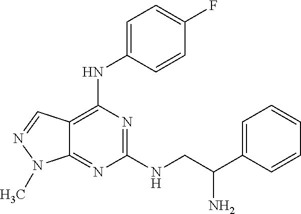 [SMILES: Cn1ncc2c(Nc3ccc(F)cc3)nc(NCC(N)c3ccccc3)nc21] | D | 3.83 | 378.2 |
| 4.11 | N6-(2-amino-2- phenyl-propyl)-1- methyl-N4-[2- (trifluoromethyl) pyrimidin-5- yl]pyrazolo[3,4- d]pyrimidine-4,6- diamine | 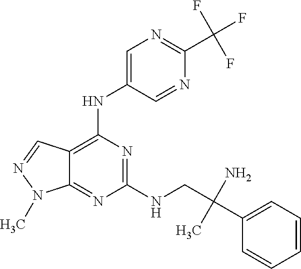 [SMILES: Cn1ncc2c(Nc3cnc(C(F)(F)F)nc3)nc(NCC(C)(N)c3ccccc3)nc21] | D | 4.03 | 444.2 |
| 4.12 | N6-(2-amino-2- phenyl-propyl)-1- methyl-N4-[4- (trifluoromethoxy) phenyl]pyrazolo[3, 4-d]pyrimidine- 4,6-diamine | 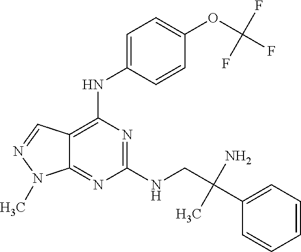 [SMILES: Cn1ncc2c(Nc3ccc(OC(F)(F)F)cc3)nc(NCC(C)(N)c3ccccc3)nc21] | C | 3.64 | 458.3 |
| 4.13 | N6-(2-amino-2- phenyl-propyl)- N4-(3,4- dichlorophenyl)-1- methyl- pyrazolo[3,4- d]pyrimidine-4,6- diamine | 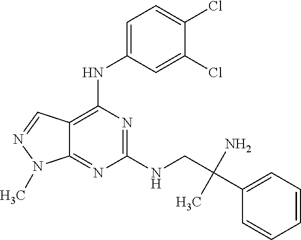 [SMILES: Cn1ncc2c(Nc3ccc(Cl)c(Cl)c3)nc(NCC(C)(N)c3ccccc3)nc21] | D | 4.57 | 442.1/ 445.1 |
| 4.14 | 6-N-(2-amino-2- phenylethyl)-4-N- [4- (difluoromethoxy) phenyl]-1- methylpyrazolo[3, 4-d]pyrimidine- 4,6-diamine | 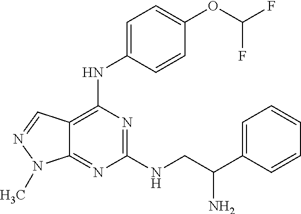 [SMILES: Cn1ncc2c(Nc3ccc(OC(F)F)cc3)nc(NCC(N)c3ccccc3)nc21] | D | 3.97 | 426.2 |
| 4.15 | 6-N-(2-amino-2- phenylpropyl)-4- N-[4- (difluoromethoxy) phenyl]-1- methylpyrazolo[3, 4-d]pyrimidine- 4,6-diamine | 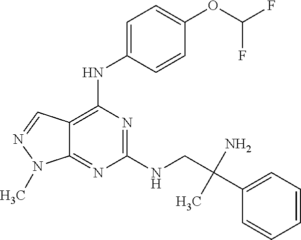 [SMILES: Cn1ncc2c(Nc3ccc(OC(F)F)cc3)nc(NCC(C)(N)c3ccccc3)nc21] | C | 3.24 | 440.3 |
| 4.16 | N6-[(2R)-2- amino-2-phenyl- ethyl]-N4-[4- (difluoromethoxy) phenyl]-1-methyl- pyrazolo[3,4- d]pyrimidine-4,6- diamine | 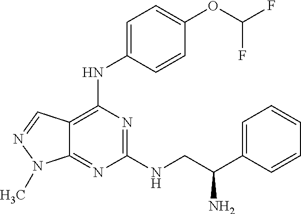 [SMILES: Cn1ncc2c(Nc3ccc(OC(F)F)cc3)nc(NC[C@H](N)c3ccccc3)nc21] | D | 4.01 | 426.2 |
| 4.17 | 6-N-(2-amino-2- phenylethyl)-1- methyl-4-N-[4- (trifluoromethyl) phenyl]pyrazolo[3, 4-d]pyrimidine- 4,6-diamine | 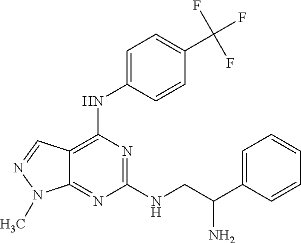 [SMILES: Cn1ncc2c(Nc3ccc(C(F)(F)F)cc3)nc(NCC(N)c3ccccc3)nc21] | D | 4.41 | 428.2 |
| 4.18 | 6-N-(2-amino-2- phenylethyl)-1,3- dimethyl-4-N-[4- (trifluoromethyl) phenyl]pyrazolo[3, 4-d]pyrimidine- 4,6-diamine | 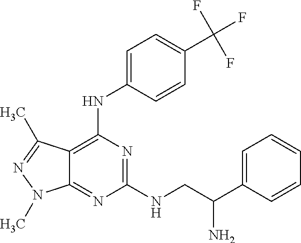 [SMILES: Cc1nn(C)c2nc(NCC(N)c3ccccc3)nc(Nc3ccc(C(F)(F)F)cc3)c12] | D | 4.38 | 442.3 |
| 4.19 | N6-(2-amino-2- phenyl-propyl)-1- methyl-N4-[4- (trifluoromethyl) phenyl]pyrazolo[3, 4-d]pyrimidine- 4,6-diamine | 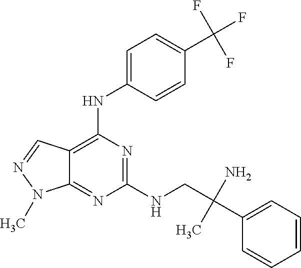 [SMILES: Cn1ncc2c(Nc3ccc(C(F)(F)F)cc3)nc(NCC(C)(N)c3ccccc3)nc21] | C | 3.60 | 442.3 |
| 4.20 | N6-(2-amino-2- phenyl-ethyl)-1- (trideuteriomethyl)- N4-[4- (trifluoromethyl) phenyl]pyrazolo[3, 4-d]pyrimidine- 4,6-diamine | 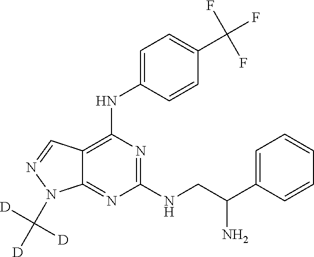 [SMILES: [2H]C([2H])([2H])n1ncc2c(Nc3ccc(C(F)(F)F)cc3)nc(NCC(N)c3ccccc3)nc21] | D | 4.32 | 431.1 |
| 4.21 | N6-(2-amino-2- phenyl-propyl)-1- (trideuteriomethyl)- N4-[4- (trifluoromethyl) phenyl]pyrazolo[3, 4-d]pyrimidine- 4,6-diamine | 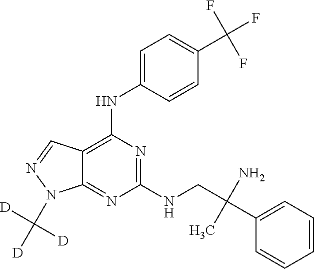 [SMILES: [2H]C([2H])([2H])n1ncc2c(Nc3ccc(C(F)(F)F)cc3)nc(NCC(C)(N)c3ccccc3)nc21] | D | 4.45 | 445.3 |
| 4.22 | 6-N-[(2R)-2- amino-2- phenylethyl]-1- methyl-4-N-[4- (trifluoromethyl) phenyl]pyrazolo[3, 4-d]pyrimidine- 4,6-diamine | 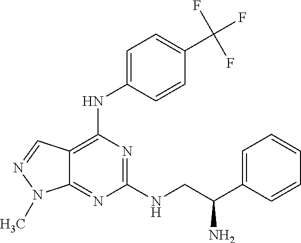 [SMILES: Cn1ncc2c(Nc3ccc(C(F)(F)F)cc3)nc(NC[C@H](N)c3ccccc3)nc21] | C | 3.40 | 428.2 |
| 4.23 | 6-N-[(2S)-2- amino-2- phenylethyl]-1- methyl-4-N-[4- (trifluoromethyl) phenyl]pyrazolo[3, 4-d]pyrimidine- 4,6-diamine | 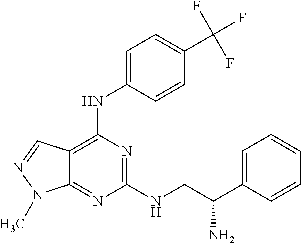 [SMILES: Cn1ncc2c(Nc3ccc(C(F)(F)F)cc3)nc(NC[C@@H](N)c3ccccc3)nc21] | C | 3.40 | 428.2 |
| 4.24 | N6-[(2R)-2- amino-2-phenyl- ethyl]-1- (trideuteriomethyl)- N4-[4- (trifluoromethyl) phenyl]pyrazolo[3, 4-d]pyrimidine- 4,6-diamine | 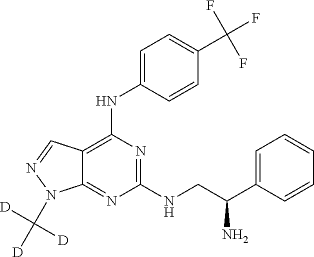 [SMILES: [2H]C([2H])([2H])n1ncc2c(Nc3ccc(C(F)(F)F)cc3)nc(NC[C@H](N)c3ccccc3)nc21] | D | 4.42 | 431.2 |
| 4.25 | 6-N-(2-amino-2- phenylethyl)-1- methyl-4-N-[6- (trifluoromethyl) pyridin-3- yl]pyrazolo[3,4- d]pyrimidine-4,6- diamine | 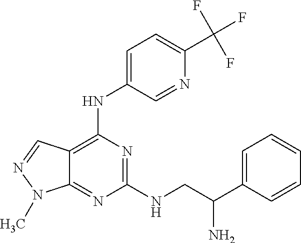 [SMILES: Cn1ncc2c(Nc3ccc(C(F)(F)F)nc3)nc(NCC(N)c3ccccc3)nc21] | D | 3.89 | 429.2 |
| 4.26 | 6-N-(2-amino-2- phenylpropyl)-1- methyl-4-N-[6- (trifluoromethyl) pyridin-3- yl]pyrazolo[3,4- d]pyrimidine-4,6- diamine | 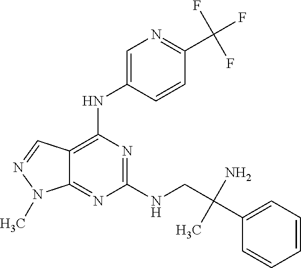 [SMILES: Cn1ncc2c(Nc3ccc(C(F)(F)F)nc3)nc(NCC(C)(N)c3ccccc3)nc21] | D | 4.07 | 443.1 |
| 4.27 | N6-(2-amino-2- phenyl-propyl)-1- (trideuteriomethyl)- N4-[6- (trifluoromethyl)- 3- pyridyl]pyrazolo [3,4-d]pyrimidine- 4,6-diamine | 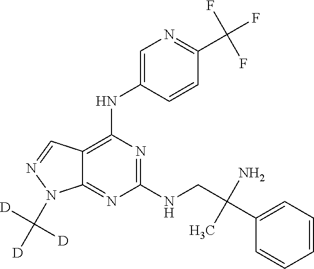 [SMILES: [2H]C([2H])([2H])n1ncc2c(Nc3ccc(C(F)(F)F)nc3)nc(NCC(C)(N)c3ccccc3)nc21] | D | 1.68 | 482.3 |
| 4.28 | N6-(2-amino-2- phenyl-ethyl)-N4- [6- (difluoromethoxy)- 3-pyridyl]-1- methyl- pyrazolo[3,4- d]pyrimidine-4,6- diamine | 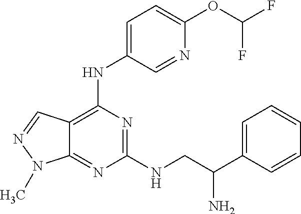 [SMILES: Cn1ncc2c(Nc3ccc(OC(F)F)nc3)nc(NCC(N)c3ccccc3)nc21] | C | 3.03 | 427.3 |
| 4.29 | N6-(2-amino-2- phenyl-propyl)- N4-[6- (difluoromethoxy)- 3-pyridyl]-1- methyl- pyrazolo[3,4- d]pyrimidine-4,6- diamine | 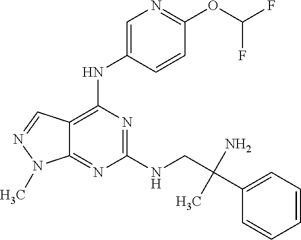 [SMILES: Cn1ncc2c(Nc3ccc(OC(F)F)nc3)nc(NCC(C)(N)c3ccccc3)nc21] | C | 3.17 | 441.3 |
| 4.30 | N6-[(2R)-2- amino-2-phenyl- ethyl]-N4-(4- chlorophenyl)-1- methyl- pyrazolo[3,4- d]pyrimidine-4,6- diamine | 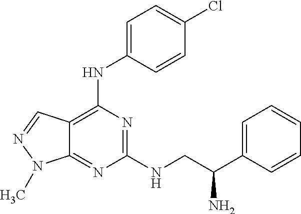 [SMILES: Cn1ncc2c(Nc3ccc(Cl)cc3)nc(NC[C@H](N)c3ccccc3)nc21] | C | 3.23 | 394.2 |
| 4.31 | N6-[(2S)-2- amino-2-phenyl- ethyl]-N4-(4- chlorophenyl)-1- methyl- pyrazolo[3,4- d]pyrimidine-4,6- diamine | 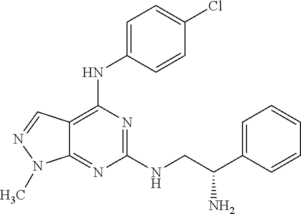 [SMILES: Cn1ncc2c(Nc3ccc(Cl)cc3)nc(NC[C@@H](N)c3ccccc3)nc21] | C | 3.23 | 394.2 |
| 4.32 | N6-[(2R)-2- amino-2-phenyl- ethyl]-N4-(4- methoxyphenyl)- 1-methyl- pyrazolo[3,4- d]pyrimidine-4,6- diamine | 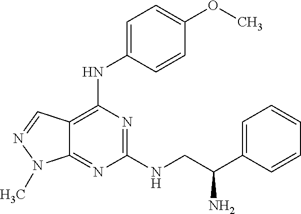 [SMILES: COc1ccc(Nc2nc(NC[C@H](N)c3ccccc3)nc3c2cnn3C)cc1] | C | 2.80 | 390.3 |
| 4.33 | N6-[(2R)-2- amino-2-phenyl- ethyl]-N4-(4- isopropoxyphenyl)- 1-methyl- pyrazolo[3,4- d]pyrimidine-4,6- diamine | 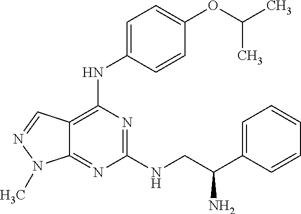 [SMILES: CC(C)Oc1ccc(Nc2nc(NC[C@H](N)c3ccccc3)nc3c2cnn3C)cc1] | C | 3.24 | 418.3 |
| 4.34 | N6-[(2R)-2- amino-2-phenyl- ethyl]-N4-(3,4- difluorophenyl)-1- methyl- pyrazolo[3,4- d]pyrimidine-4,6- diamine | 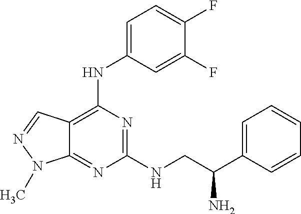 [SMILES: Cn1ncc2c(Nc3ccc(F)c(F)c3)nc(NC[C@H](N)c3ccccc3)nc21] | D | 4.04 | 396.2 |
| 4.35 | N6-[(2R)-2- amino-2-phenyl- ethyl]-N4-[4- (dimethylamino) phenyl]-1-methyl- pyrazolo[3,4- d]pyrimidine-4,6- diamine | 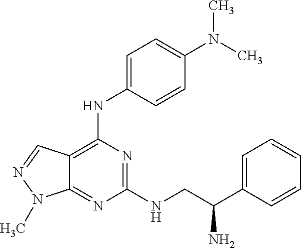 [SMILES: CN(C)c1ccc(Nc2nc(NC[C@H](N)c3ccccc3)nc3c2cnn3C)cc1] | E | 1.30 | 403.3 |
| 4.36 | 6-N-(2-amino-2- phenylethyl)-4- N,1- dimethylpyrazolo [3,4-d]pyrimidine- 4,6-diamine | 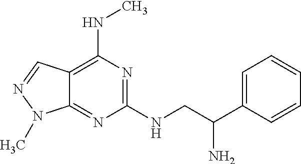 [SMILES: CNc1nc(NCC(N)c2ccccc2)nc2c1cnn2C] | D | 3.05 | 298.3 |
| 4.37 | 6-N-(2-amino-2- phenylethyl)-1- methyl-4-N- (trideuteriomethyl) pyrazolo[3,4- d]pyrimidine-4,6- diamine | 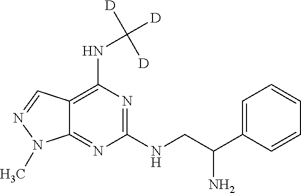 [SMILES: [2H]C([2H])([2H])Nc1nc(NCC(N)c2ccccc2)nc2c1cnn2C] | D | 3.16 | 301.3 |
| 4.38 | 6-N-(2-amino-2- phenylethyl)-4-N- methyl-1- (trideuteriomethyl) pyrazolo[3,4- d]pyrimidine-4,6- diamine | 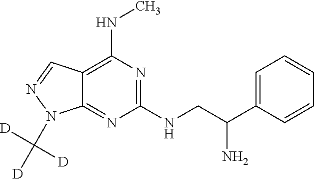 [SMILES: [2H]C([2H])([2H])n1ncc2c(NC)nc(NCC(N)c3ccccc3)nc21] | D | 3.01 | 301.2 |
| 4.39 | 6-N-(2-amino-2- phenylpropyl)-4- N,1- dimethylpyrazolo [3,4-d]pyrimidine- 4,6-diamine | 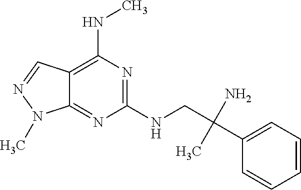 [SMILES: CNc1nc(NCC(C)(N)c2ccccc2)nc2c1cnn2C] | B | 1.36 | 312.3 |
| 4.40 | 6-N-[(2R)-2- amino-2- phenylethyl]-4- N,1- dimethylpyrazolo [3,4-d]pyrimidine- 4,6-diamine | 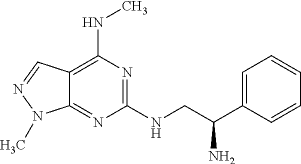 [SMILES: CNc1nc(NC[C@H](N)c2ccccc2)nc2c1cnn2C] | D | 3.02 | 298.3 |
| 4.41 | 6-N-(2-amino-2- phenylethyl)-4-N- ethyl-1- methylpyrazolo[3, 4-d]pyrimidine- 4,6-diamine | 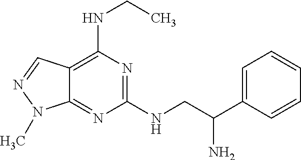 [SMILES: CCNc1nc(NCC(N)c2ccccc2)nc2c1cnn2C] | E | 1.10 | 312.2 |
| 4.42 | 6-N-(2-amino-2- phenylethyl)-4-N- ethyl-4-N,1- dimethylpyrazolo [3,4-d]pyrimidine- 4,6-diamine | 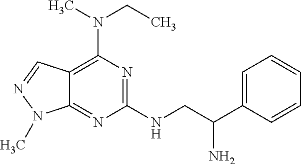 [SMILES: CCN(C)c1nc(NCC(N)c2ccccc2)nc2c1cnn2C] | D | 3.58 | 326.3 |
| 4.43 | 6-N-(2-amino-2- phenylethyl)-4-N- cyclopropyl-1- methylpyrazolo[3, 4-d]pyrimidine- 4,6-diamine | 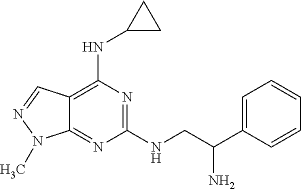 [SMILES: Cn1ncc2c(NC3CC3)nc(NCC(N)c3ccccc3)nc21] | C | 2.42 | 324.2 |
| 4.44 | 6-N-(2-amino-2- phenylethyl)-1- methyl-4-N- (2,2,2- trifluoroethyl) pyrazolo[3,4- d]pyrimidine-4,6- diamine | 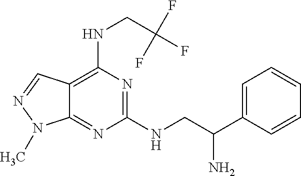 [SMILES: Cn1ncc2c(NCC(F)(F)F)nc(NCC(N)c3ccccc3)nc21] | D | 3.53 | 366.3 |
| 4.45 | N6-[(2R)-2- amino-2-phenyl- ethyl]-1-methyl- N4-(2,2,2- trifluoroethyl) pyrazolo[3,4- d]pyrimidine-4,6- diamine | 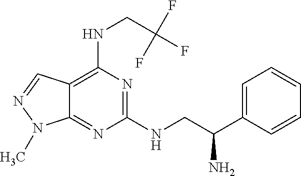 [SMILES: Cn1ncc2c(NCC(F)(F)F)nc(NC[C@H](N)c3ccccc3)nc21] | D | 3.47 | 366.3 |
| 4.46 | 6-N-(2-amino-2- phenylethyl)-1- methyl-4-N- propan-2- ylpyrazolo[3,4- d]pyrimidine-4,6- diamine | 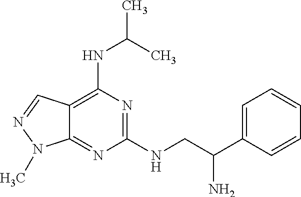 [SMILES: CC(C)Nc1nc(NCC(N)c2ccccc2)nc2c1cnn2C] | C | 2.66 | 326.2 |
| 4.47 | 6-N-[(2R)-2- amino-2- phenylethyl]-1- methyl-4-N- propan-2- ylpyrazolo[3,4- d]pyrimidine-4,6- diamine | 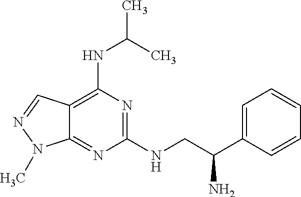 [SMILES: CC(C)Nc1nc(NC[C@H](N)c2ccccc2)nc2c1cnn2C] | D | 3.49 | 326.3 |
| 4.48 | N′-(1-methyl-4- pyrrolidin-1- ylpyrazolo[3,4- d]pyrimidin-6-yl)- 1-phenylethane- 1,2-diamine | 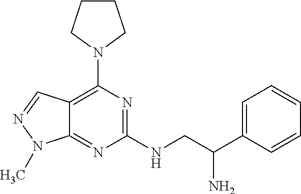 [SMILES: Cn1ncc2c(N3CCCC3)nc(NCC(N)c3ccccc3)nc21] | E | 1.19 | 338.2 |
| 4.49 | 6-N-(2-amino-2- phenylethyl)-4-N- (2-methoxyethyl)- 1- methylpyrazolo[3, 4-d]pyrimidine- 4,6-diamine | 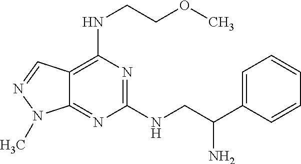 [SMILES: COCCNc1nc(NCC(N)c2ccccc2)nc2c1cnn2C] | C | 2.26 | 342.3 |
| 4.50 | 6-N-(2-amino-2- phenylethyl)-4-N- tert-butyl-1- methylpyrazolo[3, 4-d]pyrimidine- 4,6-diamine | 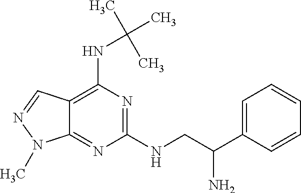 [SMILES: Cn1ncc2c(NC(C)(C)C)nc(NCC(N)c3ccccc3)nc21] | C | 2.99 | 340.3 |
| 4.51 | N6-(2-amino-2- phenyl-propyl)- N4-tert-butyl-1- methyl- pyrazolo[3,4- d]pyrimidine-4,6- diamine | 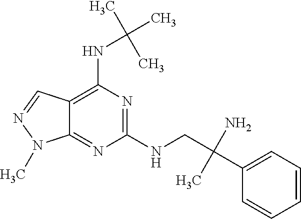 [SMILES: Cn1ncc2c(NC(C)(C)C)nc(NCC(C)(N)c3ccccc3)nc21] | D | 3.97 | 354.3 |
| 4.52 | N6-(2-amino-2- phenyl-ethyl)-N4- tert-butyl-1- (trideuteriomethyl) pyrazolo[3,4- d]pyrimidine-4,6- diamine | 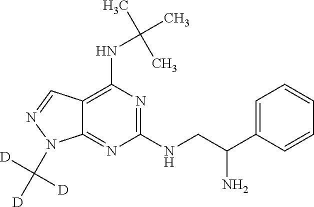 [SMILES: [2H]C([2H])([2H])n1ncc2c(NC(C)(C)C)nc(NCC(N)c3ccccc3)nc21] | D | 3.77 | 343.3 |
| 4.53 | 6-N-[(2R)-2- amino-2- phenylethyl]-4-N- tert-butyl-1- methylpyrazolo[3, 4-d]pyrimidine- 4,6-diamine | 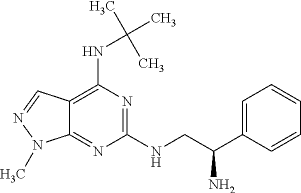 [SMILES: Cn1ncc2c(NC(C)(C)C)nc(NC[C@H](N)c3ccccc3)nc21] | D | 3.72 | 340.4 |
| 4.54 | 6-N-[(2S)-2- amino-2- phenylethyl]-4-N- tert-butyl-1- methylpyrazolo[3, 4-d]pyrimidine- 4,6-diamine | 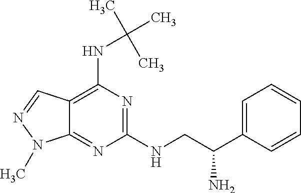 [SMILES: Cn1ncc2c(NC(C)(C)C)nc(NC[C@@H](N)c3ccccc3)nc21] | D | 3.85 | 340.3 |
| 4.55 | N6-[(2R)-2- amino-2-phenyl- ethyl]-N4-tert- butyl-1- (trideuteriomethyl) pyrazolo[3,4- d]pyrimidine-4,6- diamine | 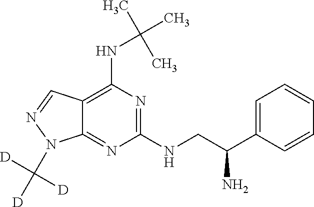 [SMILES: [2H]C([2H])([2H])n1ncc2c(NC(C)(C)C)nc(NC[C@H](N)c3ccccc3)nc21] | C | 2.91 | 343.3 |
| 4.56 | 6-N-(2-amino-2- phenylethyl)-4-N- (cyclopropylmethyl)- 1- methylpyrazolo[3, 4-d]pyrimidine- 4,6-diamine | 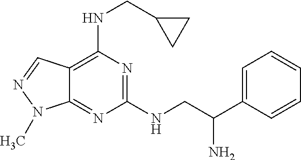 [SMILES: Cn1ncc2c(NCC3CC3)nc(NCC(N)c3ccccc3)nc21] | D | 3.62 | 338.3 |
| 4.57 | N6-(2-amino-2- phenyl-ethyl)-N4- (dicyclopropylmethyl)- 1-methyl- pyrazolo[3,4- d]pyrimidine-4,6- diamine | 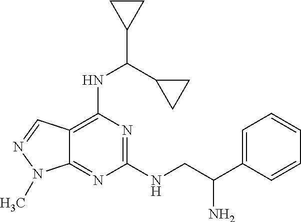 [SMILES: Cn1ncc2c(NC(C3CC3)C3CC3)nc(NCC(N)c3ccccc3)nc21] | D | 3.94 | 378.3 |
| 4.58 | N6-[(2R)-2- amino-2-phenyl- ethyl]-1-methyl- N4-[(2- methylcyclopropyl) methyl]pyrazolo [3,4-d]pyrimidine- 4,6-diamine | 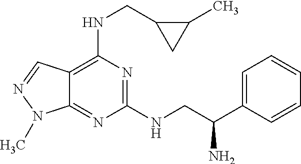 [SMILES: CC1CC1CNc1nc(NC[C@H](N)c2ccccc2)nc2c1cnn2C] | C | 3.01 | 352.3 |
| 4.59 | N6-[(2R)-2- amino-2-phenyl- ethyl]-N4-(1- cyclopropyl-1- methyl-ethyl)-1- methyl- pyrazolo[3,4- d]pyrimidine-4,6- diamine | 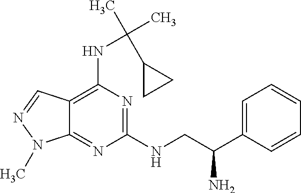 [SMILES: Cn1ncc2c(NC(C)(C)C3CC3)nc(NC[C@H](N)c3ccccc3)nc21] | C | 3.15 | 366.3 |
| 4.60 | N6-[(2R)-2- amino-2-phenyl- ethyl]-1-methyl- N4- (spiro[2.2]pentan- 2- ylmethyl)pyrazolo [3,4-d]pyrimidine- 4,6-diamine | 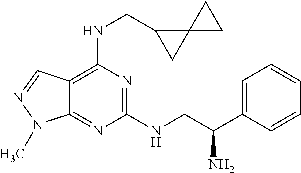 [SMILES: Cn1ncc2c(NCC3CC34CC4)nc(NC[C@H](N)c3ccccc3)nc21] | D | 4.00 | 364.4 |
| 4.61 | N6-[(2R)-2- amino-2-phenyl- ethyl]-N4-[(2,2- difluorocyclopropyl) methyl]-1- methyl- pyrazolo[3,4- d]pyrimidine-4,6- diamine | 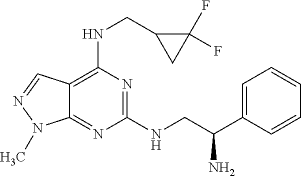 [SMILES: Cn1ncc2c(NCC3CC3(F)F)nc(NC[C@H](N)c3ccccc3)nc21] | C | 2.69 | 374.2 |
| 4.62 | N6-[(2R)-2- amino-2-phenyl- ethyl]-N4-[(2,2- dimethylcyclopropyl) methyl]-1- methyl- pyrazolo[3,4- d]pyrimidine-4,6- diamine | 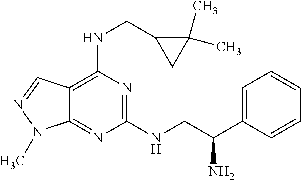 [SMILES: Cn1ncc2c(NCC3CC3(C)C)nc(NC[C@H](N)c3ccccc3)nc21] | C | 3.19 | 366.3 |
| 4.63 | 6-N-(2-amino-2- phenylethyl)-4-N- (2- bicyclo[1.1.1] pentanyl)-1- methylpyrazolo[3, 4-d]pyrimidine- 4,6-diamine | 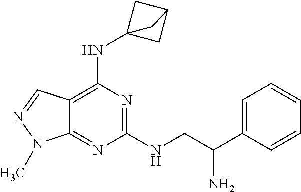 [SMILES: Cn1ncc2c(NC34CC(C3)C4)nc(NCC(N)c3ccccc3)nc21] | D | 3.42 | 350.3 |
| 4.64 | 3-[[6-[(2-amino-2- phenylethyl)amino]- 1- methylpyrazolo[3, 4-d]pyrimidin-4- yl]amino]bicyclo [1.1.1]pentane-1- carbonitrile | 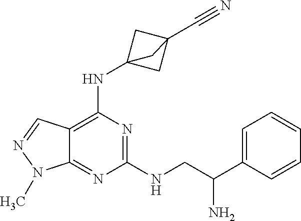 [SMILES: Cn1ncc2c(NC34CC(C#N)(C3)C4)nc(NCC(N)c3ccccc3)nc21] | D | 3.14 | 375.3 |
| 4.65 | N6-(2-amino-2- phenylethyl)-N4- {3- fluorobicyclo[1.1.1] pentan-1-yl}-1- methyl-1H- pyrazolo[3,4- d]pyrimidine-4,6- diamine | 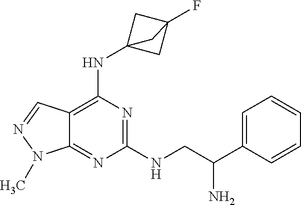 [SMILES: Cn1ncc2c(NC34CC(F)(C3)C4)nc(NCC(N)c3ccccc3)nc21] | E | 1.63 | 368.3 |
| 4.66 | N6-[(2R)-2- amino-2-phenyl- ethyl]-N4-(3- bicyclo[1.1.1] pentanyl)-1-methyl- pyrazolo[3,4- d]pyrimidine-4,6- diamine | 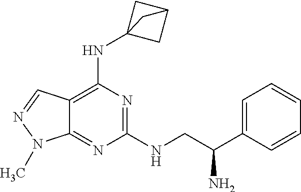 [SMILES: Cn1ncc2c(NC34CC(C3)C4)nc(NC[C@H](N)c3ccccc3)nc21] | D | 3.78 | 350.3 |
| 4.67 | N6-[(2R)-2- amino-2-phenyl- ethyl]-N4-(3- fluoro-1- bicyclo[1.1.1] pentanyl)-1-methyl- pyrazolo[3,4- d]pyrimidine-4,6- diamine | 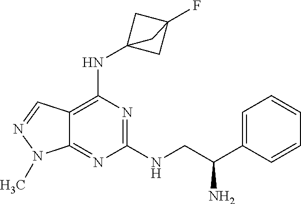 [SMILES: Cn1ncc2c(NC34CC(F)(C3)C4)nc(NC[C@H](N)c3ccccc3)nc21] | D | 3.71 | 368.2 |
| 4.68 | 6-N-(2-amino-2- phenylethyl)-4-N- (4,4- difluorocyclohexyl)- 1- methylpyrazolo[3, 4-d]pyrimidine- 4,6-diamine | 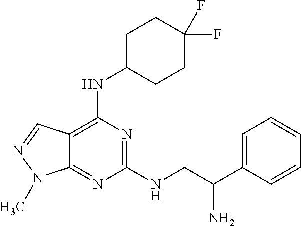 [SMILES: Cn1ncc2c(NC3CCC(F)(F)CC3)nc(NCC(N)c3ccccc3)nc21] | C | 2.96 | 402.3 |
| 4.69 | N6-[(2R)-2- amino-2-phenyl- ethyl]-1-methyl- N4- tetrahydropyran- 4-yl-pyrazolo[3,4- d]pyrimidine-4,6- diamine | 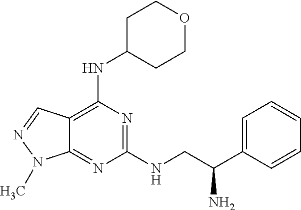 [SMILES: Cn1ncc2c(NC3CCOCC3)nc(NC[C@H](N)c3ccccc3)nc21] | D | 3.14 | 368.2 |
| 4.70 | 3-[[6-[[(2R)-2- amino-2-phenyl- ethyl]amino]-1- methyl- pyrazolo[3,4- d]pyrimidin-4- yl]amino]-3- methyl-butan-1-ol | 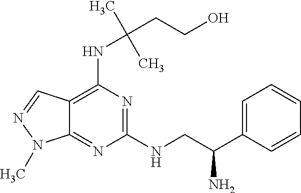 [SMILES: Cn1ncc2c(NC(C)(C)CCO)nc(NC[C@H](N)c3ccccc3)nc21] | D | 3.23 | 370.3 |
| 4.71 | 2-[[6-[[(2R)-2- amino-2-phenyl- ethyl]amino]-1- methyl- pyrazolo[3,4- d]pyrimidin-4- yl]amino]-2- methyl-propan-1- ol | 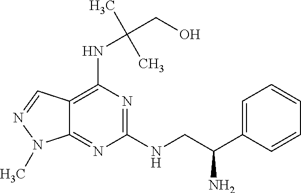 [SMILES: Cn1ncc2c(NC(C)(C)CO)nc(NC[C@H](N)c3ccccc3)nc21] | D | 3.12 | 356.2 |
| 4.72 | N6-[(2R)-2- amino-2-phenyl- ethyl]-N4-(2,2- dimethylpropyl)-1- methyl- pyrazolo[3,4- d]pyrimidine-4,6- diamine | 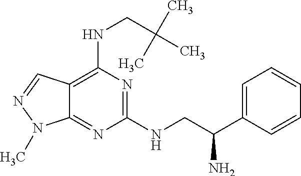 [SMILES: Cn1ncc2c(NCC(C)(C)C)nc(NC[C@H](N)c3ccccc3)nc21] | C | 3.08 | 354.3 |
| 4.73 | N6-[(2R)-2- amino-2-phenyl- ethyl]-N4-(2,2- difluoroethyl)-1- methyl- pyrazolo[3,4- d]pyrimidine-4,6- diamine | 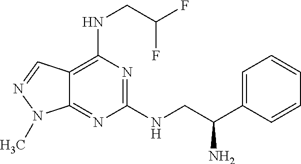 [SMILES: Cn1ncc2c(NCC(F)F)nc(NC[C@H](N)c3ccccc3)nc21] | D | 3.35 | 348.3 |
| 4.74 | N6-[(2R)-2- amino-2-phenyl- ethyl]-N4-(2,2- difluoropropyl)-1- methyl- pyrazolo[3,4- d]pyrimidine-4,6- diamine | 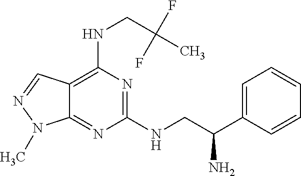 [SMILES: Cn1ncc2c(NCC(C)(F)F)nc(NC[C@H](N)c3ccccc3)nc21] | D | 3.49 | 362.3 |
| 4.75 | N6-[(2R)-2- amino-2-phenyl- ethyl]-1-methyl- N4-(2,2,2- trifluoro-1,1- dimethyl- ethyl)pyrazolo[3,4- d]pyrimidine-4,6- diamine | 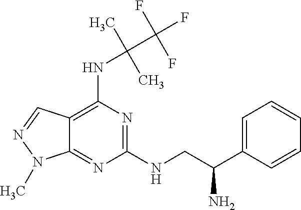 [SMILES: Cn1ncc2c(NC(C)(C)C(F)(F)F)nc(NC[C@H](N)c3ccccc3)nc21] | D | 3.95 | 394.2 |
| 4.76 | N6-[(2R)-2- amino-2-phenyl- ethyl]-1-methyl- N4-[(1S)-2,2,2- trifluoro-1-methyl- ethyl]pyrazolo[3,4- d]pyrimidine-4,6- diamine | 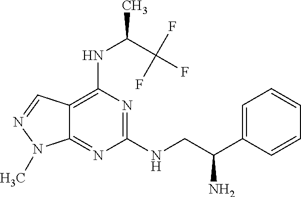 [SMILES: C[C@H](Nc1nc(NC[C@H](N)c2ccccc2)nc2c1cnn2C)C(F)(F)F] | C | 2.82 | 380.3 |
| 4.77 | N6-[(2R)-2- amino-2-phenyl- ethyl]-1-methyl- N4-(1- methylcyclopropyl) pyrazolo[3,4- d]pyrimidine-4,6- diamine | 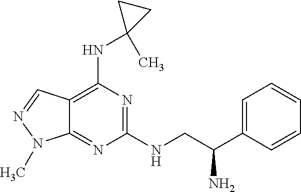 [SMILES: Cn1ncc2c(NC3(C)CC3)nc(NC[C@H](N)c3ccccc3)nc21] | C | 2.61 | 338.3 |
| 4.78 | N6-[(2R)-2- amino-2-phenyl- ethyl]-1-methyl- N4-[1- (trifluoromethyl) cyclopropyl]pyrazolo [3,4- d]pyrimidine-4,6- diamine | 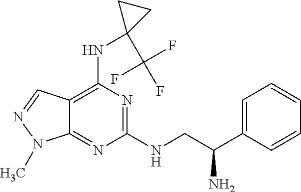 [SMILES: Cn1ncc2c(NC3(C(F)(F)F)CC3)nc(NC[C@H](N)c3ccccc3)nc21] | D | 3.71 | 392.2 |
| 4.79 | N6-[(2R)-2- amino-2-phenyl- ethyl]-N4-(1-tert- butylcyclopropyl)- 1-methyl- pyrazolo[3,4- d]pyrimidine-4,6- diamine | 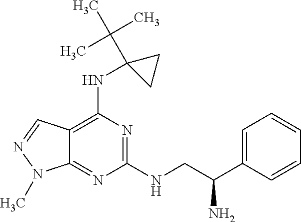 [SMILES: Cn1ncc2c(NC3(C(C)(C)C)CC3)nc(NC[C@H](N)c3ccccc3)nc21] | D | 4.02 | 380.3 |
| 4.80 | N6-[(2R)-2- amino-2-phenyl- ethyl]-1-methyl- N4- spiro[2.2]pentan- 2-yl-pyrazolo[3,4- d]pyrimidine-4,6- diamine | 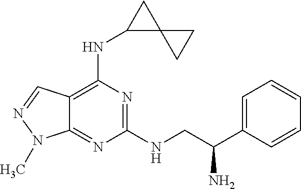 [SMILES: Cn1ncc2c(NC3CC34CC4)nc(NC[C@H](N)c3ccccc3)nc21] | D | 3.71 | 350.3 |
| 4.81 | [3-[[6-[(2-amino- 2-phenyl- ethyl)amino]-1- methyl- pyrazolo[3,4- d]pyrimidin-4- yl]amino]-1- (chloromethyl) cyclobutyl]methanol | 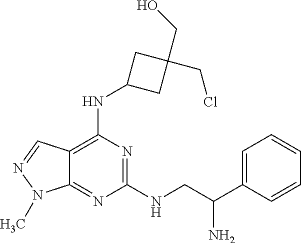 [SMILES: Cn1ncc2c(NC3CC(CO)(CCl)C3)nc(NCC(N)c3ccccc3)nc21] | D | 3.29 | 416.3/ 418.3 |
| 4.82 | N6-(2-amino-2- phenyl-ethyl)-N4- (2,2- difluorospiro[3.3] heptan-6-yl)-1- methyl- pyrazolo[3,4- d]pyrimidine-4,6- diamine | 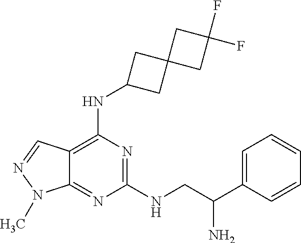 [SMILES: Cn1ncc2c(NC3CC4(C3)CC(F)(F)C4)nc(NCC(N)c3ccccc3)nc21] | D | 3.94 | 414.2 |
| 4.83 | N6-[(2R)-2- amino-2-phenyl- ethyl]-N4- cyclobutyl-1- methyl- pyrazolo[3,4- d]pyrimidine-4,6- diamine | 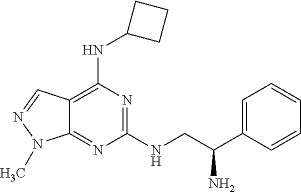 [SMILES: Cn1ncc2c(NC3CCC3)nc(NC[C@H](N)c3ccccc3)nc21] | C | 2.74 | 338.3 |
| 4.84 | N6-[(2R)-2- amino-2-phenyl- ethyl]-1-methyl- N4-(1- methylcyclobutyl) pyrazolo[3,4- d]pyrimidine-4,6- diamine | 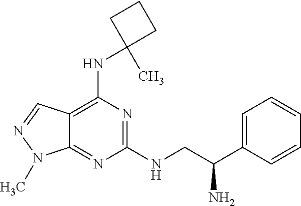 [SMILES: Cn1ncc2c(NC3(C)CCC3)nc(NC[C@H](N)c3ccccc3)nc21] | C | 2.98 | 352.3 |
| 4.85 | N6-[(2R)-2- amino-2-phenyl- ethyl]-N4-(3,3- difluorocyclobutyl)- 1-methyl- pyrazolo[3,4- d]pyrimidine-4,6- diamine | 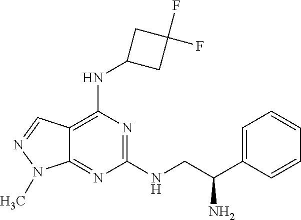 [SMILES: Cn1ncc2c(NC3CC(F)(F)C3)nc(NC[C@H](N)c3ccccc3)nc21] | C | 2.73 | 374.3 |
| 4.86 | N6-[(2R)-2- amino-2-phenyl- ethyl]-N4-(3,3- difluoro-1-methyl- cyclobutyl)-1- methyl- pyrazolo[3,4- d]pyrimidine-4,6- diamine | 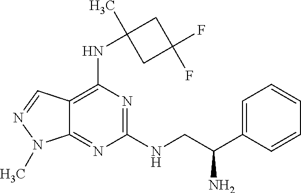 [SMILES: Cn1ncc2c(NC3(C)CC(F)(F)C3)nc(NC[C@H](N)c3ccccc3)nc21] | D | 3.79 | 388.1 |
| 4.87 | N6-[(2R)-2- amino-2-phenyl- ethyl]-1-methyl- N4-(3- methylcyclobutyl) pyrazolo[3,4- d]pyrimidine-4,6- diamine | 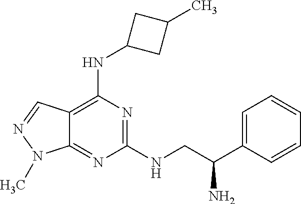 [SMILES: CC1CC(Nc2nc(NC[C@H](N)c3ccccc3)nc3c2cnn3C)C1] | D | 3.93 | 352.3 |
| 4.88 | N6-[(2R)-2- amino-2-phenyl- ethyl]-N4-(3- fluorocyclobutyl)- 1-methyl- pyrazolo[3,4- d]pyrimidine-4,6- diamine | 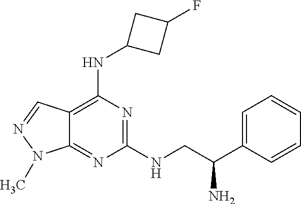 [SMILES: Cn1ncc2c(NC3CC(F)C3)nc(NC[C@H](N)c3ccccc3)nc21] | D | 3.48 | 356.3 |
| 4.89 | N6-[(2R)-2- amino-2-phenyl- ethyl]-N4-(3- fluorocyclobutyl)- 1-methyl- pyrazolo[3,4- d]pyrimidine-4,6- diamine | 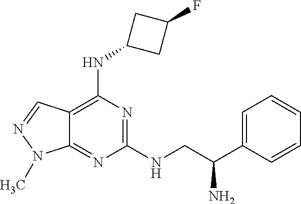 [SMILES: Cn1ncc2c(N[C@H]3C[C@H](F)C3)nc(NC[C@H](N)c3ccccc3)nc21] | D | 3.49 | 356.2 |
| 4.90 | N6-[(2R)-2- amino-2-phenyl- ethyl]-N4-(3- fluorocyclobutyl)- 1-methyl- pyrazolo[3,4- d]pyrimidine-4,6- diamine | 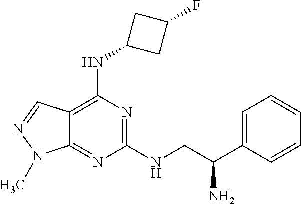 [SMILES: Cn1ncc2c(N[C@H]3C[C@@H](F)C3)nc(NC[C@H](N)c3ccccc3)nc21] | D | 3.48 | 356.2 |
| 4.91 | 3-[[6-[[(2R)-2- amino-2-phenyl- ethyl]amino]-1- methyl- pyrazolo[3,4- d]pyrimidin-4- yl]amino] cyclobutanecarbonitrile | 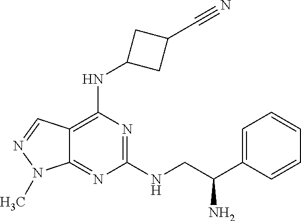 [SMILES: Cn1ncc2c(NC3CC(C#N)C3)nc(NC[C@H](N)c3ccccc3)nc21] | D | 3.26 | 363.2 |
| 4.92 | N6-[(2R)-2- amino-2-phenyl- ethyl]-N4-(3- methoxycyclobutyl)- 1-methyl- pyrazolo[3,4- d]pyrimidine-4,6- diamine | 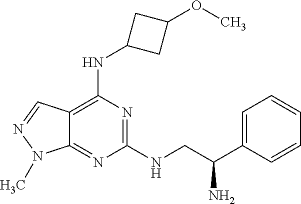 [SMILES: COC1CC(Nc2nc(NC[C@H](N)c3ccccc3)nc3c2cnn3C)C1] | C | 2.41 | 368.3 |
| 4.93 | N6-[(2R)-2- amino-2-phenyl- ethyl]-N4-(3,3- dichlorocyclobutyl)- 1-methyl- pyrazolo[3,4- d]pyrimidine-4,6- diamine | 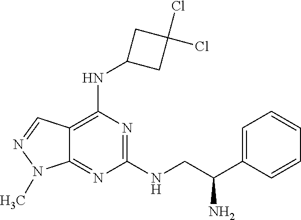 [SMILES: Cn1ncc2c(NC3CC(Cl)(Cl)C3)nc(NC[C@H](N)c3ccccc3)nc21] | D | 4.05 | 406.2/ 409.1 |
| 4.94 | N6-[(2R)-2- amino-2-phenyl- ethyl]-N4-(2,2- difluorospiro[3.3] heptan-6-yl)-1- methyl- pyrazolo[3,4- d]pyrimidine-4,6- diamine | 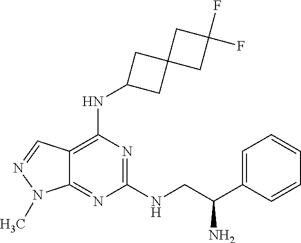 [SMILES: Cn1ncc2c(NC3CC4(C3)CC(F)(F)C4)nc(NC[C@H](N)c3ccccc3)nc21] | D | 3.97 | 414.3 |
| 4.95 | N6-[(2R)-2- amino-2-phenyl- ethyl]-1-methyl- N4-(2- oxaspiro[3.3] heptan-6- yl)pyrazolo[3,4- d]pyrimidine-4,6- diamine | 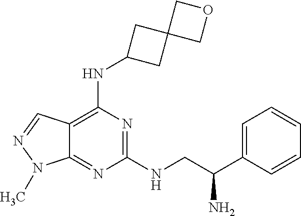 [SMILES: Cn1ncc2c(NC3CC4(COC4)C3)nc(NC[C@H](N)c3ccccc3)nc21] | D | 3.11 | 380.3 |
| 4.96 | N6-[(2R)-2- amino-2-phenyl- ethyl]-N4-(3,3- dimethylcyclobutyl)- 1-methyl- pyrazolo[3,4- d]pyrimidine-4,6- diamine | 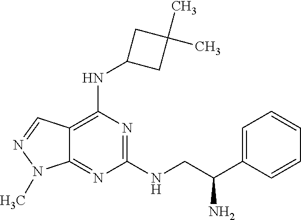 [SMILES: Cn1ncc2c(NC3CC(C)(C)C3)nc(NC[C@H](N)c3ccccc3)nc21] | D | 4.17 | 366.3 |
| 4.97 | N6-[(2R)-2- amino-2-phenyl- ethyl]-N4-(3- fluoro-3-methyl- cyclobutyl)-1- methyl- pyrazolo[3,4- d]pyrimidine-4,6- diamine | 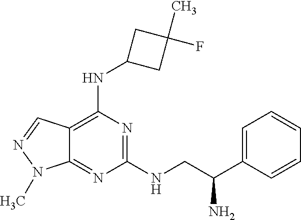 [SMILES: Cn1ncc2c(NC3CC(C)(F)C3)nc(NC[C@H](N)c3ccccc3)nc21] | D | 3.70 | 370.2 |
| 4.98 | N6-[(2R)-2- amino-2-phenyl- ethyl]-1-methyl- N4-[(1S)-2,2,2- trifluoro-1-methyl- ethyl]pyrazolo[3,4- d]pyrimidine-4,6- diamine | 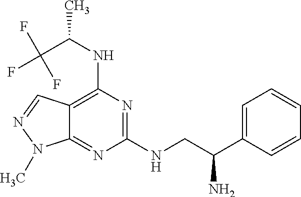 [SMILES: C[C@H](Nc1nc(NC[C@H](N)c2ccccc2)nc2c1cnn2C)C(F)(F)F] | C | 2.82 | 380.3 |
| 4.99 | N6-[(2R)-2- amino-2-phenyl- ethyl]-1-methyl- N4- spiro[3.3]heptan- 2-yl-pyrazolo[3,4- d]pyrimidine-4,6- diamine | 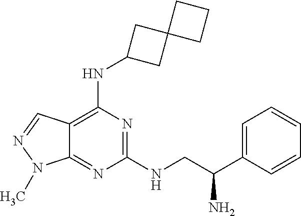 [SMILES: Cn1ncc2c(NC3CC4(CCC4)C3)nc(NC[C@H](N)c3ccccc3)nc21] | C | 3.42 | 378.3 |
| 4.100 | N6-[(2R)-2- amino-2-phenyl- ethyl]-N4-(2- methoxy-1,1- dimethyl-ethyl)-1- methyl- pyrazolo[3,4- d]pyrimidine-4,6- diamine | 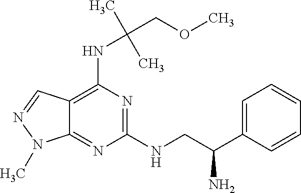 [SMILES: COCC(C)(C)Nc1nc(NC[C@H](N)c2ccccc2)nc2c1cnn2C] | D | 3.58 | 370.3 |
| 4.101 | N6-[(2R)-2- amino-2-phenyl- ethyl]-N4-(1,1- dimethylpropyl)-1- methyl- pyrazolo[3,4- d]pyrimidine-4,6- diamine | 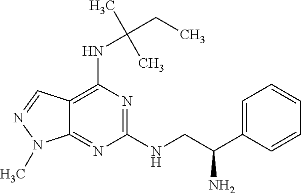 [SMILES: CCC(C)(C)Nc1nc(NC[C@H](N)c2ccccc2)nc2c1cnn2C] | D | 3.97 | 354.3 |
| 4.102 | N6-[(2R)-2- amino-2-phenyl- ethyl]-N4-[(1S)-1- cyclopropylethyl]- 1-methyl- pyrazolo[3,4- d]pyrimidine-4,6- diamine | 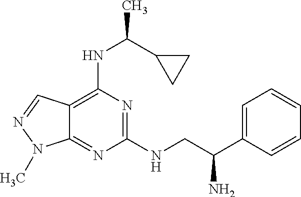 [SMILES: C[C@H](Nc1nc(NC[C@H](N)c2ccccc2)nc2c1cnn2C)C1CC1] | D | 3.73 | 352.3 |
| 4.103 | N6-[(2R)-2- amino-2-phenyl- ethyl]-N4- isobutyl-1-methyl- pyrazolo[3,4- d]pyrimidine-4,6- diamine | 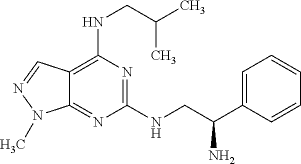 [SMILES: CC(C)CNc1nc(NC[C@H](N)c2ccccc2)nc2c1cnn2C] | D | 3.73 | 340.3 |
| 4.104 | N6-[(2R)-2- amino-2-phenyl- ethyl]-1-methyl- N4- (spiro[2.3]hexan- 2- ylmethyl)pyrazolo [3,4-d]pyrimidine- 4,6-diamine | 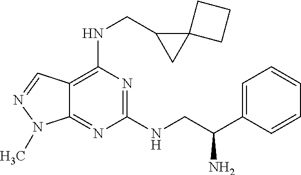 [SMILES: Cn1ncc2c(NCC3CC34CCC4)nc(NC[C@H](N)c3ccccc3)nc21] | D | 4.20 | 378.3 |
| 4.105 | N6-[(2R)-2- amino-2-phenyl- ethyl]-N4- (cyclobutylmethyl)- 1-methyl- pyrazolo[3,4- d]pyrimidine-4,6- diamine | 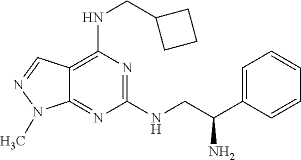 [SMILES: Cn1ncc2c(NCC3CCC3)nc(NC[C@H](N)c3ccccc3)nc21] | C | 3.04 | 352.3 |
| 4.106 | N6-[(2R)-2- amino-2-phenyl- ethyl]-N4-[(2,2- difluorocyclobutyl) methyl]-1-methyl- pyrazolo[3,4- d]pyrimidine-4,6- diamine | 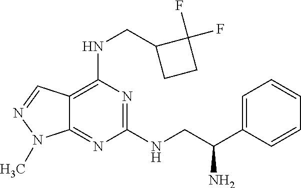 [SMILES: Cn1ncc2c(NCC3CCC3(F)F)nc(NC[C@H](N)c3ccccc3)nc21] | C | 2.85 | 388.3 |
| 4.107 | N6-[(2R)-2- amino-2-phenyl- ethyl]-N4-[(1- fluorocyclobutyl) methyl]-1-methyl- pyrazolo[3,4- d]pyrimidine-4,6- diamine | 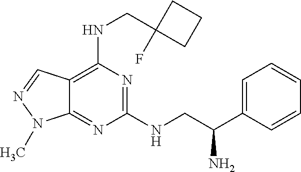 [SMILES: Cn1ncc2c(NCC3(F)CCC3)nc(NC[C@H](N)c3ccccc3)nc21] | C | 2.83 | 370.3 |
| 4.108 | N6-[(2R)-2- amino-2-phenyl- ethyl]-N4-[(3- fluorocyclobutyl) methyl]-1-methyl- pyrazolo[3,4- d]pyrimidine-4,6- diamine | 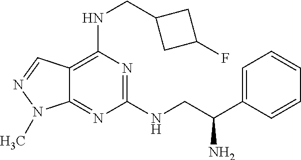 [SMILES: Cn1ncc2c(NCC3CC(F)C3)nc(NC[C@H](N)c3ccccc3)nc21] | D | 2.75 | 370.3 |
| 4.109 | N6-[(2R)-2- amino-2-phenyl- ethyl]-1-methyl- N4-[3- (trifluoromethyl) cyclobutyl]pyrazolo [3,4-d]pyrimidine- 4,6-diamine | 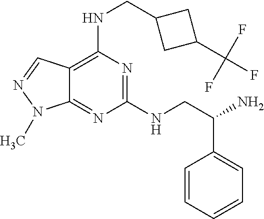 [SMILES: Cn1ncc2c(NCC3CC(C(F)(F)F)C3)nc(NC[C@H](N)c3ccccc3)nc21] | D | 3.92 | 406.2 |
| 4.110 | N6-[(2R)-2- amino-2-phenyl- ethyl]-N4-(2- fluoro-2-methyl- propyl)-1-methyl- pyrazolo[3,4- d]pyrimidine-4,6- diamine | 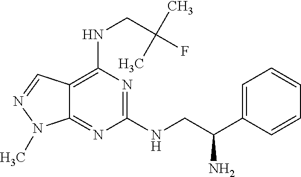 [SMILES: Cn1ncc2c(NCC(C)(C)F)nc(NC[C@H](N)c3ccccc3)nc21] | D | 3.51 | 358.3 |
| 4.111 | N6-[(2R)-2- amino-2-phenyl- ethyl]-1-methyl- N4-(1- methylcyclohexyl) pyrazolo[3,4- d]pyrimidine-4,6- diamine | 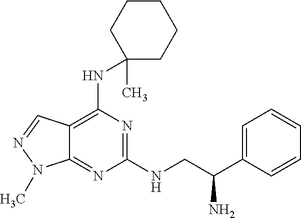 [SMILES: Cn1ncc2c(NC3(C)CCCCC3)nc(NC[C@H](N)c3ccccc3)nc21] | D | 4.26 | 380.3 |
| 4.112 | N6-[(2R)-2- amino-2-phenyl- ethyl]-N4-[(2- fluorocyclobutyl) methyl]-1-methyl- pyrazolo[3,4- d]pyrimidine-4,6- diamine | 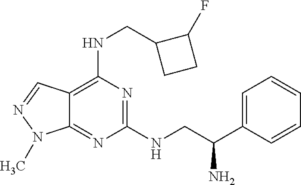 [SMILES: Cn1ncc2c(NCC3CCC3F)nc(NC[C@H](N)c3ccccc3)nc21] | D | 3.60 | 370.3 |
| 4.113 | N6-[(2R)-2- amino-2-phenyl- ethyl]-N4- cyclopentyl-1- methyl- pyrazolo[3,4- d]pyrimidine-4,6- diamine | 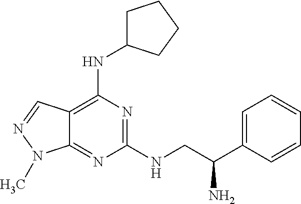 [SMILES: Cn1ncc2c(NC3CCCC3)nc(NC[C@H](N)c3ccccc3)nc21] | D | 3.80 | 352.3 |
| 4.114 | N6-[(2R)-2- amino-2-phenyl- ethyl]-1-methyl- N4-(3,3,3- trifluoropropyl) pyrazolo[3,4- d]pyrimidine-4,6- diamine | 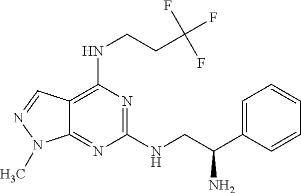 [SMILES: Cn1ncc2c(NCCC(F)(F)F)nc(NC[C@H](N)c3ccccc3)nc21] | D | 3.60 | 380.2 |
| 4.115 | N6-[(2R)-2- amino-2-phenyl- ethyl]-N4-[(2,2- dichlorocyclopropyl) methyl]-1- methyl- pyrazolo[3,4- d]pyrimidine-4,6- diamine | 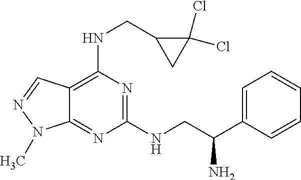 [SMILES: Cn1ncc2c(NCC3CC3(Cl)Cl)nc(NC[C@H](N)c3ccccc3)nc21] | D | 3.91 | 406.2/ 408.2 |
| 4.116 | N6-[(2R)-2- amino-2-phenyl- ethyl]-1-methyl- N4-(1- methylcyclopentyl) pyrazolo[3,4- d]pyrimidine-4,6- diamine | 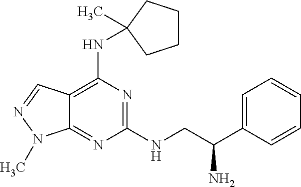 [SMILES: Cn1ncc2c(NC3(C)CCCC3)nc(NC[C@H](N)c3ccccc3)nc21] | D | 4.08 | 366.3 |
| 4.117 | N6-[(2R)-2- amino-2-phenyl- ethyl]-N4-[(1R)-2- methoxy-1- methyl-ethyl]-1- methyl- pyrazolo[3,4- d]pyrimidine-4,6- diamine | 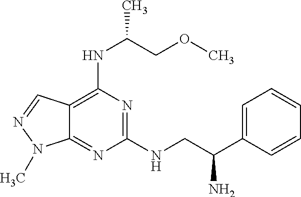 [SMILES: COC[C@@H](C)Nc1nc(NC[C@H](N)c2ccccc2)nc2c1cnn2C] | D | 3.33 | 356.2 |
| 4.118 | N6-[(2R)-2- amino-2-phenyl- ethyl]-1-methyl- N4-(1-methyl-1- tetrahydropyran- 4-yl- ethyl)pyrazolo[3,4- d]pyrimidine-4,6- diamine | 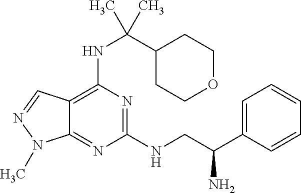 [SMILES: Cn1ncc2c(NC(C)(C)C3CCOCC3)nc(NC[C@H](N)c3ccccc3)nc21] | D | 3.48 | 410.3 |
| 4.119 | 1-methyl-N6- [(2R)-2-amino-2- phenyl-ethyl]-N4- spiro[2.3]hexan- 5-yl-pyrazolo[3,4- d]pyrimidine-4,6- diamine | 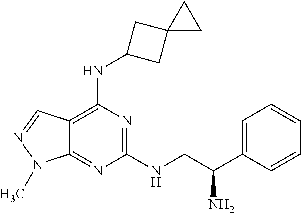 [SMILES: Cn1ncc2c(NC3CC4(CC4)C3)nc(NC[C@H](N)c3ccccc3)nc21] | C | 3.14 | 364.3 |
| 4.120 | N6-[(2R)-2- amino-2-phenyl- ethyl]-N4-(2,2- dimethylcyclopropyl)- 1-methyl- pyrazolo[3,4- d]pyrimidine-4,6- diamine | 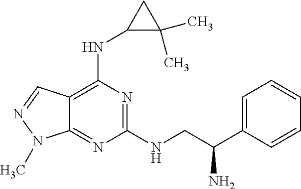 [SMILES: Cn1ncc2c(NC3CC3(C)C)nc(NC[C@H](N)c3ccccc3)nc21] | C | 2.93 | 352.4 |
| 4.122 | N6-[(2R)-2- amino-2- phenylethyl]-1- methyl-N4-[(2R)- 3-methylbutan-2- yl]-1H- pyrazolo[3,4- d]pyrimidine-4,6- diamine | 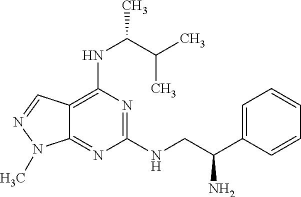 [SMILES: CC(C)[C@@H](C)Nc1nc(NC[C@H](N)c2ccccc2)nc2c1cnn2C] | D | 3.94 | 354.3 |
| 4.123 | N6-[(2R)-2- amino-2- phenylethyl]-1- methyl-N4-[(2S)- 3-methylbutan-2- yl]-1H- pyrazolo[3,4- d]pyrimidine-4,6- diamine | 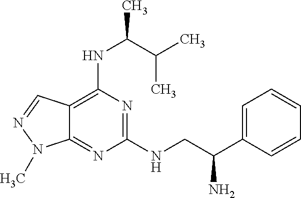 [SMILES: CC(C)[C@H](C)Nc1nc(NC[C@H](N)c2ccccc2)nc2c1cnn2C] | D | 3.87 | 354.3 |
| 4.124 | 6-[(6-{[(2R)-2- amino-2- phenylethyl]amino}- 1-methyl-1H- pyrazolo[3,4- d]pyrimidin-4- yl)amino]spiro[3.3] heptan-2-ol | 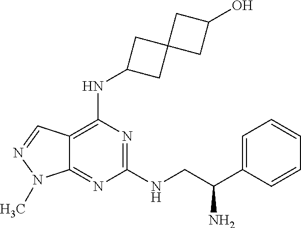 [SMILES: Cn1ncc2c(NC3CC4(CC(O)C4)C3)nc(NC[C@H](N)c3ccccc3)nc21] | C | 2.19 | 394.4 |
| 4.125 | N6-[(2R)-2- amino-2- phenylethyl]-1- methyl-N4-[4- (trifluoromethyl) cyclohexyl]-1H- pyrazolo[3,4- d]pyrimidine-4,6- diamine | 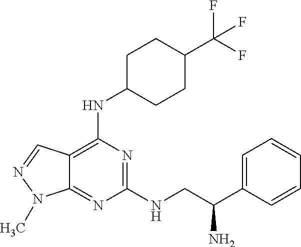 [SMILES: Cn1ncc2c(NC3CCC(C(F)(F)F)CC3)nc(NC[C@H](N)c3ccccc3)nc21] | D | 4.20 | 434.3 |
| 4.126 | N6-[(2R)-2- amino-2- phenylethyl]-N4- [(1S)-3,3- difluorocyclopentyl]- 1-methyl-1H- pyrazolo[3,4- d]pyrimidine-4,6- diamine | 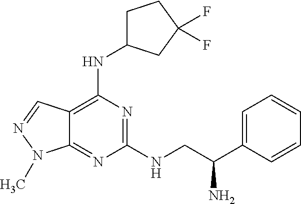 [SMILES: Cn1ncc2c(NC3CCC(F)(F)C3)nc(NC[C@H](N)c3ccccc3)nc21] | E | 1.58 | 388.3 |
| 4.127 | N6-[(2R)-2- amino-2- phenylethyl]-1- methyl-N4-(4- methyloxan-4-yl)- 1H-pyrazolo[3,4- d]pyrimidine-4,6- diamine | 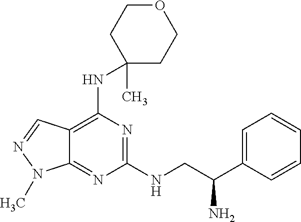 [SMILES: Cn1ncc2c(NC3(C)CCOCC3)nc(NC[C@H](N)c3ccccc3)nc21] | D | 2.50 | 382.3 |
| 4.128 | N6-[(2R)-2- amino-2- phenylethyl]-N4- tert-butyl-1,3- dimethyl-1H- pyrazolo[3,4- d]pyrimidine-4,6- diamine | 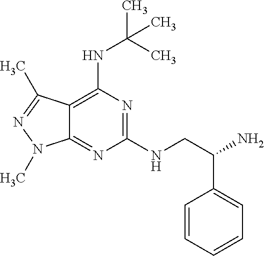 [SMILES: Cc1nn(C)c2nc(NC[C@H](N)c3ccccc3)nc(NC(C)(C)C)c12] | C | 3.18 | 354.2 |
| 4.129 | N6-[(2R)-2- amino-2- phenylethyl]-N4- [(1R)-1- cyclopropylethyl]- 1-methyl-1H- pyrazolo[3,4- d]pyrimidine-4,6- diamine | 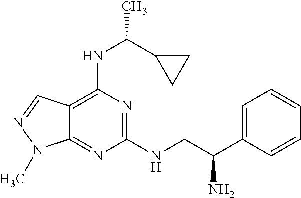 [SMILES: C[C@@H](Nc1nc(NC[C@H](N)c2ccccc2)nc2c1cnn2C)C1CC1] | D | 3.78 | 352.3 |

The synthesis of the following compounds was carried out only partially according to the General Methods described above and as such are detailed separately

### Compound 4.121: N6-[(2R)-2-amino-2-phenyl-ethyl]-N4-tert-butyl-1-cyclopropyl-pyrazolo[3,4-d]pyrimidine-4,6-diamine

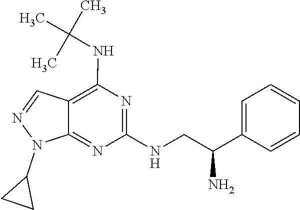 [SMILES: CC(C)(C)Nc1nc(NC[C@H](N)c2ccccc2)nc2c1cnn2C1CC1]

Step 1: To a solution of 2,4,6-trichloropyrimidine-5-carbaldehyde (1.00 g, 4.73 mmol) in ethanol (10 mL) at −78° C. was added triethylamine (1.6 mL, 11.8 mmol) and cyclopropyl hydrazine hydrochloride (0.54 g, 4.97 mmol) and the resulting yellow mixture was allowed to stir at −78° C. for 30 minutes and warmed to 0° C. for a further 2 hours. The solvent was removed in vacuo (keeping the water bath at 20° C.). The resulting yellow solid was dissolved in ethyl acetate (10 mL) and washed with saturated ammonium chloride solution (5 mL). The layers were separated and the aqueous layer extracted twice more with ethyl acetate (2×10 mL). The organic layers were combined and washed with brine (5 mL), dried over magnesium sulfate, filtered and concentrated in vacuo to give a yellow solid which was purified by silica gel chromatography eluting with 0-100% ethyl acetate:heptane to yield 4,6-dichloro-1-cyclopropyl-pyrazolo[3,4-d]pyrimidine (670 mg, 62%) as a white solid. LCMS (Analytical Method A) t_{R}=1.18 min, [M+H]⁺=228.8/230.85; 1H NMR (500 MHz, DMSO-d6) δ 8.48 (s, 1H), 3.93-3.88 (m, 1H), 1.23-1.14 (m, 4H).

Step 2: N-tert-Butyl-6-chloro-1-cyclopropyl-pyrazolo[3,4-d]pyrimidin-4-amine was synthesised according to General Method 1 and was purified via silica gel chromatography eluting with 0-50% heptane:ethyl acetate to yield the desired compound as a white solid (245 mg, 84%). LCMS (Analytical Method B) t_{R}=1.71 min, [M+H]⁺=266.3/268.2; 1H NMR (400 MHz, Chloroform-d) δ 7.73 (s, 1H), 5.25 (s, 1H), 3.79 (tt, J=7.4, 3.8 Hz, 1H), 1.58 (s, 9H), 1.31-1.25 (m, 2H), 1.16-1.10 (m, 2H).

Step 3: N6-[(2R)-2-Amino-2-phenyl-ethyl]-N4-tert-butyl-1-cyclopropyl-pyrazolo[3,4-d]pyrimidine-4,6-diamine was synthesised according to General Method 3 and was purified by high pH preparative HPLC (Method B) to yield the title compound as a white solid (39 mg, 69%). LCMS (Analytical Method D) t_{R}=4.02 min, [M+H]⁺=366.4; 1 H NMR (400 MHz, DMSO-d6) δ 7.84 (s, 1H), 7.44 (s, 2H), 7.35-7.29 (m, 2H), 7.25-7.19 (m, 1H), 6.79 (s, 1H), 6.27-6.22 (m, 1H), 4.13 (dd, J=8.1, 4.6 Hz, 1H), 3.68-3.62 (m, 1H), 3.61-3.55 (m, 1H), 3.33-3.27 (m, 1H), 1.82 (s, 2H), 1.50 (s, 9H), 1.14-1.09 (m, 2H), 0.97-0.91 (m, 2H).

### Compound 4.130: N2-[1-methyl-4-(2-methylpropyl)-1H-pyrazolo[3,4-d]pyrimidin-6-yl]-1-phenylethane-1,2-diamine

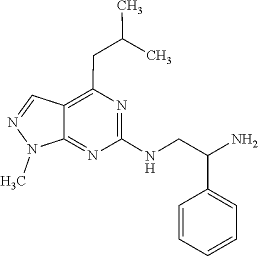 [SMILES: CC(C)Cc1nc(NCC(N)c2ccccc2)nc2c1cnn2C]

Step 1: To a stirred solution of 4,6-dichloro-1-methyl-1H-pyrazolo[3,4-d]pyrimidine (200 mg, 0.985 mmol) and iron;pentane-2,4-dione (105 mg, 0.296 mmol) in dry diethyl ether (1 mL) at 0° C. was added bromo(2-methylpropyl)magnesium (2M in diethyl ether, 0.49 mL, 0.985 mmol) dropwise and stirring continues for 2 hours at 0° C. where upon the reaction mixture was allowed to warm to ambient temperature for 18 hours. The reaction mixture was cooled to 0° C. and a further 2 equivalents of 2M bromo(2-methylpropyl)magnesium in ether was added dropwise. The reaction mixture was quenched with dropwise addition of water (10 mL) and extracted into DCM (3×10 mL). The organic layers were combined and washed with brine (20 mL), dried over magnesium sulfate, filtered and concentrated in vacuo to give the crude material as an orange oil. Purification by silica gel chromatography eluting with 100% heptane gave a mixture of the disubstituted and monosubstituted product as a colourless oil (120 mg) which was used directly in the next step without further purification. LCMS (Analytical Method B) t_{R}=1.58 min, [M+H]⁺=225.3; 1H NMR (250 MHz, Chloroform-d) δ 8.02 (s, 1H), 4.02 (d, J=2.3 Hz, 3H), 2.85 (d, J=7.3 Hz, 2H), 2.26 (dh, J=13.7, 7.0 Hz, 1H), 0.91 (dd, J=6.6, 2.4 Hz, 7H).

Step 2: A mixture of 6-chloro-4-isobutyl-1-methyl-pyrazolo[3,4-d]pyrimidine (120 mg, 0.374 mmol) and tert-butyl N-(2-amino-1-phenyl-ethyl)carbamate (177 mg, 0.748 mmol) in 1-butanol (1 mL) were stirred at 120° C. for 18 hours. The crude reaction mixture was concentrated in vacuo and purified by NP chromatography eluting with EtOAc:Heptane 5:95 to 100:0 to give a pale yellow powder which was purified further by high pH preparative HPLC (Method B) to yield the desired compound as a white powder (35 mg, 22%). LCMS (Analytical Method B) t_{R}=1.82 min, [M+H]⁺=425.3; 1H NMR (500 MHz, DMSO-d6) δ 8.03 (s, 1H), 7.46-7.17 (m, 7H), 4.91 (s, 1H), 3.84 (s, 3H), 3.63 (s, 1H), 3.43 (s, 1H), 2.80-2.64 (m, 2H), 1.33 (s, 9H), 1.09 (s, 1H), 0.92 (s, 6H).

Step 3: tert-Butyl N-[2-[(4-isobutyl-1-methyl-pyrazolo[3,4-d]pyrimidin-6-yl)amino]-1-phenyl-ethyl]carbamate (30 mg, 0.0707 mmol) was suspended in 4 M hydrogen chloride in dioxane (0.35 mL, 1.41 mmol) and the resulting reaction mixture stirred at ambient temperature for 1 hour. The solvent was removed in vacuo and was loaded onto an SCX column and eluted with 2N solution of ammonia in methanol to yield the title compound as an off white solid (22 mg, 93%). LCMS (Analytical Method E) t_{R}=4.04 min, [M+H]⁺=325.4; 1H NMR (400 MHz, DMSO-d6) δ 8.00 (s, 1H), 7.44 (d, J=7.3 Hz, 2H), 7.32 (t, J=7.6 Hz, 3H), 7.21 (t, J=7.3 Hz, 1H), 4.15 (s, 1H), 3.79 (s, 3H), 3.61 (s, 1H), 3.25 (m, 1H), 2.69 (d, J=5.7 Hz, 2H), 2.19 (s, 2H), 0.92 (d, J=6.6 Hz, 6H).

The compounds in Table 5 were prepared in an analogous manner to the above compounds or by methods detailed in the examples to follow according to either General Method 3, General Method 4 or General Method 5 using either the corresponding substituted 1-phenylethane-1,2-diamine or substituted tert-butyl N-[2-amino-1-phenylethyl]carbamate and the corresponding 4-substituted pyrazolopyrimidine intermediate which were in turn synthesised according to General Method 1 or General Method 2.

### Compound 5.01: 6-N-[2-amino-2-(2-methoxyphenyl)ethyl]-4-N-[(3-chloro-4-methylphenyl)methyl]-1-methylpyrazolo[3,4-d]pyrimidine-4,6-diamine

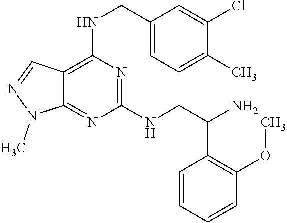 [SMILES: COc1ccccc1C(N)CNc1nc(NCc2ccc(C)c(Cl)c2)c2cnn(C)c2n1]

Step 1: A suspension of ethyl 5-amino-1-methyl-1H-pyrazole-4-carboxylate (3.26 g, 19.27 mmol) and benzoyl isothiocyanate (4.14 mL, 30.83 mmol) in anhydrous 1,4 dioxane (30 mL) was refluxed for 12 hours. After which time LCMS showed complete reaction and the reaction mixture was diluted into ethyl acetate (20 mL) and washed with saturated brine (2×10 mL). The organic layer was dried over magnesium sulfate, filtered and concentrated in vacuo to yield the crude material as an orange solid which was purified by silica gel chromatography eluting with 20-100% ethyl acetate:heptane to yield ethyl 1-methyl-5-{[(phenylformamido)methanethioyl]amino}-1H-pyrazole-4-carboxylate as a yellow solid. (6.0 g, 79%). LCMS (Analytical Method B) t_{R}=1.24 min, [M+H]⁺=333.1; 1H NMR (250 MHz, DMSO-d6) δ 12.13 (s, 1H), 12.08 (s, 1H), 8.07-7.98 (m, 2H), 7.88 (s, 1H), 7.75-7.66 (m, 1H), 7.63-7.52 (m, 2H), 4.15 (q, J=7.1 Hz, 2H), 3.73 (s, 3H), 1.19 (t, J=7.1 Hz, 3H).

Step 2: A solution of ethyl 1-methyl-5-{[(phenylformamido)methanethioyl]amino}-1H-pyrazole-4-carboxylate (6 g, 18.05 mmol) in 1M sodium hydroxide solution (55 mL) was refluxed for 10 minutes and successively diluted with water (100 mL). The solution was concentrated in vacuo to yield a white solid that was used without additional purification (3.2 g, 97%). LCMS (Analytical Method B) t_{R}=0.61 min, [M+H]+=183.0.

Step 3: To a solution of 1-methyl-6-sulfanyl-1H-pyrazolo[3,4-d]pyrimidin-4-ol (3.2 g, 17.56 mmol) in 1M NaOH (53 mL) was added iodomethane (1.09 mL, 17.56 mmol) and the resulting solution was stirred at room temperature for 2 hours. The reaction mixture was treated with glacial acetic acid and filtered to yield the desired compound as an off white solid (3.14 g, 91%). LCMS (Analytical Method D) t_{R}=1.50 min, [M+H]⁺=197.3; 1H NMR (250 MHz, DMSO-d6) δ 7.90 (s, 1H), 3.86 (s, 3H), 2.57 (s, 3H).

Step 4: To phosphorus oxychloride (2.38 mL, 25.48 mmol) at room temperature under a nitrogen atmosphere was added 1-methyl-6-(methylsulfanyl)-1H,4H,5H-pyrazolo[3,4-d]pyrimidin-4-one (500 mg, 2.55 mmol) and the reaction mixture was stirred at 110° C. for 1 hour. After cooling, the reaction mixture was concentrated in vacuo to yield a yellow solid which was used directly in the Step 5 (600 mg, 100%). 1H NMR (500 MHz, DMSO-d6) δ 8.32 (s, 1H), 3.99 (s, 3H), 2.62 (s, 3H).

Step 5: To a solution of 4-chloro-1-methyl-6-methylsulfanyl-pyrazolo[3,4-d]pyrimidine (190 mg, 0.885 mmol) in 1-butanol (2 mL) was added 1-(3-chloro-4-methylphenyl)methanamine (276 mg, 1.77 mmol) and N-ethyl-N-isopropyl-propan-2-amine and the resulting reaction mixture was heated to 110° C. for 2 hours. The solvents were evaporated and the crude product was purified by silica gel chromatography eluting with heptane:ethyl acetate (5-100%) to yield N-[(3-chloro-4-methyl-phenyl)methyl]-1-methyl-6-methylsulfanyl-pyrazolo[3,4-d]pyrimidin-4-amine (281 mg, 95%) as an off-white foam. LCMS (Analytical Method B) t_{R}=1.79 min, [M+H]⁺=334.2/336.2; 1H NMR (250 MHz, Chloroform-d) δ 7.64 (s, 1H), 7.26 (s, 1H), 7.11 (t, J=6.8 Hz, 2H), 5.59 (s, 1H), 4.68 (d, J=5.8 Hz, 2H), 3.88 (s, 3H), 2.51 (s, 3H), 2.28 (s, 3H), 1.58.

Step 6: To a solution of N-[(3-chloro-4-methyl-phenyl)methyl]-1-methyl-6-methylsulfanyl-pyrazolo[3,4-d]pyrimidin-4-amine (280 mg, 0.839 mmol) in methanol (3 mL) and THF (3 mL) was added a solution of bis(O-(hydroperoxysulfonyl)oxidanol) bis(sulfuric acid) pentapotassium (1.56 g, 2.52 mmol) in water (7 mL) and the reaction mixture was stirred at room temperature for 2 hours. The reaction mixture was partitioned between water and DCM and the aqueous layer was extracted into DCM (×3). The combined organic extracts were dried over magnesium sulfate, filtered and concentrated under reduced pressure to give N-[(3-chloro-4-methyl-phenyl)methyl]-1-methyl-6-methylsulfonyl-pyrazolo[3,4-d]pyrimidin-4-amine (257 mg, 82%) as a white solid. LCMS (Analytical Method B) t_{R}=1.53 min, [M+H]⁺=366.2; 1H NMR (250 MHz, Chloroform-d) δ 7.92 (s, 1H), 7.28 (s, 1H), 7.10 (t, J=6.0 Hz, 2H), 4.73 (d, J=5.7 Hz, 2H), 3.98 (s, 3H), 3.25 (s, 3H), 2.28 (s, 3H).

Step 7: 2-Amino-2-(2-methoxyphenyl)ethanol (1.00 g, 5.98 mmol), triethylamine (1.0 mL, 7.17 mmol) and tert-butoxycarbonyl tert-butyl carbonate (1.56 g, 7.15 mmol) were suspended in anhydrous THF (20 mL) and the reaction mixture was left to stir at room temperature for 18 hours. The reaction was quenched by the addition of water and the crude product was extracted into DCM. The aqueous extract was re-extracted into DCM (×3) and the combined organic extracts were dried over magnesium sulfate, filtered and concentrated under reduced pressure. The crude product was purified by silica gel chromatography eluting with heptane:ethyl acetate (0-100%) and the resulting yellow solid was triturated with diethyl ether to yield tert-butyl N-[2-hydroxy-1-(2-methoxyphenyl)ethyl]carbamate (533 mg, 33%) as a white solid. LCMS (Analytical Method B) t_{R}=1.45 min, [M+H]⁺=268.3; 1H NMR (250 MHz, Chloroform-d) δ 7.27-7.11 (m, 2H), 6.94-6.76 (m, 2H), 5.46 (s, 1H), 4.99 (s, 1H), 3.76 (d, J=10.9 Hz, 5H), 2.00 (d, J=15.0 Hz, 1H), 1.36 (s, 9H).

Step 8: tert-Butyl N-[2-hydroxy-1-(2-methoxyphenyl)ethyl]carbamate (520 mg, 1.95 mmol), phthalimide (430 mg, 2.92 mmol), triphenylphosphine (765 mg, 2.92 mmol) and (N{E})-N-(piperidine-1-carbonylimino)piperidine-1-carboxamide (490 mg, 1.94 mmol) were dissolved in anhydrous THF (15 mL) at 0° C. and the reaction mixture was stirred at room temperature for 18 hours. The reaction mixture was partitioned between water and DCM and the organic extracts were dried over magnesium sulfate, filtered and concentrated under reduced pressure. The crude compound was purified by silica gel chromatography eluting with heptane:ethyl acetate (0-100%) to give tert-butyl N-[2-(1,3-dioxoisoindolin-2-yl)-1-(2-methoxyphenyl)ethyl]carbamate (544 mg, 71%) as a white solid. LCMS (Analytical Method B) t_{R}=1.76 min, [M+H]⁺=397.2; 1H NMR (250 MHz, Chloroform-d) δ 7.86-7.63 (m, 4H), 7.61 (dd, J=5.4, 3.0 Hz, 2H), 7.16 (s, 1H), 6.89-6.75 (m, 2H), 5.70 (d, J=9.8 Hz, 1H), 5.24-5.10 (m, 1H), 4.11 (td, J=13.0, 12.4, 8.5 Hz, 1H), 3.87 (s, 3H), 3.80 (dd, J=13.7, 4.4 Hz, 1H), 1.15 (s, 9H).

Step 9: tert-Butyl N-[2-(1,3-dioxoisoindolin-2-yl)-1-(2-methoxyphenyl)ethyl]carbamate (544 mg, 1.37 mmol) and hydrazine (2.0 mL, 64.3 mmol) were dissolved in anhydrous THF (10 mL) at 0° C. and the reaction mixture was allowed to warm to room temperature for 18 hours. The reaction mixture was partitioned between water and DCM and the organic extracts were dried over magnesium sulfate, filtered and concentrated under reduced pressure. The crude compound was purified by silica gel chromatography eluting with heptane:ethyl acetate (0-100%) to give tert-butyl N-[2-amino-1-(2-methoxyphenyl)ethyl]carbamate (172 mg, 42%) as a white solid. LCMS (Analytical Method B) t_{R}=1.58 min, [M+H]⁺=267.3; 1H NMR (250 MHz, DMSO-d6) δ 7.19 (ddd, J=8.9, 4.3, 2.2 Hz, 3H), 7.04-6.84 (m, 2H), 4.81 (d, J=4.0 Hz, 1H), 3.80 (s, 3H), 2.67 (dd, J=13.0, 4.2 Hz, 1H), 2.55 (d, J=8.4 Hz, 1H), 1.35 (d, J=14.0 Hz, 9H), 1.21 (d, J=18.2 Hz, 2H).

Step 10: N-Ethyl-N-isopropyl-propan-2-amine (75 uL, 0.429 mmol), N-[(3-chloro-4-methyl-phenyl)methyl]-1-methyl-6-methylsulfonyl-pyrazolo[3,4-d]pyrimidin-4-amine (75 mg, 0.205 mmol) and tert-butyl N-[2-amino-1-(2-methoxyphenyl)ethyl]carbamate (110 mg, 0.413 mmol) were suspended in 1-butanol (1.5 mL) and the resulting mixture was left to stir at 125° C. for 18 hours. Purification by KP-NH silica gel chromatography eluting with heptane:ethyl acetate (0-100%) gave tert-butyl N-[2-[[4-[(3-chloro-4-methyl-phenyl)methylamino]-1-methyl-pyrazolo[3,4-d]pyrimidin-6-yl]amino]-1-(2-methoxyphenyl)ethyl]carbamate (65 mg, 57%) as a white solid. LCMS (Analytical Method B) t_{R}=1.92 min, [M+H]⁺=552.4/554.4; 1 H NMR (250 MHz, Chloroform-d) δ 7.54 (s, 1H), 7.26 (s, 1H), 7.12 (dd, J=12.8, 7.5 Hz, 4H), 6.91-6.74 (m, 2H), 5.30 (s, 1H), 5.05 (d, J=20.5 Hz, 2H), 4.62 (s, 2H), 3.80 (d, J=9.0 Hz, 6H), 3.69 (s, 2H), 2.29 (s, 3H), 1.30 (s, 9H).

Step 11: tert-Butyl N-[2-[[4-[(3-chloro-4-methyl-phenyl)methylamino]-1-methyl-pyrazolo[3,4-d]pyrimidin-6-yl]amino]-1-(2-methoxyphenyl)ethyl]carbamate (65 mg, 0.118 mmol) was suspended in 4M HCl in 1,4 dioxane (1.5 mL) and the resulting mixture was stirred at room temperature for 30 minutes. The crude compound was purified by high pH reverse phase C18 chromatography (0.1% ammonia, 0-100% acetonitrile:water) to give N6-[2-amino-2-(2-methoxyphenyl)ethyl]-N4-[(3-chloro-4-methyl-phenyl)methyl]-1-methyl-pyrazolo[3,4-d]pyrimidine-4,6-diamine (24 mg, 43%) as a fluffy white solid. LCMS (Analytical Method C) t_{R}=3.49 min, [M+H]⁺=452.4/454.3; 1H NMR (250 MHz, DMSO-d6) δ 8.21 (s, 1H), 7.79 (s, 1H), 7.37 (s, 2H), 7.34-7.13 (m, 3H), 6.92 (dd, J=14.3, 7.4 Hz, 2H), 6.48 (s, 1H), 4.63 (d, J=5.8 Hz, 2H), 4.36 (s, 1H), 3.73 (d, J=19.7 Hz, 6H), 3.64-3.47 (m, 1H), 3.29-3.11 (m, 1H), 2.30 (s, 3H).

### Compound 5.02: 6-N-(2-Amino-2-phenylethyl)-4-N-[(3-chloro-4-methylphenyl)methyl]-1-methylpyrazolo[3,4-d]pyrimidine-4,6-diamine

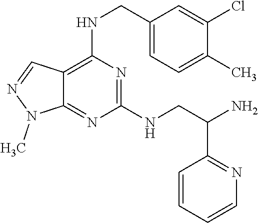 [SMILES: Cc1ccc(CNc2nc(NCC(N)c3ccccn3)nc3c2cnn3C)cc1Cl]

tert-Butyl N-[2-amino-1-(2-pyridyl)ethyl]carbamate was synthesised in an analogous manner to the diamine used in Compound 5.01 from commercially available tert-butyl N-[2-hydroxy-1-(2-pyridyl)ethyl]carbamate. LCMS (Analytical Method B) t_{R}=1.28 min, [M+H]⁺=238.3; 1H NMR (250 MHz, Chloroform-d) δ 8.48 (d, J=4.8 Hz, 1H), 7.59 (td, J=7.7, 1.8 Hz, 1H), 7.22 (s, 1H), 7.19 (s, 1H), 7.12 (ddd, J=7.5, 4.9, 1.0 Hz, 1H), 5.87 (s, 1H), 4.68 (d, J=6.6 Hz, 1H), 3.70-3.65 (m, 1H), 3.09-2.88 (m, 2H), 1.83-1.75 (m, 1H), 1.38 (s, 9H).

6-N-(2-Amino-2-phenylethyl)-4-N-[(3-chloro-4-methylphenyl)methyl]-1-methylpyrazolo[3,4-d]pyrimidine-4,6-diamine was synthesised in an analogous manner to Compound 5.01 using Steps 10 and 11 (14 mg, 25%). LCMS (Analytical Method A) t_{R}=1.82 min, [M+H]⁺=423.3/425.3; 1H NMR (250 MHz, DMSO-d6) δ 8.57 (d, J=4.9 Hz, 1H), 8.27 (s, 3H), 7.88-7.70 (m, 2H), 7.46-7.17 (m, 5H), 6.71 (s, 1H), 4.63 (d, J=5.6 Hz, 2H), 4.37 (s, 1H), 3.70 (s, 4H), 3.50 (s, 1H), 2.29 (s, 3H).

### Compound 5.03: 6-N-(2-amino-2-pyridin-2-ylethyl)-1-methyl-4-N-[4-(trifluoromethyl)phenyl]pyrazolo[3,4-d]pyrimidine-4,6-diamine

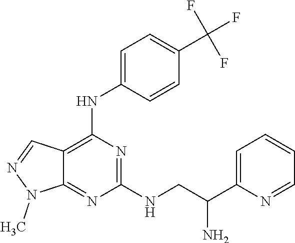 [SMILES: Cn1ncc2c(Nc3ccc(C(F)(F)F)cc3)nc(NCC(N)c3ccccn3)nc21]

Step 1: To a solution of tert-butyl N-[2-hydroxy-1-(2-pyridyl)ethyl]carbamate (1.01 g, 4.24 mmol) and triethylamine (1.18 mL, 8.47 mmol) in DCM (10 mL) was added 4-methylbenzenesulfonyl chloride (808 mg, 4.24 mmol) portionwise at 0° C. and the resulting mixture was allowed to warm to room temperature for 18 hours. The reaction mixture was partitioned between water and DCM and the aqueous portion was re-extracted into DCM (×3). The organic extracts were dried over magnesium sulfate, filtered and concentrated under reduced pressure to give [2-(tert-butoxycarbonylamino)-2-(2-pyridyl)ethyl] 4-methylbenzenesulfonate (1.36 g, 75%) as a yellow oil. LCMS (Analytical Method B) t_{R}=1.72 min, [M+H]⁺=393.2; 1H NMR (250 MHz, Chloroform-d) δ 8.38 (d, J=4.8 Hz, 1H), 7.65-7.50 (m, 3H), 7.21 (s, 2H), 7.22-7.04 (m, 2H), 5.65 (s, 1H), 4.94 (s, 1H), 4.39-4.26 (m, 1H), 4.17 (q, J=9.4, 8.0 Hz, 1H), 2.36 (s, 3H), 1.36 (s, 9H).

Step 2: To a solution of [2-(tert-butoxycarbonylamino)-2-(2-pyridyl)ethyl] 4-methylbenzenesulfonate (1.36 g, 3.47 mmol) in DMF (10 mL) was added sodium;azide (248.0 mg, 3.81 mmol) and the reaction mixture was heated at 60° C. for 18 hours. The reaction mixture was quenched by the slow addition of water and the organics were extracted into DCM (×3), dried over magnesium sulfate, filtered and concentrated under reduced pressure. The crude product was purified by silica gel chromatography eluting with heptane:ethyl acetate (5-100%) to yield tert-butyl N-[2-azido-1-(2-pyridyl)ethyl]carbamate (490 mg, 53% Yield) as a colourless oil. LCMS (Analytical Method B) t_{R}=1.55 min, [M+H]⁺=264.3; 1H NMR (250 MHz, Chloroform-d) δ 8.51 (d, J=4.9 Hz, 1H), 7.63 (td, J=7.7, 1.8 Hz, 1H), 7.23 (d, J=7.8 Hz, 1H), 7.22-7.11 (m, 1H), 5.79 (s, 1H), 4.87 (d, J=6.1 Hz, 1H), 3.62 (qd, J=12.1, 5.6 Hz, 2H), 1.39 (s, 9H).

Step 3: To a solution of tert-butyl N-[2-azido-1-(2-pyridyl)ethyl]carbamate (490 mg, 1.86 mmol) in THF (8 mL) under an atmosphere of hydrogen was added Pd/C (10% w/w, 20 mg, 0.188 mmol) and the reaction mixture was allowed to stir at room temperature for 18 hours. The reaction mixture was filtered through Celite™, washed with methanol (3×) and the solvents were removed in vacuo to give tert-butyl N-[2-amino-1-(2-pyridyl)ethyl]carbamate (441 mg, 95%) as a colourless oil. LCMS (Analytical Method B) t_{R}=1.27 min. [M+H]1=238.3; 1H NMR (250 MHz, DMSO-d6) δ 8.58-8.45 (m, 1H), 7.75 (td, J=7.7, 1.8 Hz, 1H), 7.35-7.15 (m, 3H), 4.57-4.42 (m, 1H), 2.93-2.66 (m, 2H), 1.40-1.24 (m, 11H).

Step 4: 6-N-(2-Amino-2-pyridin-2-ylethyl)-1-methyl-4-N-[4-(trifluoromethyl)phenyl]pyrazolo[3,4-d]pyrimidine-4,6-diamine was synthesised according to General Method 4 using tert-butyl N-[2-amino-1-(2-pyridyl)ethyl]carbamate and 6-chloro-1-methyl-N-[4-(trifluoromethyl)phenyl]-1H-pyrazolo[3,4-d]pyrimidin-4-amine (28 mg, 66%). LCMS (Analytical Method L) t_{R}=1.11 min, [M+H]⁺=429.55; 1H NMR (400 MHz, DMSO-d6) δ 9.91 (s, 1H), 8.53 (d, J=4.0 Hz, 1H), 8.19 (d, J=8.7 Hz, 2H), 8.05 (d, J=4.0 Hz, 1H), 7.74 (td, J=7.8, 1.6 Hz, 1H), 7.66 (d, J=8.7 Hz, 2H), 7.48 (d, J=7.8 Hz, 1H), 7.31-7.19 (m, 1H), 7.08 (s, 1H), 4.20 (dd, J=7.6, 5.4 Hz, 1H), 3.75 (m, 4H), 2.41-2.01 (s, 2H).

### Compound 5.04: 3-[1-Amino-2-[[1-methyl-4-(methylamino)pyrazolo[3,4-d]pyrimidin-6-yl]amino]ethyl]benzonitrile

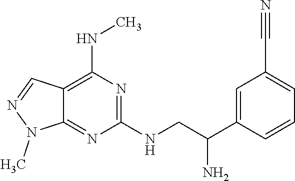 [SMILES: CNc1nc(NCC(N)c2cccc(C#N)c2)nc2c1cnn2C]

To a solution of N6-[2-amino-2-(3-bromophenyl)ethyl]-N4,1-dimethylpyrazolo[3,4-d]pyrimidine-4,6-diamine (34 mg, 0.0904 mmol) and zinc;dicyanide (11 mg, 0.0965 mmol) in degassed THF:water (1 mL) was added tBuXPhos Pd G3 (4.5 mg, 5.71 μmol) and the reaction heated to 60° C. for 18 hours. The reaction mixture was cooled to room temperature and partitioned between water (2 mL) and DCM (4 mL). The organic layer was filtered through a TELOS™ cartridge and the solvents were evaporated under reduced pressure to give the crude material as a colourless oil which was purified by high pH preparative HPLC (Method B) to give 3-[1-amino-2-[[1-methyl-4-(methylamino)pyrazolo[3,4-d]pyrimidin-6-yl]amino]ethyl]benzonitrile (11 mg, 38%) as a white powder. LCMS (Analytical Method D) t_{R}=2.87 min, [M+H]⁺=323.3; 1H NMR (500 MHz, DMSO-d6) δ 7.88 (s, 1H), 7.70 (dd, J=41.1, 8.1 Hz, 4H), 7.51 (t, J=7.7 Hz, 1H), 6.68 (s, 1H), 4.23-4.12 (m, 1H), 3.69 (s, 3H), 3.58 (s, 1H), 3.27 (d, J=6.9 Hz, 1H), 2.91 (s, 3H), 2.08 (d, J=10.7 Hz, 2H).

### Compound 5.05: N6-[2-Amino-2-(3-ethynylphenyl)ethyl]-N4-tert-butyl-1-methyl-pyrazolo[3,4-d]pyrimidine-4,6-diamine

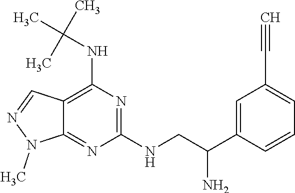 [SMILES: C#Cc1cccc(C(N)CNc2nc(NC(C)(C)C)c3cnn(C)c3n2)c1]

N6-[2-Amino-2-(3-bromophenyl)ethyl]-N4-tert-butyl-1-methyl-pyrazolo[3,4-d]pyrimidine-4,6-diamine (40 mg, 0.0956 mmol), PdCl₂dppf (8.0 mg, 0.0109 mmol), Cul (2.0 mg, 0.0104 mmol) and triethylamine (150 uL, 1.08 mmol) were combined in DMF (1 mL) and the mixture was sparged with N₂ for 5 minutes. Ethynyl(trimethyl)silane (20 uL, 0.142 mmol) was added and the mixture was further sparged with N₂ and the vessel sealed and heated to 80° C. for 3 hours. The reaction mixture was purified by high pH reverse phase C18 chromatography eluting with 0.1% ammonia, 0-100% acetonitrile:water to yield the intermediate as a brown solid which was dissolved in methanol (10 mL) and potassium carbonate (66 mg, 0.478 mmol) was added and the reaction mixture was stirred at room temperature for 1 hour to yield the desired product which was acidified with 4N HCL in dioxane (1 mL) to neutralise any residual potassium carbonate and purified by passing through a SCX column and eluting with 7N ammonia in methanol to yield N6-[2-amino-2-(3-ethynylphenyl)ethyl]-N4-tert-butyl-1-methyl-pyrazolo[3,4-d]pyrimidine-4,6-diamine (12 mg, 34%) as an off white solid. LCMS (Analytical Method D) t_{R}=3.91 min, [M+H]⁺=364.2; 1H NMR (400 MHz, DMSO-d6) δ 7.89 (s, 1H), 7.57 (s, 1H), 7.49-7.44 (m, 1H), 7.34 (dd, J=3.9, 1.3 Hz, 2H), 6.82 (s, 1H), 6.33-6.26 (m, 1H), 4.19 (dd, J=8.0, 5.1 Hz, 1H), 3.95 (s, 1H), 3.69 (s, 3H), 3.65 (dd, J=12.4, 7.0 Hz, 1H), 3.39-3.34 (m, 1H), 1.51 (s, 9H).

Various substituted amines can be synthesised in an analogous manner to the 2 Compounds described above for tert-butyl N-[2-amino-1-(2-pyridyl)ethyl]carbamate and the compounds are detailed in Table 5

TABLE 5

| Compound | | | Analytical | t_{R} | |
|---|---|---|---|---|---|
| No. | Name | Structure | Method | (min) | [M + H]⁺ |
| 5.06 | N6-[2-amino-2-(2- pyridyl)ethyl]-N4- tert-butyl-1- methyl- pyrazolo[3,4- d]pyrimidine-4,6- diamine | 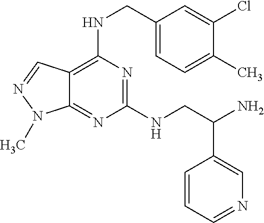 [SMILES: Cc1ccc(CNc2nc(NCC(N)c3cccnc3)nc3c2cnn3C)cc1Cl] | D | 3.21 | 341.3 |
| 5.07 | 6-N-(2-amino-2- pyridin-3-ylethyl)- 4-N-[(3-chloro-4- methylphenyl) methyl]-1- methylpyrazolo[3, 4-d]pyrimidine- 4,6-diamine | 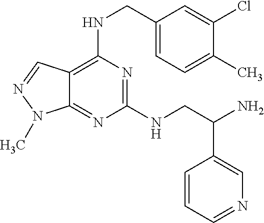 [SMILES: Cc1ccc(CNc2nc(NCC(N)c3cccnc3)nc3c2cnn3C)cc1Cl] | C | 2.68 | 423.3/ 425.3 |
| 5.08 | N6-[2-amino-2-(3- pyridyl)ethyl]-N4- tert-butyl-1- methyl- pyrazolo[3,4- d]pyrimidine-4,6- diamine | 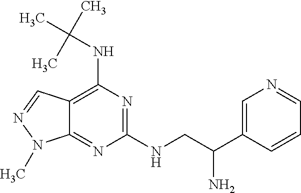 [SMILES: Cn1ncc2c(NC(C)(C)C)nc(NCC(N)c3cccnc3)nc21] | C | 2.06 | 341.3 |
| 5.09 | 6-N-[2-amino-2- (2- chlorophenyl) ethyl]-1-methyl-4-N- [4- (trifluoromethyl) phenyl]pyrazolo[3, 4-d]pyrimidine- 4,6-diamine | 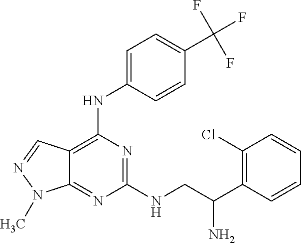 [SMILES: Cn1ncc2c(Nc3ccc(C(F)(F)F)cc3)nc(NCC(N)c3ccccc3Cl)nc21] | D | 4.52 | 462.2/ 464.2 |
| 5.10 | N6-[2-amino-2-(2- chlorophenyl)ethyl]- N4-tert-butyl-1- methyl- pyrazolo[3,4- d]pyrimidine-4,6- diamine | 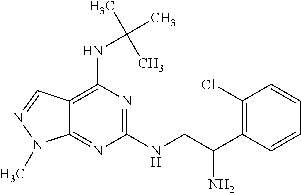 [SMILES: Cn1ncc2c(NC(C)(C)C)nc(NCC(N)c3ccccc3Cl)nc21] | D | 3.93 | 374.3 |
| 5.11 | N6-[(2R)-2- amino-2-(2- chlorophenyl)ethyl]- N4-tert-butyl-1- methyl- pyrazolo[3,4- d]pyrimidine-4,6- diamine | 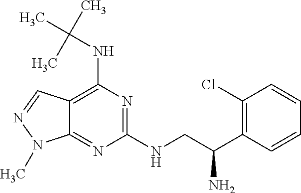 [SMILES: Cn1ncc2c(NC(C)(C)C)nc(NC[C@H](N)c3ccccc3Cl)nc21] | D | 3.95 | 374.2 |
| 5.12 | 6-N-[2-amino-2- (2- fluorophenyl)ethyl]- 4-N-[(3-chloro-4- methylphenyl) methyl]-1- methylpyrazolo[3, 4-d]pyrimidine- 4,6-diamine | 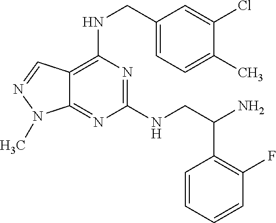 [SMILES: Cc1ccc(CNc2nc(NCC(N)c3ccccc3F)nc3c2cnn3C)cc1Cl] | D | 4.19 | 440.2/ 442.1 |
| 5.13 | N6-[2-amino-2-(2- fluorophenyl)ethyl]- N4-tert-butyl-1- methyl- pyrazolo[3,4- d]pyrimidine-4,6- diamine | 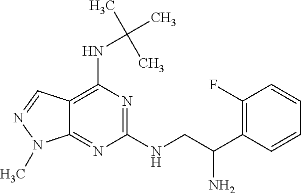 [SMILES: Cn1ncc2c(NC(C)(C)C)nc(NCC(N)c3ccccc3F)nc21] | D | 3.79 | 358.3 |
| 5.14 | N6-[2-amino-2-(2- fluorophenyl)ethyl]- N4-tert-butyl-1- (trideuteriomethyl) pyrazolo[3,4- d]pyrimidine-4,6- diamine | 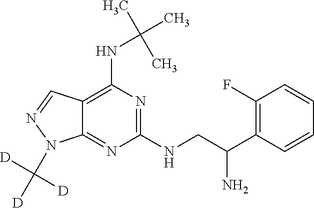 [SMILES: [2H]C([2H])([2H])n1ncc2c(NC(C)(C)C)nc(NCC(N)c3ccccc3F)nc21] | C | 2.93 | 361.2 |
| 5.15 | N6-[(2R)-2- amino-2-(3- bromophenyl)ethyl]- N4-isopropyl-1- methyl- pyrazolo[3,4- d]pyrimidine-4,6- diamine | 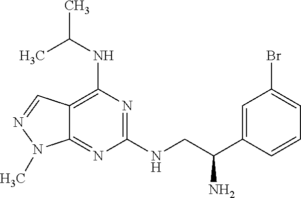 [SMILES: CC(C)Nc1nc(NC[C@H](N)c2cccc(Br)c2)nc2c1cnn2C] | D | 3.85 | 404.1/ 406.1 |
| 5.16 | N6-[(2R)-2- amino-2-(3- bromophenyl)ethyl]- 1-methyl-N4- (2,2,2-trifluoro- 1,1-dimethyl- ethyl)pyrazolo[3,4- d]pyrimidine-4,6- diamine | 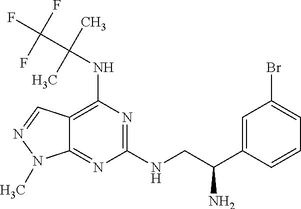 [SMILES: Cn1ncc2c(NC(C)(C)C(F)(F)F)nc(NC[C@H](N)c3cccc(Br)c3)nc21] | D | 4.23 | 472.1/ 474.1 |
| 5.17 | 6-N-[2-amino-2- (3- bromophenyl)ethyl]- 4-N,1- dimethylpyrazolo [3,4-d]pyrimidine- 4,6-diamine | 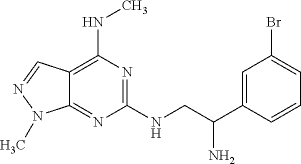 [SMILES: CNc1nc(NCC(N)c2cccc(Br)c2)nc2c1cnn2C] | C | 2.52 | 376.1/ 378.1 |
| 5.18 | N6-[2-amino-2-(3- bromophenyl)ethyl]- N4-tert-butyl-1- methyl- pyrazolo[3,4- d]pyrimidine-4,6- diamine | 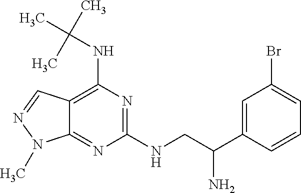 [SMILES: Cn1ncc2c(NC(C)(C)C)nc(NCC(N)c3cccc(Br)c3)nc21] | D | 4.10 | 418.2/ 420.2 |
| 5.19 | 6-N-[2-amino-2- (4- bromophenyl)ethyl]- 1-methyl-4-N- propan-2- ylpyrazolo[3,4- d]pyrimidine-4,6- diamine | 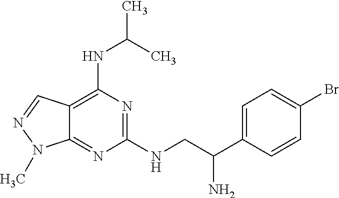 [SMILES: CC(C)Nc1nc(NCC(N)c2ccc(Br)cc2)nc2c1cnn2C] | C | 3.09 | 404.2/ 406.2 |
| 5.20 | N6-[2-amino-2-(2- bromophenyl)ethyl]- N4-tert-butyl-1- methyl- pyrazolo[3,4- d]pyrimidine-4,6- diamine | 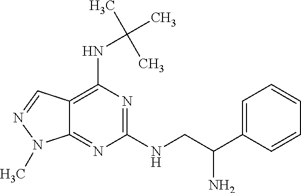 [SMILES: Cn1ncc2c(NC(C)(C)C)nc(NCC(N)c3ccccc3)nc21] | D | 4.05 | 418.2/ 420.2 |
| 5.21 | 4-[1-amino-2-[[1- methyl-4- (methylamino) pyrazolo[3,4- d]pyrimidin-6- yl]amino]ethyl] benzonitrile | 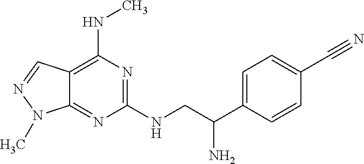 [SMILES: CNc1nc(NCC(N)c2ccc(C#N)cc2)nc2c1cnn2C] | D | 2.84 | 323.3 |
| 5.22 | 3-[1-amino-2-[[4- (tert-butylamino)- 1-methyl- pyrazolo[3,4- d]pyrimidin-6- yl]amino]ethyl] benzonitrile | 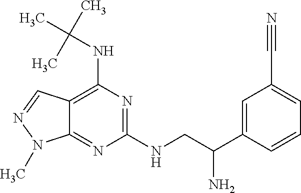 [SMILES: Cn1ncc2c(NC(C)(C)C)nc(NCC(N)c3cccc(C#N)c3)nc21] | D | 3.64 | 365.3 |
| 5.23 | 3-[(1R)-1-amino- 2-[[1-methyl-4- [(2,2,2-trifluoro- 1,1-dimethyl- ethyl)amino] pyrazolo[3,4- d]pyrimidin-6- yl]amino]ethyl] benzonitrile | 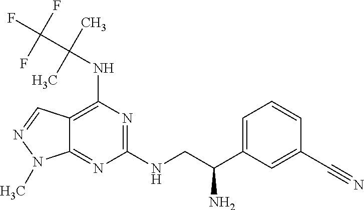 [SMILES: Cn1ncc2c(NC(C)(C)C(F)(F)F)nc(NC[C@H](N)c3cccc(C#N)c3)nc21] | D | 3.73 | 419.1 |
| 5.24 | N6-[2-amino-2-(3- pyridyl)ethyl]-N4- tert-butyl-1- methyl- pyrazolo[3,4- d]pyrimidine-4,6- diamine | 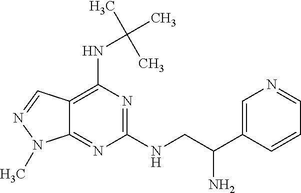 [SMILES: Cn1ncc2c(NC(C)(C)C)nc(NCC(N)c3cccnc3)nc21] | C | 2.16 | 341.3 |
| 5.25 | N6-[2-amino-2-(3- pyridyl)ethyl]-N4- (3,3- difluorocyclobutyl)- 1-methyl- pyrazolo[3,4- d]pyrimidine-4,6- diamine | 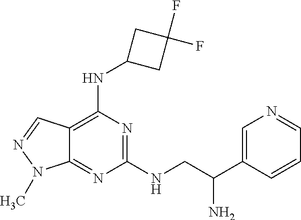 [SMILES: Cn1ncc2c(NC3CC(F)(F)C3)nc(NCC(N)c3cccnc3)nc21] | D | 2.92 | 375.3 |
| 5.26 | N6-[2-amino-2-(4- pyridyl)ethyl]-N4- tert-butyl-1- methyl- pyrazolo[3,4- d]pyrimidine-4,6- diamine | 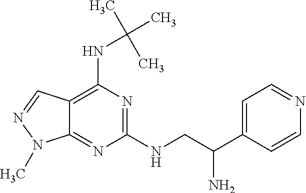 [SMILES: Cn1ncc2c(NC(C)(C)C)nc(NCC(N)c3ccncc3)nc21] | D | 2.96 | 341.3 |
| 5.27 | N6-[2-amino-2-(4- pyridyl)ethyl]-N4- (3,3- difluorocyclobutyl)- 1-methyl- pyrazolo[3,4- d]pyrimidine-4,6- diamine | 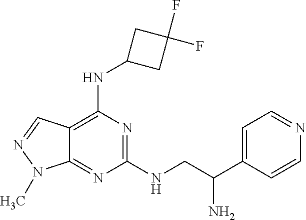 [SMILES: Cn1ncc2c(NC3CC(F)(F)C3)nc(NCC(N)c3ccncc3)nc21] | D | 2.88 | 375.3 |
| 5.28 | 4-[(1R)-1-amino- 2-{[4-(tert- butylamino)-1- methyl-1H- pyrazolo[3,4- d]pyrimidin-6- yl]amino}ethyl] benzonitrile | 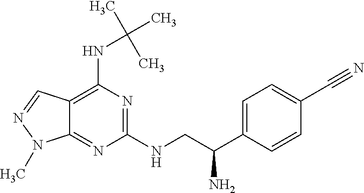 [SMILES: Cn1ncc2c(NC(C)(C)C)nc(NC[C@H](N)c3ccc(C#N)cc3)nc21] | D | 3.55 | 365.3 |
| 5.29 | N4-tert-butyl-1- methyl-N6-[(2R)- 2-(methylamino)- 2-phenylethyl]- 1H-pyrazolo[3,4- d]pyrimidine-4,6- diamine | 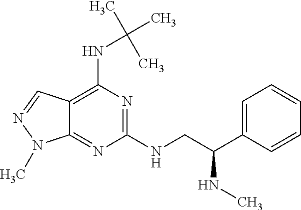 [SMILES: CN[C@@H](CNc1nc(NC(C)(C)C)c2cnn(C)c2n1)c1ccccc1] | D | 4.15 | 354.3 |

The synthesis of the following compounds was carried out only partially according to the General Methods described above and as such are detailed separately.

### Compound 5.30: N6-[2-amino-2-(4-fluorophenyl)ethyl]-N4-tert-butyl-1-methyl-1H-pyrazolo[3,4-d]pyrimidine-4,6-diamine

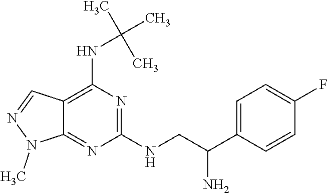 [SMILES: Cn1ncc2c(NC(C)(C)C)nc(NCC(N)c3ccc(F)cc3)nc21]

Step 1: To a solution of cobalt(II) chloride (1.50 g, 11.5 mmol) in methanol (20.0 mL) was added a solution of tert-butyl N-[cyano-(4-fluorophenyl)methyl]carbamate (1.36 g, 5.43 mmol) in methanol (34.0 mL) and the resulting mixture was cooled in an ice water bath. Sodium borohydride (1.00 g, 26.4 mmol) was added as solid in portions over 5 minutes and the reaction mixture was stirred at 0° C. for 1 hour. The reaction mixture was quenched by careful, slow addition of IPA (5 mL) and water (30 mL) at 0° C. and the mixture was stirred at ambient temperature for 20 minutes. The crude reaction mixture was filtered through a pad of celite and washed with copious amount of DCM and water and the phases were separated. The organic phase was dried over sodium sulfate and the volatiles were removed under reduced pressure to give the crude product as an amber gel which was purified by silica gel chromatography eluting with 50% heptane in EtOAc, followed by 0-20% MeOH in EtOAc, to give tert-butyl N-[2-amino-1-(4-fluorophenyl)ethyl]carbamate (653 mg, 47%) as yellow oil. LCMS (Analytical Method B) t_{R}=1.43 min. [M+H]⁺=255.3; 1H NMR (400 MHz, Chloroform-d) δ 7.26 (dd, J=5.9, 2.6 Hz, 2H), 7.11-7.00 (m, 2H), 5.32 (s, 1H), 4.65 (s, 1H), 3.00 (d, J=5.5 Hz, 1H), 1.46 (s, 9H).

Step 2: N6-[2-Amino-2-(4-fluorophenyl)ethyl]-N4-tert-butyl-1-methyl-pyrazolo[3,4-d]pyrimidine-4,6-diamine was synthesised from N-tert-butyl-6-chloro-1-methyl-pyrazolo[3,4-d]pyrimidin-4-amine and tert-butyl N-[2-amino-1-(4-fluorophenyl)ethyl]carbamate according the General Method 4. The intermediate BOC protected material was purified by silica gel chromatography eluting with 20-100% EtOAc in heptane to yield tert-butyl N-[2-[[4-(tert-butylamino)-1-methyl-pyrazolo[3,4-d]pyrimidin-6-yl]amino]-1-(4-fluorophenyl)ethyl] carbamate (100 mg, 0.201 mmol). LCMS (Analytical Method B) t_{R}=1.77 min, [M+H]⁺=458.2. SCX elution of the HCl salt followed by high pH preparative HPLC (Method B) yielded the title compound (35 mg, 45%) as a white solid. LCMS (Analytical Method D) t_{R}=3.02 min, [M+H]⁺=358.3; 1H NMR (400 MHz, DMSO-d6) δ 7.93 (s, 1H), 7.52-7.43 (m, 2H), 7.21-7.05 (m, 3H), 6.63 (s, 1H), 4.13 (s, 1H), 3.70 (s, 3H), 3.59 (s, 1H), 3.25-3.16 (m, 1H), 1.99 (s, 2H), 1.52 (s, 9H). 19F NMR (400 MHz, DMSO-d6) δ −116.7.

### Compound 5.31: N6-[2-amino-2-(3,4-difluorophenyl)ethyl]-N4-tert-butyl-1-methyl-pyrazlo[3,4-d]pyrimidine-4,6-diamine

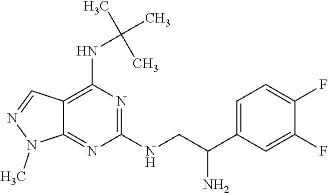 [SMILES: Cn1ncc2c(NC(C)(C)C)nc(NCC(N)c3ccc(F)c(F)c3)nc21]

Step 1: To a stirred solution of 2-amino-2-(3,4-difluorophenyl)acetonitrile;hydrochloride (1.00 g, 4.89 mmol) in THF (17 mL) was added triethylamine (0.75 mL, 5.38 mmol) and tert-butoxycarbonyl tert-butyl carbonate (1.12 g, 5.13 mmol) and the resulting reaction mixture was stirred at room temperature for 24 hours. Triethylamine (0.75 mL, 5.38 mmol) and BOC anhydride (100 mg, 0.458 mmol) was added and stirring continued for a further 24 hours. The reaction mixture was quenched with water (20 mL) and extracted into DCM (2×30 mL), the organics were passed through a phase separator and concentrated in vacuo and the crude material was purified by silica gel chromatography eluting with 0-100% EtOAc in heptane to afford tert-butyl N-[cyano-(3,4-difluorophenyl)methyl]carbamate (1.01 g, 77%) as an off white solid. LCMS (Analytical Method A) t_{R}=1.22 min, [M+H]⁺=269.0; 1H NMR (400 MHz, DMSO-d6) δ 8.36 (s, 1H), 7.60-7.46 (m, 2H), 7.35 (ddd, J=8.6, 3.9, 1.8 Hz, 1H), 5.95 (d, J=7.9 Hz, 1H), 1.40 (s, 9H).

Step 2: To a solution of tert-butyl N-[cyano-(3,4-difluorophenyl)methyl]carbamate (50 mg, 0.186 mmol) and cobalt (II) chloride (51 mg, 0.395 mmol) in methanol (2 mL) cooled to 0° C. was added sodium borohydride (70 mg, 1.85 mmol) portionwise and the resulting mixture was stirred at 0° C. for 2 hours. The reaction mixture was quenched with water (5 mL) and the crude material was extracted into 1:4 IPA/CHCl₃ (2×12 mL). Brine (5 mL) was added to aid separation as an emulsion formed, the organics were combined, dried over sodium sulfate and concentrated in vacuo. The crude material was purified by high pH C18 column chromatography eluting with 30-95% acetonitrile (0.5% ammonium hydroxide) in water (0.5% ammonium hydroxide) to afford tert-butyl N-[2-amino-1-(3,4-difluorophenyl)ethyl]carbamate (27 mg, 53%) as a white solid. LCMS (Analytical Method B) t_{R}=1.47 min, [M+H]⁺=273.3; HNMR (400 MHz, DMSO-d6) δ 7.41-7.23 (m, 3H), 7.09 (s, 1H), 4.40 (d, J=6.7 Hz, 1H), 2.66 (d, J=6.7 Hz, 2H), 1.36 (s, 9H).

Step 3: N6-[2-amino-2-(3,4-difluorophenyl)ethyl]-N4-tert-butyl-1-methyl-pyrazolo[3,4-d]pyrimidine-4,6-diamine was synthesized from N-tert-butyl-6-chloro-1-methyl-pyrazolo[3,4-d]pyrimidin-4-amine and tert-butyl N-[2-amino-1-(3,4-difluorophenyl)ethyl]carbamate according to General Method 4 and was purified by high pH preparative HPLC according to General Method B (65 mg, 33%). LCMS (Analytical Method C) t_{R}=3.11 min, [M+H]⁺=376.3; 1H NMR (500 MHz, DMSO-d6) δ 7.90 (s, 1H), 7.49-7.41 (m, 1H), 7.35 (dt, J=10.8, 8.5 Hz, 1H), 7.26-7.19 (m, 1H), 7.11 (s, 1H), 6.63 (s, 1H), 4.16-4.03 (m, 1H), 3.67 (s, 3H), 3.60-3.50 (m, 1H), 3.27-3.15 (m, 1H), 2.06 (s, 2H), 1.48 (s, 9H).

### Compound 5.32: N6-[2-amino-2-(3-cyclopropylphenyl)ethyl]-N4-tert-butyl-1-methyl-1H-pyrazolo[3,4-d]pyrimidine-4,6-diamine

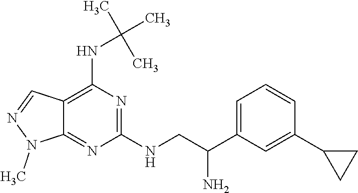 [SMILES: Cn1ncc2c(NC(C)(C)C)nc(NCC(N)c3cccc(C4CC4)c3)nc21]

Step 1: To a solution of tert-butoxycarbonyl tert-butyl carbonate (796 mg, 3.65 mmol) in DCM (12 mL) was added triethylamine (1.7 mL, 12.2 mmol) and 1-(3-bromophenyl)ethane-1,2-diamine dihydrochloride (500 mg, 1.74 mmol) and the mixture was stirred for 18 hours at 20° C. The reaction was quenched by the addition of saturated ammonium chloride solution (30 mL), diluted with DCM (30 mL) and the phases separated. The organic phase was dried over sodium sulfate, filtered and the volatiles were evaporated. The crude material was purified by silica gel chromatography eluting with 5-100% EtOAc in heptane, followed by 0-5% MeOH in DCM to afford tert-butyl N-[1-(3-bromophenyl)-2-(tert-butoxycarbonylamino)ethyl]carbamate (392 mg, 48%) as white solid. LCMS (Analytical Method B) t_{R}=1.82 min, [M+H]⁺=415.3; 1H NMR (500 MHz, Chloroform-d) δ 7.45-7.39 (m, 2H), 7.22 (d, J=4.9 Hz, 2H), 5.63 (s, 1H), 4.81-4.64 (m, 2H), 3.40 (d, J=32.3 Hz, 2H), 1.57 (s, 9H), 1.46 (s, 9H).

Step 2: Cyclopentyl(diphenyl)phosphane;dichloropalladium;iron (19 mg, 0.0253 mmol), potassium carbonate (87 mg, 0.632 mmol) and tert-butyl N-[1-(3-bromophenyl)-2-(tert-butoxycarbonylamino)ethyl]carbamate (105 mg, 0.253 mmol) were suspended in 1,4-dioxane (2.1 mL) and water (0.42 mL). The reaction mixture was degassed for 5 minutes before addition of 2-cyclopropyl-4,4,5,5-tetramethyl-1,3,2-dioxaborolane (64 mg, 0.379 mmol) and the reaction mixture was left to stir at 90° C. for 18 hours. The reaction mixture was filtered through the pad of celite and washed with EtOAc (25 mL). The volatiles were evaporated and the residue redissolved in 4M HCl in dioxane (1 mL). The mixture was stirred at ambient temperature for 18 hours. The reaction mixture was filtered through celite and washed with EtOAc (50 mL) and MeOH (10 mL). The solvents were evaporated and the material was used directly in Step 3 (22.8 mg, 51%). LCMS (Analytical Method B) t_{R}=1.61 min, [M+H]⁺=177.2.

Step 3: N6-[2-amino-2-(3-cyclopropylphenyl)ethyl]-N4-tert-butyl-1-methyl-pyrazolo[3,4-d]pyrimidine-4,6-diamine was synthesized from N-tert-butyl-6-chloro-1-methyl-pyrazolo[3,4-d]pyrimidin-4-amine and 1-(3-cyclopropylphenyl)ethane-1,2-diamine according to General Method 3 and was purified by low pH preparative HPLC (Method A) to yield the title compound as a white solid (16.1 mg, 18%). LCMS (Analytical Method C) t_{R}=3.38 min, [M+H]⁺=380.3; 1H NMR (500 MHz, DMSO-d6) δ 7.89 (s, 1H), 7.17 (d, J=4.9 Hz, 2H), 7.10 (s, 2H), 6.96-6.86 (m, 1H), 6.58 (s, 1H), 4.03 (s, 1H), 3.66 (s, 3H), 3.57 (s, 1H), 3.13 (ddd, J=13.4, 8.7, 5.1 Hz, 1H), 1.88 (ddd, J=13.4, 8.4, 5.1 Hz, 3H), 1.49 (s, 9H), 0.98-0.86 (m, 2H), 0.68-0.57 (m, 2H).

### Compound 5.33: N6-[2-amino-2-(4-cyclopropylphenyl)ethyl]-N4-tert-butyl-1-methyl-1H-pyrazolo[3,4-d]pyrimidine-4,6-diamine

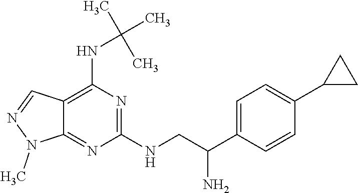 [SMILES: Cn1ncc2c(NC(C)(C)C)nc(NCC(N)c3ccc(C4CC4)cc3)nc21]

N6-[2-amino-2-(4-cyclopropylphenyl)ethyl]-N4-tert-butyl-1-methyl-1H-pyrazolo[3,4-d]pyrimidine-4,6-diamine was synthesized in an analogous manner to compound 5.32 from 1-(4-bromophenyl)ethane-1,2-diamine dihydrochloride (10 mg, 13%). LCMS (Analytical Method D) t_{R}=3.40 min, [M+H]⁺=380.4; 1H NMR (500 MHz, DMSO-d6) δ 7.89 (s, 1H), 7.11 (s, 1H), 7.01 (d, J=8.1 Hz, 2H), 6.55 (s, 1H), 4.03 (s, 1H), 3.66 (s, 3H), 3.55 (s, 1H), 3.13 (td, J=8.5, 8.1, 4.2 Hz, 1H), 1.87 (ddd, J=13.4, 8.4, 5.1 Hz, 3H), 1.48 (s, 9H), 0.95-0.85 (m, 2H), 0.67-0.56 (m, 2H).

### Compound 6.01: N′-(4-Ethoxy-1-methylpyrazolo[3,4-d]pyrimidin-6-yl)-1-phenylethane-1,2-diamine

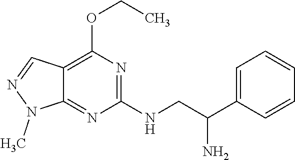 [SMILES: CCOc1nc(NCC(N)c2ccccc2)nc2c1cnn2C]

Step 1: Sodium hydride (6.5 mg, 0.271 mmol) and ethanol (16 uL, 0.274 mmol) were suspended in THF (1 mL) and 4,6-dichloro-1-methyl-1H-pyrazolo[3,4-d]pyrimidine (50 mg, 0.246 mmol) was added and the reaction mixture was stirred at room temperature for 3 hours. The reaction mixture was concentrated in vacuo and the crude product was purified by silica gel chromatography eluting with heptane:ethyl acetate (0-100%) to give 6-chloro-4-ethoxy-1-methyl-pyrazolo[3,4-d]pyrimidine (47 mg, 85%) as a white solid. LCMS (Analytical Method B) t_{R}=1.55 min, [M+H]⁺=213.2; 1H NMR (500 MHz, Chloroform-d) δ 7.93 (s, 1H), 4.57 (q, J=7.1 Hz, 2H), 3.98 (s, 3H), 1.41 (t, J=7.1 Hz, 3H).

Step 2: N′-(4-Ethoxy-1-methylpyrazolo[3,4-d]pyrimidin-6-yl)-1-phenylethane-1,2-diamine was synthesised from 6-chloro-4-ethoxy-1-methyl-pyrazolo[3,4-d]pyrimidine and tert-butyl N-(2-amino-1-phenyl-ethyl)carbamate according to General Method 4 (31 mg, 57%). LCMS (Analytical Method D) t_{R}=4.07 min, [M+H]⁺=313.2; 1H NMR (250 MHz, DMSO-d6) δ 7.78 (s, 1H), 7.44 (d, J=7.3 Hz, 2H), 7.32 (t, J=7.3 Hz, 2H), 7.28-7.15 (m, 2H), 4.48 (d, J=7.0 Hz, 2H), 4.14 (s, 1H), 3.78 (s, 3H), 3.60 (dt, J=11.0, 5.9 Hz, 1H), 3.24 (td, J=8.3, 4.2 Hz, 1H), 1.97 (s, 2H), 1.38 (t, J=7.1 Hz, 3H).

The compounds detailed in Table 6 are synthesised in a similar manner to the compound described above or via the General Methods using previously described or commercially available intermediates

TABLE 6

| Compound | | | Analytical | t_{R} | |
|---|---|---|---|---|---|
| No. | Name | Structure | Method | (min) | [M + H]⁺ |
| 6.04 | N′-(1-methyl-4- propan-2- yloxypyrazolo[3,4- d]pyrimidin-6-yl)- 1-phenylethane- 1,2-diamine | 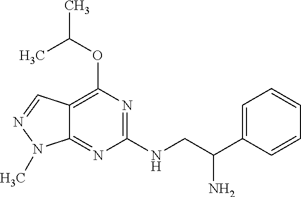 [SMILES: CC(C)Oc1nc(NCC(N)c2ccccc2)nc2c1cnn2C] | D | 3.56 | 327.3 |
| 6.05 | N′-(4-tert-butoxy- 1-methyl- pyrazolo[3,4- d]pyrimidin-6-yl)- 1-phenyl-ethane- 1,2-diamine | 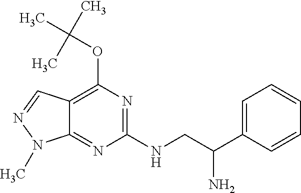 [SMILES: Cn1ncc2c(OC(C)(C)C)nc(NCC(N)c3ccccc3)nc21] | D | 4.18 | 341.3 |

The compounds detailed in Table 7 were prepared by chiral separation of previously described racemic compounds

TABLE 7

| | | | Chiral | | | | |
|---|---|---|---|---|---|---|---|
| | | | Puri- | | | | |
| Com- | | | fication/ | Chiral | Ana- | | |
| pound | | | Analytical | t_{R} | lytical | t_{R} | |
| No. | Name | Structure | Method | (min) | Method | (min) | [M + H]⁺ |
| 7.01 | 1-methyl-N6- {[(3R)- morpholin-3- yl]methyl}-N4- [4- (trifluoromethyl) phenyl]-1H- pyrazolo[3,4- d]pyrimidine- 4,6-diamine | 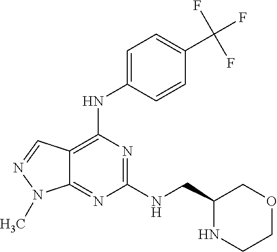 [SMILES: Cn1ncc2c(Nc3ccc(C(F)(F)F)cc3)nc(NC[C@H]3COCCN3)nc21] | C/G | 4.25 | C | 2.80 | 408.3 |
| 7.02 | 6-N-[(1S,2R)- 2- aminocyclohexyl]- 4-N-[(3- chloro-4- methylphenyl) methyl]-1- methylpyrazolo [3,4- d]pyrimidine- 4,6-diamine | 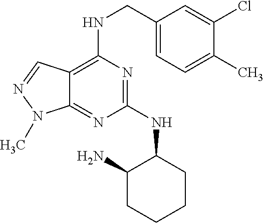 [SMILES: Cc1ccc(CNc2nc(N[C@H]3CCCC[C@H]3N)nc3c2cnn3C)cc1Cl] | D/F | 9.39 | E | 1.93 | 400.3 |
| 7.03 | 6-N-[(1R,2R)- 2- aminocyclohexyl]- 4-N-[(3- chloro-4- methylphenyl) methyl]-1- methylpyrazolo [3,4- d]pyrimidine- 4,6-diamine | 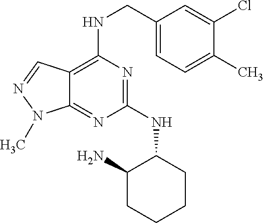 [SMILES: Cc1ccc(CNc2nc(N[C@@H]3CCCC[C@H]3N)nc3c2cnn3C)cc1Cl] | D/F | 10.73 | E | 1.93 | 400.3 |
| 7.04 | N6-[(2R)-2- amino-2- phenyl-ethyl]- N4- cyclopropyl-1- methyl- pyrazolo[3,4- d]pyrimidine- 4,6-diamine | 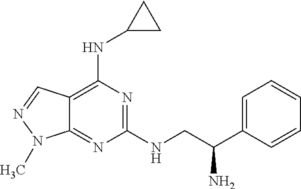 [SMILES: Cn1ncc2c(NC3CC3)nc(NC[C@H](N)c3ccccc3)nc21] | D/F | 4.73 | E | 1.14 | 324.2 |
| 7.05 | N6-[(2R)-2- amino-2- phenyl-ethyl]- N4-(4- fluorophenyl)- 1-methyl- pyrazolo[3,4- d]pyrimidine- 4,6-diamine | 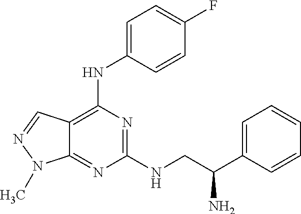 [SMILES: Cn1ncc2c(Nc3ccc(F)cc3)nc(NC[C@H](N)c3ccccc3)nc21] | E/H | 4.45 | C | 2.94 | 378.3 |
| 7.06 | N6-[(2S)-2- amino-2- phenyl-ethyl]- N4-(4- fluorophenyl)- 1-methyl- pyrazolo[3,4- d]pyrimidine- 4,6-diamine | 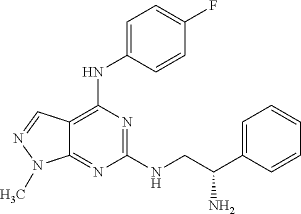 [SMILES: Cn1ncc2c(Nc3ccc(F)cc3)nc(NC[C@@H](N)c3ccccc3)nc21] | E/H | 6.71 | C | 2.94 | 378.3 |
| 7.07 | N6-[(2S)-2- amino-2- phenyl-ethyl]- 1-methyl-N4- (2,2,2- trifluoroethyl) pyrazolo[3,4- d]pyrimidine- 4,6-diamine | 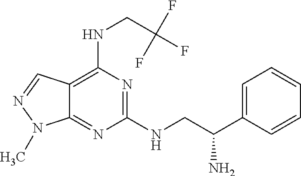 [SMILES: Cn1ncc2c(NCC(F)(F)F)nc(NC[C@@H](N)c3ccccc3)nc21] | D/G | 6.39 | D | 3.50 | 366.3 |
| 7.08 | N6-[(2R)-2- amino-2- phenyl-ethyl]- 1-methyl-N4- [6- (trifluoromethyl)- 3- pyridyl]pyrazolo [3,4- d]pyrimidine- 4,6-diamine | 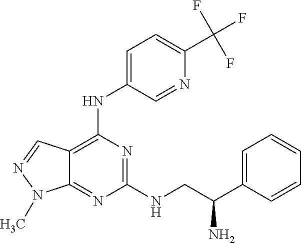 [SMILES: Cn1ncc2c(Nc3ccc(C(F)(F)F)nc3)nc(NC[C@H](N)c3ccccc3)nc21] | D/F | 3.35 | D | 3.96 | 429.2 |
| 7.09 | N6-[(2S)-2- amino-2- phenyl-ethyl]- 1-methyl-N4- [6- (trifluoromethyl)- 3- pyridyl]pyrazolo [3,4- d]pyrimidine- 4,6-diamine | 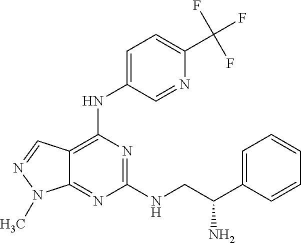 [SMILES: Cn1ncc2c(Nc3ccc(C(F)(F)F)nc3)nc(NC[C@@H](N)c3ccccc3)nc21] | D/F | 4.27 | D | 3.96 | 429.2 |
| 7.10 | rel-6-N-[(2S)- 2-amino-2- phenylpropyl]- 4-N,1- dimethylpyrazolo [3,4- d]pyrimidine- 4,6-diamine | 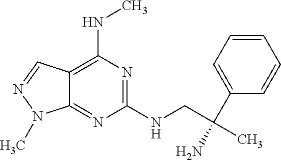 [SMILES: CNc1nc(NC[C@@](C)(N)c2ccccc2)nc2c1cnn2C] | F/K | 3.91 | C | 2.28 | 312.1 |
| 7.11 | N6-[(2S)-2- amino-2-(2- fluorophenyl) ethyl]-N4-tert- butyl-1-methyl- pyrazolo[3,4- d]pyrimidine- 4,6-diamine | 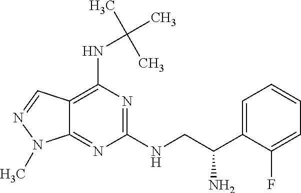 [SMILES: Cn1ncc2c(NC(C)(C)C)nc(NC[C@@H](N)c3ccccc3F)nc21] | E/H | 1.94 | E | 1.59 | 358.3 |
| 7.12 | N6-[(2R)-2- amino-2-(2- fluorophenyl) ethyl]-N4-tert- butyl-1-methyl- pyrazolo[3,4- d]pyrimidine- 4,6-diamine | 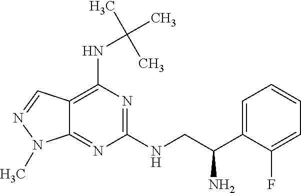 [SMILES: Cn1ncc2c(NC(C)(C)C)nc(NC[C@H](N)c3ccccc3F)nc21] | E/H | 2.77 | E | 1.65 | 358.3 |
| 7.13 | rel-N6-[(2R)-2- amino-2- phenyl-propyl]- 1-methyl-N4- [6- (trifluoromethyl)- 3- pyridyl]pyrazolo [3,4- d]pyrimidine- 4,6-diamine | 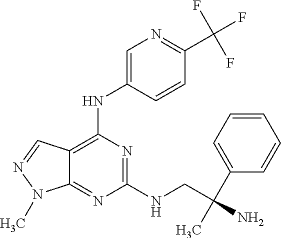 [SMILES: Cn1ncc2c(Nc3ccc(C(F)(F)F)nc3)nc(NC[C@](C)(N)c3ccccc3)nc21] | C/K | 11.10 | C | 3.18 | 443.3 |
| 7.14 | 6-N-[(2R)-2- amino-3- fluoropropyl]-4- N-[(3-chloro-4- methylphenyl) methyl]-1- methylpyrazolo [3,4- d]pyrimidine- 4,6-diamine | 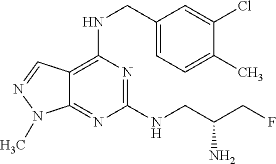 [SMILES: Cc1ccc(CNc2nc(NC[C@@H](N)CF)nc3c2cnn3C)cc1Cl] | D/F | 8.38 | C | 2.84 | 378.3/ 380.3 |
| 7.15 | rel-N6-[(2S)-2- amino-2- phenyl-propyl]- 1-methyl-N4- [6- (trifluoromethyl)- 3- pyridyl]pyrazolo [3,4- d]pyrimidine- 4,6-diamine | 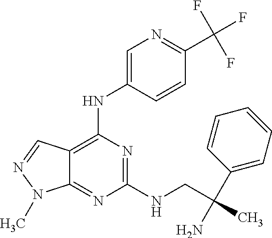 [SMILES: Cn1ncc2c(Nc3ccc(C(F)(F)F)nc3)nc(NC[C@@](C)(N)c3ccccc3)nc21] | C/K | 7.48 | C | 3.18 | 443.1 |
| 7.16 | N6-[(2R)-2- amino-2-(2- fluorophenyl) ethyl]-N4-tert- butyl-1- (trideuteriomethyl) pyrazolo[3, 4-d]pyrimidine- 4,6-diamine | 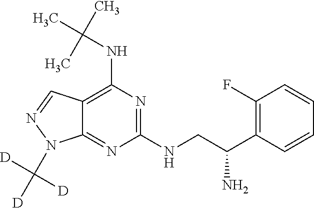 [SMILES: [2H]C([2H])([2H])n1ncc2c(NC(C)(C)C)nc(NC[C@@H](N)c3ccccc3F)nc21] | E/H | 2.84 | C | 2.93 | 361.3 |
| 7.17 | N6-[(2S)-2- amino-2-(2- fluorophenyl) ethyl]-N4-tert- butyl-1- (trideuteriomethyl) pyrazolo[3, 4-d]pyrimidine- 4,6-diamine | 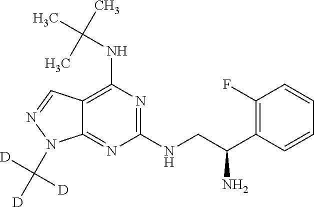 [SMILES: [2H]C([2H])([2H])n1ncc2c(NC(C)(C)C)nc(NC[C@H](N)c3ccccc3F)nc21] | E/H | 2.04 | C | 2.93 | 361.3 |
| 7.18 | N6-[(2R)-2- amino-2-(4- fluorophenyl) ethyl]-N4-tert- butyl-1-methyl- 1H- pyrazolo[3,4- d]pyrimidine- 4,6-diamine | 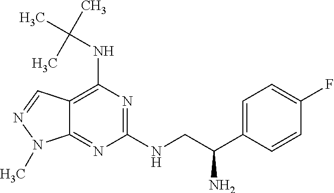 [SMILES: Cn1ncc2c(NC(C)(C)C)nc(NC[C@H](N)c3ccc(F)cc3)nc21] | F/H | 8.15 | C | 2.99 | 358.3 |
| 7.19 | N6-[(2R)-2- amino-2-(2- fluorophenyl) ethyl]-N4-tert- butyl-1-methyl- pyrazolo[3,4- d]pyrimidine- 4,6-diamine | 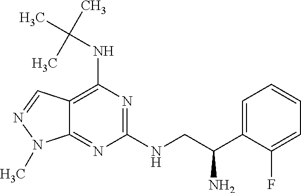 [SMILES: Cn1ncc2c(NC(C)(C)C)nc(NC[C@H](N)c3ccccc3F)nc21] | F/H | 1.94 | E | 1.59 | 358.3 |
| 7.20 | N6-[(2R)-2- amino-2-(3,4- difluorophenyl) ethyl]-N4-tert- butyl-1-methyl- 1H- pyrazolo[3,4- d]pyrimidine- 4,6-diamine | 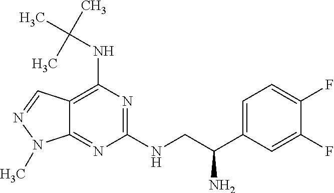 [SMILES: Cn1ncc2c(NC(C)(C)C)nc(NC[C@H](N)c3ccc(F)c(F)c3)nc21] | F/H | 4.22 | D | 3.90 | 376.4 |
| 7.21 | N6-[(2S)-2- amino-2-(3,4- difluorophenyl) ethyl]-N4-tert- butyl-1-methyl- 1H- pyrazolo[3,4- d]pyrimidine- 4,6-diamine | 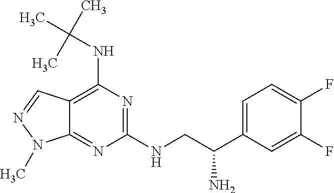 [SMILES: Cn1ncc2c(NC(C)(C)C)nc(NC[C@@H](N)c3ccc(F)c(F)c3)nc21] | F/H | 6.98 | D | 3.90 | 376.2 |
| 7.22 | N6-[(2R)-2- amino-2- cyclohexylethyl]- N4-tert-butyl- 1-methyl-1H- pyrazolo[3,4- d]pyrimidine- 4,6-diamine | 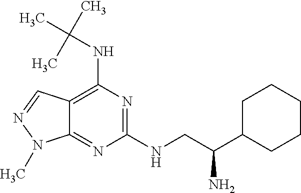 [SMILES: Cn1ncc2c(NC(C)(C)C)nc(NC[C@H](N)C3CCCCC3)nc21] | D/K | 2.52 | D | 4.74 | 346.4 |
| 7.23 | N6-[(2S)-2- amino-2- cyclohexylethyl]- N4-tert-butyl- 1-methyl-1H- pyrazolo[3,4- d]pyrimidine- 4,6-diamine | 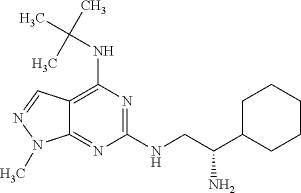 [SMILES: Cn1ncc2c(NC(C)(C)C)nc(NC[C@@H](N)C3CCCCC3)nc21] | D/K | 1.62 | D | 4.80 | 346.4 |

### Example 3—BCDIN3D Assay

The following General Methods details the assay conditions for the Biological assays.

BCDIN3D IC50 RFMS Assay with miRNA

The enzymatic assay was performed in a 384-well polypropylene plate in assay buffer with 25 mM Tris pH 8.0, 10 mM DTT, 1 mM EDTA, 0.03% BSA in a final assay volume of 20 μL. The enzyme with a final concentration of 100 nM was pre-incubated with the respective compound at 1% DMSO final assay concentration over 10 min at room temperature. The reaction was initiated by addition of 0.2 μM final assay concentration of annealed miRNA and 6 μM final concentration of SAM as substrates. After a 3 hours incubation at 37° C., the reaction was terminated with 40 μL of stop solution of 7.5% trichloroactetic acid in water containing 112.5 nM D₄-SAH and 30 nM ¹³C₁₀-SAH used as internal standards. The SAH product concentrations as well as both internal standards were analyzed by a RapidFire High Throughput Mass Spectrometry System (Agilent Technologies, Santa Clara, Calif.) using an AP14000 triple quadrupole mass spectrometer (AB Sciex Framingham, Mass.).

BCDIN3D IC50 RFMS Assay with Mini-Helix

The enzymatic assay was performed in a 384-well polypropylene plate in assay buffer with 25 mM Tris pH 8.0, 10 mM DTT, 1 mM EDTA, 0.03% BSA in a final assay volume of 20 μL. The enzyme with a final concentration of 25 nM was pre-incubated with the respective compound at 1% DMSO final assay concentration over 10 min at room temperature. The reaction was initiated by addition of 0.2 μM final assay concentration of mini-helix and 6 μM final concentration of SAM as substrates. After a 1 hr incubation at 37° C., the reaction was terminated with 40 μL of stop solution of 7.5% trichloroactetic acid in water containing 112.5 nM D₄-SAH and 30 nM ¹³C₁₀-SAH used as internal standards. The SAH product concentrations as well as both internal standards were analyzed by a RapidFire High Throughput Mass Spectrometry System (Agilent Technologies, Santa Clara, Calif.) using an AP14000 triple quadrupole mass spectrometer (AB Sciex Framingham, Mass.).

### BCDIN3D InCellPulse™ Assay

The thermal stability assay was performed with lipofectamine 3000 transiently transfected A549 using the plasmid pICP-ePL-BCDIN3 Da. This plasmid encodes for BCDIN3D N-terminally fused to a Prolabel (ePL), complementing a split β-Galactosidase Enzyme System InCELL Pulse Technology (InCELL Hunter Detection Kit, DiscoverX/Eurofins, cat: 96-0079L). 20 μl A549 cells (1500 cell/well) were added with a multidrop device to compounds to a final DMSO concentration of 0.6% in 384-well polypropylene plate format in IMDM medium and pre-incubated with the compounds for 1 h at 37° C. and 5% CO2. Subsequently, a heat shock was applied to the entire sealed plate in a 58° C. water bath for 15 min. The plates were cooled to RT, spun down and 25 μl/well of EA Detection Solution at 3.3× (InCELL Hunter Detection Kit) was added per well. 21 μl of the mixture was added to a black 384-well measurement plate (Corning, PS, black, Flat Bottom, #3575), mix for 30 minutes on a plate shaker at RT. Finally, the luminescence was measured with a Perkin Elmer EnVision with 0.2 s measurement time in luminescence mode.

### Results

The following table details the biological data from the compounds 1.01 to 1.42 detailed in Example 2.

TABLE 8

| | RapidFire | |
|---|---|---|
| | BCDIN3D | InCellPulse |
| Compound | mini-helix | BCDIN3D |
| No. | IC50 (nM) | IC50 (nM) |
| 1.01 | 1984 | 3330 |
| 1.02 | 4267 | |
| 1.03 | 975 | 917.9 |
| 1.04 | 1260 | 1530 |
| 1.05 | 2212 | 2974 |
| 1.06 | 1684 | 2930 |
| 1.07 | 4379 | |
| 1.08 | 2801 | |
| 1.09 | 4073 | |
| 1.10 | 5485 | |
| 1.11 | 762.2 | 1062 |
| 1.12 | 9517 | |
| 1.13 | 7799 | |
| 1.14 | 568.1 | 754 |
| 1.15 | 541.1 | 1020 |
| 1.16 | 743.6 | 1700 |
| 1.17 | 6898 | |
| 1.18 | 4748 | |
| 1.19 | 6385 | |
| 1.20 | 3525 | |
| 1.21 | 1149 | 8070 |
| 1.22 | 1653 | 6460 |
| 1.23 | 7725 | 8470 |
| 1.24 | 2251 | 10600 |
| 1.25 | 1659 | 7720 |
| 1.26 | 744.3 | 1870 |
| 1.27 | 2885 | 5340 |
| 1.28 | 1108 | 765 |
| 1.29 | 2905 | |
| 1.30 | 1851 | 3790 |
| 1.31 | 949.6 | 814.2 |
| 1.32 | 77.9 | 234 |
| 1.33 | 4329 | 6460 |
| 1.34 | 1141 | 1410 |
| 1.35 | 3950 | 7880 |
| 1.36 | 5660 | 10600 |
| 1.37 | 7618 | |
| 1.38 | 8630 | 4000 |
| 1.39 | 6910 | |
| 1.40 | 6190 | |
| 1.41 | 21080 | |
| 1.42 | 5760 | |

The following table details the biological data from the compounds 2.01 to 2.52 detailed in Example 2.

TABLE 9

| | RapidFire | |
|---|---|---|
| | BCDIN3D | InCellPulse |
| Compound | mini-helix | BCDIN3D |
| No. | IC50 (nM) | IC50 (nM) |
| 2.01 | 274.9 | 707.5 |
| 2.02 | 505 | 1910 |
| 2.03 | 20.67 | 282 |
| 2.04 | 60.8 | 1800 |
| 2.05 | 1670 | 5050 |
| 2.06 | 509 | 2210 |
| 2.07 | 443 | 4100 |
| 2.08 | 242 | 2720 |
| 2.09 | 39.8 | 602 |
| 2.10 | 8680 | 15700 |
| 2.11 | 490 | 2890 |
| 2.12 | 588 | 1490 |
| 2.13 | 369 | 1610 |
| 2.14 | 5960 | 9590 |
| 2.15 | 446 | 1630 |
| 2.16 | 452 | 959 |
| 2.17 | 440 | 2560 |
| 2.18 | 111 | 207 |
| 2.19 | 7120 | 11680 |
| 2.20 | 179 | 887 |
| 2.21 | 514 | 2470 |
| 2.22 | 332 | 1500 |
| 2.23 | 59.1 | 473 |
| 2.24 | 2460 | 3500 |
| 2.25 | 1540 | 3520 |
| 2.26 | 1000 | 4650 |
| 2.27 | 262 | 1810 |
| 2.28 | 57.4 | 1070 |
| 2.29 | 444 | 3000 |
| 2.30 | 545 | 1784 |
| 2.31 | 976 | 8240 |
| 2.32 | 176 | 815 |
| 2.33 | 327 | 3860 |
| 2.34 | 201 | 1420 |
| 2.35 | 260 | 1980 |
| 2.36 | 138 | 1980 |
| 2.37 | 8600 | 16500 |
| 2.38 | 1710 | |
| 2.39 | 350 | 2129 |
| 2.40 | 33.3 | 234 |
| 2.41 | 211 | 1610 |
| 2.42 | 1260 | 2990 |
| 2.43 | 2790 | 5940 |
| 2.44 | 56.55 | 329 |
| 2.45 | 424 | 2467 |
| 2.46 | 2100 | 6530 |
| 2.47 | 4520 | 8300 |
| 2.48 | 1080 | 2430 |
| 2.49 | 50 | 379 |
| 2.50 | 344 | 2980 |
| 2.51 | 179 | 252 |
| 2.52 | 6900 | 8890 |

The following table details the biological data from the compounds 3.01 to 3.30 detailed in Example 2.

TABLE 10

| | RapidFire | |
|---|---|---|
| | BCDIN3D | InCellPulse |
| Compound | mini-helix | BCDIN3D |
| No. | IC50 (nM) | IC50 (nM) |
| 3.01 | 3325 | 2280 |
| 3.02 | 6563 | |
| 3.03 | 5713 | |
| 3.04 | 664.5 | 1450 |
| 3.05 | 1440 | 1440 |
| 3.06 | 3419 | 4420 |
| 3.07 | 298.6 | 664 |
| 3.08 | 50.5 | 259 |
| 3.09 | 201 | 503 |
| 3.10 | 228 | 123 |
| 3.11 | 101 | 65.4 |
| 3.12 | 479 | 1572 |
| 3.13 | 180 | 510 |
| 3.14 | 252 | 2410 |
| 3.15 | 1630 | 6540 |
| 3.16 | 9919 | 60000 |
| 3.17 | 9566 | |
| 3.18 | 88 | 335 |
| 3.19 | 357 | 1240 |
| 3.20 | 305 | 724 |
| 3.21 | 1440 | 2000 |
| 3.22 | 1800 | 2260 |
| 3.23 | 115 | 267 |
| 3.24 | 45 | 481 |
| 3.25 | 6170 | 5500 |
| 3.26 | 3004 | 4030 |
| 3.27 | 4305 | 4790 |
| 3.28 | 383 | 4670 |
| 3.29 | 4920 | 60000 |
| 3.30 | 1140 | 1410 |

The following table details the biological data from the compounds 4.01 to 4.130 detailed in Example 2.

TABLE 11

| | RapidFire | |
|---|---|---|
| | BCDIN3D | InCellPulse |
| Compound | mini-helix | BCDIN3D |
| No. | IC50 (nM) | IC50 (nM) |
| 4.01 | 325 | 1940 |
| 4.02 | 4280 | 5670 |
| 4.03 | 62.55 | 166 |
| 4.04 | 423.9 | 820.3 |
| 4.05 | 109 | 275 |
| 4.06 | 87.2 | 139 |
| 4.07 | 132 | 282 |
| 4.08 | 1080 | 2950 |
| 4.09 | 178 | 505 |
| 4.10 | 71.7 | 323 |
| 4.11 | 168 | 2160 |
| 4.12 | 52.1 | 186 |
| 4.13 | 86.6 | 258 |
| 4.14 | 89.5 | 206 |
| 4.15 | 69.4 | 295 |
| 4.16 | 40.8 | 46.3 |
| 4.17 | 50.12 | 178 |
| 4.18 | 288 | 270 |
| 4.19 | 36.1 | 86.39 |
| 4.20 | 38.5 | 60.42 |
| 4.21 | 42.8 | 218 |
| 4.22 | 34.1 | 29.9 |
| 4.23 | 1524 | 202.4 |
| 4.24 | 13.9 | 14.4 |
| 4.25 | 50.7 | 319 |
| 4.26 | 21.7 | 384 |
| 4.27 | 46.6 | 610 |
| 4.28 | 149 | 318 |
| 4.29 | 116 | 525 |
| 4.30 | 28.6 | 62.6 |
| 4.31 | 7390 | 60000 |
| 4.32 | 95.8 | 140 |
| 4.33 | 64.4 | 38 |
| 4.34 | 118 | 162 |
| 4.35 | 143 | 141 |
| 4.36 | 554 | 1860 |
| 4.37 | 8010 | |
| 4.38 | 8100 | 9560 |
| 4.39 | 9170 | 17100 |
| 4.40 | 3630 | 4790 |
| 4.41 | 945 | 3330 |
| 4.42 | 2280 | 7570 |
| 4.43 | 2560 | 4200 |
| 4.44 | 527 | 992 |
| 4.45 | 316 | 414 |
| 4.46 | 501 | 1132 |
| 4.47 | 253 | 667 |
| 4.48 | 3860 | 7400 |
| 4.49 | 3390 | 6060 |
| 4.50 | 196 | 871 |
| 4.51 | 581 | 1810 |
| 4.52 | 169 | 536 |
| 4.53 | 118 | 282 |
| 4.54 | 1970 | 3110 |
| 4.55 | 133 | 152 |
| 4.56 | 3470 | 7270 |
| 4.57 | 6450 | 2720 |
| 4.58 | 70.4 | 184 |
| 4.59 | 109 | 77.5 |
| 4.60 | 6.1 | 44.4 |
| 4.61 | 266 | 378 |
| 4.62 | 6.1 | 112 |
| 4.63 | 189 | 889 |
| 4.64 | 202 | 949 |
| 4.65 | 230 | 616 |
| 4.66 | 128 | 137 |
| 4.67 | 179 | 168 |
| 4.68 | 1140 | 3240 |
| 4.69 | 851 | 3140 |
| 4.70 | 1300 | |
| 4.71 | 1400 | 1200 |
| 4.72 | 154.1 | 318 |
| 4.73 | 528 | 412 |
| 4.74 | 252 | 341 |
| 4.75 | 240 | 711 |
| 4.76 | 136 | 232 |
| 4.77 | 2770 | 1970 |
| 4.78 | 769 | 379 |
| 4.79 | 2300 | 3200 |
| 4.80 | 656 | 518 |
| 4.81 | 583 | 625 |
| 4.82 | 25.6 | 40.8 |
| 4.83 | 75.5 | 94.4 |
| 4.84 | 137 | 125 |
| 4.85 | 172 | 267 |
| 4.86 | 1600 | 1450 |
| 4.87 | 50.7 | 70.9 |
| 4.88 | 112 | 148 |
| 4.89 | 109 | 223 |
| 4.90 | 576 | 989 |
| 4.91 | 129 | 263 |
| 4.92 | 343 | 461 |
| 4.93 | 25.8 | 72 |
| 4.94 | 6.1 | 47 |
| 4.95 | 374 | 456 |
| 4.96 | 14 | 21.8 |
| 4.97 | 84.3 | 176 |
| 4.98 | 136 | 232 |
| 4.99 | 8 | 17 |
| 4.100 | 1330 | 1860 |
| 4.101 | 463 | 330 |
| 4.102 | 4380 | 2630 |
| 4.103 | 321 | 480 |
| 4.104 | 28 | 56 |
| 4.105 | 74 | 144 |
| 4.106 | 149 | 437 |
| 4.107 | 280 | 618 |
| 4.108 | 288 | 611 |
| 4.109 | 17 | 34 |
| 4.110 | 553 | 672 |
| 4.111 | 369 | 292 |
| 4.112 | 164 | 439 |
| 4.113 | 62 | 148 |
| 4.114 | 540 | 1560 |
| 4.115 | 39 | 271 |
| 4.116 | 151 | 123 |
| 4.117 | 783 | 608 |
| 4.118 | 2150 | 2500 |
| 4.119 | 24 | 27 |
| 4.120 | 252 | 1450 |
| 4.121 | 6450 | 4810 |
| 4.122 | 120 | 220 |
| 4.123 | 1410 | 3560 |
| 4.124 | 290 | 570 |
| 4.125 | 450 | 2310 |
| 4.126 | 450 | 580 |
| 4.127 | 6770 | 2240 |
| 4.128 | 1010 | 1290 |
| 4.129 | 180 | 290 |
| 4.130 | 6010 | 11200 |

The following table details the biological data from the compounds 5.02 to 5.33 detailed in Example 2.

TABLE 12

| | RapidFire | |
|---|---|---|
| | BCDIN3D | InCellPulse |
| Compound | mini-helix | BCDIN3D |
| No. | IC50 (nM) | IC50 (nM) |
| 5.02 | 216.6 | 944 |
| 5.03 | 89.4 | 436 |
| 5.04 | 694 | 3640 |
| 5.05 | 51.8 | 68.3 |
| 5.06 | 1100 | 1110 |
| 5.07 | 135 | 881.5 |
| 5.08 | 615 | 1170 |
| 5.09 | 127.2 | 55.93 |
| 5.10 | 495 | 462 |
| 5.11 | 321 | 457 |
| 5.12 | 345 | 643 |
| 5.13 | 383 | 525 |
| 5.14 | 294 | 429 |
| 5.15 | 28.4 | 47.1 |
| 5.16 | 41.7 | 38.5 |
| 5.17 | 470 | 1870 |
| 5.18 | 47 | 27.8 |
| 5.19 | 375 | 767.3 |
| 5.20 | 609 | 1120 |
| 5.21 | 3860 | 11100 |
| 5.22 | 42.8 | 129 |
| 5.23 | 61.7 | 124 |
| 5.25 | 1810 | 5580 |
| 5.26 | 3390 | 5570 |
| 5.27 | 2810 | 10000 |
| 5.28 | 90 | 370 |
| 5.29 | 2780 | 1690 |
| 5.30 | 370 | |
| 5.31 | 350 | |
| 5.32 | 70 | 120 |
| 5.33 | 130 | 150 |

The following table details the biological data from the compounds 6.01 to 6.05 detailed in Example 2.

TABLE 13

| | RapidFire | |
|---|---|---|
| | BCDIN3D | InCellPulse |
| Compound | mini-helix | BCDIN3D |
| No. | IC50 (nM) | IC50 (nM) |
| 6.01 | 2510 | 6610 |
| 6.04 | 2620 | 4150 |
| 6.05 | 4160 | 5140 |

The following table details the biological data from the compounds 7.02 to 7.23 detailed in Example 2.

TABLE 14

| | RapidFire | |
|---|---|---|
| | BCDIN3D | InCellPulse |
| Compound | mini-helix | BCDIN3D |
| No. | IC50 (nM) | IC50 (nM) |
| 7.02 | 998.1 | 898.8 |
| 7.03 | 8583 | |
| 7.04 | 3910 | 2970 |
| 7.05 | 241 | 169 |
| 7.06 | 2190 | 2930 |
| 7.07 | 5620 | 4120 |
| 7.08 | 33.4 | 223 |
| 7.09 | 209 | 414 |
| 7.10 | 7650 | 10800 |
| 7.11 | 181 | 4183 |
| 7.12 | 2440 | 2150 |
| 7.13 | 2620 | 6000 |
| 7.14 | 5652 | 6320 |
| 7.15 | 38.1 | 133 |
| 7.16 | 69.02 | |
| 7.17 | 981 | 2140 |
| 7.18 | 330 | 410 |
| 7.19 | 180 | |
| 7.20 | 800 | |
| 7.21 | 4.62 | |
| 7.22 | 800 | |
| 7.23 | 5170 | |

### Example 4: BCDIN3D Degradation Assay by Denaturing Polyacrylamide Gel Electrophoresis, Western Blotting and Antibody-Based Detection

The commercially available reagents used are shown in Table 15.

TABLE 15

| Reagent | Product no. | Manufacturer/supplier |
|---|---|---|
| A549, human Caucasian lung carcinoma | 86012804-1VL | Sigma-Aldrich/ECACC |
| DMEM, low glucose, GlutaMAX ™ | 21885108 | Gibco ™ |
| supplement, pyruvate | | |
| Fetal Bovine Serum (FBS) | F7524-500ML | Sigma-Aldrich |
| 6-well cell culture plates, TC treated | 734-2323 | VWR ™ |
| Dulbecco’s phosphate buffered | D8537-500ML | Sigma-Aldrich |
| saline (PBS, 1×) | | |
| RIPA buffer (1×) | 89901 | Thermo Scientific ™ |
| Pierce Protease Inhibitor Mini Tablets | A32953 | Thermo Scientific ™ |
| Protein LoBind Tubes | 0030122356 | Eppendorf |
| BCA Protein Assay Kit | 23227 | Thermo Scientific ™ |
| NuPAGE ™ LDS Sample Buffer (4×) | NP0007 | Invitrogen ™ |
| NuPAGE ™ Sample Reducing Agent (10×) | NP0009 | Invitrogen ™ |
| NuPAGE ™ 4-12% Bis-Tris Protein Gels, | NP0323BOX | Invitrogen ™ |
| 1.0 mm, 15-well | | |
| NuPAGE ™ 4-12% Bis-Tris Protein Gels, | NP0321BOX | Invitrogen ™ |
| 1.0 mm, 10-well | | |
| NuPAGE ™ MOPS SDS Running Buffer (20×) | NP0001 | Invitrogen ™ |
| Precision Plus Protein ™ Dual Color Standards | 1610374 | BIO-RAD |
| Trans-Blot ® Turbo ™ Mini Nitrocellulose | 1704158 | BIO-RAD |
| Transfer Packs | | |
| TRIS-buffered saline (TBS, 20×) pH 7.4 | J60877.K3 | Alfa Aesar |
| TWEEN ® 20 | P1379 | Sigma-Aldrich |
| Marvel Original dried skimmed milk | n/a | Premier Foods |
| Rabbit IgG HRP linked whole Ab | GENA934-1ML | GE Healthcare |
| ECL ™ Prime western blotting | GERPN2236 | GE Healthcare |
| detection reagent | | |

The following were also used in this example.
A549 complete growth medium: DMEM GutaMAX+10% Fetal Bovine Serum (FBS) H Anti-BCDIN3D affinity-purified rabbit polyclonal IgG: Custom-made antibodies were generated by immunisation of 2 rabbits with full-length recombinant BCDIN3D protein (purified from baculovirus-infected insect cells). Rabbit serum was affinity-purified using immobilised antigen. Antibody generation and purification was performed by BioGenes GmbH, Koepenicker Straße 325, 0-12555 Berlin, Germany.
BCDIN3D inhibitors (Compounds 4.3, 4.22, 4.23, 4.47, 4.53, 4.82, 4.85, 5.22 and 7.05), 10 mM in DMSO.
TBS/T buffer: sx TBS, 0.1% Tween 20

The equipment used is shown in Table 16.

TABLE 16

| Item | Product no. | Manufacturer/supplier |
|---|---|---|
| Heracell ™ 150i CO2 Incubator | 51026280 | Thermo Scientific ™ |
| Centrifuge 5424 R | 5404000460 | Eppendorf |
| Mixer HC | S8012-0000 | Starlab |
| XCell SureLock ™ Mini-Cell | EI0002 | Invitrogen ™ |
| Trans-Blot ® Turbo ™ Transfer System | 1704150 | BIO-RAD |
| PowerPac ™ Basic Power Supply | 1645050 | BIO-RAD |
| Sea-saw rocker mini | SSM4 | Stuart ® |
| ChemiDoc MP Imaging system | 17001402 | BIO-RAD |

### Method

A549 cells are cultured under standard tissue culture conditions (37° C., 5% CO₂ humidified atmosphere) using complete growth medium.

On day one, A549 cells are plated in 6-well tissue culture plates at 150,000-200,000 cells per well in 2.25 ml total volume of complete growth medium. The next day, BCDIN3D inhibitors are added as 10× working stock (10 μM or 1 μM) in 250 μl complete growth medium to achieve a final concentration of 1 μM or 100 nM, respectively (DMSO at 0.1%) and cells are incubated for 48 h without medium exchange. For protein lysate preparation, cells are washed twice with 2 ml 1×PBS each. After complete removal of PBS, 200 μl cold RIPA buffer (supplemented with Protease Inhibitors) is added and the plates are incubated for 20 minutes on ice to allow for complete cell lysis. Lysates are transferred to 1.5 ml protein LoBind Eppendorf tubes and cleared by centrifugation for 15 minutes at 20,000×g at 4° C. Cleared lysates are transferred to fresh tubes. Total protein concentration is determined by BCA protein assay according to manufacturer's instructions.

Protein lysates are diluted in RIPA lysis buffer and set up with LDS sample buffer and reducing agent to a final concentration of ca. 1 ug/ul. Samples are heated to 70° C. for 10 minutes prior to loading onto 4-12% NuPAGE Bis-Tris protein gels. Gel electrophoresis is performed using an XCell sure lock system and 1×MOPS running buffer for ca. 70 minutes at 160 V constant. After electrophoresis is complete, proteins are transferred to Nitrocellulose membrane using the TransBlot Turbo transfer system and corresponding transfer packs using the mixed MW transfer programme (2.5 A, 25V, 7 minutes).

Membranes are washed with TBS/T buffer and blocked in 5% non-fat dried milk in TBS/T buffer for 1 hour at room temperature under gentle agitation. Primary antibody (anti-BCDIN3D at 1:1,000 dilution) incubation is in 5-10 ml of TBS/T buffer, over-night at 4° C. with gentle agitation. The next day, membranes are washed four-times in 25 ml TBS/T buffer for 5 minutes at room temperature and incubated with secondary antibody (anti-rabbit HRP-linked antibody, 1:10,000) in 10 ml TBS/T for 1 hour at room temperature. After four washes (25 ml TBS/T, 5 minutes each, room temperature), western blots are developed with 2 ml ECL Prime working solution. Images are taken on a Chemidoc MP imaging system and processed using ImageJ (Fiji) software.

### Results:

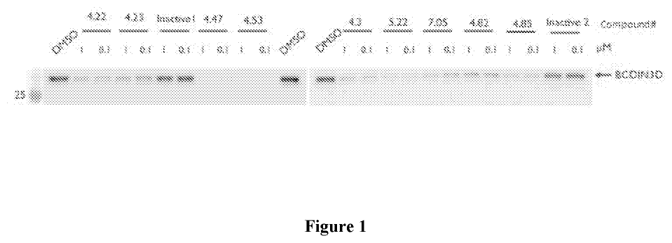

The Western Blot image acquired for Compounds 4.3, 4.22, 4.23, 4.47, 4.53, 4.82, 4.85, 5.22 and 7.05 is shown in FIG. 1.

### Example 5—BCDIN3D and Inflammation

BCDIN3D is ubiquitously expressed at low levels in human tissues, with higher expression levels observed in lymphoid cell lines such as JM1 (CCLE database https://portals.broadinstitute.org/ccle). We have observed significant downregulation of BCDIN3D expression in primary human CD4+ and CD8+ T-cells in response to T-cell activation (FIG. 2), suggesting a functional role for BCDIN3D in T-cell maintenance and regulation.

Using potent and selective BCDIN3D inhibitors, we show that pharmacological inhibition of BCDIN3D blocks IFN gamma production by activated T-cells (FIG. 3), demonstrating anti-inflammatory activity of BCDIN3D inhibitors in vitro

Testing of BCDIN3D inhibitors in an in vivo model of delayed-type hypersensitivity, a well established in vivo inflammation model dependent on T-cell and monocyte/macrophage activation, confirmed significant anti-inflammatory activity of BCDIN3D inhibitors in vivo (FIG. 4).

Together, these data support the development of BCDIN3D inhibitors for T-cell and monocyte-driven auto-immune diseases described herein, such as rheumatoid arthritis, inflammatory bowel disorders, multiple sclerosis, systemic lupus erythematosus, psoriasis, atopic dermatitis and other conditions disclosed herein.

### BCDIN3D Expression is Downregulated Upon T-Cell Activation

Method:

Naïve CD4+ and CD8+ T cells were isolated from human PBMC (3 independent donors) using a Miltenyi kit according to manufacturer's instructions. T cells were subsequently plated in standard culture medium and stimulated with anti CD3/28 for 24 h (CD4+) and 48 h (CD8+), respectively. Cells were then lysed and RNA was extracted using Rneasy kits (Quiagen) according to manufacturer's instructions. BCDIN3D transcript levels were asses by qRT-PCR.

### BCDIN3Di Blocks IFNg Production by Activated CD4+ T-Cells In Vitro

Method:

Naïve CD4+ T cells were isolated from human PBMC (3 independent donors) using a Miltenyi kit according to manufacturer's instructions. CD4+ T cells were then plated in 96-well plates in standard culture medium containing anti-IL-4; anti-IFNγ; anti-IL-12 antibodies and IL-2. Cells were stimulated with CD3/28 beads in the presence of BCDIN3D inhibitors at varying concentrations, and cultured for 7 days. On day 7, cells were restimulated with PMA and ionomycin for 4 h in the presence of brefeldin A. Cells were fixed and permeabilised and levels of intracellular IFNg were assessed by intracellular anti-IFNg staining and subsequent flow cytometry following standard protocols.

In FIG. 3, the black dashed line represents unstimulated T-cells and the black dotted line represents stimulated cells in the presence of DMSO control.

### BCDIN3Di Blocks Delayed-Type Hypersensitivity Response In Vivo

Method:

Male CD-1/ICR mice were used for this model. Storm test compounds were dosed daily at 30 mg/kg qd throughout the study (days 1-7). On day 1 of the model, mice were injected IV with washed sheep red blood cells (1×10⁶). On day 6, control mice were administered dexamethasone or vehicle and all mice were injected with SRBC's (1×10⁸) into the back left footpad. On day 7, mice were re-administered compounds and footpad thickness of both back foot pads were measured using a micrometer. Foot pad thickness per animal were normalized to the un-injected (back right) footpad.

### Numbered Paragraphs

The following numbered paragraphs serve to define particular aspects and embodiments of the invention.

Paragraph 1. A compound of the formula (I), or a pharmaceutically acceptable salt thereof,

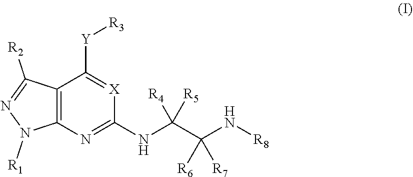

wherein:
R₁ is selected from methyl, ethyl, CH₂F, CHF₂, cyclopropyl, and CH₂CN wherein one or more hydrogen atoms in methyl, ethyl, CH₂F, CHF₂, cyclopropyl, and CH₂CN may be replaced by deuterium;
R₂ is selected from hydrogen, C₁₋₂ alkyl, C₁₋₂haloalkyl, cyano, amino, halo, trifluoromethyl, trifluoromethoxy, hydroxy, carboxy, carbamoyl, sulphamoyl, NRᵣRₛ, ORᵣ, C(O)Rᵣ, C(O)ORᵣ, OC(O)Rᵣ, C(O)N(Rᵣ)Rₛ, N(Rᵣ)C(O)Rₛ, S(O)ₐRᵣ (where a is 0, 1 or 2), SO₂N(Rᵣ)Rₛ, N(Rᵣ)SO₂Rₛ or (CH₂)_{b}NRᵣRₛ (where b is 1, 2 or 3), wherein Rᵣ and Rₛ are each independently selected from hydrogen or C₁₋₂alkyl;
R₃ is a group of the formula:

-L₁ₐ-L_{1b}-Q₁
wherein
L₁ₐ is absent or C₁₋₃alkylene, C₃₋₄cycloalkylene, optionally substituted by one or more substituents selected from C₁₋₂alkyl, halo, hydroxy, amino or oxo;
L_{1b} is absent or selected from O, S, SO, SO₂, N(Rₓ), C(O), C(O)O, OC(O), C(O)N(Rₓ), N(Rₓ)C(O), N(Rₓ)C(O)N(R_{y}), S(O)₂N(Rₓ), or N(Rₓ)SO₂, wherein Rₓ and R_{y} are each independently selected from hydrogen or C₁₋₂alkyl; and
Q₁ is hydrogen, cyano, C₁₋₆alkyl, C₃₋₁₀carbocyclic, C₂₋₃alkenyl, C₂₋₃alkynyl, aryl, heterocyclyl or heteroaryl; wherein one or more hydrogen atoms in C₁₋₆alkyl may be replaced by deuterium; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, halo, trifluoromethyl, trifluoromethoxy, amino, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, S(O)_{y}Rₜ (where y is 0, 1 or 2), SO₂N(Rₜ)Rᵤ, N(Rₜ)SO₂R, or (CH₂)_{z}NRₜRᵤ (where z is 1, 2 or 3); wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ as defined below; or
Q₁ is optionally substituted by one or more groups of the formula:

-L_{1c}-L_{1d}-Z₁
wherein
L_{1c} is absent or C₁₋₃alkylene optionally substituted by C₁₋₂alkyl or oxo;
L_{1d} is absent or selected from O, C(O), C(O)O, OC(O), C(O)N(Rₘ), N(Rₘ)C(O), N(Rₘ)C(O)N(Rₙ), S(O)₂N(Rₘ), S(O)₂, or N(Rₘ)SO₂, wherein Rₘ and Rₙ are each independently selected from hydrogen or C₁₋₂alkyl; and
Z₁ is cyano, C₁₋₆alkyl, C₃₋₁₀carbocyclic, C₂₋₃alkenyl, C₂₋₃alkynyl, aryl, heterocyclyl or heteroaryl; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, C₂₋₄alkynyl, halo, trifluoromethyl, trifluoromethoxy, amino, cyano, hydroxy, carboxy, carbamoyl, sulphamoyl, NRᵥR_{w}, ORᵥ, C(O)Rᵥ, C(O)ORᵥ, OC(O)Rᵥ, C(O)N(Rᵥ)R_{w}, N(Rᵥ)C(O)R_{w}, S(O)ₓRᵥ (where x is 0, 1 or 2), SO₂N(Rᵥ)R_{w}, N(Rᵥ)SO₂R_{w} or (CH₂)ₛNRᵥR_{w} (where s is 1, 2 or 3), wherein Rᵥ and R_{w} are each independently selected from hydrogen or C₁₋₄alkyl;
   R₄ is hydrogen;
   R₅ is selected from hydrogen and C₁₋₄alkyl;
   R₆ is selected from hydrogen and C₁₋₄ alkyl;
   R₇ is selected from hydrogen, C₁₋₄alkyl, C₃₋₆cycloalkyl, heterocyclyl, heteroaryl and aryl, wherein C₁₋₄alkyl, C₃₋₆cycloalkyl, heterocyclyl, heteroaryl or aryl is optionally substituted with one or more substituents selected from C₁₋₄alkyl, C₂₋₃alkenyl, C₂₋₃alkynyl, halo, trifluoromethyl, trifluoromethoxy, amino, cyano, hydroxy, carboxy, carbamoyl, sulphamoyl, NRₚR_{q}, ORₚ, C(O)Rₚ, C(O)ORₚ, OC(O)Rₚ, C(O)N(Rₚ)R_{q}, N(Rₚ)C(O)R_{q}, S(O)ⱼRₚ (where j is 0, 1 or 2), SO₂N(Rₚ)R_{q}, N(Rₚ)SO₂R_{q} or (CH₂)ₖNRₚR_{q} (where k is 1, 2 or 3), wherein Rₚ and R_{q} are each independently selected from hydrogen or C₁₋₄alkyl;
   R₈ is selected from hydrogen or C₁₋₄alkyl;
   or R₆ and R₇ may be linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano;
   or R₅ and R₇ may be linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano;
   or R₇ and R₃ may be linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano;
   X is selected from N and CR₉;
   R₉ is selected from hydrogen, methyl, and fluoro;
   Y is selected from O and NR₁₀; and
   R₁₀ is selected from hydrogen and C₁₋₄alkyl;
   or R₃ and R₁₀ may be linked together to form a 4-, 5-, 6- or 7-membered heterocyclic ring system.

Paragraph 2. A compound according to paragraph 1, or a pharmaceutically acceptable salt thereof, wherein R₅ is C₁₋₄ alkyl, R₆ is hydrogen and R₇ is hydrogen.

Paragraph 3. A compound according to paragraph 1, or a pharmaceutically acceptable salt thereof, wherein R₅ is hydrogen.

Paragraph 4. A compound according to paragraph 1, or a pharmaceutically acceptable salt thereof, wherein R₅ is hydrogen and R₇ is selected from C₁₋₄alkyl, C₃₋₆cycloalkyl, heteroaryl and aryl, wherein C₁₋₄alkyl, C₃₋₆cycloalkyl, heteroaryl or aryl is optionally substituted with one or more substituents selected from C₁₋₄alkyl, C₂₋₃alkynyl, halo, cyano, ORₚ, wherein Rₚ is C₁₋₄alkyl.

Paragraph 5. A compound according to paragraph 4, or a pharmaceutically acceptable salt thereof, wherein R₅ is hydrogen and R₇ is aryl optionally substituted with one or more substituents selected from C₁₋₄alkyl, C₂₋₃alkynyl, halo, cyano, ORₚ, wherein Rₚ is C₁₋₄alkyl.

Paragraph 6. A compound according to any preceeding paragraph, or a pharmaceutically acceptable salt thereof, wherein R₇ is hydrogen, methyl optionally substituted with fluoro or OMe, ethyl, —CH₂F, —CH₂OMe, cyclopropyl, cyclohexyl, phenyl,

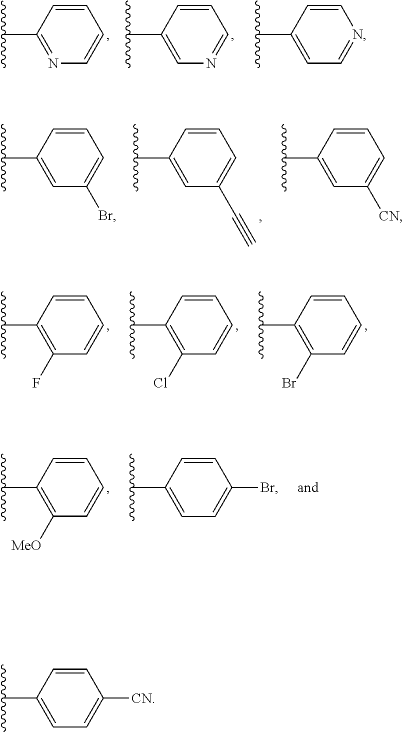

Paragraph 7. A compound according to paragraph 1, or a pharmaceutically acceptable salt thereof, wherein R₆ is hydrogen, and R₅ and R₇ are linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano.

Paragraph 8. A compound according to paragraph 1, or a pharmaceutically acceptable salt thereof, wherein R₅ is hydrogen, and R₆ and R₇ are linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂-haloalkyl, halo, and cyano.

Paragraph 9. A compound according to paragraph 8, or a pharmaceutically acceptable salt thereof, wherein R₅ is hydrogen, and R₆ and R₇ are linked together to form a cyclopropyl ring system.

Paragraph 10. A compound according to paragraph 1, or a pharmaceutically acceptable salt thereof, wherein R₅ is hydrogen, and R₇ and R₃ are linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano.

Paragraph 11. A compound according to any preceeding paragraphs, or a pharmaceutically acceptable salt thereof, wherein R, is methyl.

Paragraph 12. A compound according to any preceeding paragraphs, or a pharmaceutically acceptable salt thereof, wherein R₂ is hydrogen.

Paragraph 13. A compound according to any preceeding paragraphs, or a pharmaceutically acceptable salt thereof, wherein Y is NR₁₀ and R₁₀ is hydrogen.

Paragraph 14. A compound according to any preceeding paragraphs, or a pharmaceutically acceptable salt thereof, wherein X is N.

Paragraph 15. A compound according to any preceeding paragraphs, or a pharmaceutically acceptable salt thereof, wherein R₃ is selected from methyl, ethyl, isopropyl, t-butyl, cyclopropyl, cyclobutyl, phenyl,

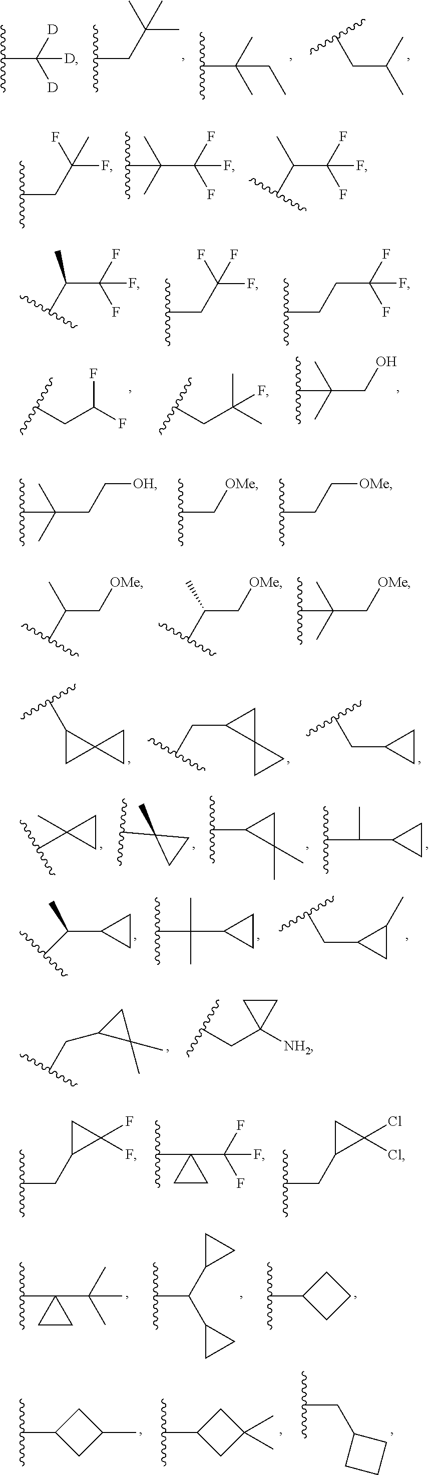

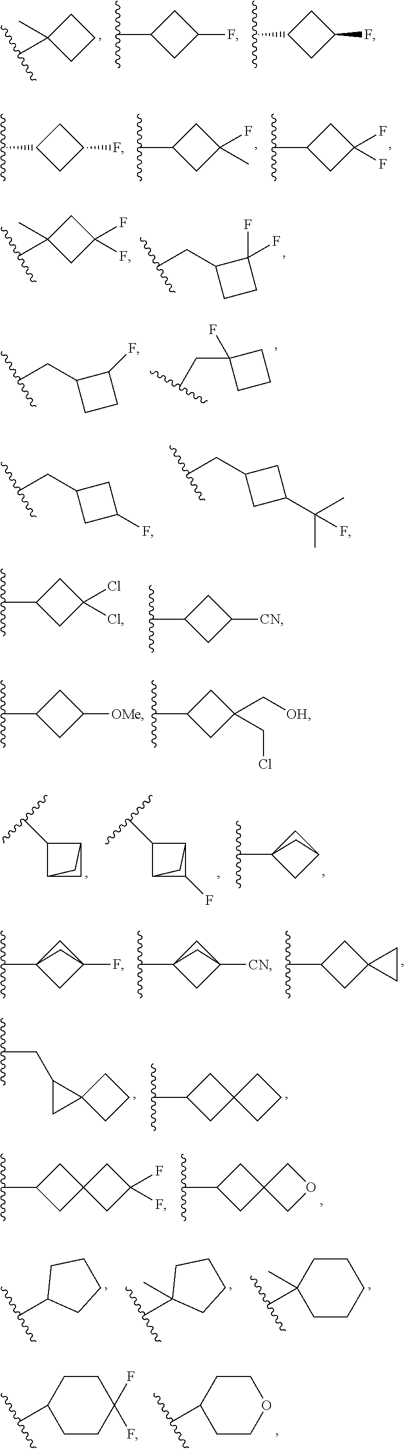

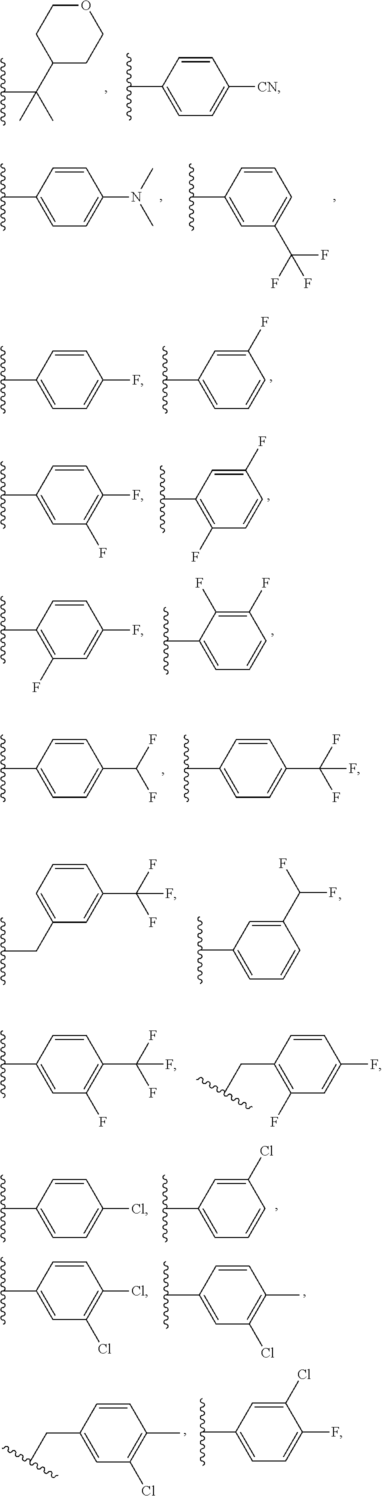

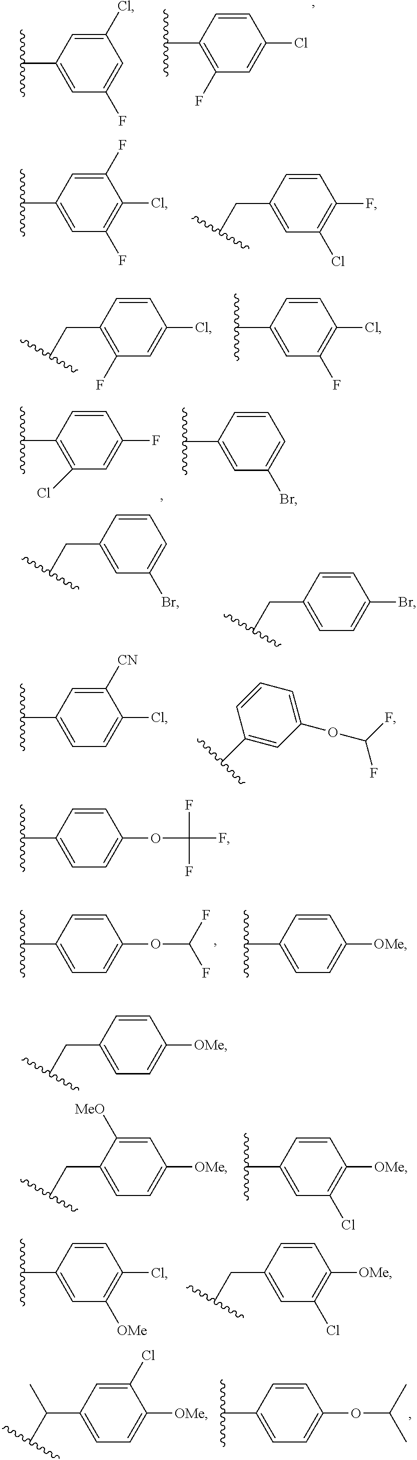

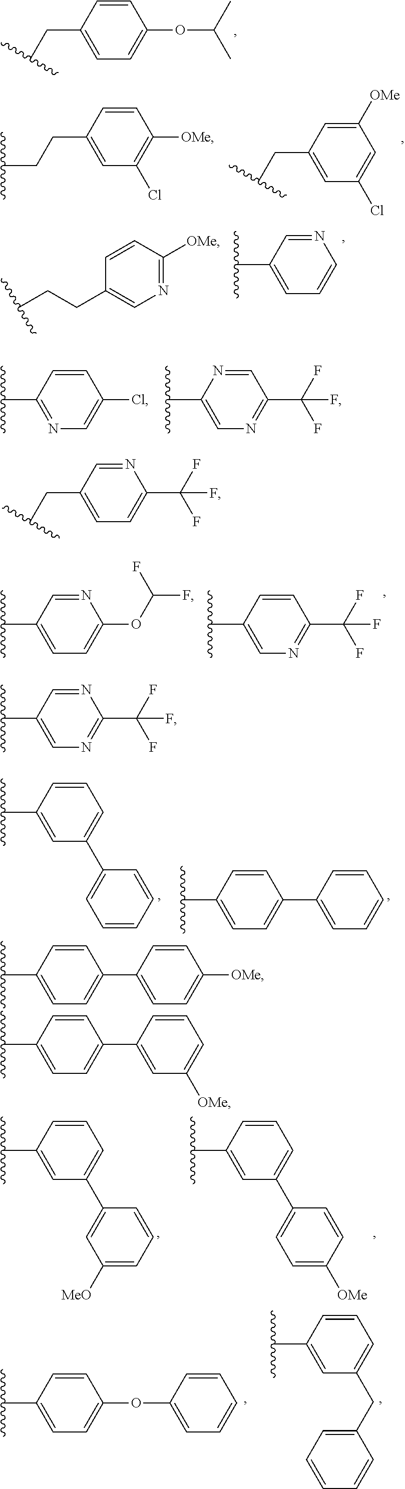

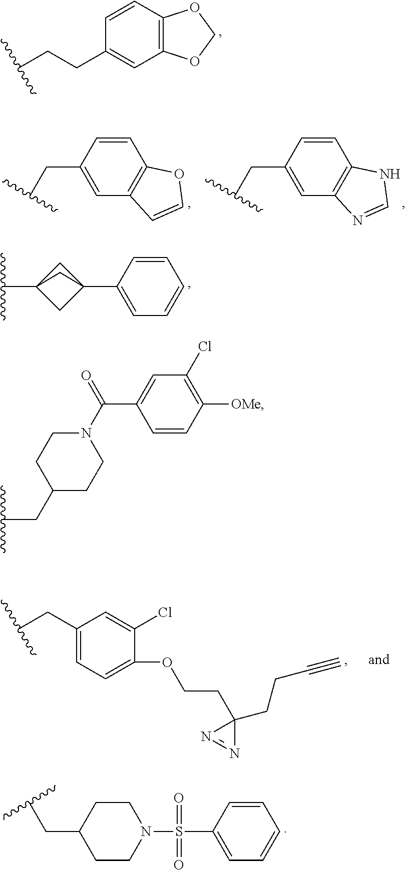

Paragraph 16. A compound, or a pharmaceutically acceptable salt thereof, selected from any one of the following:
N6-(2-Aminoethyl)-1-methyl-N4-[4-(trifluoromethyl)phenyl]pyrazolo[3,4-d]pyrimidine-4,6-diamine;
N6-(2-aminoethyl)-1-methyl-N4-[3-(trifluoromethyl)phenyl]-1H-pyrazolo[3,4-d]pyrimidine-4,6-diamine;
N6-(2-aminoethyl)-1-methyl-N4-(4-phenoxyphenyl)-1H-pyrazolo[3,4-d]pyrimidine-4,6-diamine;
N6-(2-aminoethyl)-1-methyl-N4-(3-phenoxyphenyl)-1H-pyrazolo[3,4-d]pyrimidine-4,6-diamine;
N6-(2-aminoethyl)-1-methyl-N4-[(4-phenylphenyl)methyl]pyrazolo[3,4-d]pyrimidine-4,6-diamine;
N6-(2-aminoethyl)-N4-[1-(3-chloro-4-methoxyphenyl)ethyl]-1-methyl-1H-pyrazolo[3,4-d]pyrimidine-4,6-diamine;
N6-(2-aminoethyl)-N4-[2-(6-methoxypyridin-3-yl)ethyl]-1-methyl-1H-pyrazolo[3,4-d]pyrimidine-4,6-diamine;
N6-(2-aminoethyl)-N4-[2-(3-chloro-4-methoxyphenyl)ethyl]-1-methyl-1H-pyrazolo[3,4-d]pyrimidine-4,6-diamine;
N6-(2-aminoethyl)-N4-[2-(2H-1,3-benzodioxol-5-yl)ethyl]-1-methyl-1H-pyrazolo[3,4-d]pyrimidine-4,6-diamine;
N6-(2-aminoethyl)-N4-[(1-benzofuran-5-yl)methyl]-1-methyl-1H-pyrazolo[3,4-d]pyrimidine-4,6-diamine;
N6-(2-aminoethyl)-N4-{3′-methoxy-[1,1′-biphenyl]-3-yl}-1-methyl-1H-pyrazolo[3,4-d]pyrimidine-4,6-diamine;
N6-(2-aminoethyl)-N4-{[1-(benzenesulfonyl)piperidin-4-yl]methyl}-1-methyl-1H-pyrazolo[3,4-d]pyrimidine-4,6-diamine;
N6-(2-aminoethyl)-N4-[(1H-1,3-benzodiazol-5-yl)methyl]-1-methyl-1H-pyrazolo[3,4-d]pyrimidine-4,6-diamine;
N6-(2-aminoethyl)-N4-{3′-methoxy-[1,1′-biphenyl]-4-yl}-1-methyl-1H-pyrazolo[3,4-d]pyrimidine-4,6-diamine;
N6-(2-aminoethyl)-N4-{4′-methoxy-[1,1′-biphenyl]-3-yl}-1-methyl-1H-pyrazolo[3,4-d]pyrimidine-4,6-diamine;
N6-(2-aminoethyl)-N4-{[1,1′-biphenyl]-3-yl}-1-methyl-1H-pyrazolo[3,4-d]pyrimidine-4,6-diamine;
N6-(2-aminoethyl)-N4-[(3-chloro-4-fluorophenyl)methyl]-1-methyl-1H-pyrazolo[3,4-d]pyrimidine-4,6-diamine;
N6-(2-aminoethyl)-N4-[(4-chloro-2-fluorophenyl)methyl]-1-methyl-1H-pyrazolo[3,4-d]pyrimidine-4,6-diamine;
N6-(2-aminoethyl)-N4-[(2,4-difluorophenyl)methyl]-1-methyl-1H-pyrazolo[3,4-d]pyrimidine-4,6-diamine;
N6-(2-aminoethyl)-N4-[(3-chloro-5-methoxyphenyl)methyl]-1-methyl-1H-pyrazolo[3,4-d]pyrimidine-4,6-diamine;
N6-(2-aminoethyl)-N4-(3-chloro-4-methoxyphenyl)-1-methyl-1H-pyrazolo[3,4-d]pyrimidine-4,6-diamine;
N6-(2-aminoethyl)-N4-[(3-chloro-4-methylphenyl)methyl]-1-methyl-1H-pyrazolo[3,4-d]pyrimidine-4,6-diamine;
N6-(2-aminoethyl)-1-methyl-N4-{[3-(trifluoromethyl)phenyl]methyl}-1H-pyrazolo[3,4-d]pyrimidine-4,6-diamine;
N6-(2-aminoethyl)-N4-[(2,4-dimethoxyphenyl)methyl]-1-methyl-1H-pyrazolo[3,4-d]pyrimidine-4,6-diamine;
N6-(2-aminoethyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(4-methoxyphenyl)methyl]-1-methyl-1H-pyrazolo[3,4-d]pyrimidine-4,6-diamine;
N6-(2-amino-2-methylpropyl)-N4-({[1,1′-biphenyl]-4-yl}methyl)-1-methyl-1H-pyrazolo[3,4-d]pyrimidine-4,6-diamine;
N6-(2-aminoethyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-({[1,1′-biphenyl]-4-yl}methyl)-1,3-dimethyl-1H-pyrazolo[3,4-d]pyrimidine-4,6-diamine;
N6-[(1-aminocyclohexyl)methyl]-1-methyl-N4-[4-(trifluoromethyl)phenyl]pyrazolo[3,4-d]pyrimidine-4,6-diamine;
N6-(2-amino-2-methylpropyl)-1,3-dimethyl-N4-[3-(trifluoromethyl)phenyl]-1H-pyrazolo[3,4-d]pyrimidine-4,6-diamine;
6-N-(2-aminocyclohexyl)-4-N-[(3-chloro-4-methylphenyl)methyl]-1-methylpyrazolo[3,4-d]pyrimidine-4,6-diamine;
6-N-[(4-aminooxan-4-yl)methyl]-1-methyl-4-N-[6-(trifluoromethyl)pyridin-3-yl]pyrazolo[3,4-d]pyrimidine-4,6-diamine;
N6-[(3R,4S)-4-aminotetrahydrofuran-3-yl]-1-methyl-N4-[4-(trifluoromethyl)phenyl]pyrazolo[3,4-d]pyrimidine-4,6-diamine;
N6-(2-aminoethyl)-N4-{[1-(3-chloro-4-methoxybenzoyl)piperidin-4-yl]methyl}-1-methyl-1H-pyrazolo[3,4-d]pyrimidine-4,6-diamine;
6-N-(2-Amino-2-methylpropyl)-1,3-dimethyl-4-N-[[3-(trifluoromethyl)phenyl]methyl]pyrazolo[3,4-b]pyridine-4,6-diamine;
6-N-[(1-Aminocyclopropyl)methyl]-4-N-[(3-chloro-4-methylphenyl)methyl]-1-methylpyrazolo[3,4-d]pyrimidine-4,6-diamine;
6-N-[(1-Aminocyclopropyl)methyl]-4-N-[(3-chloro-4-fluorophenyl)methyl]-1-methylpyrazolo[3,4-d]pyrimidine-4,6-diamine;
N6-[(1-aminocyclopropyl)methyl]-N4-[3-fluoro-4-(trifluoromethyl)phenyl]-1-methyl-pyrazolo[3,4-d]pyrimidine-4,6-diamine;
6-N-[(1-aminocyclopropyl)methyl]-4-N-(3-chloro-4-methoxyphenyl)-1-methylpyrazolo[3,4-d]pyrimidine-4,6-diamine;
6-N-[(1-aminocyclopropyl)methyl]-4-N-(2,4-difluorophenyl)-1-methylpyrazolo[3,4-d]pyrimidine-4,6-diamine;
6-N-[(1-aminocyclopropyl)methyl]-1-methyl-4-N-[3-(trifluoromethyl)phenyl]pyrazolo[3,4-d]pyrimidine-4,6-diamine;
6-N-[(1-aminocyclopropyl)methyl]-4-N-[3-(difluoromethyl)phenyl]-1-methylpyrazolo[3,4-d]pyrimidine-4,6-diamine;
6-N-[(1-aminocyclopropyl)methyl]-4-N-(4-fluorophenyl)-1-methylpyrazolo[3,4-d]pyrimidine-4,6-diamine;
6-N-[(1-aminocyclopropyl)methyl]-4-N-(3-chloro-4-methylphenyl)-1-methylpyrazolo[3,4-d]pyrimidine-4,6-diamine;
6-N-[(1-aminocyclopropyl)methyl]-1-methyl-4-N-pyridin-3-ylpyrazolo[3,4-d]pyrimidine-4,6-diamine;
6-N-[(1-aminocyclopropyl)methyl]-1-methyl-4-N-phenylpyrazolo[3,4-d]pyrimidine-4,6-diamine;
6-N-[(1-aminocyclopropyl)methyl]-4-N-[(3-bromophenyl)methyl]-1-methylpyrazolo[3,4-d]pyrimidine-4,6-diamine;
6-N-[(1-aminocyclopropyl)methyl]-4-N-[(4-bromophenyl)methyl]-1-methylpyrazolo[3,4-d]pyrimidine-4,6-diamine;
6-N-[(1-aminocyclopropyl)methyl]-4-N-(2-chloro-4-fluorophenyl)-1-methylpyrazolo[3,4-d]pyrimidine-4,6-diamine;
6-N-[(1-aminocyclopropyl)methyl]-4-N-(4-chloro-2-fluorophenyl)-1-methylpyrazolo[3,4-d]pyrimidine-4,6-diamine;
6-N-[(1-aminocyclopropyl)methyl]-4-N-(3-chloro-5-fluorophenyl)-1-methylpyrazolo[3,4-d]pyrimidine-4,6-diamine;
5-[[6-[(1-aminocyclopropyl)methylamino]-1-methyl-pyrazolo[3,4-d]pyrimidin-4-yl]amino]-2-chloro-benzonitrile;
N6-[(1-aminocyclopropyl)methyl]-1-methyl-N4-(1-phenyl-3-bicyclo[1.1.1]pentanyl)pyrazolo[3,4-d]pyrimidine-4,6-diamine;
6-N-[(1-aminocyclopropyl)methyl]-1-methyl-4-N-[[6-(trifluoromethyl)pyridin-3-yl]methyl]pyrazolo[3,4-d]pyrimidine-4,6-diamine;
6-N-[(1-aminocyclopropyl)methyl]-4-N-(4-chloro-3,5-difluorophenyl)-1-methylpyrazolo[3,4-d]pyrimidine-4,6-diamine;
6-N-[(1-aminocyclopropyl)methyl]-4-N-[3-(difluoromethoxy)phenyl]-1-methylpyrazolo[3,4-d]pyrimidine-4,6-diamine;
6-N-[(1-aminocyclopropyl)methyl]-1-methyl-4-N-(4-propan-2-yloxyphenyl)pyrazolo[3,4-d]pyrimidine-4,6-diamine;
6-N-[(1-aminocyclopropyl)methyl]-4-N-[(4-chloro-3-fluorophenyl)methyl]-1-methylpyrazolo[3,4-d]pyrimidine-4,6-diamine;
6-N-[(1-aminocyclopropyl)methyl]-4-N-(2,3-difluorophenyl)-1-methylpyrazolo[3,4-d]pyrimidine-4,6-diamine;
6-N-[(1-aminocyclopropyl)methyl]-4-N-(2,5-difluorophenyl)-1-methylpyrazolo[3,4-d]pyrimidine-4,6-diamine;
6-N-[(1-aminocyclopropyl)methyl]-4-N-[(2,4-difluorophenyl)methyl]-1-methylpyrazolo[3,4-d]pyrimidine-4,6-diamine;
6-N-[(1-aminocyclopropyl)methyl]-4-N-(3-chloro-4-fluorophenyl)-1-methylpyrazolo[3,4-d]pyrimidine-4,6-diamine;
6-N-[(1-aminocyclopropyl)methyl]-4-N-(4-chlorophenyl)-1-methylpyrazolo[3,4-d]pyrimidine-4,6-diamine;
6-N-[(1-aminocyclopropyl)methyl]-4-N-(3-fluorophenyl)-1-methylpyrazolo[3,4-d]pyrimidine-4,6-diamine;
6-N-[(1-aminocyclopropyl)methyl]-1,3-dimethyl-4-N-[4-(trifluoromethyl)phenyl]pyrazolo[3,4-d]pyrimidine-4,6-diamine;
4-[[6-[(1-aminocyclopropyl)methylamino]-1-methylpyrazolo[3,4-d]pyrimidin-4-yl]amino]benzonitrile;
6-N-[(1-aminocyclopropyl)methyl]-1-methyl-4-N-[4-(trifluoromethoxy)phenyl]pyrazolo[3,4-d]pyrimidine-4,6-diamine;
6-N-[(1-aminocyclopropyl)methyl]-4-N-(4-methoxyphenyl)-1-methylpyrazolo[3,4-d]pyrimidine-4,6-diamine;
6-N-[(1-aminocyclopropyl)methyl]-4-N-[4-(difluoromethoxy)phenyl]-1-methylpyrazolo[3,4-d]pyrimidine-4,6-diamine;
6-N-[(1-aminocyclopropyl)methyl]-4-N-(3-chlorophenyl)-1-methylpyrazolo[3,4-d]pyrimidine-4,6-diamine;
6-N-[(1-aminocyclopropyl)methyl]-4-N-[4-(difluoromethyl)phenyl]-1-methylpyrazolo[3,4-d]pyrimidine-4,6-diamine;
3-[[6-[(1-aminocyclopropyl)methylamino]-1-methylpyrazolo[3,4-d]pyrimidin-4-yl]amino]bicyclo[1.1.1]pentane-1-carbonitrile;
6-N-[(1-aminocyclopropyl)methyl]-4-N-(3-bicyclo[1.1.1]pentanyl)-1-methylpyrazolo[3,4-d]pyrimidine-4,6-diamine;
6-N-[(1-aminocyclopropyl)methyl]-4-N-(3,4-difluorophenyl)-1-methylpyrazolo[3,4-d]pyrimidine-4,6-diamine;
6-N-[(1-aminocyclopropyl)methyl]-4-N-(3,4-dichlorophenyl)-1-methylpyrazolo[3,4-d]pyrimidine-4,6-diamine;
6-N-[(1-aminocyclopropyl)methyl]-4-N-(4-chloro-3-methoxyphenyl)-1-methylpyrazolo[3,4-d]pyrimidine-4,6-diamine;
N6-[(1-aminocyclopropyl)methyl]-N4-[(2,4-dimethoxyphenyl)methyl]-1-methyl-pyrazolo[3,4-d]pyrimidine-4,6-diamine;
N6-[(1-aminocyclopropyl)methyl]-N4-{3-fluorobicyclo[1.1.1]pentan-1-yl}-1-methyl-1H-pyrazolo[3,4-d]pyrimidine-4,6-diamine;
6-N-[(1-aminocyclopropyl)methyl]-1-methyl-4-N-[4-(trifluoromethyl)phenyl]pyrazolo[3,4-d]pyrimidine-4,6-diamine;
6-N-[(1-aminocyclopropyl)methyl]-1-methyl-4-N-[6-(trifluoromethyl)pyridin-3-yl]pyrazolo[3,4-d]pyrimidine-4,6-diamine;
6-N-[(1-aminocyclopropyl)methyl]-4-N-(5-chloropyridin-2-yl)-1-methylpyrazolo[3,4-d]pyrimidine-4,6-diamine;
N6-[(1-aminocyclopropyl)methyl]-1-methyl-N4-[5-(trifluoromethyl)pyrazin-2-yl]pyrazolo[3,4-d]pyrimidine-4,6-diamine;
6-N-[(1-aminocyclopropyl)methyl]-1-ethyl-4-N-[4-(trifluoromethyl)phenyl]pyrazolo[3,4-d]pyrimidine-4,6-diamine;
N6-[(1-aminocyclopropyl)methyl]-1-(trideuteriomethyl)-N4-[4-(trifluoromethyl)phenyl]pyrazolo[3,4-d]pyrimidine-4,6-diamine;
N6-[(1-aminocyclopropyl)methyl]-1-(trideuteriomethyl)-N4-[6-(trifluoromethyl)-3-pyridyl]pyrazolo[3,4-d]pyrimidine-4,6-diamine;
6-N-[(1-aminocyclopropyl)methyl]-4-N-[[4-[2-(3-but-3-ynyldiazirin-3-yl)ethoxy]-3-chlorophenyl]methyl]-1-methylpyrazolo[3,4-d]pyrimidine-4,6-diamine;
N6-[(1-Aminocyclopropyl)methyl]-1-methyl-N4-[4-(trifluoromethyl)phenyl]pyrazolo[3,4-b]pyridine-4,6-diamine;
6-N-(2-aminopropyl)-4-N-[(3-chloro-4-methylphenyl)methyl]-1-methylpyrazolo[3,4-d]pyrimidine-4,6-diamine;
6-N-(1-aminopropan-2-yl)-4-N-[(3-chloro-4-methylphenyl)methyl]-1-methylpyrazolo[3,4-d]pyrimidine-4,6-diamine;
6-N-[(2S)-2-aminopropyl]-4-N-[(3-chloro-4-methylphenyl)methyl]-1-methylpyrazolo[3,4-d]pyrimidine-4,6-diamine;
6-N-[(2R)-2-aminopropyl]-4-N-[(3-chloro-4-methylphenyl)methyl]-1-methylpyrazolo[3,4-d]pyrimidine-4,6-diamine;
6-N-[(2S)-1-aminobutyl]-4-N-[(3-chloro-4-methylphenyl)methyl]-1-methylpyrazolo[3,4-d]pyrimidine-4,6-diamine;
6-N-[(2S)-2-aminobutyl]-4-N-[(3-chloro-4-methylphenyl)methyl]-1-methylpyrazolo[3,4-d]pyrimidine-4,6-diamine;
6-N-[(2R)-2-aminobutyl]-4-N-[(3-chloro-4-methylphenyl)methyl]-1-methylpyrazolo[3,4-d]pyrimidine-4,6-diamine;
N6-[(1-aminocyclobutyl)methyl]-1-methyl-N4-[4-(trifluoromethyl)phenyl]pyrazolo[3,4-d]pyrimidine-4,6-diamine;
N6-[(1-aminocyclobutyl)methyl]-N4-[4-(difluoromethoxy)phenyl]-1-methyl-pyrazolo[3,4-d]pyrimidine-4,6-diamine;
N6-(2-amino-2-cyclohexyl-ethyl)-N4-tert-butyl-1-methyl-pyrazolo[3,4-d]pyrimidine-4,6-diamine;
N6-(2-amino-2-cyclohexyl-ethyl)-1-methyl-N4-[4-(trifluoromethyl)phenyl]pyrazolo[3,4-d]pyrimidine-4,6-diamine;
6-N-[(1-amino-3,3-difluorocyclobutyl)methyl]-1-methyl-4-N-[4-(trifluoromethyl)phenyl]pyrazolo[3,4-d]pyrimidine-4,6-diamine;
6-N-(2-amino-2-cyclopropylethyl)-1-methyl-4-N-[4-(trifluoromethyl)phenyl]pyrazolo[3,4-d]pyrimidine-4,6-diamine;
6-N-[(3-aminooxolan-3-yl)methyl]-1-methyl-4-N-[4-(trifluoromethyl)phenyl]pyrazolo[3,4-d]pyrimidine-4,6-diamine;
N6-[(3-aminotetrahydrofuran-3-yl)methyl]-1-methyl-N4-[6-(trifluoromethyl)-3-pyridyl]pyrazolo[3,4-d]pyrimidine-4,6-diamine;
1-methyl-N6-[(morpholin-3-yl)methyl]-N4-[4-(trifluoromethyl)phenyl]-1H-pyrazolo[3,4-d]pyrimidine-4,6-diamine;
4-N-[(3-chloro-4-methylphenyl)methyl]-1-methyl-6-N-[[(2R)-pyrrolidin-2-yl]methyl]pyrazolo[3,4-d]pyrimidine-4,6-diamine;
6-N-[(1-aminocyclopentyl)methyl]-1-methyl-4-N-[4-(trifluoromethyl)phenyl]pyrazolo[3,4-d]pyrimidine-4,6-diamine;
6-N-[(1-aminocyclopentyl)methyl]-1-methyl-4-N-[6-(trifluoromethyl)pyridin-3-yl]pyrazolo[3,4-d]pyrimidine-4,6-diamine;
6-N-[(1-aminocyclopentyl)methyl]-4-N-[4-(difluoromethoxy)phenyl]-1-methylpyrazolo[3,4-d]pyrimidine-4,6-diamine;
6-N-[(1-aminocyclopentyl)methyl]-1-methyl-4-N-phenylpyrazolo[3,4-d]pyrimidine-4,6-diamine;
4-[[6-[(1-aminocyclopentyl)methylamino]-1-methylpyrazolo[3,4-d]pyrimidin-4-yl]amino]benzonitrile;
6-N-[(1-aminocyclopentyl)methyl]-1-methyl-4-N-[4-(trifluoromethoxy)phenyl]pyrazolo[3,4-d]pyrimidine-4,6-diamine;
N6-[(1-aminocyclobutyl)methyl]-1-(trideuteriomethyl)-N4-[4-(trifluoromethyl)phenyl]pyrazolo[3,4-d]pyrimidine-4,6-diamine;
N6-(2-Amino-3-fluoropropyl)-N4-[(3-chloro-4-methylphenyl)methyl]-1-methyl-1H-pyrazolo[3,4-d]pyrimidine-4,6-diamine;
6-N-(2-amino-3-fluoropropyl)-1-methyl-4-N-[4-(trifluoromethyl)phenyl]pyrazolo[3,4-d]pyrimidine-4,6-diamine;
6-N-(2-amino-3-methoxypropyl)-4-N-[(3-chloro-4-methylphenyl)methyl]-1-methylpyrazolo[3,4-d]pyrimidine-4,6-diamine;
6-N-[(3-Aminooxetan-3-yl)methyl]-1-methyl-4-N-[4-(trifluoromethyl)phenyl]pyrazolo[3,4-d]pyrimidine-4,6-diamine;
6-N-[(3-Aminooxetan-3-yl)methyl]-1-methyl-4-N-phenylpyrazolo[3,4-d]pyrimidine-4,6-diamine;
N6-(2-Amino-2-phenyl-propyl)-N4-tert-butyl-1-(trideuteriomethyl)pyrazolo[3,4-d]pyrimidine-4,6-diamine;
6-N-(2-Amino-2-phenylethyl)-1-ethyl-4-N-propan-2-ylpyrazolo[3,4-d]pyrimidine-4,6-diamine;
6-N-(2-amino-2-phenylethyl)-4-N-[(3-chloro-4-methylphenyl)methyl]-1-methylpyrazolo[3,4-d]pyrimidine-4,6-diamine;
6-N-(2-amino-2-phenylethyl)-4-N-[(2,4-difluorophenyl)methyl]-1-methylpyrazolo[3,4-d]pyrimidine-4,6-diamine;
6-N-(2-amino-2-phenylethyl)-4-N-[(3-chloro-4-fluorophenyl)methyl]-1-methylpyrazolo[3,4-d]pyrimidine-4,6-diamine;
6-N-(2-amino-2-phenylethyl)-4-N-[(3-bromophenyl)methyl]-1-methylpyrazolo[3,4-d]pyrimidine-4,6-diamine;
6-N-(2-amino-2-phenylethyl)-4-N-[(4-bromophenyl)methyl]-1-methylpyrazolo[3,4-d]pyrimidine-4,6-diamine;
6-N-(2-amino-2-phenylethyl)-1-methyl-4-N-[[6-(trifluoromethyl)pyridin-3-yl]methyl]pyrazolo[3,4-d]pyrimidine-4,6-diamine;
N6-(2-amino-2-phenyl-ethyl)-N4-[(2,4-dimethoxyphenyl)methyl]-1-methyl-pyrazolo[3,4-d]pyrimidine-4,6-diamine;
6-N-(2-amino-2-phenylethyl)-4-N-(4-fluorophenyl)-1-methylpyrazolo[3,4-d]pyrimidine-4,6-diamine;
N6-(2-amino-2-phenyl-propyl)-1-methyl-N4-[2-(trifluoromethyl)pyrimidin-5-yl]pyrazolo[3,4-d]pyrimidine-4,6-diamine;
N6-(2-amino-2-phenyl-propyl)-1-methyl-N4-[4-(trifluoromethoxy)phenyl]pyrazolo[3,4-d]pyrimidine-4,6-diamine;
N6-(2-amino-2-phenyl-propyl)-N4-(3,4-dichlorophenyl)-1-methyl-pyrazolo[3,4-d]pyrimidine-4,6-diamine;
6-N-(2-amino-2-phenylethyl)-4-N-[4-(difluoromethoxy)phenyl]-1-methylpyrazolo[3,4-d]pyrimidine-4,6-diamine;
6-N-(2-amino-2-phenylpropyl)-4-N-[4-(difluoromethoxy)phenyl]-1-methylpyrazolo[3,4-d]pyrimidine-4,6-diamine;
N6-[(2R)-2-amino-2-phenyl-ethyl]-N4-[4-(difluoromethoxy)phenyl]-1-methyl-pyrazolo[3,4-d]pyrimidine-4,6-diamine;
6-N-(2-amino-2-phenylethyl)-1-methyl-4-N-[4-(trifluoromethyl)phenyl]pyrazolo[3,4-d]pyrimidine-4,6-diamine;
6-N-(2-amino-2-phenylethyl)-1,3-dimethyl-4-N-[4-(trifluoromethyl)phenyl]pyrazolo[3,4-d]pyrimidine-4,6-diamine;
N6-(2-amino-2-phenyl-propyl)-1-methyl-N4-[4-(trifluoromethyl)phenyl]pyrazolo[3,4-d]pyrimidine-4,6-diamine;
N6-(2-amino-2-phenyl-ethyl)-1-(trideuteriomethyl)-N4-[4-(trifluoromethyl)phenyl]pyrazolo[3,4-d]pyrimidine-4,6-diamine;
N6-(2-amino-2-phenyl-propyl)-1-(trideuteriomethyl)-N4-[4-(trifluoromethyl)phenyl]pyrazolo[3,4-d]pyrimidine-4,6-diamine;
6-N-[(2R)-2-amino-2-phenylethyl]-1-methyl-4-N-[4-(trifluoromethyl)phenyl]pyrazolo[3,4-d]pyrimidine-4,6-diamine;
6-N-[(2S)-2-amino-2-phenylethyl]-1-methyl-4-N-[4-(trifluoromethyl)phenyl]pyrazolo[3,4-d]pyrimidine-4,6-diamine;
N6-[(2R)-2-amino-2-phenyl-ethyl]-1-(trideuteriomethyl)-N4-[4-(trifluoromethyl)phenyl]pyrazolo[3,4-d]pyrimidine-4,6-diamine;
6-N-(2-amino-2-phenylethyl)-1-methyl-4-N-[6-(trifluoromethyl)pyridin-3-yl]pyrazolo[3,4-d]pyrimidine-4,6-diamine;
6-N-(2-amino-2-phenylpropyl)-1-methyl-4-N-[6-(trifluoromethyl)pyridin-3-yl]pyrazolo[3,4-d]pyrimidine-4,6-diamine;
N6-(2-amino-2-phenyl-propyl)-1-(trideuteriomethyl)-N4-[6-(trifluoromethyl)-3-pyridyl]pyrazolo[3,4-d]pyrimidine-4,6-diamine;
N6-(2-amino-2-phenyl-ethyl)-N4-[6-(difluoromethoxy)-3-pyridyl]-1-methyl-pyrazolo[3,4-d]pyrimidine-4,6-diamine;
N6-(2-amino-2-phenyl-propyl)-N4-[6-(difluoromethoxy)-3-pyridyl]-1-methyl-pyrazolo[3,4-d]pyrimidine-4,6-diamine;
N6-[(2R)-2-amino-2-phenyl-ethyl]-N4-(4-chlorophenyl)-1-methyl-pyrazolo[3,4-d]pyrimidine-4,6-diamine;
N6-[(2S)-2-amino-2-phenyl-ethyl]-N4-(4-chlorophenyl)-1-methyl-pyrazolo[3,4-d]pyrimidine-4,6-diamine;
N6-[(2R)-2-amino-2-phenyl-ethyl]-N4-(4-methoxyphenyl)-1-methyl-pyrazolo[3,4-d]pyrimidine-4,6-diamine;
N6-[(2R)-2-amino-2-phenyl-ethyl]-N4-(4-isopropoxyphenyl)-1-methyl-pyrazolo[3,4-d]pyrimidine-4,6-diamine;
N6-[(2R)-2-amino-2-phenyl-ethyl]-N4-(3,4-difluorophenyl)-1-methyl-pyrazolo[3,4-d]pyrimidine-4,6-diamine;
N6-[(2R)-2-amino-2-phenyl-ethyl]-N4-[4-(dimethylamino)phenyl]-1-methyl-pyrazolo[3,4-d]pyrimidine-4,6-diamine;
6-N-(2-amino-2-phenylethyl)-4-N,1-dimethylpyrazolo[3,4-d]pyrimidine-4,6-diamine;
6-N-(2-amino-2-phenylethyl)-1-methyl-4-N-(trideuteriomethyl)pyrazolo[3,4-d]pyrimidine-4,6-diamine;
6-N-(2-amino-2-phenylethyl)-4-N-methyl-1-(trideuteriomethyl)pyrazolo[3,4-d]pyrimidine-4,6-diamine;
6-N-(2-amino-2-phenylpropyl)-4-N,1-dimethylpyrazolo[3,4-d]pyrimidine-4,6-diamine;
6-N-[(2R)-2-amino-2-phenylethyl]-4-N,1-dimethylpyrazolo[3,4-d]pyrimidine-4,6-diamine;
6-N-(2-amino-2-phenylethyl)-4-N-ethyl-1-methylpyrazolo[3,4-d]pyrimidine-4,6-diamine;
6-N-(2-amino-2-phenylethyl)-4-N-ethyl-4-N,1-dimethylpyrazolo[3,4-d]pyrimidine-4,6-diamine;
6-N-(2-amino-2-phenylethyl)-4-N-cyclopropyl-1-methylpyrazolo[3,4-d]pyrimidine-4,6-diamine;
6-N-(2-amino-2-phenylethyl)-1-methyl-4-N-(2,2,2-trifluoroethyl)pyrazolo[3,4-d]pyrimidine-4,6-diamine;
N6-[(2R)-2-amino-2-phenyl-ethyl]-1-methyl-N4-(2,2,2-trifluoroethyl)pyrazolo[3,4-d]pyrimidine-4,6-diamine;
6-N-(2-amino-2-phenylethyl)-1-methyl-4-N-propan-2-ylpyrazolo[3,4-d]pyrimidine-4,6-diamine;
6-N-[(2R)-2-amino-2-phenylethyl]-1-methyl-4-N-propan-2-ylpyrazolo[3,4-d]pyrimidine-4,6-diamine;
N′-(1-methyl-4-pyrrolidin-1-ylpyrazolo[3,4-d]pyrimidin-6-yl)-1-phenylethane-1,2-diamine;
6-N-(2-amino-2-phenylethyl)-4-N-(2-methoxyethyl)-1-methylpyrazolo[3,4-d]pyrimidine-4,6-diamine;
6-N-(2-amino-2-phenylethyl)-4-N-tert-butyl-1-methylpyrazolo[3,4-d]pyrimidine-4,6-diamine:
N6-(2-amino-2-phenyl-propyl)-N4-tert-butyl-1-methyl-pyrazolo[3,4-d]pyrimidine-4,6-diamine;
N6-(2-amino-2-phenyl-ethyl)-N4-tert-butyl-1-(trideuteriomethyl)pyrazolo[3,4-d]pyrimidine-4,6-diamine;
6-N-[(2R)-2-amino-2-phenylethyl]-4-N-tert-butyl-1-methylpyrazolo[3,4-d]pyrimidine-4,6-diamine;
6-N-[(2S)-2-amino-2-phenylethyl]-4-N-tert-butyl-1-methylpyrazolo[3,4-d]pyrimidine-4,6-diamine;
N6-[(2R)-2-amino-2-phenyl-ethyl]-N4-tert-butyl-1-(trideuteriomethyl)pyrazolo[3,4-d]pyrimidine-4,6-diamine;
6-N-(2-amino-2-phenylethyl)-4-N-(cyclopropylmethyl)-1-methylpyrazolo[3,4-d]pyrimidine-4,6-diamine;
N6-(2-amino-2-phenyl-ethyl)-N4-(dicyclopropylmethyl)-1-methyl-pyrazolo[3,4-d]pyrimidine-4,6-diamine;
N6-[(2R)-2-amino-2-phenyl-ethyl]-1-methyl-N4-[(2-methylcyclopropyl)methyl]pyrazolo[3,4-d]pyrimidine-4,6-diamine;
N6-[(2R)-2-amino-2-phenyl-ethyl]-N4-(1-cyclopropyl-1-methyl-ethyl)-1-methyl-pyrazolo[3,4-d]pyrimidine-4,6-diamine;
N6-[(2R)-2-amino-2-phenyl-ethyl]-1-methyl-N4-(spiro[2.2]pentan-2-ylmethyl) pyrazolo[3,4-d]pyrimidine-4,6-diamine;
N6-[(2R)-2-amino-2-phenyl-ethyl]-N4-[(2,2-difluorocyclopropyl)methyl]-1-methyl-pyrazolo[3,4-d]pyrimidine-4,6-diamine;
N6-[(2R)-2-amino-2-phenyl-ethyl]-N4-[(2,2-dimethylcyclopropyl)methyl]-1-methyl-pyrazolo[3,4-d]pyrimidine-4,6-diamine;
6-N-(2-amino-2-phenylethyl)-4-N-(2-bicyclo[1.1.1]pentanyl)-1-methylpyrazolo[3,4-d]pyrimidine-4,6-diamine;
3-[[6-[(2-amino-2-phenylethyl)amino]-1-methylpyrazolo[3,4-d]pyrimidin-4-yl]amino]bicyclo[1.1.1]pentane-1-carbonitrile;
N6-(2-amino-2-phenylethyl)-N4-{3-fluorobicyclo[1.1.1]pentan-1-yl}-1-methyl-1H-pyrazolo[3,4-d]pyrimidine-4,6-diamine;
N6-[(2R)-2-amino-2-phenyl-ethyl]-N4-(3-bicyclo[1.1.1]pentanyl)-1-methyl-pyrazolo[3,4-d]pyrimidine-4,6-diamine;
N6-[(2R)-2-amino-2-phenyl-ethyl]-N4-(3-fluoro-1-bicyclo[1.1.1]pentanyl)-1-methyl-pyrazolo[3,4-d]pyrimidine-4,6-diamine;
6-N-(2-amino-2-phenylethyl)-4-N-(4,4-difluorocyclohexyl)-1-methylpyrazolo[3,4-d]pyrimidine-4,6-diamine;
N6-[(2R)-2-amino-2-phenyl-ethyl]-1-methyl-N4-tetrahydropyran-4-yl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]-3-methyl-butan-1-ol;
2-[[6-[[(2R)-2-amino-2-phenyl-ethyl]amino]-1-methyl-pyrazolo[3,4-d]pyrimidin-4-yl]amino]-2-methyl-propan-1-ol;
N6-[(2R)-2-amino-2-phenyl-ethyl]-N4-(2,2-dimethylpropyl)-1-methyl-pyrazolo[3,4-d]pyrimidine-4,6-diamine;
N6-[(2R)-2-amino-2-phenyl-ethyl]-N4-(2,2-difluoroethyl)-1-methyl-pyrazolo[3,4-d]pyrimidine-4,6-diamine;
N6-[(2R)-2-amino-2-phenyl-ethyl]-N4-(2,2-difluoropropyl)-1-methyl-pyrazolo[3,4-d]pyrimidine-4,6-diamine;
N6-[(2R)-2-amino-2-phenyl-ethyl]-1-methyl-N4-(2,2,2-trifluoro-1,1-dimethyl-ethyl)pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-(1-methylcyclopropyl)pyrazolo[3,4-d]pyrimidine-4,6-diamine;
N6-[(2R)-2-amino-2-phenyl-ethyl]-1-methyl-N4-[1-(trifluoromethyl)cyclopropyl]pyrazolo[3,4-d]pyrimidine-4,6-diamine;
N6-[(2R)-2-amino-2-phenyl-ethyl]-N4-(1-tert-butylcyclopropyl)-1-methyl-pyrazolo[3,4-d]pyrimidine-4,6-diamine;
N6-[(2R)-2-amino-2-phenyl-ethyl]-1-methyl-N4-spiro[2.2]pentan-2-yl-pyrazolo[3,4-d]pyrimidine-4,6-diamine;
[3-[[6-[(2-amino-2-phenyl-ethyl)amino]-1-methyl-pyrazolo[3,4-d]pyrimidin-4-yl]amino]-1-(chloromethyl)cyclobutyl]methanol;
N6-(2-amino-2-phenyl-ethyl)-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-N4-cyclobutyl-1-methyl-pyrazolo[3,4-d]pyrimidine-4,6-diamine;
N6-[(2R)-2-amino-2-phenyl-ethyl]-1-methyl-N4-(1-methylcyclobutyl) pyrazolo[3,4-d]pyrimidine-4,6-diamine;
N6-[(2R)-2-amino-2-phenyl-ethyl]-N4-(3,3-difluorocyclobutyl)-1-methyl-pyrazolo[3,4-d]pyrimidine-4,6-diamine;
N6-[(2R)-2-amino-2-phenyl-ethyl]-N4-(3,3-difluoro-1-methyl-cyclobutyl)-1-methyl-pyrazolo[3,4-d]pyrimidine-4,6-diamine;
N6-[(2R)-2-amino-2-phenyl-ethyl]-1-methyl-N4-(3-methylcyclobutyl)pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]cyclobutanecarbonitrile;
N6-[(2R)-2-amino-2-phenyl-ethyl]-N4-(3-methoxycyclobutyl)-1-methyl-pyrazolo[3,4-d]pyrimidine-4,6-diamine;
N6-[(2R)-2-amino-2-phenyl-ethyl]-N4-(3,3-dichlorocyclobutyl)-1-methyl-pyrazolo[3,4-d]pyrimidine-4,6-diamine;
N6-[(2R)-2-amino-2-phenyl-ethyl]-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-1-methyl-N4-(2-oxaspiro[3.3]heptan-6-yl)pyrazolo[3,4-d]pyrimidine-4,6-diamine;
N6-[(2R)-2-amino-2-phenyl-ethyl]-N4-(3,3-dimethylcyclobutyl)-1-methyl-pyrazolo[3,4-d]pyrimidine-4,6-diamine;
N6-[(2R)-2-amino-2-phenyl-ethyl]-N4-(3-fluoro-3-methyl-cyclobutyl)-1-methyl-pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-spiro[3.3]heptan-2-yl-pyrazolo[3,4-d]pyrimidine-4,6-diamine;
N6-[(2R)-2-amino-2-phenyl-ethyl]-N4-(2-methoxy-1,1-dimethyl-ethyl)-1-methyl-pyrazolo[3,4-d]pyrimidine-4,6-diamine;
N6-[(2R)-2-amino-2-phenyl-ethyl]-N4-(1,1-dimethylpropyl)-1-methyl-pyrazolo[3,4-d]pyrimidine-4,6-diamine;
N6-[(2R)-2-amino-2-phenyl-ethyl]-N4-[(1S)-1-cyclopropylethyl]-1-methyl-pyrazolo[3,4-d]pyrimidine-4,6-diamine;
N6-[(2R)-2-amino-2-phenyl-ethyl]-N4-isobutyl-1-methyl-pyrazolo[3,4-d]pyrimidine-4,6-diamine;
N6-[(2R)-2-amino-2-phenyl-ethyl]-1-methyl-N4-(spiro[2.3]hexan-2-ylmethyl)pyrazolo[3,4-d]pyrimidine-4,6-diamine:
N6-[(2R)-2-amino-2-phenyl-ethyl]-N4-(cyclobutylmethyl)-1-methyl-pyrazolo[3,4-d]pyrimidine-4,6-diamine;
N6-[(2R)-2-amino-2-phenyl-ethyl]-N4-[(2,2-difluorocyclobutyl)methyl]-1-methyl-pyrazolo[3,4-d]pyrimidine-4,6-diamine;
N6-[(2R)-2-amino-2-phenyl-ethyl]-N4-[(1-fluorocyclobutyl)methyl]-1-methyl-pyrazolo[3,4-d]pyrimidine-4,6-diamine;
N6-[(2R)-2-amino-2-phenyl-ethyl]-N4-[(3-fluorocyclobutyl)methyl]-1-methyl-pyrazolo[3,4-d]pyrimidine-4,6-diamine;
N6-[(2R)-2-amino-2-phenyl-ethyl]-1-methyl-N4-[3-(trifluoromethyl)cyclobutyl]pyrazolo[3,4-d]pyrimidine-4,6-diamine;
N6-[(2R)-2-amino-2-phenyl-ethyl]-N4-(2-fluoro-2-methyl-propyl)-1-methyl-pyrazolo[3,4-d]pyrimidine-4,6-diamine;
N6-[(2R)-2-amino-2-phenyl-ethyl]-1-methyl-N4-(1-methylcyclohexyl)pyrazolo[3,4-d]pyrimidine-4,6-diamine;
N6-[(2R)-2-amino-2-phenyl-ethyl]-N4-[(2-fluorocyclobutyl)methyl]-1-methyl-pyrazolo[3,4-d]pyrimidine-4,6-diamine;
N6-[(2R)-2-amino-2-phenyl-ethyl]-N4-cyclopentyl-1-methyl-pyrazolo[3,4-d]pyrimidine-4,6-diamine;
N6-[(2R)-2-amino-2-phenyl-ethyl]-1-methyl-N4-(3,3,3-trifluoropropyl)pyrazolo[3,4-d]pyrimidine-4,6-diamine;
N6-[(2R)-2-amino-2-phenyl-ethyl]-N4-[(2,2-dichlorocyclopropyl)methyl]-1-methyl-pyrazolo[3,4-d]pyrimidine-4,6-diamine;
N6-[(2R)-2-amino-2-phenyl-ethyl]-1-methyl-N4-(1-methylcyclopentyl)pyrazolo[3,4-d]pyrimidine-4,6-diamine;
N6-[(2R)-2-amino-2-phenyl-ethyl]-N4-[(1R)-2-methoxy-1-methyl-ethyl]-1-methyl-pyrazolo[3,4-d]pyrimidine-4,6-diamine;
N6-[(2R)-2-amino-2-phenyl-ethyl]-1-methyl-N4-(1-methyl-1-tetrahydropyran-4-yl-ethyl)pyrazolo[3,4-d]pyrimidine-4,6-diamine;
1-methyl-N6-[(2R)-2-amino-2-phenyl-ethyl]-N4-spiro[2.3]hexan-5-yl-pyrazolo[3,4-d]pyrimidine-4,6-diamine;
N6-[(2R)-2-amino-2-phenyl-ethyl]-N4-(2,2-dimethylcyclopropyl)-1-methyl-pyrazolo[3,4-d]pyrimidine-4,6-diamine;
N6-[(2R)-2-amino-2-phenyl-ethyl]-N4-tert-butyl-1-cyclopropyl-pyrazolo[3,4-d]pyrimidine-4,6-diamine;
6-N-[2-amino-2-(2-methoxyphenyl)ethyl]-4-N-[(3-chloro-4-methylphenyl)methyl]-1-methylpyrazolo[3,4-d]pyrimidine-4,6-diamine;
6-N-(2-Amino-2-phenylethyl)-4-N-[(3-chloro-4-methylphenyl)methyl]-1-methylpyrazolo[3,4-d]pyrimidine-4,6-diamine;
6-N-(2-amino-2-pyridin-2-ylethyl)-1-methyl-4-N-[4-(trifluoromethyl)phenyl]pyrazolo[3,4-d]pyrimidine-4,6-diamine;
3-[1-Amino-2-[[1-methyl-4-(methylamino)pyrazolo[3,4-d]pyrimidin-6-yl]amino]ethyl]benzonitrile;
N6-[2-Amino-2-(3-ethynylphenyl)ethyl]-N4-tert-butyl-1-methyl-pyrazolo[3,4-d]pyrimidine-4,6-diamine;
N6-[2-amino-2-(2-pyridyl)ethyl]-N4-tert-butyl-1-methyl-pyrazolo[3,4-d]pyrimidine-4,6-diamine;
6-N-(2-amino-2-pyridin-3-ylethyl)-4-N-[(3-chloro-4-methylphenyl)methyl]-1-methylpyrazolo[3,4-d]pyrimidine-4,6-diamine;
N6-[2-amino-2-(3-pyridyl)ethyl]-N4-tert-butyl-1-methyl-pyrazolo[3,4-d]pyrimidine-4,6-diamine;
6-N-[2-amino-2-(2-chlorophenyl)ethyl]-1-methyl-4-N-[4-(trifluoromethyl)phenyl]pyrazolo[3,4-d]pyrimidine-4,6-diamine;
N6-[2-amino-2-(2-chlorophenyl)ethyl]-N4-tert-butyl-1-methyl-pyrazolo[3,4-d]pyrimidine-4,6-diamine;
N6-[(2R)-2-amino-2-(2-chlorophenyl)ethyl]-N4-tert-butyl-1-methyl-pyrazolo[3,4-d]pyrimidine-4,6-diamine;
6-N-[2-amino-2-(2-fluorophenyl)ethyl]-4-N-[(3-chloro-4-methylphenyl)methyl]-1-methylpyrazolo[3,4-d]pyrimidine-4,6-diamine;
N6-[2-amino-2-(2-fluorophenyl)ethyl]-N4-tert-butyl-1-methyl-pyrazolo[3,4-d]pyrimidine-4,6-diamine;
N6-[2-amino-2-(2-fluorophenyl)ethyl]-N4-tert-butyl-1-(trideuteriomethyl) pyrazolo[3,4-d]pyrimidine-4,6-diamine;
N6-[(2R)-2-amino-2-(3-bromophenyl)ethyl]-N4-isopropyl-1-methyl-pyrazolo[3,4-d]pyrimidine-4,6-diamine;
N6-[(2R)-2-amino-2-(3-bromophenyl)ethyl]-1-methyl-N4-(2,2,2-trifluoro-1,1-dimethyl-ethyl)pyrazolo[3,4-d]pyrimidine-4,6-diamine;
6-N-[2-amino-2-(3-bromophenyl)ethyl]-4-N,1-dimethylpyrazolo[3,4-d]pyrimidine-4,6-diamine;
N6-[2-amino-2-(3-bromophenyl)ethyl]-N4-tert-butyl-1-methyl-pyrazolo[3,4-d]pyrimidine-4,6-diamine;
6-N-[2-amino-2-(4-bromophenyl)ethyl]-1-methyl-4-N-propan-2-ylpyrazolo[3,4-d]pyrimidine-4,6-diamine;
N6-[2-amino-2-(2-bromophenyl)ethyl]-N4-tert-butyl-1-methyl-pyrazolo[3,4-d]pyrimidine-4,6-diamine;
4-[1-amino-2-[[1-methyl-4-(methylamino)pyrazolo[3,4-d]pyrimidin-6-yl]amino]ethyl]benzonitrile;
3-[1-amino-2-[[4-(tert-butylamino)-1-methyl-pyrazolo[3,4-d]pyrimidin-6-yl]amino]ethyl]benzonitrile;
3-[(1R)-1-amino-2-[[1-methyl-4-[(2,2,2-trifluoro-1,1-dimethyl-ethyl)amino]pyrazolo[3,4-d]pyrimidin-6-yl]amino]ethyl]benzonitrile;
N6-[2-amino-2-(3-pyridyl)ethyl]-N4-tert-butyl-1-methyl-pyrazolo[3,4-d]pyrimidine-4,6-diamine;
N6-[2-amino-2-(3-pyridyl)ethyl]-N4-(3,3-difluorocyclobutyl)-1-methyl-pyrazolo[3,4-d]pyrimidine-4,6-diamine;
N6-[2-amino-2-(4-pyridyl)ethyl]-N4-tert-butyl-1-methyl-pyrazolo[3,4-d]pyrimidine-4,6-diamine;
N6-[2-amino-2-(4-pyridyl)ethyl]-N4-(3,3-difluorocyclobutyl)-1-methyl-pyrazolo[3,4-d]pyrimidine-4,6-diamine;
N′-(4-Ethoxy-1-methylpyrazolo[3,4-d]pyrimidin-6-yl)-1-phenylethane-1,2-diamine;
N′-(1-methyl-4-propan-2-yloxypyrazolo[3,4-d]pyrimidin-6-yl)-1-phenylethane-1,2-diamine;
N′-(4-tert-butoxy-1-methyl-pyrazolo[3,4-d]pyrimidin-6-yl)-1-phenyl-ethane-1,2-diamine;
1-methyl-N6-{[(3R)-morpholin-3-yl]methyl}-N4-[4-(trifluoromethyl)phenyl]-1H-pyrazolo[3,4-d]pyrimidine-4,6-diamine;
6-N-[(1S,2R)-2-aminocyclohexyl]-4-N-[(3-chloro-4-methylphenyl)methyl]-1-methylpyrazolo[3,4-d]pyrimidine-4,6-diamine;
6-N-[(1R,2R)-2-aminocyclohexyl]-4-N-[(3-chloro-4-methylphenyl)methyl]-1-methylpyrazolo[3,4-d]pyrimidine-4,6-diamine;
N6-[(2R)-2-amino-2-phenyl-ethyl]-N4-cyclopropyl-1-methyl-pyrazolo[3,4-d]pyrimidine-4,6-diamine;
N6-[(2R)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-1-methyl-N4-(2,2,2-trifluoroethyl)pyrazolo[3,4-d]pyrimidine-4,6-diamine;
N6-[(2R)-2-amino-2-phenyl-ethyl]-1-methyl-N4-[6-(trifluoromethyl)-3-pyridyl]pyrazolo[3,4-d]pyrimidine-4,6-diamine;
N6-[(2S)-2-amino-2-phenyl-ethyl]-1-methyl-N4-[6-(trifluoromethyl)-3-pyridyl]pyrazolo[3,4-d]pyrimidine-4,6-diamine;
rel-6-N-[(2S)-2-amino-2-phenylpropyl]-4-N,1-dimethylpyrazolo[3,4-d]pyrimidine-4,6-diamine;
N6-[(2S)-2-amino-2-(2-fluorophenyl)ethyl]-N4-tert-butyl-1-methyl-pyrazolo[3,4-d]pyrimidine-4,6-diamine;
N6-[(2R)-2-amino-2-(2-fluorophenyl)ethyl]-N4-tert-butyl-1-methyl-pyrazolo[3,4-d]pyrimidine-4,6-diamine;
rel-N6-[(2R)-2-amino-2-phenyl-propyl]-1-methyl-N4-[6-(trifluoromethyl)-3-pyridyl]pyrazolo[3,4-d]pyrimidine-4,6-diamine;
6-N-[(2R)-2-amino-3-fluoropropyl]-4-N-[(3-chloro-4-methylphenyl)methyl]-1-methylpyrazolo[3,4-d]pyrimidine-4,6-diamine;
rel-N6-[(2S)-2-amino-2-phenyl-propyl]-1-methyl-N4-[6-(trifluoromethyl)-3-pyridyl]pyrazolo[3,4-d]pyrimidine-4,6-diamine;
N6-[(2R)-2-amino-2-(2-fluorophenyl)ethyl]-N4-tert-butyl-1-(trideuteriomethyl)pyrazolo[3,4-d]pyrimidine-4,6-diamine; and
N6-[(2S)-2-amino-2-(2-fluorophenyl)ethyl]-N4-tert-butyl-1-(trideuteriomethyl)pyrazolo[3,4-d]pyrimidine-4,6-diamine.

Paragraph 17. A pharmaceutical composition comprising a compound according to any one of paragraphs 1 to 16, or a pharmaceutically acceptable salt thereof, and one or more pharmaceutically acceptable excipients.

Paragraph 18. A compound according to any one of paragraphs 1 to 16, or a pharmaceutically acceptable salt thereof, or a pharmaceutical composition according to paragraph 17, for use in therapy.

Paragraph 19. A compound according to any one of paragraphs 1 to 16, or a pharmaceutically acceptable salt thereof, or a pharmaceutical composition according to paragraph 17, for use in the treatment of a proliferative condition.

Paragraph 20. A compound according to any one of paragraphs 1 to 16, or a pharmaceutically acceptable salt thereof, or a pharmaceutical composition according to paragraph 17, for use in the treatment of cancer.

Paragraph 21. A compound according to any one of paragraphs 1 to 16, or a pharmaceutically acceptable salt there, or a pharmaceutical composition according to paragraph 17, for use in the treatment of breast cancer.

Paragraph 22. A compound according to any one of paragraphs 1 to 16, or a pharmaceutically acceptable salt thereof, or a pharmaceutical composition according to paragraph 17, for use in the inhibition and/or degradation of BCDIN3D activity.

Paragraph 23. Use of a compound according to any one of paragraphs 1 to 16, or a pharmaceutically acceptable salt thereof, in the manufacture of a medicament for the treatment of a proliferative condition.

Paragraph 24. Use of a compound according to any one of paragraphs 1 to 16, or a pharmaceutically acceptable salt thereof, in the manufacture of a medicament for the treatment of cancer.

Paragraph 25. Use of a compound according to any one of paragraphs 1 to 16, or a pharmaceutically acceptable salt thereof, in the manufacture of a medicament for the treatment of breast cancer.

Paragraph 26. Use of a compound according to any one of paragraphs 1 to 16, or a pharmaceutically acceptable salt thereof, in the manufacture of a medicament for the inhibition and/or degradation of BCDIN3D activity.

Paragraph 27. A method of treating a proliferative disorder, said method comprising administering to a subject in need thereof a therapeutically effective amount of a compound according to any one of paragraphs 1 to 16, or a pharmaceutically acceptable salt thereof, or a pharmaceutical composition according to paragraph 17.

Paragraph 28. A method of treating cancer, said method comprising administering to a subject in need thereof a therapeutically effective amount of a compound according to any one of paragraphs 1 to 16, or a pharmaceutically acceptable salt thereof, or a pharmaceutical composition according to paragraph 17.

Paragraph 29. A method of treating breast cancer, said method comprising administering to a subject in need thereof a therapeutically effective amount of a compound according to any one of paragraphs 1 to 16, or a pharmaceutically acceptable salt thereof, or a pharmaceutical composition according to paragraph 17.

Paragraph 30. A method of inhibiting and/or degrading BCDIN3D activity in vitro or in vivo, said method comprising contacting a cell with an effective amount of a compound according to any one of paragraphs 1 to 16, or a pharmaceutically acceptable salt thereof, or a pharmaceutical composition according to paragraph 17.

Paragraph 31. A method of inhibiting and/or degrading metastasis in vitro or in vivo, said method comprising contacting a cell with an effective amount of a compound according to any one of paragraphs 1 to 16, or a pharmaceutically acceptable salt thereof, or a pharmaceutical composition according to paragraph 17.

Paragraph 32. A combination comprising a compound according to any one of paragraphs 1 to 16, or a pharmaceutically acceptable salt there, with one or more additional therapeutic agents.

1. A compound of the formula (I), or a pharmaceutically acceptable salt thereof, 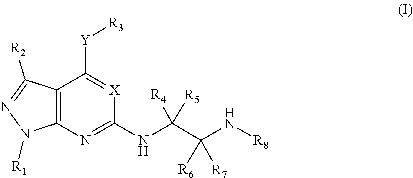
wherein:
R₁ is selected from methyl, ethyl, CH₂F, CHF₂, cyclopropyl, and CH₂CN wherein one or more hydrogen atoms in methyl, ethyl, CH₂F, CHF₂, cyclopropyl, and CH₂CN may be replaced by deuterium;
R₂ is selected from hydrogen, C₁₋₂ alkyl, C₁₋₂haloalkyl, cyano, amino, halo, trifluoromethyl, trifluoromethoxy, hydroxy, carboxy, carbamoyl, sulphamoyl, NRᵣRₛ, ORᵣ, C(O)Rᵣ, C(O)ORᵣ, OC(O)Rᵣ, C(O)N(Rᵣ)Rₛ, N(Rᵣ)C(O)Rₛ, S(O)ₐRᵣ (where a is 0, 1 or 2), SO₂N(Rᵣ)Rₛ, N(Rᵣ)SO₂Rₛ or (CH₂)_{b}NRᵣRₛ (where b is 1, 2 or 3), wherein Rᵣ and Rₛ are each independently selected from hydrogen or C₁₋₂alkyl;
R₃ is a group of the formula:
-L₁ₐ-L_{1b}-Q₁
wherein
L₁ₐ is absent or C₁₋₃alkylene, C₃₋₄cycloalkylene, optionally substituted by one or more substituents selected from C₁₋₂alkyl, halo, hydroxy, amino or oxo;
L_{1b} is absent or selected from O, S, SO, SO₂, N(Rₓ), C(O), C(O)O, OC(O), C(O)N(Rₓ), N(Rₓ)C(O), N(Rₓ)C(O)N(R_{y}), S(O)₂N(Rₓ), or N(Rₓ)SO₂, wherein Rₓ and R_{y} are each independently selected from hydrogen or C₁₋₂alkyl; and
Q₁ is hydrogen, cyano, C₁₋₆alkyl, C₃₋₁₀carbocyclic, C₂₋₃alkenyl, C₂₋₃alkynyl, aryl, heterocyclyl or heteroaryl; wherein one or more hydrogen atoms in C₁₋₆alkyl may be replaced by deuterium; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, halo, trifluoromethyl, trifluoromethoxy, amino, cyano, hydroxy, carboxy, carbamoyl, sulphamoyl, NRₜRᵤ, ORₜ, C(O)Rₜ, C(O)ORₜ, OC(O)Rₜ, C(O)N(Rₜ)Rᵤ, N(Rₜ)C(O)Rᵤ, S(O)_{y}Rₜ (where y is 0, 1 or 2), SO₂N(Rₜ)Rᵤ, N(Rₜ)SO₂Rᵤ or (CH₂)_{z}NRₜRᵤ (where z is 1, 2 or 3); wherein Rₜ and Rᵤ are each independently selected from hydrogen or C₁₋₄alkyl wherein the C₁₋₄alkyl is optionally substituted with one or more halo substituents or -L_{1c}-L_{1d}-Z₁ as defined below; or
Q₁ is optionally substituted by one or more groups of the formula:
-L_{1c}-L_{1d}-Z₁
wherein
L_{1c} is absent, C₁₋₃alkylene or O—C₁₋₃alkylene, wherein C₁₋₃alkylene or O—C₁₋₃alkylene are optionally substituted by C₁₋₂alkyl or oxo;
L_{1d} is absent or selected from O, C(O), C(O)O, OC(O), C(O)N(Rₘ), N(Rₘ)C(O), N(Rₘ)C(O)N(Rₙ), S(O)₂N(Rₘ), S(O)₂, or N(Rₘ)SO₂, wherein Rₘ and Rₙ are each independently selected from hydrogen or C₁₋₂alkyl; and
Z₁ is cyano, C₁₋₆alkyl, C₃₋₁₀carbocyclic, C₂₋₃alkenyl, C₂₋₃alkynyl, aryl, heterocyclyl or heteroaryl; and wherein C₁₋₆alkyl, C₃₋₁₀carbocyclic, C₂₋₃alkenyl, C₂₋₃alkynyl, aryl, heterocyclyl or heteroaryl are optionally substituted by one or more substituents selected from C₁₋₄alkyl, C₂₋₄alkynyl, halo, trifluoromethyl, trifluoromethoxy, amino, cyano, hydroxy, carboxy, carbamoyl, sulphamoyl, NRᵥR_{w}, ORᵥ, C(O)Rᵥ, C(O)ORᵥ, OC(O)Rᵥ, C(O)N(Rᵥ)R_{w}, N(Rᵥ)C(O)R_{w}, S(O)ₓRᵥ (where x is 0, 1 or 2), SO₂N(Rᵥ)R_{w}, N(Rᵥ)SO₂R_{w} or (CH₂)ₛNRᵥR_{w} (where s is 1, 2 or 3), wherein Rᵥ and R_{w} are each independently selected from hydrogen or C₁₋₄alkyl;
R₄ is hydrogen;
R₅ is selected from hydrogen and C₁₋₄alkyl;
R₆ is selected from hydrogen and C₁₋₄ alkyl;
R₇ is selected from hydrogen, C₁₋₄alkyl, C₃₋₆cycloalkyl, heterocyclyl, heteroaryl and aryl, wherein C₁₋₄alkyl, C₃₋₆cycloalkyl, heterocyclyl, heteroaryl or aryl is optionally substituted with one or more substituents selected from C₁₋₄alkyl, C₂₋₃alkenyl, C₂₋₃alkynyl, halo, trifluoromethyl, trifluoromethoxy, amino, cyano, hydroxy, carboxy, carbamoyl, sulphamoyl, NRₚR_{q}, ORₚ, C(O)Rₚ, C(O)ORₚ, OC(O)Rₚ, C(O)N(Rₚ)R_{q}, N(Rₚ)C(O)R_{q}, S(O)ⱼRₚ (where j is 0, 1 or 2), SO₂N(Rₚ)R_{q}, N(Rₚ)SO₂R_{q} or (CH₂)ₖNRₚR_{q} (where k is 1, 2 or 3), wherein Rₚ and R_{q} are each independently selected from hydrogen or C₁₋₄alkyl;
R₈ is selected from hydrogen or C₁₋₄alkyl;
or R₆ and R₇ may be linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂-haloalkyl, halo, and cyano;
or R₅ and R₇ may be linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano;
or R₇ and R₈ may be linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano;
X is selected from N and CR₉;
R₉ is selected from hydrogen, methyl, and fluoro;
Y is selected from O and NR₁₀; and
R₁₀ is selected from hydrogen and C₁₋₄alkyl;
or R₃ and R₁₀ may be linked together to form a 4-, 5-, 6- or 7-membered heterocyclic ring system.

2. A compound according to claim 1, or a pharmaceutically acceptable salt thereof, wherein R₅ is C₁₋₄ alkyl, R₆ is hydrogen and R₇ is hydrogen.

3. A compound according to claim 1, or a pharmaceutically acceptable salt thereof, wherein R₅ is hydrogen.

4. A compound according to claim 1, or a pharmaceutically acceptable salt thereof, wherein either:
R₆ is hydrogen and R₇ is hydrogen, methyl, ethyl, cyclopropyl, cyclohexyl, pyridyl, and phenyl, wherein methyl, cyclopropyl, cyclohexyl, pyridyl or phenyl is optionally substituted with one or more substituents selected from methyl, ethynyl, fluoro, chloro, bromo, cyano and ORₚ, wherein Rₚ is independently selected from hydrogen or methyl; or R₆ and R₇ are linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano.

5. A compound according to claim 4, or a pharmaceutically acceptable salt thereof, wherein R₆ is hydrogen and R₇ is selected from hydrogen, methyl optionally substituted with fluoro or OMe, ethyl, —CH₂F, —CH₂OMe, cyclopropyl, cyclohexyl, phenyl,

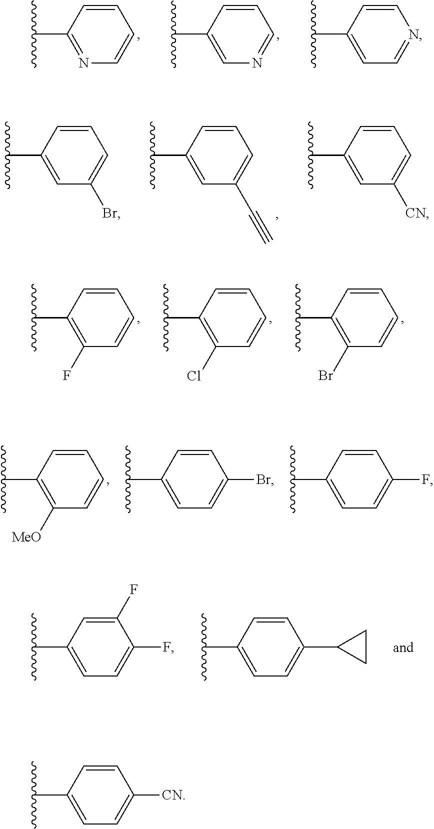

6. A compound according to claim 4, wherein R₆ and R₇ are linked together to form a 3-, 4-, 5-, or 6-membered carbocyclic ring system, wherein the 3-, 4-, 5-, or 6-membered carbocyclic heterocyclic ring system is optionally substituted with one or more fluoro.

7. A compound according to claim 1, or a pharmaceutically acceptable salt thereof, wherein R₆ is hydrogen, and R₅ and R₇ are linked together to form a 5- or 6-membered carbocyclic or heterocyclic ring system wherein the 5- or 6-membered carbocyclic or heterocyclic ring system is optionally substituted with one or more substituents selected from C₁₋₂alkyl, C₁₋₂alkyoxy, C₁₋₂haloalkyl, halo, and cyano.

8. A compound according to claim 1, or a pharmaceutically acceptable salt thereof, wherein R₅ is hydrogen, and R₆ and R₇ are linked together to form a 3-, 4-, 5-, or 6-membered carbocyclic or heterocyclic ring system, wherein the 3-, 4-, 5-, or 6-membered carbocyclic or heterocyclic ring system is optionally substituted with one or more substituents selected from C₁₋₂alkyl, C₁₋₂alkyoxy, C₁₋₂haloalkyl, halo, and cyano.

9. A compound according to claim 8, or a pharmaceutically acceptable salt thereof, wherein R₅ is hydrogen, and R₆ and R₇ are linked together to form a cyclopropyl ring system.

10. A compound according to claim 1, or a pharmaceutically acceptable salt thereof, wherein R₅ is hydrogen, and R₇ and R₈ are linked together to form a 4-, 5-, 6- or 7-membered heterocyclic ring system wherein the 4-, 5-, 6- or 7-membered heterocylic ring system is optionally substituted with one or more substituents selected from C₁₋₂alkyl, C₁₋₂alkyoxy, C₁₋₂haloalkyl, halo, and cyano.

11. A compound according to claim 1, or a pharmaceutically acceptable salt thereof, wherein R₁ is methyl.

12. A compound according to claim 1, or a pharmaceutically acceptable salt thereof, wherein R₂ is hydrogen.

13. A compound according to claim 1, or a pharmaceutically acceptable salt thereof, wherein Y is NR₁₀ and R₁₀ is hydrogen.

14. A compound according to claim 1, or a pharmaceutically acceptable salt thereof, wherein X is N.

15. A compound according to claim 1, or a pharmaceutically acceptable salt thereof, wherein R₃ is selected from: methyl, ethyl, isopropyl, t-butyl, cyclopropyl, cyclobutyl, phenyl,

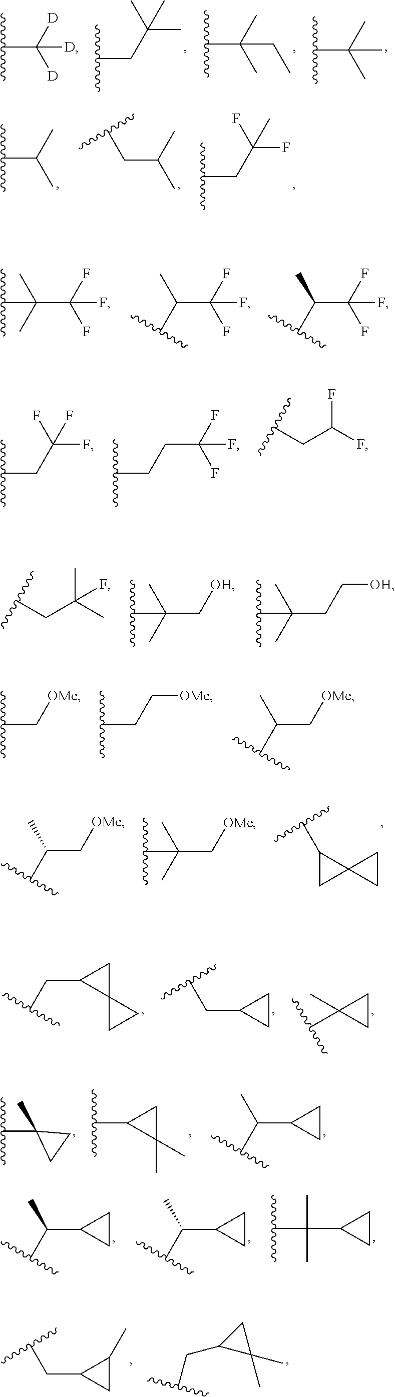

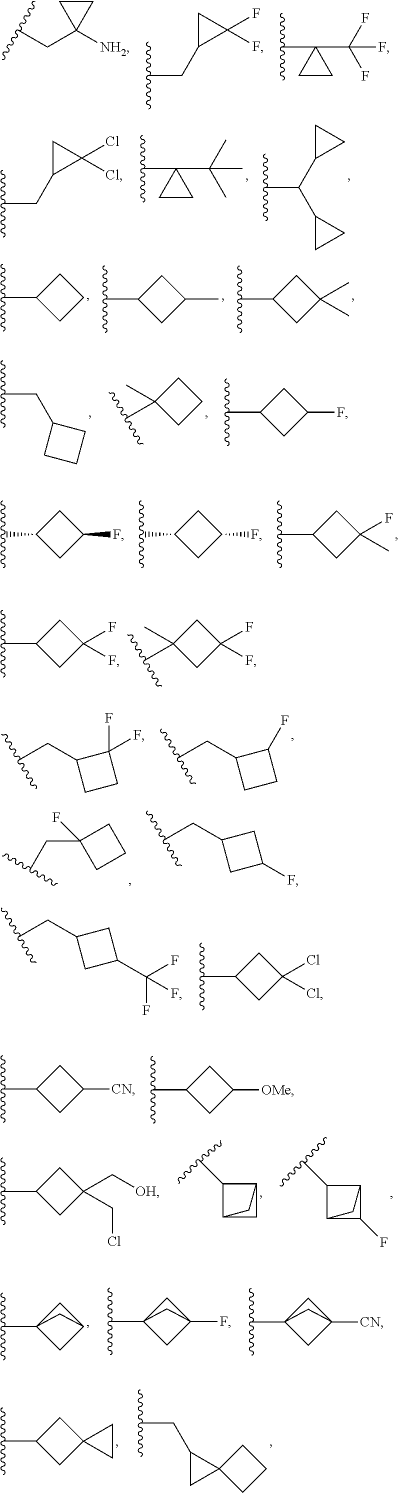

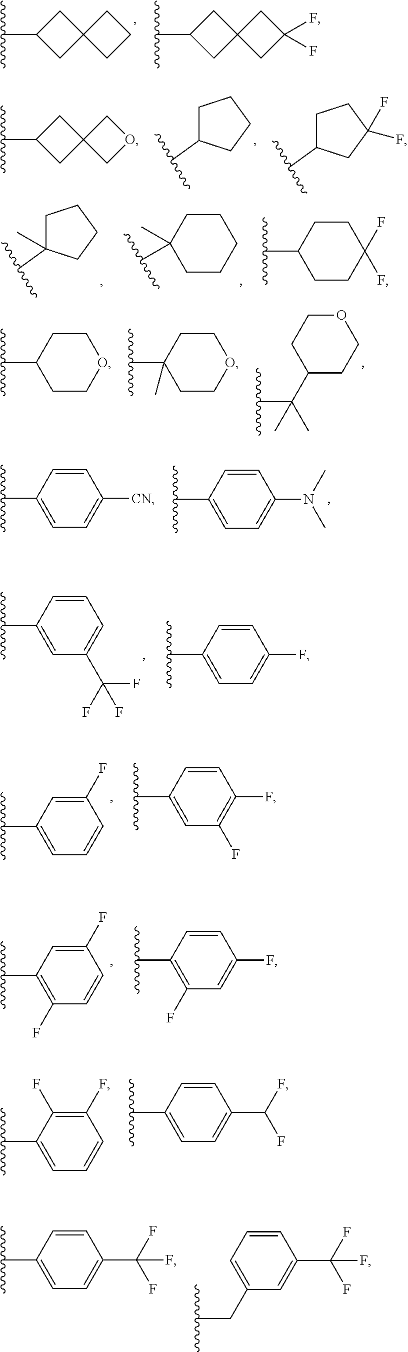

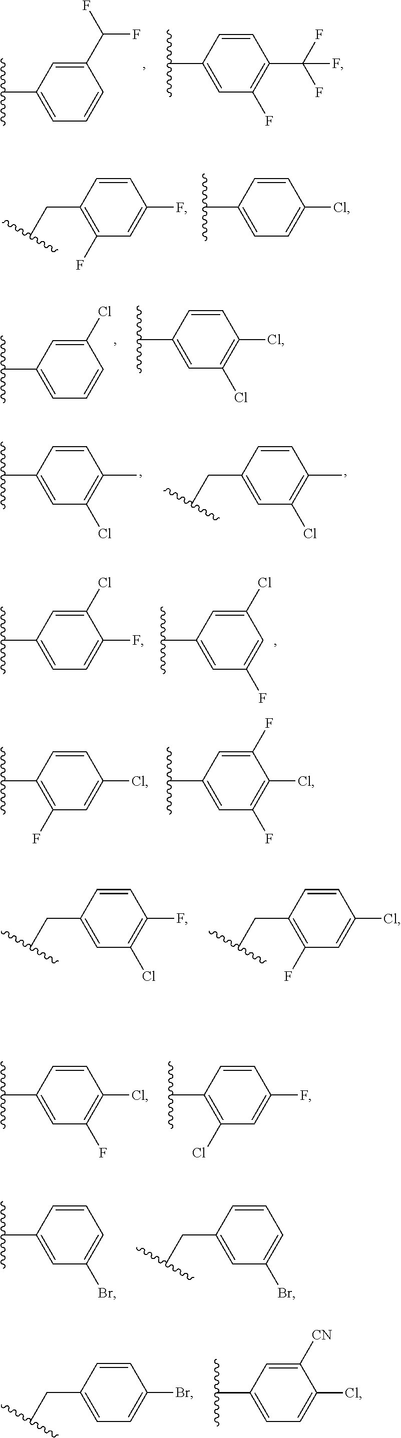

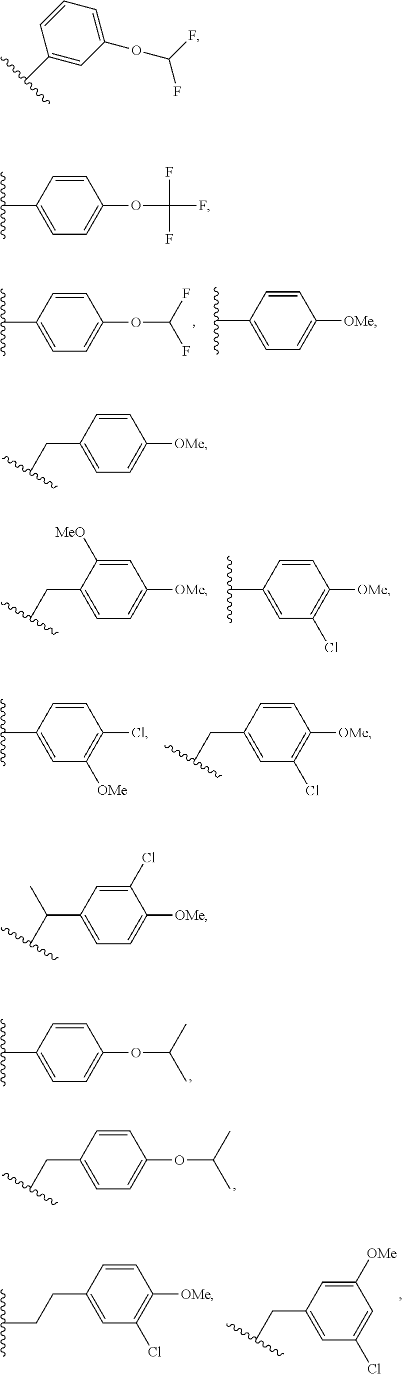

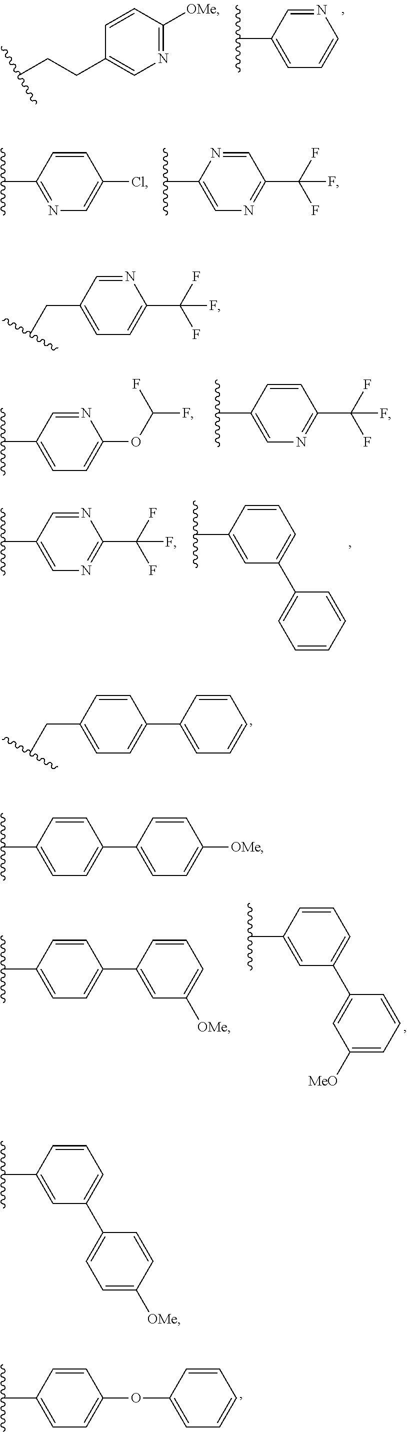

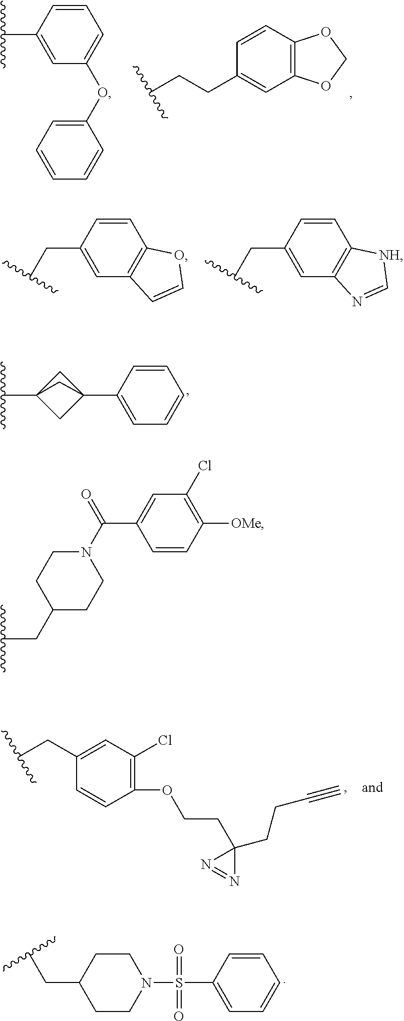

16. A compound, or a pharmaceutically acceptable salt thereof, selected from any one of the following:
N6-(2-Aminoethyl)-1-methyl-N4-[4-(trifluoromethyl)phenyl]pyrazolo[3,4-d]pyrimidine-4,6-diamine;
N6-(2-aminoethyl)-1-methyl-N4-[3-(trifluoromethyl)phenyl]-1H-pyrazolo[3,4-d]pyrimidine-4,6-diamine;
N6-(2-aminoethyl)-1-methyl-N4-(4-phenoxyphenyl)-1H-pyrazolo[3,4-d]pyrimidine-4,6-diamine;
N6-(2-aminoethyl)-1-methyl-N4-(3-phenoxyphenyl)-1H-pyrazolo[3,4-d]pyrimidine-4,6-diamine;
N6-(2-aminoethyl)-1-methyl-N4-[(4-phenylphenyl)methyl]pyrazolo[3,4-d]pyrimidine-4,6-diamine;
N6-(2-aminoethyl)-N4-[1-(3-chloro-4-methoxyphenyl)ethyl]-1-methyl-1H-pyrazolo[3,4-d]pyrimidine-4,6-diamine;
N6-(2-aminoethyl)-N4-[2-(6-methoxypyridin-3-yl)ethyl]-1-methyl-1H-pyrazolo[3,4-d]pyrimidine-4,6-diamine;
N6-(2-aminoethyl)-N4-[2-(3-chloro-4-methoxyphenyl)ethyl]-1-methyl-1H-pyrazolo[3,4-d]pyrimidine-4,6-diamine;
N6-(2-aminoethyl)-N4-[2-(2H-1,3-benzodioxol-5-yl)ethyl]-1-methyl-1H-pyrazolo[3,4-d]pyrimidine-4,6-diamine;
N6-(2-aminoethyl)-N4-[(1-benzofuran-5-yl)methyl]-1-methyl-1H-pyrazolo[3,4-d]pyrimidine-4,6-diamine;
N6-(2-aminoethyl)-N4-{3′-methoxy-[1,1′-biphenyl]-3-yl}-1-methyl-1H-pyrazolo[3,4-d]pyrimidine-4,6-diamine;
N6-(2-aminoethyl)-N4-{[1-(benzenesulfonyl)piperidin-4-yl]methyl}-1-methyl-1H-pyrazolo[3,4-d]pyrimidine-4,6-diamine;
N6-(2-aminoethyl)-N4-[(1H-1,3-benzodiazol-5-yl)methyl]-1-methyl-1H-pyrazolo[3,4-d]pyrimidine-4,6-diamine;
N6-(2-aminoethyl)-N4-{3′-methoxy-[1,1′-biphenyl]-4-yl}-1-methyl-1H-pyrazolo[3,4-d]pyrimidine-4,6-diamine;
N6-(2-aminoethyl)-N4-{4′-methoxy-[1,1′-biphenyl]-3-yl}-1-methyl-1H-pyrazolo[3,4-d]pyrimidine-4,6-diamine;
N6-(2-aminoethyl)-N4-{[1,1′-biphenyl]-3-yl}-1-methyl-1H-pyrazolo[3,4-d]pyrimidine-4,6-diamine;
N6-(2-aminoethyl)-N4-[(3-chloro-4-fluorophenyl)methyl]-1-methyl-1H-pyrazolo[3,4-d]pyrimidine-4,6-diamine;
N6-(2-aminoethyl)-N4-[(4-chloro-2-fluorophenyl)methyl]-1-methyl-1H-pyrazolo[3,4-d]pyrimidine-4,6-diamine;
N6-(2-aminoethyl)-N4-[(2,4-difluorophenyl)methyl]-1-methyl-1H-pyrazolo[3,4-d]pyrimidine-4,6-diamine;
N6-(2-aminoethyl)-N4-[(3-chloro-5-methoxyphenyl)methyl]-1-methyl-1H-pyrazolo[3,4-d]pyrimidine-4,6-diamine;
N6-(2-aminoethyl)-N4-(3-chloro-4-methoxyphenyl)-1-methyl-1H-pyrazolo[3,4-d]pyrimidine-4,6-diamine;
N6-(2-aminoethyl)-N4-[(3-chloro-4-methylphenyl)methyl]-1-methyl-1H-pyrazolo[3,4-d]pyrimidine-4,6-diamine;
N6-(2-aminoethyl)-1-methyl-N4-{[3-(trifluoromethyl)phenyl]methyl}-1H-pyrazolo[3,4-d]pyrimidine-4,6-diamine;
N6-(2-aminoethyl)-N4-[(2,4-dimethoxyphenyl)methyl]-1-methyl-1H-pyrazolo[3,4-d]pyrimidine-4,6-diamine;
N6-(2-aminoethyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(3-chloro-4-methoxyphenyl)methyl]-1-methyl-1H-pyrazolo[3,4-d]pyrimidine-4,6-diamine;
N6-(2-amino-2-methylpropyl)-N4-[(4-methoxyphenyl)methyl]-1-methyl-1H-pyrazolo[3,4-d]pyrimidine-4,6-diamine;
N6-(2-amino-2-methylpropyl)-N4-({[1,1′-biphenyl]-4-yl}methyl)-1-methyl-1H-pyrazolo[3,4-d]pyrimidine-4,6-diamine;
N6-(2-aminoethyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-[(3-chloro-4-methoxyphenyl)methyl]-1,3-dimethyl-1H-pyrazolo[3,4-d]pyrimidine-4,6-diamine;
N6-(2-amino-2-methylpropyl)-N4-({[1,1′-biphenyl]-4-yl}methyl)-1,3-dimethyl-1H-pyrazolo[3,4-d]pyrimidine-4,6-diamine;
N6-[(1-aminocyclohexyl)methyl]-1-methyl-N4-[4-(trifluoromethyl)phenyl]pyrazolo[3,4-d]pyrimidine-4,6-diamine;
N6-(2-amino-2-methylpropyl)-1,3-dimethyl-N4-[3-(trifluoromethyl)phenyl]-1H-pyrazolo[3,4-d]pyrimidine-4,6-diamine;
6-N-(2-aminocyclohexyl)-4-N-[(3-chloro-4-methylphenyl)methyl]-1-methylpyrazolo[3,4-d]pyrimidine-4,6-diamine;
6-N-[(4-aminooxan-4-yl)methyl]-1-methyl-4-N-[6-(trifluoromethyl)pyridin-3-yl]pyrazolo[3,4-d]pyrimidine-4,6-diamine;
N6-[(3R,4S)-4-aminotetrahydrofuran-3-yl]-1-methyl-N4-[4-(trifluoromethyl)phenyl]pyrazolo[3,4-d]pyrimidine-4,6-diamine;
N6-(2-aminoethyl)-N4-[(4-methoxyphenyl)methyl]-1-methyl-1H-pyrazolo[3,4-d]pyrimidine-4,6-diamine;
tert-butyl 4-[({6-[(2-aminoethyl)amino]-1-methyl-1H-pyrazolo[3,4-d]pyrimidin-4-yl}amino)methyl]piperidine-1-carboxylate;
N6-(2-aminoethyl)-N4-[(1-benzylpiperidin-4-yl)methyl]-1-methyl-1H-pyrazolo[3,4-d]pyrimidine-4,6-diamine;
N6-(2-aminoethyl)-N4-[(1-benzoylpiperidin-4-yl)methyl]-1-methyl-1H-pyrazolo[3,4-d]pyrimidine-4,6-diamine;
N6-(2-aminoethyl)-N4-{[1-(3-chloro-4-methoxybenzoyl)piperidin-4-yl]methyl}-1-methyl-1H-pyrazolo[3,4-d]pyrimidine-4,6-diamine;
6-N-(2-Amino-2-methylpropyl)-1,3-dimethyl-4-N-[[3-(trifluoromethyl)phenyl]methyl]pyrazolo[3,4-b]pyridine-4,6-diamine;
6-N-[(1-Aminocyclopropyl)methyl]-4-N-[(3-chloro-4-methylphenyl)methyl]-1-methylpyrazolo[3,4-d]pyrimidine-4,6-diamine;
6-N-[(1-Aminocyclopropyl)methyl]-4-N-[(3-chloro-4-fluorophenyl)methyl]-1-methylpyrazolo[3,4-d]pyrimidine-4,6-diamine;
N6-[(1-aminocyclopropyl)methyl]-N4-[3-fluoro-4-(trifluoromethyl)phenyl]-1-methyl-pyrazolo[3,4-d]pyrimidine-4,6-diamine;
6-N-[(1-aminocyclopropyl)methyl]-4-N-(3-chloro-4-methoxyphenyl)-1-methylpyrazolo[3,4-d]pyrimidine-4,6-diamine;
6-N-[(1-aminocyclopropyl)methyl]-4-N-(2,4-difluorophenyl)-1-methylpyrazolo[3,4-d]pyrimidine-4,6-diamine;
6-N-[(1-aminocyclopropyl)methyl]-1-methyl-4-N-[3-(trifluoromethyl)phenyl]pyrazolo[3,4-d]pyrimidine-4,6-diamine;
6-N-[(1-aminocyclopropyl)methyl]-4-N-[3-(difluoromethyl)phenyl]-1-methylpyrazolo[3,4-d]pyrimidine-4,6-diamine;
6-N-[(1-aminocyclopropyl)methyl]-4-N-(4-fluorophenyl)-1-methylpyrazolo[3,4-d]pyrimidine-4,6-diamine;
6-N-[(1-aminocyclopropyl)methyl]-4-N-(3-chloro-4-methylphenyl)-1-methylpyrazolo[3,4-d]pyrimidine-4,6-diamine;
6-N-[(1-aminocyclopropyl)methyl]-1-methyl-4-N-pyridin-3-ylpyrazolo[3,4-d]pyrimidine-4,6-diamine;
6-N-[(1-aminocyclopropyl)methyl]-1-methyl-4-N-phenylpyrazolo[3,4-d]pyrimidine-4,6-diamine;
6-N-[(1-aminocyclopropyl)methyl]-4-N-[(3-bromophenyl)methyl]-1-methylpyrazolo[3,4-d]pyrimidine-4,6-diamine;
6-N-[(1-aminocyclopropyl)methyl]-4-N-[(4-bromophenyl)methyl]-1-methylpyrazolo[3,4-d]pyrimidine-4,6-diamine;
6-N-[(1-aminocyclopropyl)methyl]-4-N-(2-chloro-4-fluorophenyl)-1-methylpyrazolo[3,4-d]pyrimidine-4,6-diamine;
6-N-[(1-aminocyclopropyl)methyl]-4-N-(4-chloro-2-fluorophenyl)-1-methylpyrazolo[3,4-d]pyrimidine-4,6-diamine;
6-N-[(1-aminocyclopropyl)methyl]-4-N-(3-chloro-5-fluorophenyl)-1-methylpyrazolo[3,4-d]pyrimidine-4,6-diamine;
5-[[6-[(1-aminocyclopropyl)methylamino]-1-methyl-pyrazolo[3,4-d]pyrimidin-4-yl]amino]-2-chloro-benzonitrile;
N6-[(1-aminocyclopropyl)methyl]-1-methyl-N4-(1-phenyl-3-bicyclo[1.1.1]pentanyl)pyrazolo[3,4-d]pyrimidine-4,6-diamine;
6-N-[(1-aminocyclopropyl)methyl]-1-methyl-4-N-[[6-(trifluoromethyl)pyridin-3-yl]methyl]pyrazolo[3,4-d]pyrimidine-4,6-diamine;
6-N-[(1-aminocyclopropyl)methyl]-4-N-(4-chloro-3,5-difluorophenyl)-1-methylpyrazolo[3,4-d]pyrimidine-4,6-diamine;
6-N-[(1-aminocyclopropyl)methyl]-4-N-[3-(difluoromethoxy)phenyl]-1-methylpyrazolo[3,4-d]pyrimidine-4,6-diamine;
6-N-[(1-aminocyclopropyl)methyl]-1-methyl-4-N-(4-propan-2-yloxyphenyl)pyrazolo[3,4-d]pyrimidine-4,6-diamine;
6-N-[(1-aminocyclopropyl)methyl]-4-N-[(4-chloro-3-fluorophenyl)methyl]-1-methylpyrazolo[3,4-d]pyrimidine-4,6-diamine;
6-N-[(1-aminocyclopropyl)methyl]-4-N-(2,3-difluorophenyl)-1-methylpyrazolo[3,4-d]pyrimidine-4,6-diamine;
6-N-[(1-aminocyclopropyl)methyl]-4-N-(2,5-difluorophenyl)-1-methylpyrazolo[3,4-d]pyrimidine-4,6-diamine;
6-N-[(1-aminocyclopropyl)methyl]-4-N-[(2,4-difluorophenyl)methyl]-1-methylpyrazolo[3,4-d]pyrimidine-4,6-diamine;
6-N-[(1-aminocyclopropyl)methyl]-4-N-(3-chloro-4-fluorophenyl)-1-methylpyrazolo[3,4-d]pyrimidine-4,6-diamine;
6-N-[(1-aminocyclopropyl)methyl]-4-N-(4-chlorophenyl)-1-methylpyrazolo[3,4-d]pyrimidine-4,6-diamine;
6-N-[(1-aminocyclopropyl)methyl]-4-N-(3-fluorophenyl)-1-methylpyrazolo[3,4-d]pyrimidine-4,6-diamine;
6-N-[(1-aminocyclopropyl)methyl]-1,3-dimethyl-4-N-[4-(trifluoromethyl)phenyl]pyrazolo[3,4-d]pyrimidine-4,6-diamine;
4-[[6-[(1-aminocyclopropyl)methylamino]-1-methylpyrazolo[3,4-d]pyrimidin-4-yl]amino]benzonitrile;
6-N-[(1-aminocyclopropyl)methyl]-1-methyl-4-N-[4-(trifluoromethoxy)phenyl]pyrazolo[3,4-d]pyrimidine-4,6-diamine;
6-N-[(1-aminocyclopropyl)methyl]-4-N-(4-methoxyphenyl)-1-methylpyrazolo[3,4-d]pyrimidine-4,6-diamine;
6-N-[(1-aminocyclopropyl)methyl]-4-N-[4-(difluoromethoxy)phenyl]-1-methylpyrazolo[3,4-d]pyrimidine-4,6-diamine;
6-N-[(1-aminocyclopropyl)methyl]-4-N-(3-chlorophenyl)-1-methylpyrazolo[3,4-d]pyrimidine-4,6-diamine;
6-N-[(1-aminocyclopropyl)methyl]-4-N-[4-(difluoromethyl)phenyl]-1-methylpyrazolo[3,4-d]pyrimidine-4,6-diamine;
3-[[6-[(1-aminocyclopropyl)methylamino]-1-methylpyrazolo[3,4-d]pyrimidin-4-yl]amino]bicyclo[1.1.1]pentane-1-carbonitrile;
6-N-[(1-aminocyclopropyl)methyl]-4-N-(3-bicyclo[1.1.1]pentanyl)-1-methylpyrazolo[3,4-d]pyrimidine-4,6-diamine;
6-N-[(1-aminocyclopropyl)methyl]-4-N-(3,4-difluorophenyl)-1-methylpyrazolo[3,4-d]pyrimidine-4,6-diamine;
6-N-[(1-aminocyclopropyl)methyl]-4-N-(3,4-dichlorophenyl)-1-methylpyrazolo[3,4-d]pyrimidine-4,6-diamine;
6-N-[(1-aminocyclopropyl)methyl]-4-N-(4-chloro-3-methoxyphenyl)-1-methylpyrazolo[3,4-d]pyrimidine-4,6-diamine;
N6-[(1-aminocyclopropyl)methyl]-N4-[(2,4-dimethoxyphenyl)methyl]-1-methyl-pyrazolo[3,4-d]pyrimidine-4,6-diamine;
N6-[(1-aminocyclopropyl)methyl]-N4-{3-fluorobicyclo[1.1.1]pentan-1-yl}-1-methyl-1H-pyrazolo[3,4-d]pyrimidine-4,6-diamine;
6-N-[(1-aminocyclopropyl)methyl]-1-methyl-4-N-[4-(trifluoromethyl)phenyl]pyrazolo[3,4-d]pyrimidine-4,6-diamine;
6-N-[(1-aminocyclopropyl)methyl]-1-methyl-4-N-[6-(trifluoromethyl)pyridin-3-yl]pyrazolo[3,4-d]pyrimidine-4,6-diamine;
6-N-[(1-aminocyclopropyl)methyl]-4-N-(5-chloropyridin-2-yl)-1-methylpyrazolo[3,4-d]pyrimidine-4,6-diamine;
N6-[(1-aminocyclopropyl)methyl]-1-methyl-N4-[5-(trifluoromethyl)pyrazin-2-yl]pyrazolo[3,4-d]pyrimidine-4,6-diamine;
6-N-[(1-aminocyclopropyl)methyl]-1-ethyl-4-N-[4-(trifluoromethyl)phenyl]pyrazolo[3,4-d]pyrimidine-4,6-diamine;
N6-[(1-aminocyclopropyl)methyl]-1-(trideuteriomethyl)-N4-[4-(trifluoromethyl)phenyl]pyrazolo[3,4-d]pyrimidine-4,6-diamine;
N6-[(1-aminocyclopropyl)methyl]-1-(trideuteriomethyl)-N4-[6-(trifluoromethyl)-3-pyridyl]pyrazolo[3,4-d]pyrimidine-4,6-diamine;
6-N-[(1-aminocyclopropyl)methyl]-4-N-[[4-[2-(3-but-3-ynyldiazirin-3-yl)ethoxy]-3-chlorophenyl]methyl]-1-methylpyrazolo[3,4-d]pyrimidine-4,6-diamine
Compound 2.52: N6-[(1-Aminocyclopropyl)methyl]-1-methyl-N4-[4-(trifluoromethyl)phenyl]pyrazolo[3,4-b]pyridine-4,6-diamine;
6-N-(2-aminopropyl)-4-N-[(3-chloro-4-methylphenyl)methyl]-1-methylpyrazolo[3,4-d]pyrimidine-4,6-diamine;
6-N-(1-aminopropan-2-yl)-4-N-[(3-chloro-4-methylphenyl)methyl]-1-methylpyrazolo[3,4-d]pyrimidine-4,6-diamine;
6-N-[(2S)-2-aminopropyl]-4-N-[(3-chloro-4-methylphenyl)methyl]-1-methylpyrazolo[3,4-d]pyrimidine-4,6-diamine;
6-N-[(2R)-2-aminopropyl]-4-N-[(3-chloro-4-methylphenyl)methyl]-1-methylpyrazolo[3,4-d]pyrimidine-4,6-diamine;
6-N-[(2S)-1-aminobutyl]-4-N-[(3-chloro-4-methylphenyl)methyl]-1-methylpyrazolo[3,4-d]pyrimidine-4,6-diamine;
6-N-[(2S)-2-aminobutyl]-4-N-[(3-chloro-4-methylphenyl)methyl]-1-methylpyrazolo[3,4-d]pyrimidine-4,6-diamine;
6-N-[(2R)-2-aminobutyl]-4-N-[(3-chloro-4-methylphenyl)methyl]-1-methylpyrazolo[3,4-d]pyrimidine-4,6-diamine;
N6-[(1-aminocyclobutyl)methyl]-1-methyl-N4-[4-(trifluoromethyl)phenyl]pyrazolo[3,4-d]pyrimidine-4,6-diamine;
N6-[(1-aminocyclobutyl)methyl]-N4-[4-(difluoromethoxy)phenyl]-1-methyl-pyrazolo[3,4-d]pyrimidine-4,6-diamine;
N6-(2-amino-2-cyclohexyl-ethyl)-N4-tert-butyl-1-methyl-pyrazolo[3,4-d]pyrimidine-4,6-diamine;
N6-(2-amino-2-cyclohexyl-ethyl)-1-methyl-N4-[4-(trifluoromethyl)phenyl]pyrazolo[3,4-d]pyrimidine-4,6-diamine;
6-N-[(1-amino-3,3-difluorocyclobutyl)methyl]-1-methyl-4-N-[4-(trifluoromethyl)phenyl]pyrazolo[3,4-d]pyrimidine-4,6-diamine;
6-N-(2-amino-2-cyclopropylethyl)-1-methyl-4-N-[4-(trifluoromethyl)phenyl]pyrazolo[3,4-d]pyrimidine-4,6-diamine;
6-N-[(3-aminooxolan-3-yl)methyl]-1-methyl-4-N-[4-(trifluoromethyl)phenyl]pyrazolo[3,4-d]pyrimidine-4,6-diamine;
N6-[(3-aminotetrahydrofuran-3-yl)methyl]-1-methyl-N4-[6-(trifluoromethyl)-3-pyridyl]pyrazolo[3,4-d]pyrimidine-4,6-diamine;
1-methyl-N6-[(morpholin-3-yl)methyl]-N4-[4-(trifluoromethyl)phenyl]-1H-pyrazolo[3,4-d]pyrimidine-4,6-diamine;
4-N-[(3-chloro-4-methylphenyl)methyl]-1-methyl-6-N-[[(2R)-pyrrolidin-2-yl]methyl]pyrazolo[3,4-d]pyrimidine-4,6-diamine;
6-N-[(1-aminocyclopentyl)methyl]-1-methyl-4-N-[4-(trifluoromethyl)phenyl]pyrazolo[3,4-d]pyrimidine-4,6-diamine;
6-N-[(1-aminocyclopentyl)methyl]-1-methyl-4-N-[6-(trifluoromethyl)pyridin-3-yl]pyrazolo[3,4-d]pyrimidine-4,6-diamine;
6-N-[(1-aminocyclopentyl)methyl]-4-N-[4-(difluoromethoxy)phenyl]-1-methylpyrazolo[3,4-d]pyrimidine-4,6-diamine;
6-N-[(1-aminocyclopentyl)methyl]-1-methyl-4-N-phenylpyrazolo[3,4-d]pyrimidine-4,6-diamine;
4-[[6-[(1-aminocyclopentyl)methylamino]-1-methylpyrazolo[3,4-d]pyrimidin-4-yl]amino]benzonitrile;
6-N-[(1-aminocyclopentyl)methyl]-1-methyl-4-N-[4-(trifluoromethoxy)phenyl]pyrazolo[3,4-d]pyrimidine-4,6-diamine;
N6-[(1-aminocyclobutyl)methyl]-1-(trideuteriomethyl)-N4-[4-(trifluoromethyl)phenyl]pyrazolo[3,4-d]pyrimidine-4,6-diamine;
N6-(2-aminocyclohexyl)-N4-[(3-chloro-4-methylphenyl)methyl]-1-methyl-1H-pyrazolo[3,4-d]pyrimidine-4,6-diamine;
N6-(2-Amino-3-fluoropropyl)-N4-[(3-chloro-4-methylphenyl)methyl]-1-methyl-1H-pyrazolo[3,4-d]pyrimidine-4,6-diamine;
6-N-(2-amino-3-fluoropropyl)-1-methyl-4-N-[4-(trifluoromethyl)phenyl]pyrazolo[3,4-d]pyrimidine-4,6-diamine;
6-N-(2-amino-3-methoxypropyl)-4-N-[(3-chloro-4-methylphenyl)methyl]-1-methylpyrazolo[3,4-d]pyrimidine-4,6-diamine;
6-N-[(3-Aminooxetan-3-yl)methyl]-1-methyl-4-N-[4-(trifluoromethyl)phenyl]pyrazolo[3,4-d]pyrimidine-4,6-diamine;
6-N-[(3-Aminooxetan-3-yl)methyl]-1-methyl-4-N-phenylpyrazolo[3,4-d]pyrimidine-4,6-diamine;
N6-(2-Amino-2-phenyl-propyl)-N4-tert-butyl-1-(trideuteriomethyl)pyrazolo[3,4-d]pyrimidine-4,6-diamine;
6-N-(2-Amino-2-phenylethyl)-1-ethyl-4-N-propan-2-ylpyrazolo[3,4-d]pyrimidine-4,6-diamine;
6-N-(2-amino-2-phenylethyl)-4-N-[(3-chloro-4-methylphenyl)methyl]-1-methylpyrazolo[3,4-d]pyrimidine-4,6-diamine;
6-N-(2-amino-2-phenylethyl)-4-N-[(2,4-difluorophenyl)methyl]-1-methylpyrazolo[3,4-d]pyrimidine-4,6-diamine;
6-N-(2-amino-2-phenylethyl)-4-N-[(3-chloro-4-fluorophenyl)methyl]-1-methylpyrazolo[3,4-d]pyrimidine-4,6-diamine;
6-N-(2-amino-2-phenylethyl)-4-N-[(3-bromophenyl)methyl]-1-methylpyrazolo[3,4-d]pyrimidine-4,6-diamine;
6-N-(2-amino-2-phenylethyl)-4-N-[(4-bromophenyl)methyl]-1-methylpyrazolo[3,4-d]pyrimidine-4,6-diamine;
6-N-(2-amino-2-phenylethyl)-1-methyl-4-N-[[6-(trifluoromethyl)pyridin-3-yl]methyl]pyrazolo[3,4-d]pyrimidine-4,6-diamine;
N6-(2-amino-2-phenyl-ethyl)-N4-[(2,4-dimethoxyphenyl)methyl]-1-methyl-pyrazolo[3,4-d]pyrimidine-4,6-diamine;
6-N-(2-amino-2-phenylethyl)-4-N-(4-fluorophenyl)-1-methylpyrazolo[3,4-d]pyrimidine-4,6-diamine;
N6-(2-amino-2-phenyl-propyl)-1-methyl-N4-[2-(trifluoromethyl)pyrimidin-5-yl]pyrazolo[3,4-d]pyrimidine-4,6-diamine;
N6-(2-amino-2-phenyl-propyl)-1-methyl-N4-[4-(trifluoromethoxy)phenyl]pyrazolo[3,4-d]pyrimidine-4,6-diamine;
N6-(2-amino-2-phenyl-propyl)-N4-(3,4-dichlorophenyl)-1-methyl-pyrazolo[3,4-d]pyrimidine-4,6-diamine;
6-N-(2-amino-2-phenylethyl)-4-N-[4-(difluoromethoxy)phenyl]-1-methylpyrazolo[3,4-d]pyrimidine-4,6-diamine;
6-N-(2-amino-2-phenylpropyl)-4-N-[4-(difluoromethoxy)phenyl]-1-methylpyrazolo[3,4-d]pyrimidine-4,6-diamine;
N6-[(2R)-2-amino-2-phenyl-ethyl]-N4-[4-(difluoromethoxy)phenyl]-1-methyl-pyrazolo[3,4-d]pyrimidine-4,6-diamine;
6-N-(2-amino-2-phenylethyl)-1-methyl-4-N-[4-(trifluoromethyl)phenyl]pyrazolo[3,4-d]pyrimidine-4,6-diamine;
6-N-(2-amino-2-phenylethyl)-1,3-dimethyl-4-N-[4-(trifluoromethyl)phenyl]pyrazolo[3,4-d]pyrimidine-4,6-diamine;
N6-(2-amino-2-phenyl-propyl)-1-methyl-N4-[4-(trifluoromethyl)phenyl]pyrazolo[3,4-d]pyrimidine-4,6-diamine;
N6-(2-amino-2-phenyl-ethyl)-1-(trideuteriomethyl)-N4-[4-(trifluoromethyl)phenyl]pyrazolo[3,4-d]pyrimidine-4,6-diamine;
N6-(2-amino-2-phenyl-propyl)-1-(trideuteriomethyl)-N4-[4-(trifluoromethyl)phenyl]pyrazolo[3,4-d]pyrimidine-4,6-diamine;
6-N-[(2R)-2-amino-2-phenylethyl]-1-methyl-4-N-[4-(trifluoromethyl)phenyl]pyrazolo[3,4-d]pyrimidine-4,6-diamine;
6-N-[(2S)-2-amino-2-phenylethyl]-1-methyl-4-N-[4-(trifluoromethyl)phenyl]pyrazolo[3,4-d]pyrimidine-4,6-diamine;
N6-[(2R)-2-amino-2-phenyl-ethyl]-1-(trideuteriomethyl)-N4-[4-(trifluoromethyl)phenyl]pyrazolo[3,4-d]pyrimidine-4,6-diamine;
6-N-(2-amino-2-phenylethyl)-1-methyl-4-N-[6-(trifluoromethyl)pyridin-3-yl]pyrazolo[3,4-d]pyrimidine-4,6-diamine;
6-N-(2-amino-2-phenylpropyl)-1-methyl-4-N-[6-(trifluoromethyl)pyridin-3-yl]pyrazolo[3,4-d]pyrimidine-4,6-diamine;
N6-(2-amino-2-phenyl-propyl)-1-(trideuteriomethyl)-N4-[6-(trifluoromethyl)-3-pyridyl]pyrazolo[3,4-d]pyrimidine-4,6-diamine;
N6-(2-amino-2-phenyl-ethyl)-N4-[6-(difluoromethoxy)-3-pyridyl]-1-methyl-pyrazolo[3,4-d]pyrimidine-4,6-diamine;
N6-(2-amino-2-phenyl-propyl)-N4-[6-(difluoromethoxy)-3-pyridyl]-1-methyl-pyrazolo[3,4-d]pyrimidine-4,6-diamine;
N6-[(2R)-2-amino-2-phenyl-ethyl]-N4-(4-chlorophenyl)-1-methyl-pyrazolo[3,4-d]pyrimidine-4,6-diamine;
N6-[(2S)-2-amino-2-phenyl-ethyl]-N4-(4-chlorophenyl)-1-methyl-pyrazolo[3,4-d]pyrimidine-4,6-diamine;
N6-[(2R)-2-amino-2-phenyl-ethyl]-N4-(4-methoxyphenyl)-1-methyl-pyrazolo[3,4-d]pyrimidine-4,6-diamine;
N6-[(2R)-2-amino-2-phenyl-ethyl]-N4-(4-isopropoxyphenyl)-1-methyl-pyrazolo[3,4-d]pyrimidine-4,6-diamine;
N6-[(2R)-2-amino-2-phenyl-ethyl]-N4-(3,4-difluorophenyl)-1-methyl-pyrazolo[3,4-d]pyrimidine-4,6-diamine;
N6-[(2R)-2-amino-2-phenyl-ethyl]-N4-[4-(dimethylamino)phenyl]-1-methyl-pyrazolo[3,4-d]pyrimidine-4,6-diamine;
6-N-(2-amino-2-phenylethyl)-4-N,1-dimethylpyrazolo[3,4-d]pyrimidine-4,6-diamine;
6-N-(2-amino-2-phenylethyl)-1-methyl-4-N-(trideuteriomethyl)pyrazolo[3,4-d]pyrimidine-4,6-diamine;
6-N-(2-amino-2-phenylethyl)-4-N-methyl-1-(trideuteriomethyl)pyrazolo[3,4-d]pyrimidine-4,6-diamine;
6-N-(2-amino-2-phenylpropyl)-4-N,1-dimethylpyrazolo[3,4-d]pyrimidine-4,6-diamine;
6-N-[(2R)-2-amino-2-phenylethyl]-4-N,1-dimethylpyrazolo[3,4-d]pyrimidine-4,6-diamine;
6-N-(2-amino-2-phenylethyl)-4-N-ethyl-1-methylpyrazolo[3,4-d]pyrimidine-4,6-diamine;
6-N-(2-amino-2-phenylethyl)-4-N-ethyl-4-N,1-dimethylpyrazolo[3,4-d]pyrimidine-4,6-diamine;
6-N-(2-amino-2-phenylethyl)-4-N-cyclopropyl-1-methylpyrazolo[3,4-d]pyrimidine-4,6-diamine;
6-N-(2-amino-2-phenylethyl)-1-methyl-4-N-(2,2,2-trifluoroethyl)pyrazolo[3,4-d]pyrimidine-4,6-diamine;
N6-[(2R)-2-amino-2-phenyl-ethyl]-1-methyl-N4-(2,2,2-trifluoroethyl)pyrazolo[3,4-d]pyrimidine-4,6-diamine;
6-N-(2-amino-2-phenylethyl)-1-methyl-4-N-propan-2-ylpyrazolo[3,4-d]pyrimidine-4,6-diamine;
6-N-[(2R)-2-amino-2-phenylethyl]-1-methyl-4-N-propan-2-ylpyrazolo[3,4-d]pyrimidine-4,6-diamine;
N′-(1-methyl-4-pyrrolidin-1-ylpyrazolo[3,4-d]pyrimidin-6-yl)-1-phenylethane-1,2-diamine;
6-N-(2-amino-2-phenylethyl)-4-N-(2-methoxyethyl)-1-methylpyrazolo[3,4-d]pyrimidine-4,6-diamine;
6-N-(2-amino-2-phenylethyl)-4-N-tert-butyl-1-methylpyrazolo[3,4-d]pyrimidine-4,6-diamine;
N6-(2-amino-2-phenyl-propyl)-N4-tert-butyl-1-methyl-pyrazolo[3,4-d]pyrimidine-4,6-diamine;
N6-(2-amino-2-phenyl-ethyl)-N4-tert-butyl-1-(trideuteriomethyl)pyrazolo[3,4-d]pyrimidine-4,6-diamine;
6-N-[(2R)-2-amino-2-phenylethyl]-4-N-tert-butyl-1-methylpyrazolo[3,4-d]pyrimidine-4,6-diamine;
6-N-[(2S)-2-amino-2-phenylethyl]-4-N-tert-butyl-1-methylpyrazolo[3,4-d]pyrimidine-4,6-diamine;
N6-[(2R)-2-amino-2-phenyl-ethyl]-N4-tert-butyl-1-(trideuteriomethyl)pyrazolo[3,4-d]pyrimidine-4,6-diamine;
6-N-(2-amino-2-phenylethyl)-4-N-(cyclopropylmethyl)-1-methylpyrazolo[3,4-d]pyrimidine-4,6-diamine;
N6-(2-amino-2-phenyl-ethyl)-N4-(dicyclopropylmethyl)-1-methyl-pyrazolo[3,4-d]pyrimidine-4,6-diamine;
N6-[(2R)-2-amino-2-phenyl-ethyl]-1-methyl-N4-[(2-methylcyclopropyl)methyl]pyrazolo[3,4-d]pyrimidine-4,6-diamine;
N6-[(2R)-2-amino-2-phenyl-ethyl]-N4-(1-cyclopropyl-1-methyl-ethyl)-1-methyl-pyrazolo[3,4-d]pyrimidine-4,6-diamine;
N6-[(2R)-2-amino-2-phenyl-ethyl]-1-methyl-N4-(spiro[2.2]pentan-2-ylmethyl)pyrazolo[3,4-d]pyrimidine-4,6-diamine;
N6-[(2R)-2-amino-2-phenyl-ethyl]-N4-[(2,2-difluorocyclopropyl)methyl]-1-methyl-pyrazolo[3,4-d]pyrimidine-4,6-diamine;
N6-[(2R)-2-amino-2-phenyl-ethyl]-N4-[(2,2-dimethylcyclopropyl)methyl]-1-methyl-pyrazolo[3,4-d]pyrimidine-4,6-diamine;
6-N-(2-amino-2-phenylethyl)-4-N-(2-bicyclo[1.1.1]pentanyl)-1-methylpyrazolo[3,4-d]pyrimidine-4,6-diamine;
3-[[6-[(2-amino-2-phenylethyl)amino]-1-methylpyrazolo[3,4-d]pyrimidin-4-yl]amino]bicyclo[1.1.1]pentane-1-carbonitrile;
N6-(2-amino-2-phenylethyl)-N4-{3-fluorobicyclo[1.1.1]pentan-1-yl}-1-methyl-1H-pyrazolo[3,4-d]pyrimidine-4,6-diamine;
N6-[(2R)-2-amino-2-phenyl-ethyl]-N4-(3-bicyclo[1.1.1]pentanyl)-1-methyl-pyrazolo[3,4-d]pyrimidine-4,6-diamine;
N6-[(2R)-2-amino-2-phenyl-ethyl]-N4-(3-fluoro-1-bicyclo[1.1.1]pentanyl)-1-methyl-pyrazolo[3,4-d]pyrimidine-4,6-diamine;
6-N-(2-amino-2-phenylethyl)-4-N-(4,4-difluorocyclohexyl)-1-methylpyrazolo[3,4-d]pyrimidine-4,6-diamine;
N6-[(2R)-2-amino-2-phenyl-ethyl]-1-methyl-N4-tetrahydropyran-4-yl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]-3-methyl-butan-1-ol;
2-[[6-[[(2R)-2-amino-2-phenyl-ethyl]amino]-1-methyl-pyrazolo[3,4-d]pyrimidin-4-yl]amino]-2-methyl-propan-1-ol;
N6-[(2R)-2-amino-2-phenyl-ethyl]-N4-(2,2-dimethylpropyl)-1-methyl-pyrazolo[3,4-d]pyrimidine-4,6-diamine;
N6-[(2R)-2-amino-2-phenyl-ethyl]-N4-(2,2-difluoroethyl)-1-methyl-pyrazolo[3,4-d]pyrimidine-4,6-diamine;
N6-[(2R)-2-amino-2-phenyl-ethyl]-N4-(2,2-difluoropropyl)-1-methyl-pyrazolo[3,4-d]pyrimidine-4,6-diamine;
N6-[(2R)-2-amino-2-phenyl-ethyl]-1-methyl-N4-(2,2,2-trifluoro-1,1-dimethyl-ethyl)pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-(1-methylcyclopropyl)pyrazolo[3,4-d]pyrimidine-4,6-diamine;
N6-[(2R)-2-amino-2-phenyl-ethyl]-1-methyl-N4-[1-(trifluoromethyl)cyclopropyl]pyrazolo[3,4-d]pyrimidine-4,6-diamine;
N6-[(2R)-2-amino-2-phenyl-ethyl]-N4-(1-tert-butylcyclopropyl)-1-methyl-pyrazolo[3,4-d]pyrimidine-4,6-diamine;
N6-[(2R)-2-amino-2-phenyl-ethyl]-1-methyl-N4-spiro[2.2]pentan-2-yl-pyrazolo[3,4-d]pyrimidine-4,6-diamine;
[3-[[6-[(2-amino-2-phenyl-ethyl)amino]-1-methyl-pyrazolo[3,4-d]pyrimidin-4-yl]amino]-1-(chloromethyl)cyclobutyl]methanol;
N6-(2-amino-2-phenyl-ethyl)-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-N4-cyclobutyl-1-methyl-pyrazolo[3,4-d]pyrimidine-4,6-diamine;
N6-[(2R)-2-amino-2-phenyl-ethyl]-1-methyl-N4-(1-methylcyclobutyl)pyrazolo[3,4-d]pyrimidine-4,6-diamine;
N6-[(2R)-2-amino-2-phenyl-ethyl]-N4-(3,3-difluorocyclobutyl)-1-methyl-pyrazolo[3,4-d]pyrimidine-4,6-diamine;
N6-[(2R)-2-amino-2-phenyl-ethyl]-N4-(3,3-difluoro-1-methyl-cyclobutyl)-1-methyl-pyrazolo[3,4-d]pyrimidine-4,6-diamine;
N6-[(2R)-2-amino-2-phenyl-ethyl]-1-methyl-N4-(3-methylcyclobutyl)pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
N6-[(2R)-2-amino-2-phenyl-ethyl]-N4-(3-fluorocyclobutyl)-1-methyl-pyrazolo[3,4-d]pyrimidine-4,6-diamine;
3-[[6-[[(2R)-2-amino-2-phenyl-ethyl]amino]-1-methyl-pyrazolo[3,4-d]pyrimidin-4-yl]amino]cyclobutanecarbonitrile;
N6-[(2R)-2-amino-2-phenyl-ethyl]-N4-(3-methoxycyclobutyl)-1-methyl-pyrazolo[3,4-d]pyrimidine-4,6-diamine;
N6-[(2R)-2-amino-2-phenyl-ethyl]-N4-(3,3-dichlorocyclobutyl)-1-methyl-pyrazolo[3,4-d]pyrimidine-4,6-diamine;
N6-[(2R)-2-amino-2-phenyl-ethyl]-N4-(2,2-difluorospiro[3.3]heptan-6-yl)-1-methyl-pyrazolo[3,4-d]pyrimidine-4,6-diamine;
N6-[(2R)-2-amino-2-phenyl-ethyl]-1-methyl-N4-(2-oxaspiro[3.3]heptan-6-yl)pyrazolo[3,4-d]pyrimidine-4,6-diamine;
N6-[(2R)-2-amino-2-phenyl-ethyl]-N4-(3,3-dimethylcyclobutyl)-1-methyl-pyrazolo[3,4-d]pyrimidine-4,6-diamine;
N6-[(2R)-2-amino-2-phenyl-ethyl]-N4-(3-fluoro-3-methyl-cyclobutyl)-1-methyl-pyrazolo[3,4-d]pyrimidine-4,6-diamine;
N6-[(2R)-2-amino-2-phenyl-ethyl]-1-methyl-N4-[(1S)-2,2,2-trifluoro-1-methyl-ethyl]pyrazolo[3,4-d]pyrimidine-4,6-diamine;
N6-[(2R)-2-amino-2-phenyl-ethyl]-1-methyl-N4-spiro[3.3]heptan-2-yl-pyrazolo[3,4-d]pyrimidine-4,6-diamine;
N6-[(2R)-2-amino-2-phenyl-ethyl]-N4-(2-methoxy-1,1-dimethyl-ethyl)-1-methyl-pyrazolo[3,4-d]pyrimidine-4,6-diamine;
N6-[(2R)-2-amino-2-phenyl-ethyl]-N4-(1,1-dimethylpropyl)-1-methyl-pyrazolo[3,4-d]pyrimidine-4,6-diamine;
N6-[(2R)-2-amino-2-phenyl-ethyl]-N4-[(1S)-1-cyclopropylethyl]-1-methyl-pyrazolo[3,4-d]pyrimidine-4,6-diamine;
N6-[(2R)-2-amino-2-phenyl-ethyl]-N4-isobutyl-1-methyl-pyrazolo[3,4-d]pyrimidine-4,6-diamine;
N6-[(2R)-2-amino-2-phenyl-ethyl]-1-methyl-N4-(spiro[2.3]hexan-2-ylmethyl)pyrazolo[3,4-d]pyrimidine-4,6-diamine;
N6-[(2R)-2-amino-2-phenyl-ethyl]-N4-(cyclobutylmethyl)-1-methyl-pyrazolo[3,4-d]pyrimidine-4,6-diamine;
N6-[(2R)-2-amino-2-phenyl-ethyl]-N4-[(2,2-difluorocyclobutyl)methyl]-1-methyl-pyrazolo[3,4-d]pyrimidine-4,6-diamine;
N6-[(2R)-2-amino-2-phenyl-ethyl]-N4-[(1-fluorocyclobutyl)methyl]-1-methyl-pyrazolo[3,4-d]pyrimidine-4,6-diamine;
N6-[(2R)-2-amino-2-phenyl-ethyl]-N4-[(3-fluorocyclobutyl)methyl]-1-methyl-pyrazolo[3,4-d]pyrimidine-4,6-diamine;
N6-[(2R)-2-amino-2-phenyl-ethyl]-1-methyl-N4-[3-(trifluoromethyl)cyclobutyl]pyrazolo[3,4-d]pyrimidine-4,6-diamine;
N6-[(2R)-2-amino-2-phenyl-ethyl]-N4-(2-fluoro-2-methyl-propyl)-1-methyl-pyrazolo[3,4-d]pyrimidine-4,6-diamine;
N6-[(2R)-2-amino-2-phenyl-ethyl]-1-methyl-N4-(1-methylcyclohexyl)pyrazolo[3,4-d]pyrimidine-4,6-diamine;
N6-[(2R)-2-amino-2-phenyl-ethyl]-N4-[(2-fluorocyclobutyl)methyl]-1-methyl-pyrazolo[3,4-d]pyrimidine-4,6-diamine;
N6-[(2R)-2-amino-2-phenyl-ethyl]-N4-cyclopentyl-1-methyl-pyrazolo[3,4-d]pyrimidine-4,6-diamine;
N6-[(2R)-2-amino-2-phenyl-ethyl]-1-methyl-N4-(3,3,3-trifluoropropyl)pyrazolo[3,4-d]pyrimidine-4,6-diamine;
N6-[(2R)-2-amino-2-phenyl-ethyl]-N4-[(2,2-dichlorocyclopropyl)methyl]-1-methyl-pyrazolo[3,4-d]pyrimidine-4,6-diamine;
N6-[(2R)-2-amino-2-phenyl-ethyl]-1-methyl-N4-(1-methylcyclopentyl)pyrazolo[3,4-d]pyrimidine-4,6-diamine;
N6-[(2R)-2-amino-2-phenyl-ethyl]-N4-[(1R)-2-methoxy-1-methyl-ethyl]-1-methyl-pyrazolo[3,4-d]pyrimidine-4,6-diamine;
N6-[(2R)-2-amino-2-phenyl-ethyl]-1-methyl-N4-(1-methyl-1-tetrahydropyran-4-yl-ethyl)pyrazolo[3,4-d]pyrimidine-4,6-diamine;
1-methyl-N6-[(2R)-2-amino-2-phenyl-ethyl]-N4-spiro[2.3]hexan-5-yl-pyrazolo[3,4-d]pyrimidine-4,6-diamine;
N6-[(2R)-2-amino-2-phenyl-ethyl]-N4-(2,2-dimethylcyclopropyl)-1-methyl-pyrazolo[3,4-d]pyrimidine-4,6-diamine;
N6-[(2R)-2-amino-2-phenylethyl]-1-methyl-N4-[(2R)-3-methylbutan-2-yl]-1H-pyrazolo[3,4-d]pyrimidine-4,6-diamine;
N6-[(2R)-2-amino-2-phenylethyl]-1-methyl-N4-[(2S)-3-methylbutan-2-yl]-1H-pyrazolo[3,4-d]pyrimidine-4,6-diamine;
6-[(6-{[(2R)-2-amino-2-phenylethyl]amino}-1-methyl-1H-pyrazolo[3,4-d]pyrimidin-4-yl)amino]spiro[3.3]heptan-2-ol;
N6-[(2R)-2-amino-2-phenylethyl]-1-methyl-N4-[4-(trifluoromethyl)cyclohexyl]-1H-pyrazolo[3,4-d]pyrimidine-4,6-diamine;
N6-[(2R)-2-amino-2-phenylethyl]-N4-[(1S)-3,3-difluorocyclopentyl]-1-methyl-1H-pyrazolo[3,4-d]pyrimidine-4,6-diamine;
N6-[(2R)-2-amino-2-phenylethyl]-1-methyl-N4-(4-methyloxan-4-yl)-1H-pyrazolo[3,4-d]pyrimidine-4,6-diamine;
N6-[(2R)-2-amino-2-phenylethyl]-N4-tert-butyl-1,3-dimethyl-1H-pyrazolo[3,4-d]pyrimidine-4,6-diamine;
N6-[(2R)-2-amino-2-phenylethyl]-N4-[(1R)-1-cyclopropylethyl]-1-methyl-1H-pyrazolo[3,4-d]pyrimidine-4,6-diamine;
N6-[(2R)-2-amino-2-phenyl-ethyl]-N4-tert-butyl-1-cyclopropyl-pyrazolo[3,4-d]pyrimidine-4,6-diamine;
N2-[1-methyl-4-(2-methylpropyl)-1H-pyrazolo[3,4-d]pyrimidin-6-yl]-1-phenylethane-1,2-diamine;
6-N-[2-amino-2-(2-methoxyphenyl)ethyl]-4-N-[(3-chloro-4-methylphenyl)methyl]-1-methylpyrazolo[3,4-d]pyrimidine-4,6-diamine;
6-N-(2-Amino-2-phenylethyl)-4-N-[(3-chloro-4-methylphenyl)methyl]-1-methylpyrazolo[3,4-d]pyrimidine-4,6-diamine;
6-N-(2-amino-2-pyridin-2-ylethyl)-1-methyl-4-N-[4-(trifluoromethyl)phenyl]pyrazolo[3,4-d]pyrimidine-4,6-diamine;
3-[1-Amino-2-[[1-methyl-4-(methylamino)pyrazolo[3,4-d]pyrimidin-6-yl]amino]ethyl]benzonitrile;
N6-[2-Amino-2-(3-ethynylphenyl)ethyl]-N4-tert-butyl-1-methyl-pyrazolo[3,4-d]pyrimidine-4,6-diamine;
N6-[2-amino-2-(2-pyridyl)ethyl]-N4-tert-butyl-1-methyl-pyrazolo[3,4-d]pyrimidine-4,6-diamine;
6-N-(2-amino-2-pyridin-3-ylethyl)-4-N-[(3-chloro-4-methylphenyl)methyl]-1-methylpyrazolo[3,4-d]pyrimidine-4,6-diamine;
N6-[2-amino-2-(3-pyridyl)ethyl]-N4-tert-butyl-1-methyl-pyrazolo[3,4-d]pyrimidine-4,6-diamine;
6-N-[2-amino-2-(2-chlorophenyl)ethyl]-1-methyl-4-N-[4-(trifluoromethyl)phenyl]pyrazolo[3,4-d]pyrimidine-4,6-diamine;
N6-[2-amino-2-(2-chlorophenyl)ethyl]-N4-tert-butyl-1-methyl-pyrazolo[3,4-d]pyrimidine-4,6-diamine;
N6-[(2R)-2-amino-2-(2-chlorophenyl)ethyl]-N4-tert-butyl-1-methyl-pyrazolo[3,4-d]pyrimidine-4,6-diamine;
6-N-[2-amino-2-(2-fluorophenyl)ethyl]-4-N-[(3-chloro-4-methylphenyl)methyl]-1-methylpyrazolo[3,4-d]pyrimidine-4,6-diamine;
N6-[2-amino-2-(2-fluorophenyl)ethyl]-N4-tert-butyl-1-methyl-pyrazolo[3,4-d]pyrimidine-4,6-diamine;
N6-[2-amino-2-(2-fluorophenyl)ethyl]-N4-tert-butyl-1-(trideuteriomethyl)pyrazolo[3,4-d]pyrimidine-4,6-diamine;
N6-[(2R)-2-amino-2-(3-bromophenyl)ethyl]-N4-isopropyl-1-methyl-pyrazolo[3,4-d]pyrimidine-4,6-diamine;
N6-[(2R)-2-amino-2-(3-bromophenyl)ethyl]-1-methyl-N4-(2,2,2-trifluoro-1,1-dimethyl-ethyl)pyrazolo[3,4-d]pyrimidine-4,6-diamine;
6-N-[2-amino-2-(3-bromophenyl)ethyl]-4-N,1-dimethylpyrazolo[3,4-d]pyrimidine-4,6-diamine;
N6-[2-amino-2-(3-bromophenyl)ethyl]-N4-tert-butyl-1-methyl-pyrazolo[3,4-d]pyrimidine-4,6-diamine;
6-N-[2-amino-2-(4-bromophenyl)ethyl]-1-methyl-4-N-propan-2-ylpyrazolo[3,4-d]pyrimidine-4,6-diamine;
N6-[2-amino-2-(2-bromophenyl)ethyl]-N4-tert-butyl-1-methyl-pyrazolo[3,4-d]pyrimidine-4,6-diamine;
4-[1-amino-2-[[1-methyl-4-(methylamino)pyrazolo[3,4-d]pyrimidin-6-yl]amino]ethyl]benzonitrile;
3-[1-amino-2-[[4-(tert-butylamino)-1-methyl-pyrazolo[3,4-d]pyrimidin-6-yl]amino]ethyl]benzonitrile;
3-[(1R)-1-amino-2-[[1-methyl-4-[(2,2,2-trifluoro-1,1-dimethyl-ethyl)amino]pyrazolo[3,4-d]pyrimidin-6-yl]amino]ethyl]benzonitrile;
N6-[2-amino-2-(3-pyridyl)ethyl]-N4-tert-butyl-1-methyl-pyrazolo[3,4-d]pyrimidine-4,6-diamine;
N6-[2-amino-2-(3-pyridyl)ethyl]-N4-(3,3-difluorocyclobutyl)-1-methyl-pyrazolo[3,4-d]pyrimidine-4,6-diamine;
N6-[2-amino-2-(4-pyridyl)ethyl]-N4-tert-butyl-1-methyl-pyrazolo[3,4-d]pyrimidine-4,6-diamine;
N6-[2-amino-2-(4-pyridyl)ethyl]-N4-(3,3-difluorocyclobutyl)-1-methyl-pyrazolo[3,4-d]pyrimidine-4,6-diamine;
4-[(1R)-1-amino-2-{[4-(tert-butylamino)-1-methyl-1H-pyrazolo[3,4-d]pyrimidin-6-yl]amino}ethyl]benzonitrile;
N4-tert-butyl-1-methyl-N6-[(2R)-2-(methylamino)-2-phenylethyl]-1H-pyrazolo[3,4-d]pyrimidine-4,6-diamine;
N6-[2-amino-2-(4-fluorophenyl)ethyl]-N4-tert-butyl-1-methyl-1H-pyrazolo[3,4-d]pyrimidine-4,6-diamine;
N6-[2-amino-2-(3,4-difluorophenyl)ethyl]-N4-tert-butyl-1-methyl-pyrazlo[3,4-d]pyrimidine-4,6-diamine;
N6-[2-amino-2-(3-cyclopropylphenyl)ethyl]-N4-tert-butyl-1-methyl-1H-pyrazolo[3,4-d]pyrimidine-4,6-diamine;
N6-[2-amino-2-(4-cyclopropylphenyl)ethyl]-N4-tert-butyl-1-methyl-1H-pyrazolo[3,4-d]pyrimidine-4,6-diamine;
N′-(4-Ethoxy-1-methylpyrazolo[3,4-d]pyrimidin-6-yl)-1-phenylethane-1,2-diamine;
N′-(1-methyl-4-propan-2-yloxypyrazolo[3,4-d]pyrimidin-6-yl)-1-phenylethane-1,2-diamine;
N′-(4-tert-butoxy-1-methyl-pyrazolo[3,4-d]pyrimidin-6-yl)-1-phenyl-ethane-1,2-diamine;
1-methyl-N6-{[(3R)-morpholin-3-yl]methyl}-N4-[4-(trifluoromethyl)phenyl]-1H-pyrazolo[3,4-d]pyrimidine-4,6-diamine;
6-N-[(1S,2R)-2-aminocyclohexyl]-4-N-[(3-chloro-4-methylphenyl)methyl]-1-methylpyrazolo[3,4-d]pyrimidine-4,6-diamine;
6-N-[(1R,2R)-2-aminocyclohexyl]-4-N-[(3-chloro-4-methylphenyl)methyl]-1-methylpyrazolo[3,4-d]pyrimidine-4,6-diamine;
N6-[(2R)-2-amino-2-phenyl-ethyl]-N4-cyclopropyl-1-methyl-pyrazolo[3,4-d]pyrimidine-4,6-diamine;
N6-[(2R)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-N4-(4-fluorophenyl)-1-methyl-pyrazolo[3,4-d]pyrimidine-4,6-diamine;
N6-[(2S)-2-amino-2-phenyl-ethyl]-1-methyl-N4-(2,2,2-trifluoroethyl)pyrazolo[3,4-d]pyrimidine-4,6-diamine;
N6-[(2R)-2-amino-2-phenyl-ethyl]-1-methyl-N4-[6-(trifluoromethyl)-3-pyridyl]pyrazolo[3,4-d]pyrimidine-4,6-diamine;
N6-[(2S)-2-amino-2-phenyl-ethyl]-1-methyl-N4-[6-(trifluoromethyl)-3-pyridyl]pyrazolo[3,4-d]pyrimidine-4,6-diamine;
rel-6-N-[(2S)-2-amino-2-phenylpropyl]-4-N,1-dimethylpyrazolo[3,4-d]pyrimidine-4,6-diamine;
N6-[(2S)-2-amino-2-(2-fluorophenyl)ethyl]-N4-tert-butyl-1-methyl-pyrazolo[3,4-d]pyrimidine-4,6-diamine;
N6-[(2R)-2-amino-2-(2-fluorophenyl)ethyl]-N4-tert-butyl-1-methyl-pyrazolo[3,4-d]pyrimidine-4,6-diamine;
rel-N6-[(2R)-2-amino-2-phenyl-propyl]-1-methyl-N4-[6-(trifluoromethyl)-3-pyridyl]pyrazolo[3,4-d]pyrimidine-4,6-diamine;
6-N-[(2R)-2-amino-3-fluoropropyl]-4-N-[(3-chloro-4-methylphenyl)methyl]-1-methylpyrazolo[3,4-d]pyrimidine-4,6-diamine;
rel-N6-[(2S)-2-amino-2-phenyl-propyl]-1-methyl-N4-[6-(trifluoromethyl)-3-pyridyl]pyrazolo[3,4-d]pyrimidine-4,6-diamine;
N6-[(2R)-2-amino-2-(2-fluorophenyl)ethyl]-N4-tert-butyl-1-(trideuteriomethyl)pyrazolo[3,4-d]pyrimidine-4,6-diamine;
N6-[(2S)-2-amino-2-(2-fluorophenyl)ethyl]-N4-tert-butyl-1-(trideuteriomethyl)pyrazolo[3,4-d]pyrimidine-4,6-diamine;
N6-[(2R)-2-amino-2-(4-fluorophenyl)ethyl]-N4-tert-butyl-1-methyl-1H-pyrazolo[3,4-d]pyrimidine-4,6-diamine;
N6-[(2R)-2-amino-2-(2-fluorophenyl)ethyl]-N4-tert-butyl-1-methyl-pyrazolo[3,4-d]pyrimidine-4,6-diamine;
N6-[(2R)-2-amino-2-(3,4-difluorophenyl)ethyl]-N4-tert-butyl-1-methyl-1H-pyrazolo[3,4-d]pyrimidine-4,6-diamine;
N6-[(2S)-2-amino-2-(3,4-difluorophenyl)ethyl]-N4-tert-butyl-1-methyl-1H-pyrazolo[3,4-d]pyrimidine-4,6-diamine;
N6-[(2R)-2-amino-2-cyclohexylethyl]-N4-tert-butyl-1-methyl-1H-pyrazolo[3,4-d]pyrimidine-4,6-diamine; and
N6-[(2S)-2-amino-2-cyclohexylethyl]-N4-tert-butyl-1-methyl-1H-pyrazolo[3,4-d]pyrimidine-4,6-diamine.

17. A pharmaceutical composition comprising a compound according to claim 1, or a pharmaceutically acceptable salt thereof, and one or more pharmaceutically acceptable excipients.

18-25. (canceled)

26. A method of treating
i) a proliferative disorder,
ii) cancer or inflammation, or
iii) breast cancer
said method comprising administering to a subject in need thereof a therapeutically effective amount of a compound according to claim 1, or a pharmaceutically acceptable salt thereof.

27-28. (canceled)

29. A method of inhibiting and/or degrading:
a. BCDIN3D activity in vitro or in vivo; or
b. metastasis in vitro or in vivo;
said method comprising contacting a cell with an effective amount of a compound according to claim 1, or a pharmaceutically acceptable salt thereof.

30. (canceled)

31. A combination comprising a compound according to claim 1, or a pharmaceutically acceptable salt there, with one or more additional therapeutic agents.
